SEQUENCE LISTING (189 sequences)

SEQ ID NO: 1 - protein, 117 aa, synthetic construct
  REGION 1..117: note = Synthetic: 1A12 VH
EVQLLESGGGLVKPGGSLRLSCAASGFTFSSYAMHWVRQAPGKGLEWVAVISYDGSNKYYADSVKGRFTISRDDSKNTLYLQMNSLKTEDTAVYYCARHGWDAFDIWGQGTLVTVSS

SEQ ID NO: 2 - protein, 107 aa, synthetic construct
  REGION 1..107: note = Synthetic: 1A12 VL
DIQMTQSPSSLSASVGDRVTITCRASQDISNRLNWYQQKPGKAPKLLIYLASSLQGGVPSRFSGSGSGTDFTLTISSLQPEDFATYYCQQAISFPLTFGGGTKVEIK

SEQ ID NO: 3 - protein, 121 aa, synthetic construct
  REGION 1..121: note = Synthetic: 1D3 VH
EVQLLESGGGLVQPGGSLRLSCAASGFTFNTHAMHWVRQAPGKGLEWVSAISGSGGSTYYADSVKGRFTISRDISKNTLYLQMNSLRAEDTAVYYCARLLGRFGEYGMDVWGQGTTVTVSS

SEQ ID NO: 4 - protein, 10 aa, synthetic construct
  REGION 1..10: note = Synthetic: 1D3 CDR1H
GFTFNTHAMH

SEQ ID NO: 5 - protein, 17 aa, synthetic construct
  REGION 1..17: note = Synthetic: 1D3 CDR2H
AISGSGGSTYYADSVKG

SEQ ID NO: 6 - protein, 12 aa, synthetic construct
  REGION 1..12: note = Synthetic: 1D3 CDR3H
LLGRFGEYGMDV

SEQ ID NO: 7 - protein, 107 aa, synthetic construct
  REGION 1..107: note = Synthetic: 1D3 VL
DIQMTQSPSSLSASVGDRVTITCRASRSIYTWLAWYQQKPGKAPKLLIYDASTLQSGVPSRFSGSGSGTDFTLTISSLQPEDFATYYCQQSYSTPYTFGGGTKVEIK

SEQ ID NO: 8 - protein, 11 aa, synthetic construct
  REGION 1..11: note = Synthetic: 1D3 CDR1L
RASRSIYTWLA

SEQ ID NO: 9 - protein, 5 aa, synthetic construct
  REGION 1..5: note = Synthetic: 1D3 CDR2L
STLQS

SEQ ID NO: 10 - protein, 9 aa, synthetic construct
  REGION 1..9: note = Synthetic: 1D3 CDR3L
QQSYSTPYT

SEQ ID NO: 11 - protein, 116 aa, synthetic construct
  REGION 1..116: note = Synthetic: 1F11 VH
QVQLVQSGAEVKKPGSSVKVSCKASGDTFTTYQIHWVRQAPGQGLEWMGGIMPIFGTTKYAQNFQGRVTITADESTSTAYMELSSLRSEDTAVYYCARRNYGMDVWGQGTTVTVSS

SEQ ID NO: 12 - protein, 113 aa, synthetic construct
  REGION 1..113: note = Synthetic: 1F11 VL
DIVMTQSPDSLAVSLGERATINCKSSQSLLYRPVNKNFLAWYQQKPGQPPKLLIYWASTRGSGVPDRFSGSGSGTDFTLTISSLQAEDVATYFCQQYYSTPITFGQGTKVEIK

SEQ ID NO: 13 - protein, 116 aa, synthetic construct
  REGION 1..116: note = Synthetic: 3D1 VH
QVQLVQSGAEVKKPGSSVKVSCKASGYSFISYAITWVRQAPGQGLEWIGGINPIFGTAKYAQKFQGRVTITADESTSTAYMELSSLRSEDTAVYYCAKGQALIQHWGQGTLVTVSS

SEQ ID NO: 14 - protein, 113 aa, synthetic construct
  REGION 1..113: note = Synthetic: 3D1 VL
DIVMTQSPDSLAVSLGERATINCKSSQSVLSSSNNKNYLAWYQQKPGQPPKLLIYWASTRESGVPDRFSGSGSGTDFTLTISSLQAEDVAVYYCQQYYSTPLAFGQGTKVEIK

SEQ ID NO: 15 - protein, 116 aa, synthetic construct
  REGION 1..116: note = Synthetic: 3F2 VH
QVQLVQSGAEVKKPGASVKVSCKASGYTFTAQYMYWVRQAPGQGLEWMGRINPTSGNTVYAQKLQGRVTMTRDTSTSTVYMELSSLRSEDTAVYYCARGINWFDPWGQGTLVTVSS

SEQ ID NO: 16 - protein, 10 aa, synthetic construct
  REGION 1..10: note = Synthetic: 3F2 CDR1H
GYTFTAQYMY

SEQ ID NO: 17 - protein, 17 aa, synthetic construct
  REGION 1..17: note = Synthetic: 3F2 CDR2H
RINPTSGNTVYAQKLQG

SEQ ID NO: 18 - protein, 7 aa, synthetic construct
  REGION 1..7: note = Synthetic: 3F2 CDR3H
GINWFDP

SEQ ID NO: 19 - protein, 107 aa, synthetic construct [sequence omitted]

SEQ ID NO: 20 - protein, 11 aa, synthetic construct
  REGION 1..11: note = Synthetic: 3F2 CDR1L
RASQSISSYLN

SEQ ID NO: 21 - protein, 5 aa, synthetic construct
  REGION 1..5: note = Synthetic: 3F2 CDR2L
SSLQS

SEQ ID NO: 22 - protein, 9 aa, synthetic construct
  REGION 1..9: note = Synthetic: 3F2 CDR3L
QQSYSTPLT

SEQ ID NO: 23 - protein, 118 aa, synthetic construct
  REGION 1..118: note = Synthetic: 4H6 VH
EVQLLESGGGLVQPGGSLRLSCAASGFTFSSYGMHWVRQAPGKGLEWVSGISGSGGSTYYADSVKGRFTISRDNSKNTLYLQMNSLRAEDTAVYYCARTQWRASLDPWGQGTLVNVSS

SEQ ID NO: 24 - protein, 107 aa, synthetic construct
  REGION 1..107: note = Synthetic: 4H6 VL
DIQMTQSPSSLSASVGDRVTITCRASQDISYWLAWYQQKPGKAPKLLIYGASTLQSGVPSRFSGSGSGTDFTLTISSLQPEDFATYYCQQTTMSPYTFGQGTKVEIK

SEQ ID NO: 25 - protein, 120 aa, synthetic construct
  REGION 1..120: note = Synthetic: 6C3 VH
EVQLLESGGGLVQPGGSLRLSCAASGFTFSSYAMHWVRQAPGKGLEWVASISGTGFTTRYADSVKGRFTISRDNSKNTLYLQMNSLRAEDTAVYYCARGGWFRDTPVDYWGQGTLVTVSS

SEQ ID NO: 26 - protein, 112 aa, synthetic construct
  REGION 1..112: note = Synthetic: 6C3 VL
DIVMTQSPLSLPVTPGEPASISCRSSQSLLHSNGYNYLDWYLQKPGQSPQLLIYGASSLRSGVPDRFSGSGSGTDFTLKISRVEAZDVGVYYCMQASHWPPTFGQGTRLEIK

SEQ ID NO: 27 - protein, 115 aa, synthetic construct
  REGION 1..115: note = Synthetic: 9H2 VH
QVQLVQSGAEVKKPGSSVKVSCKASGGTFSTYAINWVRQAPGQGLEWMGGINPMFGTARYAQKFQGRVTITADESTSTAYMELSSLRSEDTAVYYCARGRLHRHWGQGTLVTVSS

SEQ ID NO: 28 - protein, 113 aa, synthetic construct
  REGION 1..113: note = Synthetic: 9H2 VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYTTPITFGQGTRLEIK

SEQ ID NO: 29 - protein, 122 aa, synthetic construct
  REGION 1..122: note = Synthetic: 4B9 VH
QVQLVQSGAEVKKPGASVKVSCKASGYTFTSYYMHWVRQAPGQGLEWMGWMNPNSGNTGYAQKFQGRVTMTRDTSTSTVYMELSSLRSEDTAVYYCARGQGRYLNYYYMDVWGKGTTVTVSS

SEQ ID NO: 30 - protein, 107 aa, synthetic construct
  REGION 1..107: note = Synthetic: 4B9 VL
DIQMTQSPSSLSASVGDRVTITCRVSQGISNYLAWYQQKPGKAPKLLIYDASSLQSGVPSRFSGSGSGTDFTLTISSLQPEDFATYYCQQSYSTPFTFGPGTKVDIK

SEQ ID NO: 31 - protein, 118 aa, synthetic construct
  REGION 1..118: note = Synthetic: 15B11 VH
QVQLVQSGAEVKKPGASVKVSCKASGYTFTGYYIHWVRQAPGQGLEWMGWMNPHSGHTGYAQNLQGRVTMTRDTSTSTVYMELSSLRSEDTAVYYCARVKVTTGFDYWGQGTLVTVSS

SEQ ID NO: 32 - protein, 112 aa, synthetic construct
  REGION 1..112: note = Synthetic: 15B11 VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGTHWPPTFGQGTKLEIK

SEQ ID NO: 33 - protein, 118 aa, synthetic construct
  REGION 1..118: note = Synthetic: 24B6 VH
QVQLVQSGAEVKKPGASVKVSCKASGYTFTGYYMHWVRQAPGQGLEWIGWMNPNSGDTGYAQKFQGRVTMTRDTSTSTVYMELSSLRSEDTAVYYCAKVGAYGGMDVWGQGTTVTVSS

SEQ ID NO: 34 - protein, 112 aa, synthetic construct
  REGION 1..112: note = Synthetic: 24B6 VL
DIVMTQSPLSLPVTPGEPASISCRSSQSLLHSNGYNYLDWYLQKPGQSPQLLIYGGSNRASGVPDRFSGSGSGTDFTLKISRVEAEDVGVYYCMQGTHWPPTFGPGTKVDIK

SEQ ID NO: 35 - protein, 119 aa, synthetic construct
  REGION 1..119: note = Synthetic: 24F4 VH
QVQLVQSGAEVKKPGSSVKVSCKASGYRFTGDYIHWVRQAPGQGLEWMGWMNPNSGNTGLAQKFQGRVTITADESTSTAYMELSSLRSEDTAVYYCARVLGFSGGMDVWGQGTTVTVSS

SEQ ID NO: 36 - protein, 112 aa, synthetic construct
  REGION 1..112: note = Synthetic: 24F4 VL
DIVMTQSPLSLPVTPGEPASISCRSSQSLLHSNGYNYLDWYLQKPGQSPQLLIYAASSLRSGVPDRFSGSGSGTDFTLKISRVEAEDVGVYYCMQGTHWPPTFGQGTKLEIK

SEQ ID NO: 37 - protein, 118 aa, synthetic construct
  REGION 1..118: note = Synthetic: 30G4 VH
QVQLVQSGAEVKKPGASVKVSCKASGYTFSSYYMHWVRQAPGQGLEWMGWMNPNSGDTGYAQKFQGRVTMTRDTSTSTVYMELSSLRSEDTAVYYCARVPETGGMDVWGQGTTVTVSS

SEQ ID NO: 38 - protein, 112 aa, synthetic construct
  REGION 1..112: note = Synthetic: 30G4 VL
DIVMTQSPLSLPVTPGEPASISCRSSQSLLHSNGYNYLDWYLQKPGQSPQLLIYGASSLHSGVPDRFSGSGSGTDFTLKISRVEAEDVGVYYCMQGTHWPPTFGQGTKVEIK

SEQ ID NO: 39 - protein, 127 aa, synthetic construct
  REGION 1..127: note = Synthetic: 5E4 VH
QVQLVQSGAEVKKPGASVKVSCKASGYTFTSYDINWVRQAPGQGLEWMGWMNPNSGNTGYAQKFQGRVTMTRDTSTSTVYMELSSLRSEDTAVYYCARGELWFGEFDYYYYYGMDVWGQGTTVTVSS

SEQ ID NO: 40 - protein, 107 aa, synthetic construct
  REGION 1..107: note = Synthetic: 5E4 VL
DIQMTQSPSSLSASVGDRVTITCRASQSISSYLNWYQQKPGKAPKLLIYGASSLQSGVPSRFSGSGSGTDFTLTISSLQPEDFATYYCQQADRIPFTFGRGTKVEIK

SEQ ID NO: 41 - protein, 120 aa, synthetic construct
  REGION 1..120: note = Synthetic: 5F4 VH
EVQLLESGGGLVKPGGSLRLSCAASGFTFSSYAMHWVRQAPGKGLEWVSAIGGSGRTTYYADSVKGRFTISRDDSKNTLYLQMNSLKTEDTAVYYCARERGYSYGQIDYWGQGTLVTVSS

SEQ ID NO: 42 - protein, 112 aa, synthetic construct
  REGION 1..112: note = Synthetic: 5F4 VL
DIVMTQSPLSLPVTPGEPASISCRSSQSLLHSNGYNYLDWYLQKPGQSPQLLIYAASSLQSGVPDRFSGSGSGTDFTLKISRVEAEDVGVYYCMQGAHWPLTFGQGTKVEIK

SEQ ID NO: 43 - protein, 119 aa, synthetic construct
  REGION 1..119: note = Synthetic: 5G6 VH
QVQLVQSGAEVKKPGASVKVSCKASGYTFTSYYMHWVRQAPGQGLEWLGWVNPNTGTTRFAQKFQGRVTMTRDTSTSTVYMELSSLRSEDTAVYYCARDANYYYGMDVWGQGTMVTVSS

SEQ ID NO: 44 - protein, 114 aa, synthetic construct
  REGION 1..114: note = Synthetic: 5G6 VL
DIVMTQSPDSLAVSLGERATINCKSSQSVLYSSNNKNYLAWYQQKPGQPPKLLIYWASTRESGVPDRFSGSGSGTDFTLTISSLQAEDVATYYCQKYNSALPITFGQGTRLEIK

SEQ ID NO: 45 - protein, 120 aa, synthetic construct
  REGION 1..120: note = Synthetic: 9D11 VH
QVQLVQSGAEVKKPGASVKVSCKASGYTFTSYGISWVRQAPGQGLEWVGVINPNGGTTTYAQTFQGRVTMTRDTSTSTVYMELSSLRSEDTAVYYCARTGYSSGWAFDYWGRGTLVTVSS

SEQ ID NO: 46 - protein, 10 aa, synthetic construct
  REGION 1..10: note = Synthetic: 9D11 CDR1H
GYTFTSYGIS

SEQ ID NO: 47 - protein, 17 aa, synthetic construct
  REGION 1..17: note = Synthetic: 9D11 CDR2H
VINPNGGTTTYAQTFQG

SEQ ID NO: 48 - protein, 11 aa, synthetic construct
  REGION 1..11: note = Synthetic: 9D11 CDR3H
TGYSSGWAFDY

SEQ ID NO: 49 - protein, 112 aa, synthetic construct
  REGION 1..112: note = Synthetic: 9D11 VL
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYFSTPSFGQGTKVEIK

SEQ ID NO: 50 - protein, 17 aa, synthetic construct
  REGION 1..17: note = Synthetic: 9D11 CDR1L
KSSQSVLYSSNNKNYLA

SEQ ID NO: 51 - protein, 5 aa, synthetic construct
  REGION 1..5: note = Synthetic: 9D11 CDR2L
STRES

SEQ ID NO: 52 - protein, 8 aa, synthetic construct
  REGION 1..8: note = Synthetic: 9D11 CDR3L
QQYFSTPS

SEQ ID NO: 53 - protein, 119 aa, synthetic construct
  REGION 1..119: note = Synthetic: 9E1 VH
QVQLVQSGAEVKKPGASVKVSCKASGYTFTGYYLHWVRQAPGQGLEWMGWINPNSGGTNSAQRFQGRVTMTRDTSTSTVYMELSSLRSEDTAVYYCARVRGYEGGMDVWGQGTTVTVSS

SEQ ID NO: 54 - protein, 112 aa, synthetic construct
  REGION 1..112: note = Synthetic: 9E1 VL
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQGSHWPPTFGQGTRLEIK

SEQ ID NO: 55 - protein, 118 aa, synthetic construct
  REGION 1..118: note = Synthetic: 31D7 VH
EVQLLESGGGLVQPGGSXRLSCAASGFTFSSYGMHWVRQAPGKGLEWVSSISSSSSYIYYADSVKGRFTISRDNSKNTLYLQMNSLRAEDTAVYYCARIGSGGSLDYWGQGTLVTVSS

SEQ ID NO: 56 - protein, 107 aa, synthetic construct
  REGION 1..107: note = Synthetic: 31D7 VL
DIQMTQSPSSLSASVGDRVTITCRASQSISNWLAWYQQKPGKAPKLLIYAASSLQSGVPSRFSGSGSGTDFTLTISSLQPEDFATYYCQQSYTIPITFGQGTRLEIK

SEQ ID NO: 57 - protein, 116 aa, synthetic construct
  REGION 1..116: note = Synthetic: 39A11 VH
EVQLLESGGGLVQPGGSLRLSCAASGFTFSSYGMHWVRQAPGKGLEWVASISSSSGYIYYADSVKGRFTISRDNSKNTLYLQMNSLRAEDTAVYYCARASSGMDVWGQGTTVTVSS

SEQ ID NO: 58 - protein, 10 aa, synthetic construct
  REGION 1..10: note = Synthetic: 39A11 CDR1H
GFTFSSYGMH

SEQ ID NO: 59 - protein, 17 aa, synthetic construct
  REGION 1..17: note = Synthetic: 39A11 CDR2H
SISSSSGYIYYADSVKG

SEQ ID NO: 60 - protein, 7 aa, synthetic construct
  REGION 1..7: note = Synthetic: 39A11 CDR3H
ASSGMDV

SEQ ID NO: 61 - protein, 107 aa, synthetic construct
  REGION 1..107: note = Synthetic: 39A11 VL
DIQMTQSPSSLSASVGDRVTITCRASQTIRSYLNWYQQKPGKAPKLLIYAASHLQSGVPSRFSGSGSGTDFTLTISSLQPEDFATYYCQQSYTTPYTFGQGTKLEIK

SEQ ID NO: 62 - protein, 11 aa, synthetic construct
  REGION 1..11: note = Synthetic: 39A11 CDR1L
RASQTIRSYLN

SEQ ID NO: 63 - protein, 5 aa, synthetic construct
  REGION 1..5: note = Synthetic: 39A11 CDR2L
SHLQS

SEQ ID NO: 64 - protein, 9 aa, synthetic construct
  REGION 1..9: note = Synthetic: 39A11 CDR3L
QQSYTTPYT

SEQ ID NO: 65 - protein, 106 aa, synthetic construct
  REGION 1..106: note = Synthetic: 49A2 VL
DIQMTQSPSSLSASVGDRVTITCRASQNIDTYVNWYQQKPGKAPKLLIYAASRLHTVPSRFSGSGSGTDFTLTISSLQPEDFATYYCQQSYTSPFTFGGGTKVEIK

SEQ ID NO: 66 - protein, 11 aa, synthetic construct
  REGION 1..11: note = Synthetic: 49A2 CDR1L
RASQNIDTYVN

SEQ ID NO: 67 - protein, 5 aa, synthetic construct
  REGION 1..5: note = Synthetic: 49A2 CDR2L
SRLHT

SEQ ID NO: 68 - protein, 9 aa, synthetic construct
  REGION 1..9: note = Synthetic: 49A2 CDR3L
QQSYTSPFT

SEQ ID NO: 69 - protein, 107 aa, synthetic construct
  REGION 1..107: note = Synthetic: 50A10 VL
DIQMTQSPSSLSASVGDRVTITCQASQDISNYINWYQQKPGKAPKLLIYAASRLQSGVPSRFSGSGSGTDFTLTISSLQPEDFATYYCQQSYRSPFTFGQGTKLEIK

SEQ ID NO: 70 - protein, 11 aa, synthetic construct
  REGION 1..11: note = Synthetic: 50A10 CDR1L
QASQDISNYIN

SEQ ID NO: 71 - protein, 5 aa, synthetic construct
  REGION 1..5: note = Synthetic: 50A10 CDR2L
SRLQS

SEQ ID NO: 72 - protein, 9 aa, synthetic construct
  REGION 1..9: note = Synthetic: 50A10 CDR3L
QQSYRSPFT

SEQ ID NO: 73 - protein, 11 aa, synthetic construct
  REGION 1..11: note = Synthetic: 3F2 CDR motif CDR1L
  VARIANT 1: note = X is R or Q
  VARIANT 5: note = X is D, N, or S
  VARIANT 7: note = X is D or S
  VARIANT 8: note = X is N, S, or T
  VARIANT 10: note = X is I, L, or V
XASQXIXXYXN

SEQ ID NO: 74 - skipped

SEQ ID NO: 75 - protein, 9 aa, synthetic construct
  REGION 1..9: note = Synthetic: 3F2 CDR motif CDR3L
  VARIANT 5: note = X is R, S, or T
  VARIANT 6: note = X is S or T
  VARIANT 8: note = X is F or L
QQSYXXPXT

SEQ ID NO: 76 - protein, 107 aa, synthetic construct
  REGION 1..107: note = Synthetic: 45A2 VL
DIQMTQSPSSLSASVGDRVTITCRASQNIYTWLAWYQQKPGKAPKLLIYDATNLPTGVPSRFSGSGSGTDFTLTISSLQPEDFATYYCQQSYSTRWTFGGGTKVEIK

SEQ ID NO: 77 - protein, 11 aa, synthetic construct
  REGION 1..11: note = Synthetic: 45A2 CDR1L
RASQNIYTWLA

SEQ ID NO: 78 - protein, 5 aa, synthetic construct
  REGION 1..5: note = Synthetic: 45A2 CDR2L
TNLPT

SEQ ID NO: 79 - protein, 9 aa, synthetic construct
  REGION 1..9: note = Synthetic: 45A2 CDR3L
QQSYSTRWT

SEQ ID NO: 80 - protein, 107 aa, synthetic construct
  REGION 1..107: note = Synthetic: 47B2 VL
DIQMTQSPSSLSASVGDRVTITCRASQTVYTWLAWYQQKPGKAPKLLIYDATNLATGVPSRFSGSGSGTDFTLTISSLQPEDFATYYCQQSYSTSWTFGGGTKVEIK

SEQ ID NO: 81 - protein, 11 aa, synthetic construct
  REGION 1..11: note = Synthetic: 47B2 CDR1L
RASQTVYTWLA

SEQ ID NO: 82 - protein, 5 aa, synthetic construct
  REGION 1..5: note = Synthetic: 47B2 CDR2L
TNLAT

SEQ ID NO: 83 - protein, 9 aa, synthetic construct
  REGION 1..9: note = Synthetic: 47B2 CDR3L
QQSYSTSWT

SEQ ID NO: 84 - protein, 11 aa, synthetic construct
  REGION 1..11: note = Synthetic: 1D3 CDR motif CDR1L
  VARIANT 4: note = X is Q or R
  VARIANT 5: note = X is N, S, or T
  VARIANT 6: note = X is I or V
RASXXXYTWLA

SEQ ID NO: 85 - skipped

SEQ ID NO: 86 - protein, 9 aa, synthetic construct
  REGION 1..9: note = Synthetic: 1D3 CDR motif CDR3L
  VARIANT 7: note = X is P, R, or S
  VARIANT 8: note = X is W or Y
QQSYSTXXT

SEQ ID NO: 87 - protein, 112 aa, synthetic construct
  REGION 1..112: note = Synthetic: 67E12 VL
DIVMTQSPDSLAVSLGERATINCKSSQSVLYSSNNKNYLAWYQQKPGQPPKLLIYWASKRVSGVPDRFSGSGSGTDFTLTISSLQAEDVAVYYCQQYFDSPTFGQGTKVEIK

SEQ ID NO: 88 - protein, 17 aa, synthetic construct
  REGION 1..17: note = Synthetic: 67E12 CDR1L
KSSQSVLYSSNNKNYLA

SEQ ID NO: 89 - protein, 5 aa, synthetic construct
  REGION 1..5: note = Synthetic: 67E12 CDR2L
SKRVS

SEQ ID NO: 90 - protein, 8 aa, synthetic construct
  REGION 1..8: note = Synthetic: 67E12 CDR3L
QQYFDSPT

SEQ ID NO: 91 - protein, 112 aa, synthetic construct
  REGION 1..112: note = Synthetic: 67C6 VL
DIVMTQSPDSLAVSLGERATINCKSSQSVLSSSNNKNYLAWYQQKPGQPPKLLIYWASTRQSGVPDRFSGSGSGTDFTLTISSLQAEDVAVYYCQQYYSDPTFGQGTKVEIK

SEQ ID NO: 92 - protein, 17 aa, synthetic construct
  REGION 1..17: note = Synthetic: 67C6 CDR1L
KSSQSVLSSSNNKNYLA

SEQ ID NO: 93 - protein, 5 aa, synthetic construct
  REGION 1..5: note = Synthetic: 67C6 CDR2L
STRQS

SEQ ID NO: 94 - protein, 8 aa, synthetic construct
  REGION 1..8: note = Synthetic: 67C6 CDR3L
QQYYSDPT

SEQ ID NO: 95 - protein, 112 aa, synthetic construct
  REGION 1..112: note = Synthetic: 67C3 VL
DIVMTQSPDSLAVSLGERATINCKSSQSVLYSSNNKNYLAWYQQKPGQPPKLLIYWASTRASGVPDRFSGSGSGTDFTLTISSLQAEDVAVYYCQQYYDTPTFGQGTKVEIK

SEQ ID NO: 96 - protein, 17 aa, synthetic construct
  REGION 1..17: note = Synthetic: 67C3 CDR1L
KSSQSVLYSSNNKNYLA

SEQ ID NO: 97 - protein, 5 aa, synthetic construct
  REGION 1..5: note = Synthetic: 67C3 CDR2L
STRAS

SEQ ID NO: 98 - protein, 8 aa, synthetic construct
  REGION 1..8: note = Synthetic: 67C3 CDR3L
QQYYDTPT

SEQ ID NO: 99 - protein, 112 aa, synthetic construct
  REGION 1..112: note = Synthetic: 67G3 VL
DIVMTQSPDSLAVSLGERATINCKSSQSVLSSSNNKNYLAWYQQKPGQPPKLLIYWASTRQSGVPDRFSGSGSGTDFTLTISSLQAEDVAVYYCQQYYTSPTFGQGTKVEIK

SEQ ID NO: 100 - protein, 17 aa, synthetic construct
  REGION 1..17: note = Synthetic: 67G3 CDR1L
KSSQSVLSSSNNKNYLA

SEQ ID NO: 101 - protein, 5 aa, synthetic construct
  REGION 1..5: note = Synthetic: 67G3 CDR2L
STRQS

SEQ ID NO: 102 - protein, 8 aa, synthetic construct
  REGION 1..8: note = Synthetic: 67G3 CDR3L
QQYYTSPT

SEQ ID NO: 103 - protein, 112 aa, synthetic construct
  REGION 1..112: note = Synthetic: 67H9 VL
DIVMTQSPDSLAVSLGERATINCRSSQSVLYSSNNKNYLAWYQQKPGQPPKLLIYWASNRKSGVPDRFSGSGSGTDFTLTISSLQAEDVAVYYCQQYYSAPTFGQGTKVEIK

SEQ ID NO: 104 - protein, 17 aa, synthetic construct
  REGION 1..17: note = Synthetic: 67H9 CDR1L
RSSQSVLYSSNNKNYLA

SEQ ID NO: 105 - protein, 5 aa, synthetic construct
  REGION 1..5: note = Synthetic: 67H9 CDR2L
SNRKS

SEQ ID NO: 106 - protein, 8 aa, synthetic construct
  REGION 1..8: note = Synthetic: 67H9 CDR3L
QQYYSAPT

SEQ ID NO: 107 - protein, 112 aa, synthetic construct
  REGION 1..112: note = Synthetic: 68F5 VL
DIVMTQSPDSLAVSLGERATINCKSSRSVLSRSNNKNYLAWYQQKPGQPPKLLIYWASTRQFGVPDRFSGSGSGTDFTLTISSLQAEDVAVYYCQQYYDTPTFGQGTKVEIK

SEQ ID NO: 108 - protein, 17 aa, synthetic construct
  REGION 1..17: note = Synthetic: 68F5 CDR1L
KSSRSVLSRSNNKNYLA

SEQ ID NO: 109 - protein, 5 aa, synthetic construct
  REGION 1..5: note = Synthetic: 68F5 CDR2L
STRQF

SEQ ID NO: 110 - protein, 8 aa, synthetic construct
  REGION 1..8: note = Synthetic: 68F5 CDR3L
QQYYDTPT

SEQ ID NO: 111 - protein, 17 aa, synthetic construct
  REGION 1..17: note = Synthetic: 9D11 CDR motif CDR1L
  VARIANT 1: note = X is K or R
  VARIANT 4: note = X is Q or R
  VARIANT 8: note = X is S or Y
  VARIANT 9: note = X is R or S
XSSXSVLXXSNNKNYLA

SEQ ID NO: 112 - skipped

SEQ ID NO: 113 - protein, 8 aa, synthetic construct
  REGION 1..8: note = Synthetic: 9D11 CDR motif CDR3L
  VARIANT 4: note = X is F or Y
  VARIANT 5: note = X is D, S, or T
  VARIANT 6: note = X is A, D, S, or T
  VARIANT 8: note = X is S or T
QQYXXXPX

SEQ ID NO: 114 - protein, 107 aa, synthetic construct
  REGION 1..107: note = Synthetic: 57H3 VL
DIQMTQSPSSLSASVGDRVTITCQASQDIRKYLNWYQQKPGKAPKLLIYAASTRESGVPSRFSGSGSGTDFTLTISSLQPEDFATYYCQQYYSLPLTFGQGTKLEIK

SEQ ID NO: 115 - protein, 11 aa, synthetic construct
  REGION 1..11: note = Synthetic: 57H3 CDR1L
QASQDIRKYLN

SEQ ID NO: 116 - protein, 5 aa, synthetic construct
  REGION 1..5: note = Synthetic: 57H3 CDR2L
STRES

SEQ ID NO: 117 - protein, 9 aa, synthetic construct
  REGION 1..9: note = Synthetic: 57H3 CDR3L
QQYYSLPLT

SEQ ID NO: 118 - protein, 107 aa, synthetic construct
  REGION 1..107: note = Synthetic: 57G8 VL
DIQMTQSPSSLSASVGDRVTITCRASQSIRSYLNWYQQKPGKAPKLLIYYASSLQSGVPSRFSGSGSGTDFTLTISSLQPEDFATYYCQQYYSTPLTFGQGTKVEIK

SEQ ID NO: 119 - protein, 11 aa, synthetic construct
  REGION 1..11: note = Synthetic: 57G8 CDR1L
RASQSIRSYLN

SEQ ID NO: 120 - protein, 5 aa, synthetic construct
  REGION 1..5: note = Synthetic: 57G8 CDR2L
SSLQS

SEQ ID NO: 121 - protein, 9 aa, synthetic construct
  REGION 1..9: note = Synthetic: 57G8 CDR3L
QQYYSTPLT

SEQ ID NO: 122 - protein, 107 aa, synthetic construct
  REGION 1..107: note = Synthetic: 56A9 VL
DIQMTQSPSSLSASVGDRVTITCRASQSISSYLNWYQQKPGKAPKLLIYAASIRESGVPSRFSGSGSGTDFTLTISSLQPEDFATYYCQQYYTTPLTFGQGTKLEIK

SEQ ID NO: 123 - protein, 11 aa, synthetic construct
  REGION 1..11: note = Synthetic: 56A9 CDR1L
RASQSISSYLN

SEQ ID NO: 124 - protein, 5 aa, synthetic construct
  REGION 1..5: note = Synthetic: 56A9 CDR2L
SIRES

SEQ ID NO: 125 - protein, 9 aa, synthetic construct
  REGION 1..9: note = Synthetic: 56A9 CDR3L
QQYYTTPLT

SEQ ID NO: 126 - protein, 107 aa, synthetic construct
  REGION 1..107: note = Synthetic: 56H7 VL
DIQMTQSPSSLSASVGDRVTITCRASQSISSYLNWYQQKPGKAPKLLIYAASNLQSGVPSRFSGSGSGTDFTLTISSLQPEDFATYYCQQYYTTPLTFGQGTKLEIK

SEQ ID NO: 127 - protein, 11 aa, synthetic construct
  REGION 1..11: note = Synthetic: 56H7 CDR1L
RASQSISSYLN

SEQ ID NO: 128 - protein, 5 aa, synthetic construct
  REGION 1..5: note = Synthetic: 56H7 CDR2L
SNLQS

SEQ ID NO: 129 - protein, 9 aa, synthetic construct
  REGION 1..9: note = Synthetic: 56H7 CDR3L
QQYYTTPLT

SEQ ID NO: 130 - protein, 107 aa, synthetic construct
  REGION 1..107: note = Synthetic: 62F9 VL
DIQMTQSPSSLSASVGDRVTITCRASQSVSSAVAWYQQKPGKAPKLLIYAASIRESGVPSRFSGSGSGTDFTLTISSLQPEDFATYYCQQYYSTPLTFGQGTKLEIK

SEQ ID NO: 131 - protein, 11 aa, synthetic construct
  REGION 1..11: note = Synthetic: 62F9 CDR1L
RASQSVSSAVA

SEQ ID NO: 132 - protein, 5 aa, synthetic construct
  REGION 1..5: note = Synthetic: 62F9 CDR2L
SIRES

SEQ ID NO: 133 - protein, 9 aa, synthetic construct
  REGION 1..9: note = Synthetic: 62F9 CDR3L
QQYYSTPLT

SEQ ID NO: 134-135 - skipped (2 entries)

SEQ ID NO: 136 - protein, 9 aa, synthetic construct
  REGION 1..9: note = Synthetic: 39A11 CDR motif CDR3L
  VARIANT 3: note = X is S or Y
  VARIANT 5: note = X is S or T
  VARIANT 6: note = X is L or T
  VARIANT 8: note = X is L or Y
QQXYXXPXT

SEQ ID NO: 137 - protein, 120 aa, synthetic construct
  REGION 1..120: note = Synthetic: Control antibody 1 VH
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSS

SEQ ID NO: 138 - protein, 107 aa, synthetic construct
  REGION 1..107: note = Synthetic: Control antibody 1 VL
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIK

SEQ ID NO: 139 - protein, 115 aa, synthetic construct
  REGION 1..115: note = Synthetic: Control antibody 2 (antibody 6H3 described in US9,574,000) VH
EVQLQQSGPVLVKPGTSVKMSCKASGYTFTDYYMNWVKQSHGKSLEWIGVINPYNGDTTYNQKFKGKATLTVDKSSSTAYMEVNSLTFEDSAVYYCARYPESTYWGQGTLVTVSA

SEQ ID NO: 140 - protein, 112 aa, synthetic construct
  REGION 1..112: note = Synthetic: Control antibody 2 (antibody 6H3 described in US9,574,000) VL
DVVMTQTPLSLPVSLGDQASISCRSSQSLVHINGNTYLHWYLQKPGQSPKVLIYKVSNRFSGVPDRFSGSGSGTDFTLKISRVEAEDLGVYFCSQSTHVPLTFGAGTKLELK

SEQ ID NO: 141 - protein, 115 aa, synthetic construct
  REGION 1..115: note = Synthetic: DP47 VH
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSS

SEQ ID NO: 142 - protein, 108 aa, synthetic construct
  REGION 1..108: note = Synthetic: DP47 VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIK

SEQ ID NO: 143 - protein, 214 aa, synthetic construct
  REGION 1..214: note = Synthetic: 20502 VL
EIVMTQSPATLSVSPGERATLSCRASQSVSSNLAWYQQKPGQAPRLLIYGASTRATGIPARFSGSGSGTEFTLTISSLQSEDFAVYYCQQYHSFPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 144 - protein, 214 aa, synthetic construct
  REGION 1..214: note = Synthetic: 57G8 VL
DIQMTQSPSSLSASVGDRVTITCRASQSIRSYLNWYQQKPGKAPKLLIYYASSLQSGVPSRFSGSGSGTDFTLTISSLQPEDFATYYCQQYYSTPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 145 - protein, 214 aa, synthetic construct
  REGION 1..214: note = Synthetic: 50A10 VL
DIQMTQSPSSLSASVGDRVTITCQASQDISNYINWYQQKPGKAPKLLIYAASRLQSGVPSRFSGSGSGTDFTLTISSLQPEDFATYYCQQSYRSPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 146 - protein, 215 aa, synthetic construct
  REGION 1..215: note = Synthetic: DP47 VL
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 147 - protein, 450 aa, synthetic construct
  REGION 1..450: note = Synthetic: 20502 VH
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 148 - protein, 446 aa, synthetic construct
  REGION 1..446: note = Synthetic: 39A11 VH
EVQLLESGGGLVQPGGSLRLSCAASGFTFSSYGMHWVRQAPGKGLEWVASISSSSGYIYYADSVKGRFTISRDNSKNTLYLQMNSLRAEDTAVYYCARASSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 446 aa, synthetic construct
  REGION 1..446: note = Synthetic: 39A11 VH LS mutation
EVQLLESGGGLVQPGGSLRLSCAASGFTFSSYGMHWVRQAPGKGLEWVASISSSSGYIYYADSVKGRFTISRDNSKNTLYLQMNSLRAEDTAVYYCARASSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 150 - protein, 446 aa, synthetic construct
  REGION 1..446: note = Synthetic: 3F2 VH
QVQLVQSGAEVKKPGASVKVSCKASGYTFTAQYMYWVRQAPGQGLEWMGRINPTSGNTVYAQKLQGRVTMTRDTSTSTVYMELSSLRSEDTAVYYCARGINWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 446 aa, synthetic construct
  REGION 1..446: note = Synthetic: 3F2 VH LS mutation
QVQLVQSGAEVKKPGASVKVSCKASGYTFTAQYMYWVRQAPGQGLEWMGRINPTSGNTVYAQKLQGRVTMTRDTSTSTVYMELSSLRSEDTAVYYCARGINWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 152 - protein, 445 aa, synthetic construct
  REGION 1..445: note = Synthetic: DP47 HC1
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 445 aa, synthetic construct
  REGION 1..445: note = Synthetic: LS mutation
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHSHYTQKSLSLSPGK

SEQ ID NO: 154 - protein, 659 aa, synthetic construct
  REGION 1..659: note = Synthetic: 20502 VH IL-15R-alpha sushi domain Linker IL-15
QLQLQESGPGLVKPSETLSLTCTVSGGSIKSGSYYWGWIRQPPGKGLEWIGNIYYSGSTYYNPSLRSRVTISVDTSKNQFSLKLSSVTAADTAVYYCAREGSYPNQFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVESLIILANNSLSSNGNVTESGCKECEELEEKNIKEFLQSFVHIVQMFINTS

SEQ ID NO: 155 - protein, 655 aa, synthetic construct
  REGION 1..655: note = Synthetic: 39A11 VH IL-15R-alpha sushi domain Linker IL-15
EVQLLESGGGLVQPGGSLRLSCAASGFTFSSYGMHWVRQAPGKGLEWVASISSSSGYIYYADSVKGRFTISRDNSKNTLYLQMNSLRAEDTAVYYCARASSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVESLIILANNSLSSNGNVTESGCKECEELEEKNIKEFLQSFVHIVQMFINTS

SEQ ID NO: 156 - protein, 655 aa, synthetic construct
  REGION 1..655: note = Synthetic: 39A11 VH LS mutation IL-15R-alpha sushi domain Linker IL-15
EVQLLESGGGLVQPGGSLRLSCAASGFTFSSYGMHWVRQAPGKGLEWVASISSSSGYIYYADSVKGRFTISRDNSKNTLYLQMNSLRAEDTAVYYCARASSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVESLIILANNSLSSNGNVTESGCKECEELEEKNIKEFLQSFVHIVQMFINTS

SEQ ID NO: 157 - protein, 655 aa, synthetic construct
  REGION 1..655: note = Synthetic: 3F2 VH IL-15R-alpha sushi domain Linker IL-15
QVQLVQSGAEVKKPGASVKVSCKASGYTFTAQYMYWVRQAPGQGLEWMGRINPTSGNTVYAQKLQGRVTMTRDTSTSTVYMELSSLRSEDTAVYYCARGINWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVESLIILANNSLSSNGNVTESGCKECEELEEKNIKEFLQSFVHIVQMFINTS

SEQ ID NO: 158 - protein, 655 aa, synthetic construct
  REGION 1..655: note = Synthetic: 3F2 VH LS mutation IL-15R-alpha sushi domain Linker IL-15
QVQLVQSGAEVKKPGASVKVSCKASGYTFTAQYMYWVRQAPGQGLEWMGRINPTSGNTVYAQKLQGRVTMTRDTSTSTVYMELSSLRSEDTAVYYCARGINWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVESLIILANNSLSSNGNVTESGCKECEELEEKNIKEFLQSFVHIVQMFINTS

SEQ ID NO: 159 - protein, 654 aa, synthetic construct
  REGION 1..654: note = Synthetic: DP47 VH IL-15R-alpha sushi domain Linker IL-15
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVESLIILANNSLSSNGNVTESGCKECEELEEKNIKEFLQSFVHIVQMFINTS

SEQ ID NO: 160 - protein, 654 aa, synthetic construct
  REGION 1..654: note = Synthetic: LS mutation IL-15R-alpha sushi domain Linker IL-15
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKGSGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHSHYTQKSLSLSPGSCPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVESLIILANNSLSSNGNVTESGCKECEELEEKNIKEFLQSFVHIVQMFINTS

SEQ ID NO: 161 - protein, 282 aa, Homo sapiens
  REGION 1..282: note = misc_feature - human B7-H4 (NCBI accession #: NP_078902) signal peptide IgV domain IgC domain transmembrane region
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 162 - protein, 283 aa, Mus musculus
  REGION 1..283: note = misc_feature - Murine B7-H4 (NCBI accession#: Q7TSP5, Phe29-Pro258) signal peptide IgV domain IgC domain transmembrane region
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGPSPCVFSSAFVAGWALLSLSCCLMLR

SEQ ID NO: 163 - protein, 330 aa, Homo sapiens [sequence omitted]

SEQ ID NO: 164 - protein, 162 aa, Homo sapiens
  REGION 1..162: note = misc_feature - Full-length human Il-15 (NCBI accession #: P40933) mature protein
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 165 - protein, 114 aa, Homo sapiens
  REGION 1..114: note = misc_feature - Human IL-15 (mature protein)
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 166 - protein, 114 aa, Homo sapiens
  REGION 1..114: note = misc_feature - Human IL-15 N65S (mature protein)
NWVNVISDLKKIEDLIQSMHIDATLYTESDVHPSCKVTAMKCFLLELQVISLESGDASIHDTVESLIILANNSLSSNGNVTESGCKECEELEEKNIKEFLQSFVHIVQMFINTS

SEQ ID NO: 167 - protein, 61 aa, Homo sapiens
  REGION 1..61: note = misc_feature - IL-15R-alpha sushi domain (residues 33-93 of NCBI accession #: EAW86418.1)
CPPPMSVEHADIWVKSYSLYSRERYICNSGFKRKAGTSSLTECVLNKATNVAHWTTPSLKC

SEQ ID NO: 168 - protein, 20 aa, synthetic construct
  REGION 1..20: note = Synthetic: Artificial sequence
SGGSGGGGSGGGSGGGGSLQ

SEQ ID NO: 169 - protein, 209 aa, synthetic construct
  REGION 1..209: note = Synthetic: IL-15R-alpha sushi domain + linker + Il-15 N65S
CPPPMSVEHADIWVKSYSLYSRERYICNSGFKRKAGTSSLTECVLNKATNVAHWTTPSLKCIRDPALVHQRPAPPSGGSGGGGSGGGSGGGGSLQNWVNVISDLKKIEDLIQSMHIDATLYTESDVHPSCKVTAMKCFLLELQVISLESGDASIHDTVESLIILANNSLSSNGNVTESGCKECEELEEKNIKEFLQSFVHIVQMFINTS

SEQ ID NO: 170 - protein, 34 aa, synthetic construct
  REGION 1..34: note = Synthetic: Artificial sequence
IRDPALVHQRPAPPSGGSGGGGSGGGSGGGGSLQ

SEQ ID NO: 171 - protein, 14 aa, synthetic construct
  REGION 1..14: note = Synthetic: Artificial sequence
IRDPALVHQRPAPP

SEQ ID NO: 172 - protein, 5 aa, synthetic construct
  REGION 1..5: note = Synthetic: 3F2 CDR1H
AQYMY

SEQ ID NO: 173 - protein, 17 aa, synthetic construct
  REGION 1..17: note = Synthetic: 3F2 CDR2H
RINPTSGNTVYAQKLQG

SEQ ID NO: 174 - protein, 7 aa, synthetic construct
  REGION 1..7: note = Synthetic: 3F2 CDR3H
GINWFDP

SEQ ID NO: 175 - protein, 7 aa, synthetic construct
  REGION 1..7: note = Synthetic: 50A10 CDR1L
SQDISNY

SEQ ID NO: 176 - skipped

SEQ ID NO: 177 - protein, 6 aa, synthetic construct
  REGION 1..6: note = Synthetic: 50A10 CDR3L
SYRSPF

SEQ ID NO: 178 - protein, 8 aa, synthetic construct
  REGION 1..8: note = Synthetic: 3F2 CDR1H
GYTFTAQY

SEQ ID NO: 179 - protein, 8 aa, synthetic construct
  REGION 1..8: note = Synthetic: 3F2 CDR2H
INPTSGNT

SEQ ID NO: 180 - protein, 9 aa, synthetic construct
  REGION 1..9: note = Synthetic: 3F2 CDR3H
ARGINWFDP

SEQ ID NO: 181 - protein, 7 aa, synthetic construct
  REGION 1..7: note = Synthetic: 50A10 CDR1L
SQDISNY

SEQ ID NO: 182 - skipped

SEQ ID NO: 183 - protein, 6 aa, synthetic construct
  REGION 1..6: note = Synthetic: 50A10 CDR3L
SYRSPF

SEQ ID NO: 184 - protein, 7 aa, synthetic construct
  REGION 1..7: note = Synthetic: 3F2 CDR1H
GYTFTAQ

SEQ ID NO: 185 - protein, 4 aa, synthetic construct
  REGION 1..4: note = Synthetic: 3F2 CDR2H
PTSG

SEQ ID NO: 186 - protein, 5 aa, synthetic construct
  REGION 1..5: note = Synthetic: 3F2 CDR3H
INWFD

SEQ ID NO: 187 - protein, 11 aa, synthetic construct
  REGION 1..11: note = Synthetic: 50A10 CDR1L
QASQDISNYIN

SEQ ID NO: 188 - protein, 7 aa, synthetic construct
  REGION 1..7: note = Synthetic: 50A10 CDR2L
AASRLQS

SEQ ID NO: 189 - protein, 9 aa, synthetic construct
  REGION 1..9: note = Synthetic: 50A10 CDR3L
QQSYRSPFT

(19) United States
(12) Patent Application Publication
(10) Pub. No.: US 2023/0312724 A1
(43) Pub. Date: Oct. 5, 2023

(54) B7-H4 Antibodies and Anti-B7-H4 Antibody/IL-15 Fusion Proteins

(71) Applicant: Kadmon Corporation, LLC, Bridgewater, NJ (US)

(72) Inventors: Dan Lu, Montvale, NJ (US); Jeegar P. Patel, Hillsborough, NJ (US); Tzu-Pei Chang, New York, NY (US); Zhanna Polonskaya, Mountainside, NJ (US); Faical Miyara, Bronxville, NY (US)

(73) Assignee: Kadmon Corporation, LLC, Bridgewater, NJ (US)

(21) Appl. No.: 18/059,548

(22) Filed: Nov. 29, 2022

Related U.S. Application Data
(60) Provisional application No. 63/284,937, filed on Dec. 1, 2021.

(51) Int. Cl. C07K 16/28; C07K 14/54; A61P 35/00; C12N 15/85

(52) U.S. Cl. CPC C07K 16/2827; C07K 14/5443; A61P 35/00; C12N 15/85; C07K 2317/526; C07K 2319/00; C12N 2800/107

(57) ABSTRACT

Provided herein are recombinant antibodies, antigen-binding fragments thereof, and fusion proteins thereof useful for binding to and inhibiting B7-H4. Also provided are nucleic acid molecules encoding the antibodies, antigen-binding fragments thereof, and fusion proteins thereof disclosed herein and therapeutic compositions thereof. Disclosed are further methods of using the disclosed antibodies, antigen-binding fragments thereof, and fusion proteins thereof for the treatment of disease.

### SEQUENCE LISTING

The present application contains a Sequence Listing which has been submitted electronically in XML format. Said XML copy, created on Nov. 28, 2022, is named “01183-0210-00PCT-KAD” and is 255,595 bytes in size. The information in the electronic format of the sequence listing is incorporated herein by reference in its entirety.

### FIELD

The present disclosure relates generally to the field of molecular biology and medicine. More particularly, the disclosure provides fusion proteins comprising an anti-B7-H4 antibody, or antigen-binding fragment thereof, linked to an IL-15 polypeptide, which in turn is linked to an IL-15 receptor alpha (IL-15Rα) polypeptide comprising the IL-15Rα sushi domain. In addition, the disclosure provides antibodies, and antigen-binding fragments thereof, that specifically bind to B7-H4 as well as fusion proteins comprising such anti-B7-H4 antibodies, or B7-H4 binding fragments thereof. Also disclosed are therapeutic compositions comprising the antibody fusions or antibodies for treating disease.

### BACKGROUND

B7-H4 is a co-inhibitory ligand of the B7 family expressed only on tumor cells, antigen-presenting cells (APCs), and TAMs (tumor-associated macrophages). B7-H4 binds to an unidentified cognate receptor on T cells. While PD-L1 expression is associated with immunologically “hot” tumors, B7-H4 expression marks “cold” environments. B7-H4 expression is negatively correlated with PD-L1 expression. Importantly, overexpression of B7-H4 correlates with advanced disease stage and poor prognosis in cancer patients. B7-H4 expressed by tumors and TAMs inhibits the activity of anti-tumor T cells, promotes an exhausted, dysfunctional state in T cells, promotes tumor-associated neutrophils (myeloid-derived suppressor cells), and induces regulatory T cells (T_{regs}), IL-6 and IL-10 production.

IL-15 is a 12.5 kDa glycoprotein with 114 amino acids that belongs to the four α-helix bundle family of cytokines. This family also includes IL-2, IL-4, IL-7, IL-9, granulocyte colony-stimulating factor (G-CSF), and GM-CSF. IL-15 is secreted by macrophages, dendritic cells, and monocytes. IL-15 can stimulate central memory CD8 cells to exert immunity without modulating effects on other T cells. Additionally, IL-15 can activate natural killer (NK) cells and effector and memory CD8 T cells and can rescue T cells from apoptosis induced by T_{regs}. Administration of IL-15 is also associated with a lower risk of inducing systemic toxicity at a higher dose compared to other cytokines. Human IL-15 can be soluble or membrane-bound. The membrane-bound IL-15, which is the major form of IL-15, is either formed by binding of IL-15 to cellular membrane directly or by presentation of IL-15 by the membrane-bound IL-15R receptor. The IL-15 receptor is composed of three subunits: IL-15Rα, IL-15Rβ, and IL-15Rγ. IL-15 typically forms a complex with IL-15 receptor α expressed on APCs prior to binding to functional IL-15Rβ and γ units on T cells and NK cells. IL-15 can bind to IL-15Rα receptor alone with affinity (K_{D}≈10 pM). It can also bind to IL-15Rβγc signaling complex with lower affinity (K_{D}≈1 nM). The sushi domain (29.5 kDa) of the IL-15Rα plays a critical role in complex formation of IL-15 and IL-15Rα. One of the limitations with systemic IL-15 treatment is its very short half-life in vivo. Therefore, there is a need to generate a suitable immune-stimulatory form of IL-15/IL-15Rα that has a longer half-life in vivo while retaining its ability to modulate the immune response. Additionally, there is a need for effective IL-15 antagonists that can be selectively targeted to the disease site to avoid unwanted systemic toxicities and provide a more effective therapeutic benefit.

Combination therapies involving B7-H4 antibodies with various cytokines such as IL-2, IL-15, IL-21, tumor necrosis factor (TNF), and granulocyte-macrophage colony-stimulating factor (GM-CSF) may have some efficacy in treating cancer and infection. However, these therapies are limited by the systemic toxicity that is associated both with the high blood concentrations of cytokines that is required to obtain efficacy and with the lack of specificity of the administered cytokine for affected cells and tissues.

Accordingly, there is a significant unmet need to develop new strategies to target various effector molecules to a disease site to provide therapeutic benefit without the side effects associated with non-specific immune activity.

### SUMMARY

Provided herein are fusion proteins comprising an anti-B7-H4 antibody, or antigen-binding fragment thereof, linked to an IL-15 polypeptide, which in turn is linked to an IL-15 receptor alpha (IL-15Rα) polypeptide comprising the IL-15Rα sushi domain. The disclosure provides antibodies, and antigen-binding fragments thereof, that specifically bind to B7-H4 as well as fusion proteins comprising such anti-B7-H4 antibodies, or B7-H4 binding fragments thereof. Also disclosed are methods of using the disclosed anti-B7-H4 antibodies, or antigen-binding fragments thereof, or fusion proteins for treating a subject in need thereof.

In one aspect, provided is an anti-B7-H4 antibody, or antigen-binding fragment thereof, wherein the antibody, or antigen-binding fragment thereof, comprises a heavy chain variable region and a light chain variable region, wherein each of the heavy chain and the light chain variable regions comprise a CDR1, CDR2, and CDR3, and wherein:
a. CDR1H comprises SEQ ID NO:16, CDR2H comprises SEQ ID NO:17, CDR3H comprises SEQ ID NO:18, and wherein:
   i. CDR1L comprises SEQ ID NO:20, CDR2L comprises SEQ ID NO:21, and CDR3L comprises SEQ ID NO:22;
   ii. CDR1L comprises SEQ ID NO:66, CDR2L comprises SEQ ID NO:67, and CDR3L comprises SEQ ID NO:68; or
   iii. CDR1L comprises SEQ ID NO:70, CDR2L comprises SEQ ID NO:71, and CDR3L comprises SEQ ID NO:72; or
b. CDR1H comprises SEQ ID NO:4, CDR2H comprises SEQ ID NO:5, CDR3H comprises SEQ ID NO:6, and wherein:
   i. CDR1L comprises SEQ ID NO: 8, CDR2L comprises SEQ ID NO: 9, and CDR3L comprises SEQ ID NO: 10;
   ii. CDR1L comprises SEQ ID NO: 77, CDR2L comprises SEQ ID NO: 78, and CDR3L comprises SEQ ID NO: 79; or
   iii. CDR1L comprises SEQ ID NO: 81, CDR2L comprises SEQ ID NO:82, and CDR3L comprises SEQ ID NO:83.

In one embodiment, provided is an anti-B7-H4 antibody, or antigen-binding fragment thereof, wherein the CDR1H comprises SEQ ID NO:16, CDR2H comprises SEQ ID NO:17, CDR3H comprises SEQ ID NO:18, CDR1L comprises SEQ ID NO:70, CDR2L comprises SEQ ID NO:71, and CDR3L comprises SEQ ID NO:72.

In one embodiment, provided is an anti-B7-H4 antibody, or antigen-binding fragment thereof, wherein:
a. the heavy chain variable region comprises SEQ ID NO: 15, or a sequence that is at least 90%, at least 95%, at least 96%, at least 97%, at least 98%, or at least 99% identical to SEQ ID NO: 15; and wherein
   i. the light chain variable region comprises SEQ ID NO: x, or a sequence that is at least 90%, at least 95%, at least 96%, at least 97%, at least 98%, or at least 99% identical to SEQ ID NO: 19;
   ii. the light chain variable region comprises SEQ ID NO: 65, or a sequence that is at least 90%, at least 95%, at least 96%, at least 97%, at least 98%, or at least 99% identical to SEQ ID NO: 65; or
   iii. the light chain variable region comprises SEQ ID NO: 69, or a sequence that is at least 90%, at least 95%, at least 96%, at least 97%, at least 98%, or at least 99% identical to SEQ ID NO: 69; or
b. the heavy chain variable region comprises SEQ ID NO: 3, or a sequence that is at least 90%, at least 95%, at least 96%, at least 97%, at least 98%, or at least 99% identical to SEQ ID NO: 3; and wherein:
   i. the light chain variable region comprises SEQ ID NO: 7, or a sequence that is at least 90%, at least 95%, at least 96%, at least 97%, at least 98%, or at least 99% identical to SEQ ID NO: 7;
   ii. the light chain variable region comprises SEQ ID NO: 76, or a sequence that is at least 90%, at least 95%, at least 96%, at least 97%, at least 98%, or at least 99% identical to SEQ ID NO: 76; or
   iii. the light chain variable region comprises SEQ ID NO: 80, or a sequence that is at least 90%, at least 95%, at least 96%, at least 97%, at least 98%, or at least 99% identical to SEQ ID NO: 80.

In one embodiment, provided is an anti-B7-H4 antibody, or antigen-binding fragment thereof, wherein the heavy chain variable region comprises SEQ ID NO: 15, or a sequence that is at least 90%, at least 95%, at least 96%, at least 97%, at least 98%, or at least 99% identical to SEQ ID NO: 15; and the light chain variable region comprises SEQ ID NO: 69, or a sequence that is at least 90%, at least 95%, at least 96%, at least 97%, at least 98%, or at least 99% identical to SEQ ID NO: 69.

In one embodiment, provided is an anti-B7-H4 antibody, or antigen-binding fragment thereof, wherein:
a. the heavy chain variable region comprises SEQ ID NO: 15 and wherein:
   i. the light chain variable region comprises SEQ ID NO: 19;
   ii. the light chain variable region comprises SEQ ID NO: 65;
   iii. the light chain variable region comprises SEQ ID NO: 69; and
b. the heavy chain variable region comprises SEQ ID NO: 3 and wherein:
   i. the light chain variable region comprises SEQ ID NO: 7;
   ii. the light chain variable region comprises SEQ ID NO: 76; or
   iii. the light chain variable region comprises SEQ ID NO: 80.

In one embodiment, provided is an anti-B7-H4 antibody, or antigen-binding fragment thereof, wherein the heavy chain variable region comprises SEQ ID NO: 15 and the light chain variable region comprises SEQ ID NO: 69.

In one embodiment, provided is an anti-B7-H4 antibody, or antigen-binding fragment thereof, wherein the antibody, or antigen-binding fragment thereof, binds to the IgV domain of human hB7-H4.

In one aspect, provided is an anti-B7-H4 antibody, or antigen-binding fragment thereof, wherein the antibody, or antigen-binding fragment thereof, comprises a heavy chain variable region and a light chain variable region, wherein each of the heavy chain and the light chain variable regions comprise a CDR1, CDR2, and CDR3, and wherein CDR1H comprises SEQ ID NO:46, CDR2H comprises SEQ ID NO:47, CDR3H comprises SEQ ID NO:48 and wherein:
a. CDR1L comprises SEQ ID NO:50, CDR2L comprises SEQ ID NO:51, and CDR3L comprises SEQ ID NO:52;
b. CDR1L comprises SEQ ID NO:88, CDR2L comprises SEQ ID NO:89, and CDR3L comprises SEQ ID NO:90;
c. CDR1L comprises SEQ ID NO:92, CDR2L comprises SEQ ID NO:93, and CDR3L comprises SEQ ID NO:94;
d. CDR1L comprises SEQ ID NO:96, CDR2L comprises SEQ ID NO:97, and CDR3L comprises SEQ ID NO:98;
e. CDR1L comprises SEQ ID NO:100, CDR2L comprises SEQ ID NO:101, and CDR3L comprises SEQ ID NO:102;
f. CDR1L comprises SEQ ID NO:104, CDR2L comprises SEQ ID NO:105, and CDR3L comprises SEQ ID NO:106; or
g. CDR1L comprises SEQ ID NO:108, CDR2L comprises SEQ ID NO:109, and CDR3L comprises SEQ ID NO:110.

In one embodiment, provided is an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:108, CDR2L comprises SEQ ID NO:109, and CDR3L comprises SEQ ID NO:110.

In one embodiment, provided is an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein:
a. the light chain variable region comprises SEQ ID NO:49, or a sequence that is at least 90%, at least 95%, at least 96%, at least 97%, at least 98%, or at least 99% identical to SEQ ID NO:49;
b. the light chain variable region comprises SEQ ID NO:87, or a sequence that is at least 90%, at least 95%, at least 96%, at least 97%, at least 98%, or at least 99% identical to SEQ ID NO:87;
c. the light chain variable region comprises SEQ ID NO:91, or a sequence that is at least 90%, at least 95%, at least 96%, at least 97%, at least 98%, or at least 99% identical to SEQ ID NO:91;
d. the light chain variable region comprises SEQ ID NO:95, or a sequence that is at least 90%, at least 95%, at least 96%, at least 97%, at least 98%, or at least 99% identical to SEQ ID NO:95;
e. the light chain variable region comprises SEQ ID NO:99, or a sequence that is at least 90%, at least 95%, at least 96%, at least 97%, at least 98%, or at least 99% identical to SEQ ID NO:99;
f. the light chain variable region comprises SEQ ID NO:103, or a sequence that is at least 90%, at least 95%, at least 96%, at least 97%, at least 98%, or at least 99% identical to SEQ ID NO:103; or
g. the light chain variable region comprises SEQ ID NO:107, or a sequence that is at least 90%, at least 95%, at least 96%, at least 97%, at least 98%, or at least 99% identical to SEQ ID NO:107.

In one embodiment, provided is an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45; and the light chain variable region comprises SEQ ID NO:107, or a sequence that is at least 90%, at least 95%, at least 96%, at least 97%, at least 98%, or at least 99% identical to SEQ ID NO:107.

In one embodiment, provided is an anti-B7-H4 antibody, or antigen-binding fragment thereof, wherein the heavy chain variable region comprises SEQ ID NO: 45 and wherein:
a. the light chain variable region comprises SEQ ID NO:49;
b. the light chain variable region comprises SEQ ID NO:87;
c. the light chain variable region comprises SEQ ID NO:91;
d. the light chain variable region comprises SEQ ID NO:95;
e. the light chain variable region comprises SEQ ID NO:99;
f. the light chain variable region comprises SEQ ID NO: 103; or
g. the light chain variable region comprises SEQ ID NO:107.

In one embodiment, provided is an anti-B7-H4 antibody, or antigen-binding fragment thereof, wherein the heavy chain variable region comprises SEQ ID NO:45 and the light chain variable region comprises SEQ ID NO:107.

In one embodiment, provided is an anti-B7-H4 antibody, or antigen-binding fragment thereof, wherein the antibody, or antigen-binding fragment thereof, binds to the IgC domain of human and murine hB7-H4.

In one aspect, provided is an anti-B7-H4 antibody, or antigen-binding fragment thereof, wherein the antibody, or antigen-binding fragment thereof, comprises a heavy chain variable region and a light chain variable region, wherein each of the heavy chain and the light chain variable regions comprise a CDR1, CDR2, and CDR3, wherein CDR1H comprises SEQ ID NO:58, CDR2H comprises SEQ ID NO:59, CDR3H comprises SEQ ID NO:60, and wherein:
a. CDR1L comprises SEQ ID NO:62, CDR2L comprises SEQ ID NO:63, and CDR3L comprises SEQ ID NO:64;
b. CDR1L comprises SEQ ID NO:115, CDR2L comprises SEQ ID NO:116, and CDR3L comprises SEQ ID NO:117;
c. CDR1L comprises SEQ ID NO:119, CDR2L comprises SEQ ID NO:120, and CDR3L comprises SEQ ID NO:121;
d. CDR1L comprises SEQ ID NO:123, CDR2L comprises SEQ ID NO:124, and CDR3L comprises SEQ ID NO:125;
e. CDR1L comprises SEQ ID NO:127, CDR2L comprises SEQ ID NO:128, and CDR3L comprises SEQ ID NO:129; or
f. CDR1L comprises SEQ ID NO:131, CDR2L comprises SEQ ID NO:132, and CDR3L comprises SEQ ID NO:133.

In one embodiment, provided is anti-B7-H4 antibody, or antigen-binding fragment thereof, wherein the CDR1H comprises SEQ ID NO:58, CDR2H comprises SEQ ID NO:59, CDR3H comprises SEQ ID NO:60, CDR1L comprises SEQ ID NO:119, CDR2L comprises SEQ ID NO:120, and CDR3L comprises SEQ ID NO:121.

In one embodiment, provided is an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein:
a. the light chain variable region comprises SEQ ID NO:61, or a sequence that is at least 90%, at least 95%, at least 96%, at least 97%, at least 98%, or at least 99% identical to SEQ ID NO:61;
b. the light chain variable region comprises SEQ ID NO:114, or a sequence that is at least 90%, at least 95%, at least 96%, at least 97%, at least 98%, or at least 99% identical to SEQ ID NO:114;
c. the light chain variable region comprises SEQ ID NO:118, or a sequence that is at least 90%, at least 95%, at least 96%, at least 97%, at least 98%, or at least 99% identical to SEQ ID NO:118;
d. the light chain variable region comprises SEQ ID NO:122, or a sequence that is at least 90%, at least 95%, at least 96%, at least 97%, at least 98%, or at least 99% identical to SEQ ID NO:122;
e. the light chain variable region comprises SEQ ID NO:126, or a sequence that is at least 90%, at least 95%, at least 96%, at least 97%, at least 98%, or at least 99% identical to SEQ ID NO:126; or
f. the light chain variable region comprises SEQ ID NO:130, or a sequence that is at least 90%, at least 95%, at least 96%, at least 97%, at least 98%, or at least 99% identical to SEQ ID NO:130.

In one embodiment, provided is an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57; and the light chain variable region comprises SEQ ID NO:118, or a sequence that is at least 90%, at least 95%, at least 96%, at least 97%, at least 98%, or at least 99% identical to SEQ ID NO:118.

In one embodiment, provided is an anti-B7-H4 antibody, or antigen-binding fragment thereof, wherein the heavy chain variable region comprises SEQ ID NO:57 and wherein:
a. the light chain variable region comprises SEQ ID NO:61;
b. the light chain variable region comprises SEQ ID NO:114;
c. the light chain variable region comprises SEQ ID NO: 118;
d. the light chain variable region comprises SEQ ID NO: 122;
e. the light chain variable region comprises SEQ ID NO: 126; or
f. the light chain variable region comprises SEQ ID NO: 130.

In one embodiment, provided is an anti-B7-H4 antibody, or antigen-binding fragment thereof, of wherein the heavy chain variable region comprises SEQ ID NO:57 and the light chain variable region comprises SEQ ID NO:118.

In one embodiment, provided is an anti-B7-H4 antibody, or antigen-binding fragment thereof, wherein the antibody, or antigen-binding fragment thereof, binds to the IgV domain of human and murine hB7-H4.

In one embodiment, provided is an anti-B7-H4 antibody, or antigen-binding fragment thereof, that binds to the same epitope on B7-H4 as the anti-B7-H4 antibody, or an antigen-binding fragment thereof disclosed herein.

In one embodiment, provided is an anti-B7-H4 antibody, or antigen-binding fragment thereof, wherein the antibody or antigen-binding fragment is a chimeric antibody, a CDR-grafted antibody, or a humanized antibody or antigen-binding fragment thereof.

In one embodiment, provided is an anti-B7-H4 antibody, or antigen-binding fragment thereof, wherein the antibody or antigen-binding fragment is a multispecific or a bispecific antibody or antigen-binding fragment thereof.

In one embodiment, provided is an anti-B7-H4 antibody or antigen-binding fragment thereof, wherein the antibody or antigen-binding fragment is an scFv, Fv, Fab′, Fab, F(ab′)₂, or diabody.

In one embodiment, provided is an anti-B7-H4 antibody, or antigen-binding fragment thereof, wherein the antibody is an IgG class immunoglobulin.

In one embodiment, provided is an anti-B7-H4 antibody, or an antigen-binding fragment thereof, wherein the antibody or antigen-binding fragment has isotype IgG1.

In one embodiment, provided is an anti-B7-H4 antibody, or antigen-binding fragment thereof, wherein the antibody or antigen-binding fragment is deglycosylated.

In one embodiment, provided is an anti-B7-H4 antibody, or antigen-binding fragment thereof, wherein the antibody or antigen-binding fragment comprises a first and a second heavy chain constant region, and wherein the antibody, or antigen-binding fragment thereof, comprises at least one modification in the CH3 domains of the first and the second heavy chain constant region causing heterodimerization. In one embodiment, the modification in the CH3 domain of first heavy chain constant region is different from the modification in the CH3 domain of the second heavy chain constant region. In one embodiment, the first heavy chain constant region comprises one or more amino acid substitutions selected from the group consisting of S354C, T366W, Y349C, T366S, L368A, and Y407V (Kabat EU index numbering) and the second heavy chain constant region comprises one or more amino acid substitutions selected from the group consisting of S354C, T366W, Y349C, T366S, L368A, and Y407V (Kabat EU index numbering). In some embodiments, (i) the first heavy chain constant region comprises one or more amino acid substitutions selected from the group consisting of S354C and T366W (Kabat EU index numbering) and the second heavy chain constant region comprises one or more amino acid substitutions selected from the group consisting of Y349C, T366S, L368A, and Y407V (Kabat EU index numbering); or (ii) the second heavy chain constant region comprises one or more amino acid substitutions selected from the group consisting of S354C and T366W (Kabat EU index numbering) and the first heavy chain constant region comprises one or more amino acid substitutions selected from the group consisting of Y349C, T366S, L368A, and Y407V (Kabat EU index numbering). In some embodiments, (i) the first heavy chain constant region comprises amino acid substitutions S354C and T366W (Kabat EU index numbering) and the second heavy chain constant region comprises amino acid substitutions Y349C, T366S, L368A, and Y407V (Kabat EU index numbering); or (ii) the second heavy chain constant region comprises amino acid substitutions S354C and T366W (Kabat EU index numbering) and the first heavy chain constant region comprises amino acid substitutions Y349C, T366S, L368A, and Y407V (Kabat EU index numbering). In one embodiment, the first heavy chain constant region, the second heavy chain constant region, or both comprise amino acid substitutions M428L and N434S (Kabat EU index numbering).

In one aspect, provided is a fusion protein comprising:
a. an anti-B7-H4 antibody, or antigen-binding fragment thereof,
b. an IL-15Rα sushi domain polypeptide comprising SEQ ID NO:167, or an amino acid sequence that at least 90%, at least 95%, at least 96%, at least 97%, at least 98%, or at least 99% identical to SEQ ID NO:167; and
c. an IL-15 polypeptide comprising SEQ ID NO:166, or an amino acid sequence that is at least 90%, at least 95%, at least 96%, at least 97%, at least 98%, or at least 99% identical to SEQ ID NO:166.

In some embodiments, the fusion protein comprises a heavy chain CDR1 (CDR1H), CDR2 (CDR2H) and CDR3 (CDR3H) and a light chain CDR1 (CDR1L), CDR2 (CDR2L), and CDR3 (CDR3L), wherein CDR1H comprises SEQ ID NO:16, CDR2H comprises SEQ ID NO:17, CDR3H comprises SEQ ID NO:18, CDR1L comprises SEQ ID NO:70, CDR2L comprises SEQ ID NO:71, and CDR3L comprises SEQ ID NO:72. In some embodiments, the fusion protein comprises a heavy chain CDR1 (CDR1H), CDR2 (CDR2H) and CDR3 (CDR3H) and a light chain CDR1 (CDR1L), CDR2 (CDR2L), and CDR3 (CDR3L), wherein CDR1H comprises SEQ ID NO:172, CDR2H comprises SEQ ID NO:173, CDR3H comprises SEQ ID NO:174, CDR1L comprises SEQ ID NO:175, CDR2L comprises SEQ ID NO:176, and CDR3L comprises SEQ ID NO:177. In some embodiments, the fusion protein comprises a heavy chain CDR1 (CDR1H), CDR2 (CDR2H) and CDR3 (CDR3H) and a light chain CDR1 (CDR1L), CDR2 (CDR2L), and CDR3 (CDR3L), wherein CDR1H comprises SEQ ID NO:178, CDR2H comprises SEQ ID NO:179, CDR3H comprises SEQ ID NO:180, CDR1L comprises SEQ ID NO:181, CDR2L comprises SEQ ID NO:182, and CDR3L comprises SEQ ID NO:183. In some embodiments, the fusion protein comprises a heavy chain CDR1 (CDR1H), CDR2 (CDR2H) and CDR3 (CDR3H) and a light chain CDR1 (CDR1L), CDR2 (CDR2L), and CDR3 (CDR3L), wherein CDR1H comprises SEQ ID NO:184, CDR2H comprises SEQ ID NO:185, CDR3H comprises SEQ ID NO:186, CDR1L comprises SEQ ID NO:187, CDR2L comprises SEQ ID NO:188, and CDR3L comprises SEQ ID NO:189.

In one embodiment, the IL-15Rα sushi domain polypeptide comprises SEQ ID NO:167. In one embodiment, the IL-15Rα sushi domain polypeptide consists of amino acid sequence of SEQ ID NO:167. In one embodiment, the IL-15 polypeptide comprises SEQ ID NO:166. In one embodiment, the IL-15 polypeptide consists of SEQ ID NO:166. In one embodiment, the IL-15Rα sushi domain polypeptide is fused to the N-terminus of the IL-15 polypeptide.

In one embodiment, the anti-B7-H4 antibody, or antigen-binding fragment thereof, comprises a first and a second heavy chain constant region, and wherein the IL-15Rα sushi domain polypeptide and the IL-15 polypeptide are fused to the C-terminus of the first heavy chain constant region. In one embodiment, the fusion protein comprises a linker joining (i) the IL-15Rα sushi domain polypeptide and the IL-15 polypeptide and (ii) the C-terminus of the constant region. In some embodiments, the linker is between 25-35 amino acids long. In some embodiments, the linker consists predominantly of Gly (G), Asn (N), Ser (S), Thr (T), Ala (A), Leu (L), and Gln (Q). In one embodiment, the linker comprises SEQ ID NO:168. In one embodiment, the fusion protein comprises SEQ ID NO:169. In one embodiment, the first and the second heavy chain constant regions comprise at least one modification in the CH3 domains of the first and the second heavy chain constant region causing heterodimerization. In one embodiment, the modification in the CH3 domain of first heavy chain constant region is different from the modification in the CH3 domain of the second heavy chain constant region. In one embodiment, the first heavy chain constant region comprises one or more amino acid substitutions selected from the group consisting of S354C, T366W, Y349C, T366S, L368A, and Y407V (Kabat EU index numbering). In some embodiments, (i) the first heavy chain constant region comprises one or more amino acid substitutions selected from the group consisting of S354C and T366W (Kabat EU index numbering) and the second heavy chain constant region comprises one or more amino acid substitutions selected from the group consisting of Y349C, T366S, L368A, and Y407V (Kabat EU index numbering); or (ii) the second heavy chain constant region comprises one or more amino acid substitutions selected from the group consisting of S354C and T366W (Kabat EU index numbering) and the first heavy chain constant region comprises one or more amino acid substitutions selected from the group consisting of Y349C, T366S, L368A, and Y407V (Kabat EU index numbering). In some embodiments, (i) the first heavy chain constant region comprises amino acid substitutions S354C and T366W (Kabat EU index numbering) and the second heavy chain constant region comprises amino acid substitutions Y349C, T366S, L368A, and Y407V (Kabat EU index numbering); or (ii) the second heavy chain constant region comprises amino acid substitutions S354C and T366W (Kabat EU index numbering) and the first heavy chain constant region comprises amino acid substitutions Y349C, T366S, L368A, and Y407V (Kabat EU index numbering). In one embodiment, the first heavy chain constant region, the second heavy chain constant region, or both comprise amino acid substitutions M428L and N434S (Kabat EU index numbering). In one embodiment, the fusion protein comprises not more than one IL-15Rα sushi domain polypeptide and not more than one IL-15 polypeptide. In some embodiments, the fusion protein comprises an anti-B7-H4 antibody, or antigen-binding fragment thereof, disclosed herein.

In one aspect, provided is a fusion protein comprising:
a. a light chain comprising SEQ ID NO:69 and/or SEQ ID NO:145;
b. a first heavy chain comprising SEQ ID NO:150 or SEQ ID NO:151; and
c. a second heavy chain comprising SEQ ID NO:157 or SEQ ID NO:158.

In one embodiment, the first heavy chain comprises SEQ ID NO:150; and the second heavy chain comprises SEQ ID NO:157. In one embodiment, the first heavy chain comprises SEQ ID NO:151; and the second heavy chain comprises SEQ ID NO:158.

In one aspect, provided is a fusion protein comprising:
a. a light chain comprising SEQ ID NO:118 and/or SEQ ID NO:144;
b. a first heavy chain comprising SEQ ID NO:148 or SEQ ID NO:149; and
c. a second heavy chain comprising SEQ ID NO:155 or SEQ ID NO:156.

In one embodiment, the first heavy chain comprises SEQ ID NO: 148; and the second heavy chain comprises SEQ ID NO:155.

In one embodiment, the first heavy chain comprises SEQ ID NO:149; and the second heavy chain comprises SEQ ID NO:156.

In one aspect, provided is a fusion protein comprising: a light chain comprising SEQ ID NO:145; a first heavy chain comprising SEQ ID NO:150; and a second heavy chain comprising SEQ ID NO:157.

In one aspect, provided is a nucleic acid sequence encoding an anti-B7-H4 antibody, or antigen-binding fragment thereof, or a fusion protein disclosed herein.

Provided herein is a vector comprising a nucleic acid disclosed herein. Provided herein is a set of vectors comprising two or more nucleic acid molecules disclosed herein.

Provided herein is a cell comprising a nucleic acid or a vector disclosed herein. Provided herein is a cell expressing an anti-B7-H4 antibody, or antigen-binding fragment thereof, or a fusion protein disclosed herein. Provided herein is a T cell expressing an anti-B7-H4 antibody, or antigen-binding fragment thereof, or a fusion protein disclosed herein. Provided herein is a T cell expressing an anti-B7-H4 binding protein comprising the heavy and light variable chains of an anti-B7-H4 antibody, or antigen-binding fragment thereof, disclosed herein. In some embodiments, the cell is isolated.

In some embodiments, the anti-B7-H4 antibody, or antigen-binding fragment thereof, or the fusion protein is conjugated to one or more of a cytotoxin, a fluorescent label, and an imaging agent.

Provided herein is a pharmaceutical composition comprising (i) an anti-B7-H4 antibody, or antigen-binding fragment thereof, or a fusion protein disclosed herein and (ii) a pharmaceutically acceptable carrier.

Provided herein is a method of producing an anti-B7-H4 antibody, or antigen-binding fragment thereof, or a fusion protein disclosed herein, the method comprising culturing the cell disclosed herein under conditions so that the anti-B7-H4 antibody, or antigen-binding fragment thereof, or fusion protein is produced.

Provided herein is a method of inhibiting binding of B7-H4 to a ligand of B7-H4 in a subject in need thereof, the method comprising administering to the subject an effective amount of an anti-B7-H4 antibody, or antigen-binding fragment thereof, or a fusion protein disclosed herein.

Provided herein is method of increasing T cell activation in a subject in need thereof, the method comprising administering to the subject an effective amount of an anti-B7-H4 antibody, or antigen-binding fragment thereof, or a fusion protein disclosed herein.

Provided herein is method of increasing CD8+ T cell proliferation in a subject in need thereof, the method comprising administering to the subject an effective amount of an anti-B7-H4 antibody, or antigen-binding fragment thereof, or a fusion protein disclosed herein.

Provided herein is method of inducing antibody dependent cell mediated cytotoxicity (ADCC) in a B7-H4-expressing cell in a subject in need thereof, the method comprising administering to the subject an effective amount of an anti-B7-H4 antibody, or antigen-binding fragment thereof, or a fusion protein disclosed herein.

Provided herein is method of stimulating the immune system in a subject in need thereof, the method comprising administering to the subject an effective amount of an anti-B7-H4 antibody, or antigen-binding fragment thereof, or a fusion protein disclosed herein.

Provided herein is method of treating cancer in a subject in need thereof, the method comprising administering to the subject an effective amount of an anti-B7-H4 antibody, or antigen-binding fragment thereof, or a fusion protein disclosed herein. In some embodiments, the cancer is ovarian cancer, melanoma, pancreatic cancer, thyroid cancer, lung cancer, colorectal cancer, squamous cancer, prostate cancer, breast cancer, bladder cancer, or gastric cancer. In some embodiments, the cancer is triple-negative breast cancer or ovarian cancer.

Provided herein is method of reducing tumor growth in a subject in need thereof, the method comprising administering to the subject an effective amount of an anti-B7-H4 antibody, or antigen-binding fragment thereof, or a fusion protein disclosed herein.

Provided herein is method of reducing tumor metastasis in a subject in need thereof, the method comprising administering to the subject an effective amount of an anti-B7-H4 antibody, or antigen-binding fragment thereof, or a fusion protein disclosed herein.

In one embodiment, the method further comprises administering an additional therapeutic agent or additional therapy. In some embodiments, the additional therapeutic agent is selected from the group consisting of a cancer vaccine, a checkpoint inhibitor, an antibody to a tumor-specific antigen, Bacillus Calmette-Guerin vaccine, a cytotoxin, an interleukin 6 receptor (IL-6R) inhibitor, an interleukin 4 receptor (IL-4R) inhibitor, an IL-10 inhibitor, IL-2, IL-7, IL-21, IL-15, an antibody-drug conjugate, an anti-inflammatory drug, and a dietary supplement. In some embodiments, the checkpoint inhibitor is a CTLA-4, a PD-1, a PD-L1, or a PD-L2 inhibitor. In some embodiments, the additional therapeutic agent is an inhibitor of LAG3, TIGIT, LAP, Podoplanin, Protein C receptor, ICOS, GITR, CD226 or CD160. In some embodiments, the additional therapy is chemotherapy, radiotherapy, or surgery. In some embodiments, the additional therapeutic agent or additional therapy is administered concurrently or consecutively with the anti-B7-H4 antibody, or antigen-binding fragment thereof, or the fusion protein. In some embodiments, the additional therapeutic agent is administered separately or as a mixture with the anti-B7-H4 antibody, or an antigen-binding fragment thereof, or the fusion protein.

In one embodiment, the subject has upregulated expression of B7-H4, or the subject has been identified as positive for expression of B7-H4. In one embodiment, the subject does not respond to therapy with a checkpoint inhibitor or initially responds to checkpoint inhibitor treatment but has later become resistant to checkpoint inhibitor blockade. In one embodiment, the subject is a human.

In one aspect, provided is a method of detecting B7-H4 in a sample, the method comprising contacting the sample with an anti-B7-H4 antibody, or antigen-binding fragment thereof, or a fusion protein disclosed herein.

### BRIEF DESCRIPTION OF THE FIGURES

FIG. 1A illustrates the B7-H4's unique structure, which includes a highly glycosylated IgC and a very short cytoplasmic tail. FIG. 1B shows an exemplary fusion protein disclosed herein.

FIG. 2 illustrates the process of obtaining high affinity B7-H4 antibodies by panning of fully human scFv phage display libraries (Superman 2.0 from Distributed Bio) on B7-H4, followed by affinity maturation (light chain shuffling).

FIGS. 3A, 3B, 3C, 3D, 3E, and 3F illustrate binding of antibodies 1D3, 3F2, 1A12, 1F11, and 6C3 to both soluble and cellular B7-H4. ELISA experiments were performed to determine binding to hB7-H4-Fc (FIG. 3A), hB7-H4-his (FIG. 3B), mB7-H4-Fc (FIG. 3C), and hB7-H4 IgV domain (FIG. 3D). Flow cytometry experiments were performed to determine binding to CHO cells expressing hB7-H4 (FIG. 3E) or mB7-H4 (FIG. 3F). Antibodies 1D3, 3F2, 1A12, 1F11, and 6C3 bound to both soluble and cellular hB7-H4, specifically to the IgV domain of hB7-H4.

FIGS. 4A, 4B, 4C, 4D, 4E, and 4F illustrate binding of antibodies 6C3, 1D3, 3D1, and 4H6 to both soluble and cellular B7-H4. ELISA experiments were performed to determine binding to hB7-H4-Fc (FIG. 4A), hB7-H4-his (FIG. 4B), hB7-H4 IgV domain (FIG. 4C), and mB7-H4 IgV domain (FIG. 4D). Flow cytrometry experiments were performed to determine binding to CHO cells expressing hB7-H4 (FIG. 4E) or mB7-H4 (FIG. 4F). Antibodies 6C3, 1D3, 3D1, and 4H6 bound to both soluble and cellular hB7-H4, specifically to the IgV domain of hB7-H4. A cross-reactive IgV binding antibody was used as a control.

FIGS. 5A, 5B, 5C, 5D, 5E, 5F, and 5G illustrate binding of antibody 9H2 to both soluble and cellular B7-H4. ELISA experiments were performed to determine binding to hB7-H4-Fc (FIG. 5A), hB7-H4-his (FIG. 5B), mB7-H4-Fc domain (FIG. 5C), hB7-H4 IgV domain (FIG. 5D), and hB7-H4 IgC domain (FIG. 5E). Flow cytrometry experiments were performed to determine binding to CHO cells expressing hB7-H4 (FIG. 5F) or mB7-H4 (FIG. 5G). Antibody 9H2 bound to both soluble and cellular hB7-H4, specifically to the IgV domain of hB7-H4. A cross-reactive IgV binding antibody and an IgC binding antibody were used as controls.

FIGS. 6A, 6B, 6C, 6D, 6E, and 6F illustrate binding of antibodies 5G6, 9E1, 5F4, 5E4, and 4B9 to both soluble and cellular B7-H4. ELISA experiments were performed to determine binding to hB7-H4-Fc (FIG. 6A), mB7-H4-hFc (FIG. 6B), hB7-H4 IgV domain (FIG. 6C), and mB7-H4 IgV domain (FIG. 6D). Flow cytrometry experiments were performed to determine binding to CHO cells expressing hB7-H4 (FIG. 6E) or mB7-H4 (FIG. 6F). Antibodies 5E4, 5F4, 5G6, and 9E1 bound to the IgC domain of both human and mouse B7-H4. Antibody 4B9 bound to hB7-H4-Fc strongly but did not bind to mB7-H4. Binding of antibody 4B9 required both IgV and IgC. A cross-reactive IgV binding antibody was used as a control.

FIGS. 7A, 7B, 7C, 7D, 7E, 7F, 7G, and 7H illustrate binding of antibodies 9D11, 24B6, 15B11, 30G4, and 24F4 to both soluble and cellular B7-H4. ELISA experiments were performed to determine binding to hB7-H4-Fc (FIG. 7A), to hB7-H4-His (FIG. 7B), mB7-H4-hFc (FIG. 7C), mB7-H4-His (FIG. 7D), hB7-H4 IgC domain (FIG. 7E), and mB7-H4 IgC domain (FIG. 7F). Flow cytrometry experiments were performed to determine binding to CHO cells expressing hB7-H4 (FIG. 7G) or mB7-H4 (FIG. 7H). Antibodies 9D11, 24B6, 15B11, 30G4, and 24F4 bound to the IgC domain of both human and mouse B7-H4. A cross-reactive IgC binding antibody was used as a control.

FIGS. 8A, 8B, 8C, 8D, 8E, 8F, 8G, and 8H illustrate binding of antibodies 39A11 and 31D7 to both soluble and cellular B7-H4. ELISA experiments were performed to determine binding to hB7-H4-Fc (FIG. 8A), to hB7-H4-His (FIG. 8B), mB7-H4-hFc (FIG. 8C), mB7-H4-His (FIG. 8D), hB7-H4 IgV domain (FIG. 8E), hB7-H4 IgC domain (FIG. 8F), mB7-H4 IgV domain (FIG. 8G), and mB7-H4 IgC domain (FIG. 8H). Flow cytrometry experiments were performed to determine binding to CHO cells expressing hB7-H4 (FIG. 8I) or mB7-H4 (FIG. 8J). Antibodies 39A11 and 31D7 bound to the IgV domain of both human and mouse B7-H4. A cross-reactive IgV binding antibody was used as a control.

FIGS. 9A, 9B, 9C, 9D, 9E, 9F, 9G, 9H illustrate binding of antibodies 1D3/1D3, 1D3/1D3 derivative 1D3/45A2, 1D3/1D3 derivative 1D3/47B2, 3F2/3F2, 3F2/3F2 derivative 3F2/50A10, and 3F2/3F2 derivative 3F2/49A2 to soluble B7-H4. ELISA experiments were performed to determine binding to hB7-H4-Fc (FIG. 9A), mB7-H4-Fc (FIG. 9B), hB7-H4-his (FIG. 9C), mB7-H4-his (FIG. 9D), hB7-H4 IgV domain (FIG. 9E), mB7-H4 IgV domain (FIG. 9F), hB7-H4 IgC domain (FIG. 9G), mB7-H4 IgC domain (FIG. 9H). 3F2 derivatives 3F2/50A10 and 3F2/49A2 have the same heavy chain sequence as 3F2, but a different light chain sequence than 3F2. 1D3 derivatives 1D3/47B2 and 1D3/45A2 have the same heavy chain sequence as 1D3, but a different light chain sequence than 1D3. Antibodies C3 and 49A2 have the same VH and VL sequences.

FIGS. 10A, 10B, 10C, 10D illustrate binding of antibodies 1D3/1D3, 1D3/1D3 derivative 1D3/45A2, 1D3/1D3 derivative 1D3/47B2, 3F2/3F2, 3F2/3F2 derivative 3F2/50A10, and 3F2/3F2 derivative 3F2/49A2 to cell-bound B7-H4. Flow cytometry experiments were performed to determine binding to CHO cells expressing hB7-H4 (FIG. 10A), CHO cells expressing mB7-H4 (FIG. 10B), triple negative breast cancer cell line MDA-MB-468 cells expressing hB7-H4 (FIG. 10C), and ovary cancer cell line SK-BR-3 cells expressing hB7-H4 (FIG. 10D).

FIGS. 11A, 11B, 11C, 11D, 11E, and 11F illustrate binding of antibody 9D11/9D11 and 9D11/9D11 derivatives 9D11/67H9, 9D11/67C3, 9D11/67C6, 9D11/67E12, and 9D11/67G3 to soluble B7-H4. ELISA experiments were performed to determine binding to hB7-H4-Fc (FIG. 11A), mB7-H4-Fc (FIG. 11B), hB7-H4 IgC domain (FIG. 11C), mB7-H4 IgC domain (FIG. 11D), hB7-H4 IgV domain (FIG. 11E), and mB7-H4 IgV domain (FIG. 11F). 9D11 derivatives 9D11/67E12, 9D11/67C3, 9D11/67C6, 9D11/67H9, 9D11/67G3, and 9D11/68F5 have the same heavy chain sequence as 9D11, but a different light chain sequence than 9D11.

FIGS. 12A, 12B, 12C, and 12D illustrate binding of 9D11/9D11 derivatives 9D11/67C3, 9D11/67E12, and 9D11/68F5 to soluble B7-H4. ELISA experiments were performed to determine binding to hB7-H4-Fc (FIG. 12A), mB7-H4-Fc (FIG. 12B), hB7-H4-his (FIG. 12C), and hB7-H4-his (FIG. 12D). 9D111 derivatives 9D11/67C3, 9D11/67E12, and 9D11/68F5 have the same heavy chain sequence as 9D11, but a different light chain sequence than 9D11.

FIGS. 13A and 13B illustrate binding of antibodies 9D11/9D11 and 9D11/9D11 derivatives 9D11/67C3, 9D11/67C6, 9D11/67E12, 9D11/67G3, 9D11/67H9, and 9D11/68F5 to cell-bound B7-H4. Flow cytrometry experiments were performed to determine binding to CT26 cells expressing mB7-H4 (FIG. 13A) and SK-BR-3 cells expressing hB7-H4 (FIG. 13B). 9D11 derivatives 9D11/67C3, 9D11/67C6, 9D11/67E12, 9D11/67G3, 9D11/67H9, and 9D11/68F5 have the same heavy chain sequence as 9D11, but a different light chain sequence than 9D11.

FIGS. 14A, 14B, 14C, 14D, 14E, and 14F illustrate binding of antibody 39A11/39A11 and 39A11/39A11 derivatives 39A11/62F9, 39A11/56H7, 39A11/57G8, 39A11/57H3, and 39A11/56A9 to soluble B7-H4. ELISA experiments were performed to determine binding to hB7-H4-Fc (FIG. 14A), mB7-H4-Fc (FIG. 14B), hB7-H4 IgV domain (FIG. 14C), mB7-H4 IgV domain (FIG. 14D), hB7-H4 IgC domain (FIG. 14E), and mB7-H4 IgC domain (FIG. 14F). 39A11 derivatives 39A11/62F9, 39A11/56H7, 39A11/57G8, 39A11/57H3, and 39A11/56A9 have the same heavy chain sequence as 39A11, but different a light chain sequence than 39A11.

FIGS. 15A and 15B illustrate binding of antibodies 39A11/39A11 and 39A11/39A11 derivatives 39A11/62F9, 39A11/56H7, 39A11/57G8, 39A11/57H3, and 39A11/56A9 to cell-bound B7-H4. Flow cytrometry experiments were performed to determine binding to CT26 cells expressing mB7-H4 (FIG. 15A) and SK-BR-3 cells expressing hB7-H4 (FIG. 15B). 39A11 derivatives 39A11/62F9, 39A11/56H7, 39A11/57G8, 39A11/57H3, and 39A11/56A9 have the same heavy chain sequence as 39A11, but a different light chain sequence than 39A11.

FIGS. 16A, 16B, 16C, 16D, 16E, and 16F illustrate binding of antibodies 3F2/50A10 (hB7-H4 IgV domain binding antibody), 39A11/57G8 (h/mB7-H4 IgV domain binding antibody), and 9D11/68F5 (h/mB7-H4 IgC domain binding antibody) to soluble B7-H4. ELISA experiments were performed to determine binding to hB7-H4-Fc (FIG. 16A), mB7-H4-Fc (FIG. 16B), hB7-H4 IgV domain (FIG. 16C), mB7-H4 IgV domain (FIG. 16D), hB7-H4 IgC domain (FIG. 16E), and mB7-H4 IgC domain (FIG. 16F). Antibodies binding to IgV or IgC, respectively, were used as controls.

FIGS. 17A, 17B, and 17C illustrate binding of antibodies 3F2/50A10 (hB7-H4 IgV domain binding antibody), 39A11/57G8 (h/mB7-H4 IgV domain binding antibody), and 9D11/68F5 (h/mB7-H4 IgC domain binding antibody) to cell-bound B7-H4. Flow cytrometry experiments were performed to determine binding to SK-BR-3 cells expressing hB7-H4 (FIG. 17A), CT26 cells expressing mB7-H4 (FIG. 17B), and MDA-MB-468 cells expressing hB7-H4 (FIG. 17C).

FIGS. 18A and 18B illustrate the internalization of anti-B7-H4 antibodies antibodies 3F2/50A10 (hB7-H4 IgV domain binding antibody), 39A11/57G8 (h/mB7-H4 IgV domain binding antibody), and 9D11/68F5 (h/mB7-H4 IgC domain binding antibody) after binding to their hB7-H4. Antibodies were mixed with an antibody labeling reagent (i.e., an Fc-region targeting Fab fragment conjugated to a pH-sensitive fluorescent probe). A fluorogenic signal is observed as the Fab-Ab complex is internalized and processed via acidic (pH 4.5-5.5) lysosomes and endosomes. Internalization is shown for SK-BR-3 cells expressing hB7-H4 (FIG. 18A) and MDA-MB-468 cells expressing hB7-H4 (FIG. 18B). IgV or IgC binding antibodies, respectively, were used as controls.

FIGS. 19A and 19B illustrate restoration T cell cytokine secretion function (here IFNγ secretion) by antibodies 3F2/50A10 (hB7-H4 IgV domain binding antibody), 39A11/57G8 (h/mB7-H4 IgV domain binding antibody) (FIG. 19A), and 9D11/68F5 (h/mB7-H4 IgC domain binding antibody) (FIG. 19B). IgV or IgC binding antibodies, respectively, were used as controls. Also included was an irrelevant control antibody.

FIGS. 20A and 20B illustrate that antibodies 3F2/50A10 (hB7-H4 IgV domain binding antibody), 39A11/57G8 (h/mB7-H4 IgV domain binding antibody), and 9D11/68F5 (h/mB7-H4 IgC domain binding antibody) induce antibody-dependent cell-mediated cytotoxicity (ADCC) in tumor cells. Tumor cell killing is shown for SK-BR-3 cells expressing hB7-H4 (FIG. 20A) and MDA-MB-468 cells expressing hB7-H4 (FIG. 20B).

FIGS. 21A and 21B illustrate that IgV domain binding antibodies 3F2/50A10 and 39A11/57G8 kill tumor cells in an IncuCyte® immune cell killing assay. An SKBR3/hPBMC co-culture model was set-up for analyzing ADCC, in which SK-BR-3 cancer cells were mixed with hPBMC in a ratio of 1:5 (FIG. 21A) or 1:10 (FIG. 21B), respectively. DP47 served as a control antibody.

FIGS. 22A, 22B, 22C, 22D, 22E, and 22F illustrate binding of antibodies 39A11/57G8 and a positive control antibody (IgV binding antibody) as well as binding of fusion proteins comprising antibody 39A11/57G8 (i.e., fusion protein 57G8/IL15) or the positive control antibody (i.e., fusion protein (+)Abl/IL15), respectively to B7-H4. ELISA experiments were performed to determine binding to hB7-H4-Fc (FIG. 22A), hB7-H4-his (FIG. 22B), hB7-H4 IgV domain (FIG. 22C), mB7-H4-Fc (FIG. 22D), mB7-H4-his (FIG. 22E), mB7-H4 IgV domain (FIG. 22F).

FIGS. 23A, 23B, 23C, 23D, 23E, and 23F illustrate binding of antibodies 3F2/50A10 and a positive control antibody (IgV binding antibody) as well as binding of fusion proteins comprising antibody 3F2/50A10 (i.e., fusion protein 50A10/IL15) or the positive control antibody (i.e., fusion protein (+)Abl/IL15), respectively to B7-H4. ELISA experiments were performed to determine binding to hB7-H4-Fc (FIG. 23A), hB7-H4-his (FIG. 23B), hB7-H4 IgV domain (FIG. 23C), mB7-H4-Fc (FIG. 23D), mB7-H4-his (FIG. 23E), mB7-H4 IgV domain (FIG. 23F).

FIGS. 24A, 24B, 24C, and 24D, illustrate binding of antibodies 3F2/50A10, 39A11/57G8 and a positive control antibody (binding to B7-H5 IgV domain) as well as binding of fusion proteins comprising antibody 3F2/50A10 (i.e., fusion protein 50A10/IL15), antibody 39A11/57G8 (i.e., fusion protein 57G8/IL15), or the positive control antibody (i.e., fusion protein (+)Abl/IL15) to cellular B7-H4. Flow cytrometry experiments were performed to determine binding to SK-BR-3 cells expressing hB7-H4 (FIG. 24A and FIG. 24B) or CT26 cells expressing mB7-H4 (FIG. 24C and FIG. 24D).

FIGS. 25A, 25B, 25C, and 25D illustrate binding of antibodies 3F2/50A10, 39A11/57G8 and a negative control antibody DP47 as well as binding of fusion proteins comprising antibody 3F2/50A10 (i.e., fusion protein 50A10/IL15), antibody 39A11/57G8 (i.e., fusion protein 57G8/IL15), or the negative control antibody (i.e., fusion protein DP47/IL15) to cellular B7-H4 expressed on MDA-MB-468 cells (FIGS. 25A and 25B) or MX-1 (FIGS. 25C and 25D) as determined by flow cytrometry.

FIGS. 26A and 26B illustrate tumor cell killing by antibodies 3F2/50A10, 39A11/57G8, and negative control antibody DP47 as well as tumor cell killing by fusion proteins comprising antibody 3F2/50A10 (i.e., fusion protein 50A10/IL15), antibody 39A11/57G8 (i.e., fusion protein 57G8/IL15), or the negative control antibody (i.e., fusion protein DP47/IL15) using an IncuCyte® immune cell killing assay. Killing of SD-BR-3 cells is shown for 3F2/50A10 and fusion protein 50A10/IL15 (FIG. 26A) and for 39A11/57G8 and fusion protein 57G8/IL15 (FIG. 26B). Killing of MDA-MB-468 cells is shown for 3F2/50A10 and fusion protein 50A10/IL15 (FIG. 26C). FIG. 26D shows the end point values for the curves shown in FIG. 26C. FIG. 26E shows apoptosis of MDA-MB-468 cells after treatment with 50A10/IL-15, 57G8/IL-15, 57G8 and 50A1.

FIGS. 27A, 27B, and 27C illustrate the effect of antibodies antibody 39A11/57G8 and 3F2/50A10 as well as fusion proteins 57G8/IL15 and 50A10/IL15 on proliferation of hPBMC (FIG. 27A) and specifically CD8+ T cells (FIG. 27B) and CD4+ T cells (FIG. 27C). Antibody DP47 and an IL-15 fusion protein comprising DP47 were used as controls.

FIGS. 28A and 28B illustrate the ability of antibodies 39A11/57G8 and 3F2/50A10 as well as fusion proteins 57G8/IL15 and 50A10/IL115 to activate the p-STAT5 signal pathway through IL-2 receptors. FIG. 28A illustrates the STAT5 signaling pathway. FIG. 28B shows activation of the pathway by the indicates antibodies and fusion proteins. Antibody DP47 and an IL-15 fusion protein comprising DP47 were used as controls.

FIGS. 29A and 29B illustrate that antibodies 39A11/57G8 and 3F2/50A10 as well as fusion proteins 57G8/IL15 and 50A10/IL15 induce antibody-dependent cell-mediated cytotoxicity (ADCC) in tumor cells. Tumor cell killing is shown for SK-BR-3 cells expressing hB7-H4 (FIG. 29A) and MDA-MB-468 cells expressing hB7-H4 (FIG. 29B).

FIGS. 30A, 30B, 30C, and 30D, illustrate binding of fusion proteins 57G8/IL-15, 57G8/IL-15_LS, 50A10/IL-15 and 50A10/IL-15_LS to soluble B7-H4. The “LS” mutation (M428L/N434S) in the constant region of an antibody increases affinity to FcRn and lower the Koffrate at pH 6, thus leading to an extend serum half-life. ELISA experiments were performed to determine binding to hB7-H4-Fc (FIG. 30A), mB7-H4-Fc (FIG. 30B), hB7-H4his (FIG. 30C), and mB7-H4his (FIG. 30D). An IL-15 fusion protein comprising DP47 was used as a control.

FIGS. 31A and 31B illustrate binding of fusion proteins 57G8/IL-15, 57G8/IL-15_LS, 50A10/IL-15 and 50A10/IL-15_LS to cell-bound B7-H4. Flow cytrometry experiments were performed to determine binding to SK-BR-3 cells expressing hB7-H4 (FIG. 31A) and CT26 cells expressing mB7-H4 (FIG. 31B). An IL-15 fusion protein comprising DP47 was used as a control.

FIG. 32 illustrates the ability of fusion proteins 57G8/IL-15, 57G8/IL-15_LS, 50A10/IL-15 and 50A10/IL-15_LS to induce proliferation of IL-2 dependent M07e cells. An IL-15 fusion protein comprising DP47 was used as a control.

### DETAILED DESCRIPTION

Fusion Proteins

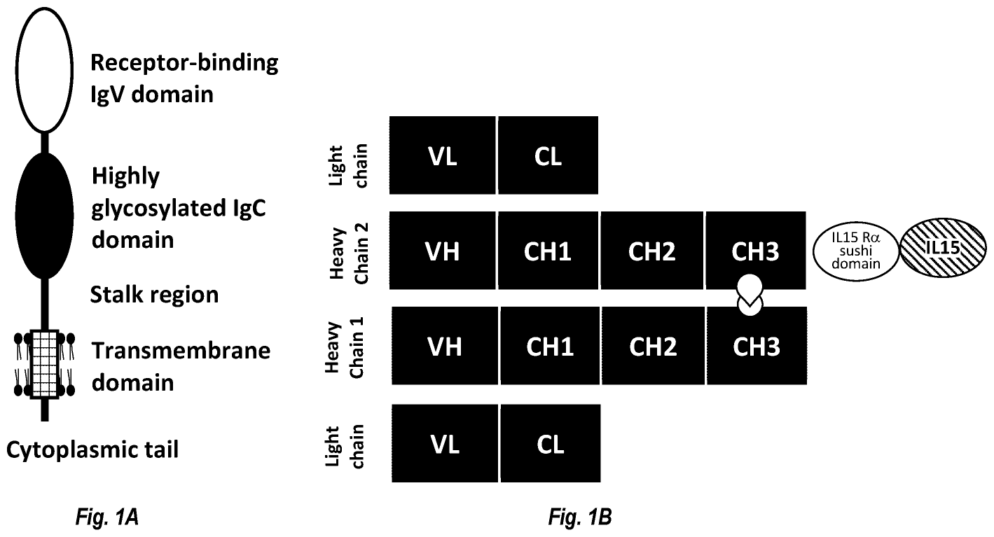

B7-H4 (also known as V-set domain-containing T-cell activation inhibitor 1, also known as B7 homolog 4, B7h.5, immune costimulatory protein B7-H4, protein B7S1, or T-cell costimulatory molecule B7x) is an immune regulatory molecule that shares homology with other B7 family members, include PD-L1. B7-H4 is a type I transmembrane protein comprised of both IgV and IgC ectodomains. While B7-H4 expression in healthy tissues is relatively limited at the protein level, B7-H4 is expressed in several solid tumors. Expression of B7-H4 in tumors tends to correlate with poor prognosis. B7-H4 has a unique structure comprising a highly glycosylated IgC domain and very short cytoplasmic tail (FIG. 1A).

In one aspect, the disclosure provides fusion proteins comprising an antibody, or antigen-binding fragment thereof, that binds to B7-H4. This portion of the fusion protein can be any antibody, or antibody fragment thereof, that specifically binds B7-H4, including those comprising the corresponding heavy and light chain variable regions or CDRs of the antibodies provided in Tables 1, 5, 7, 9 and 16-20, or otherwise described herein.

Disclosed herein are fusion proteins comprising a stimulatory domain. As used herein, a “stimulatory domain” is a domain that promotes an immune response. The stimulatory domain may stimulate an immune response mediated by, for example, inducing T cell or NK cell activity and/or proliferation. In embodiments, the stimulatory domain stimulates cells that respond to an interleukin or an interferon, such as, without limitation, IL-2, IL-7, IL-15, and IL-21. In one embodiment, the stimulatory domain binds to and stimulates a receptor that is responsive to an interleukin or an interferon, such as, without limitation, IL-2, IL-7, IL-13, IL-15, and IL-21. The stimulatory domain can also be a hybrid domain that is a hetero-complex of two or more ligands covalently linked to each other.

In embodiments, the stimulatory domain includes a sequence or domain that promotes IL-15 stimulation of the IL-15 receptor (IL-15R).

In one embodiment, the stimulatory domain that promotes IL-15R stimulation comprises IL-15 or an IL-15 derivative.

In one embodiment, the stimulatory domain that promotes IL-15R stimulation comprises an IL-15Rα polypeptide comprising the IL-15Rα sushi domain or a derivative thereof. In an embodiment, the stimulatory domain comprises the sushi domain of the IL-15Rα chain.

In an embodiment, the stimulatory domain comprises IL-15 or a derivative thereof, whose binding may be enhanced by the presence of an IL-15Rα polypeptide comprising the IL-15Rα sushi domain or a derivative thereof. In one embodiment, the stimulatory domain comprises a complex of IL-15 or a derivative thereof and an IL-15Rα polypeptide comprising the IL-15Rα sushi domain or a derivative thereof. In some embodiment, the stimulatory domain comprises an IL-15 or a derivative thereof and an IL-15Rα polypeptide comprising the IL-15Rα sushi domain or a derivative thereof, wherein the two polypeptides are covalently linked by a linker.

In one embodiment, the IL-15 or derivative thereof is located N-terminally of the IL-15Rα polypeptide comprising the IL-15Rα sushi domain or derivative thereof. In one embodiment, the IL-15 or derivative thereof is located C-terminally of the IL-15Rα polypeptide comprising the IL-15Rα sushi domain or derivative thereof. In one embodiment, the stimulatory domain comprises the sequence SEQ ID NO:169.

Provided herein is a fusion protein which comprises (1) an anti-B7H4 binding domain that blocks binding of B7-H4 to its ligand and inhibits immunosuppression, and (2) a stimulatory domain that promotes an immune response, wherein the fusion proteins can provide for increased immune cell activity, compared to two distinct molecules that provide the beforementioned functions separately. Specifically, the experiments disclosed herein demonstrate that fusion proteins containing both a B7-H4 binding domain that blocks binding of B7-H4 to its ligand, and an stimulatory domain IL-15 or a derivative thereof and an IL-15Rα polypeptide comprising the IL-15Rα sushi domain or a derivative thereof, killed tumor cells, induced T cell proliferation, and activated the STAT5 pathway downstream of IL-2Rβγ.

The fusion proteins provided herein may exhibit (a) binding to B7-H4 and blocking of downstream signaling events; (b) blocking binding of B7-H4 to its cognate ligand; (c) increasing T cell proliferation; (d) upregulating the T cell-mediated immune response; (e) stimulating cytokine secretion; (f) reducing inhibitory signal transduction through B7-H4; and/or (g) killing tumor cells. The fusion proteins disclosed herein exhibit potent binding and inhibitory activities and are useful for therapeutic and diagnostics uses.

In certain embodiments, the fusion protein comprises a stimulatory domain disclosed herein that is covalently linked to an anti-B7-H4 antibody, or antigen-binding fragment thereof, disclosed herein via a flexible linker. In some embodiments, provided herein is a fusion protein wherein the stimulatory domain disclosed herein is fused directly to an anti-B7-H4 antibody, or antigen-binding fragment thereof, disclosed herein.

As used herein, “covalently linked” or “fused” refers to the association of two or more polypeptides through a covalent bond. In some embodiments, two polypeptides that are covalently linked are fused to each other directly, i.e., without any additional polypeptide sequence between the first and the second peptide. Accordingly, in some embodiments, the N-terminus of the first polypeptide is fused directly to the C-terminus of the second polypeptide or vice versa. In other embodiments, the two polypeptides that are covalently linked are part of a continuous polypeptide chain, but are not directly fused to each other (i.e., the two polypeptides may be separated by one or more amino acids, a linker or another polypeptide). The term “covalently linked” does not imply a specific orientation of the two or more polypeptides that are fused to each other.

IL-15 is a 14-15 kDa cytokine with structural similarity to IL-2. IL-15 is also known as MGC9721. A variety of cell types constitutively produce IL-15 mRNA, and these include monocytes, macrophages, DCs, keratinocytes, epidermal skin cells, fibroblasts, various epithelial cells, bone marrow stromal cells, and nerve cells. In addition, IL-15 mRNA is also produced in kidney, placenta, lung, heart, skeletal muscle, and brain tissues. However, only monocytes, DCs, epithelial cells, bone marrow stromal cells, fibroblasts, and very few other cells and tissues secrete detectable levels of IL-15. IL-15 and IL-2 are found to bind the same hematopoietin subunits and share many biological activities. IL-15 regulates T and NK cell activation and proliferation, and the number of CD8+ memory cells is affected by a balance between IL-15 and IL-2. In embodiments, the IL-15 or IL-15 derivative disclosed herein has at least 10%, at least 20%, at least 30%, at least 40%, at least 50%, at least 60%, at least 70%, at least 80%, or at least 90% of the activity of human IL-15. IL-15 may be a mammalian IL-15, preferably a primate IL-15, and more preferably a human IL-15. The human IL-15 amino acid sequence is provided as SEQ ID NO:164 (full length protein) and SEQ ID NO:165 (mature protein).

The term “IL-15 derivative” refers to a polypeptide having at least 80%, at least 85%, at least 90%, at least 95%, at least 96%, at least 97%, at least 98%, or at least 99% sequence identity with SEQ ID NO:164 (mature form of human IL-15) or with SEQ ID NO:165 (mature form of human IL-15 with N65S mutation). Techniques for making such derivatives are known in the art. In some embodiments, the IL-15 or IL-15 derivative sequence may comprise one or more amino acid substitutions. In embodiments, the fusion protein disclosed herein comprises an IL-15 polypeptide comprising N65S. In embodiments, the fusion protein disclosed herein comprises an IL-15 polypeptide comprising SEQ ID NO:165.

The IL-15 receptor, i.e., IL-15 receptor complex, specifically binds IL-15 with high affinity and consists of a unique interleukin 15 receptor asubunit, IL-2/IL-15Rβ, and the common γ-chain/IL-2Rγ subunit. IL-15Rα is expressed by mitogen-activated macrophages, NK cells, and CD4+ and CD8+ T cells. The human IL-15Rα consists of seven exons, and alternative mRNA splicing may result in eight molecular IL-15Rα isoforms with different extra or intracellular domains. Full-length isoforms consist of an extracellular portion containing a conserved protein binding motif (sushi domain), a trans-membrane domain, and an intracellular tail. As used herein, the term “sushi domain” of IL-15Rα refers to a domain beginning at the first cysteine residue (C1) after the signal peptide of IL-15Rα and ending at the fourth cysteine residue (C4) after said signal peptide. The sushi domain corresponding to a portion of the extracellular region of IL-15Rα is involved in binding to IL-15. The sushi domain in the present disclosure has at least 10%, at least 20%, at least 30%, at least 40%, at least 50%, at least 60%, at least 70%, at least 80%, or at least 90% of the activity of the sushi domain of the human IL-15Rα chain. The sushi domain amino acid sequence of human IL-15Rα is provided in SEQ ID NO:167.

The term “IL-15Rα sushi domain derivative” or “IL-15Rα sushi domain variant” refers to a polypeptide having at least 80%, at least 85%, at least 90%, at least 95%, at least 96%, at least 97%, at least 98%, or at least 99% sequence identity with the sequence of the human IL-15Rα sushi domain (SEQ ID NO:167). Techniques for making such derivatives or variants are known in the art. All such derivatives comprise the four cysteine residues of the sushi domain of IL-15Rα. In some such derivatives, naturally occurring amino acids may be replaced by chemically modified amino acids to alter the polypeptide half-life. In some embodiments, the IL-15Rα sushi domain or IL-15Rα sushi domain derivative sequence may comprise one or more amino acid substitutions.

IL-15 binds with high affinity to IL-15Rα, which then associates with an IL-15Rβ/γc complex expressed by the same target cell (cis-presentation). IL-15Rα is also known to trans-present IL-15 with high affinity to a different target cell expressing the IL-15Rβ/γ(c) complex (trans-presentation). IL-15 cis- and trans-presentation mechanisms lead to different dynamics of receptor activation and signal transduction, with cis-presentation inducing fast and transient responses, and trans-presentation inducing slower, more persistent ones.

In some embodiments, the fusion proteins disclosed herein comprise a stimulatory domain, which comprises a hybrid domain comprising the sushi domain of the IL-15Rα chain attached to IL-15 by a linker. In certain embodiments, the stimulatory domain disclosed herein is covalently linked to an anti-B7-H4 antibody, or antigen-binding fragment thereof, disclosed herein via a second linker. In other embodiments, the stimulatory domain exemplified herein is directly fused to an anti B7-H4 antibody or antigen-binding fragment disclosed herein.

In one embodiment, provided is an “C-terminal fusion protein,” wherein a stimulatory domain is linked directly or via a linker (e.g., via one or more peptides) to the C-terminus of an anti-B7-H4 binding portion of the fusion protein (e.g., the C-terminus of a heavy chain of an anti-B7-H4 antibody disclosed herein).

C-terminal fusion proteins include but are not limited to the following (exemplary) fusion proteins (components recited from N- to C-terminus, “-” indicated direct covalent linkage or linkage via a linker (e.g., via one or more peptides):
(a) (heavy chain of anti-B7-H4 antibody or antigen binding fragment thereof)—(IL-15 or derivative thereof)
(b) (heavy chain of anti-B7-H4 antibody or antigen binding fragment thereof)—(an IL-15Rα polypeptide comprising the IL-15Rα sushi domain or a derivative thereof); and
(c) (heavy chain of anti-B7-H4 antibody or antigen binding fragment thereof)—(IL-15Rα polypeptide comprising the IL-15Rα sushi domain or a derivative thereof)—(IL-15 or derivative thereof)

C-terminal fusion proteins may comprise more than one stimulatory domain.

In one embodiment, provided is a fusion protein, wherein the stimulatory domain is covalently linked to the C-terminus one (and only one) of the heavy chains of the anti-B7-H4 antibody or antigen binding fragment thereof.

Provided herein is a fusion protein comprising (1) a stimulatory domain, (2) a first heavy chain of an anti-B7-H4 antibody or antigen binding fragment thereof, wherein the stimulatory domain is covalently linked to the C-terminus of the first heavy chain, and (3) a second heavy chain of an anti-B7-H4 antibody or antigen binding fragment thereof, wherein the second heavy chain is not linked to a stimulatory domain. In some embodiments, the stimulatory domain comprises (i) IL-15 or a derivative thereof, or (ii) an IL-15Rα polypeptide comprising the IL-15Rα sushi domain or a derivative thereof, or (iii) both.

FIG. 1B depicts a schematic diagram of a non-limiting example of a fusion protein described herein.

In one aspect, provided is a fusion protein comprising:
(a) an anti-B7-H4 antibody, or antigen-binding fragment thereof,
(b) an IL-15Rα sushi domain polypeptide, and
(c) an IL-15 polypeptide.

In one aspect, provided is a fusion protein comprising:
(a) an anti-B7-H4 antibody, or antigen-binding fragment thereof,
(b) an IL-15Rα sushi domain polypeptide,
(c) an IL-15 polypeptide, and
(d) a linker joining the IL-15Rα sushi domain polypeptide and the IL-15 polypeptide.

In one aspect, provided is a fusion protein comprising:
(a) an anti-B7-H4 antibody, or antigen-binding fragment thereof,
(b) a linker joining the anti-B7-H4 antibody, or antigen-binding fragment thereof, and an IL-15Rα sushi domain polypeptide,
(c) the IL-15Rα sushi domain polypeptide,
(d) a linker joining the IL-15Rα sushi domain polypeptide and an IL-15 polypeptide, and
(e) the IL-15 polypeptide.

In one aspect, provided is a fusion protein comprising:
(a) an anti-B7-H4 antibody, or antigen-binding fragment thereof,
(b) an IL-15Rα sushi domain polypeptide comprising SEQ ID NO:167, or an amino acid sequence that at least 80%, at least 85%, at least 90%, at least 95%, at least 96%, at least 97%, at least 98%, or at least 99% identical to SEQ ID NO:167; and
(c) an IL-15 polypeptide comprising SEQ ID NO:166, or an amino acid sequence that is at least 80%, at least 85%, at least 90%, at least 95%, at least 96%, at least 97%, at least 98%, or at least 99% identical to SEQ ID NO:166.

In one aspect, provided is a fusion protein comprising:
(a) an anti-B7-H4 antibody, or antigen-binding fragment thereof,
(b) an IL-15Rα sushi domain polypeptide comprising SEQ ID NO:167, or an amino acid sequence that at least 80%, at least 85%, at least 90%, at least 95%, at least 96%, at least 97%, at least 98%, or at least 99% identical to SEQ ID NO:167;
(c) an IL-15 polypeptide comprising SEQ ID NO:166, or an amino acid sequence that is at least 80%, at least 85%, at least 90%, at least 95%, at least 96%, at least 97%, at least 98%, or at least 99% identical to SEQ ID NO:166, and
(d) a linker joining the IL-15Rα sushi domain polypeptide and the IL-15 polypeptide.

In one embodiment, provided is a fusion protein comprising from N- to C-terminus
(a) a variable heavy chain of an anti-B7-H4 antibody, or antigen-binding fragment thereof,
(b) a constant heavy chain,
(c) an IL-15Rα sushi domain polypeptide comprising SEQ ID NO:167, or an amino acid sequence that at least 80%, at least 85%, at least 90%, at least 95%, at least 96%, at least 97%, at least 98%, or at least 99% identical to SEQ ID NO:167,
(d) a linker joining the IL-15Rα sushi domain polypeptide and an IL-15 polypeptide, and
(e) the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166.

In one embodiment, provided is a fusion protein comprising from N- to C-terminus
(a) a variable heavy chain of an anti-B7-H4 antibody, or antigen-binding fragment thereof,
(b) a constant heavy chain,
(c) a linker joining the constant heavy chain and an IL-15Rα sushi domain polypeptide,
(d) the IL-15Rα sushi domain polypeptide, wherein the IL-15Rα sushi domain polypeptide comprises SEQ ID NO: 167, or an amino acid sequence that at least 80%, at least 85%, at least 90%, at least 95%, at least 96%, at least 97%, at least 98%, or at least 99% identical to SEQ ID NO: 167,
(e) a linker joining the IL-15Rα sushi domain polypeptide and an IL-15 polypeptide, and
(f) the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO: 166.

In one embodiment, provided is a fusion protein comprising
(a) a first chain comprising from N- to C-terminus:
   a. a variable heavy chain of an anti-B7-H4 antibody, or antigen-binding fragment thereof,
   b. a constant heavy chain,
   c. an IL-15Rα sushi domain polypeptide comprising SEQ ID NO:167, or an amino acid sequence that at least 80%, at least 85%, at least 90%, at least 95%, at least 96%, at least 97%, at least 98%, or at least 99% identical to SEQ ID NO:167,
   d. a linker joining the IL-15Rα sushi domain polypeptide and an IL-15 polypeptide, and
   e. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable light chain of an anti-B7-H4 antibody, or antigen-binding fragment thereof, and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof,
   b. a constant heavy chain,
   c. a linker joining the constant heavy chain and an IL-15Rα sushi domain polypeptide,
   d. the IL-15Rα sushi domain polypeptide, wherein the IL-15Rα sushi domain polypeptide comprises SEQ ID NO:167, or an amino acid sequence that at least 80%, at least 85%, at least 90%, at least 95%, at least 96%, at least 97%, at least 98%, or at least 99% identical to SEQ ID NO:167,
   e. a linker joining the IL-15Rα sushi domain polypeptide and an IL-15 polypeptide, and
   f. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166, and
(b) a second chain comprising from N- to C-terminus:
   a. a variable light chain of an anti-B7-H4 antibody, or antigen-binding fragment thereof, and
   b. a constant light chain;
optionally wherein the constant heavy chain comprises one or modifications causing heterodimerization.

In one embodiment, provided is a fusion protein comprising
(a) a first heavy chain comprising from N- to C-terminus:
   a. a first variable heavy chain of an anti-B7-H4 antibody, or antigen-binding fragment thereof,
   b. a first constant heavy chain,
   c. an IL-15Rα sushi domain polypeptide comprising SEQ ID NO:167, or an amino acid sequence that at least 80%, at least 85%, at least 90%, at least 95%, at least 96%, at least 97%, at least 98%, or at least 99% identical to SEQ ID NO:167,
   d. a linker joining the IL-15Rα sushi domain polypeptide and an IL-15 polypeptide, and
   e. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166;
(b) a second heavy chain comprising from N- to C-terminus:
   a. a second variable heavy chain of an anti-B7-H4 antibody, or antigen-binding fragment thereof, and
   b. a second constant heavy chain; and
(c) a light chain comprising from N- to C-terminus:
   a. a variable light chain of an anti-B7-H4 antibody, or antigen-binding fragment thereof, and
   b. a constant light chain;
optionally wherein the first and the second constant heavy chains comprise one or modifications causing heterodimerization.

In one embodiment, provided is a fusion protein comprising
(a) a first heavy chain comprising from N- to C-terminus:
   a. a first variable heavy chain of an anti-B7-H4 antibody, or antigen-binding fragment thereof,
   b. a first constant heavy chain,
   c. a linker joining the constant heavy chain and an IL-15Rα sushi domain polypeptide,
   d. the IL-15Rα sushi domain polypeptide, wherein the IL-15Rα sushi domain polypeptide comprises SEQ ID NO:167, or an amino acid sequence that at least 80%, at least 85%, at least 90%, at least 95%, at least 96%, at least 97%, at least 98%, or at least 99% identical to SEQ ID NO:167,
   e. a linker joining the IL-15Rα sushi domain polypeptide and an IL-15 polypeptide, and
   f. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166;
(b) a second heavy chain comprising from N- to C-terminus:
   a. a second variable heavy chain of an anti-B7-H4 antibody, or antigen-binding fragment thereof, and
   b. a second constant heavy chain; and
(c) a light chain comprising from N- to C-terminus:
   a. a variable light chain of an anti-B7-H4 antibody, or antigen-binding fragment thereof, and
   b. a constant light chain;
optionally wherein the first and the second constant heavy chains comprise one or modifications causing heterodimerization.

In one embodiment, the IL-15Rα sushi domain polypeptide comprises SEQ ID NO:167. In one embodiment, the IL-15Rα sushi domain polypeptide consists of SEQ ID NO:167.

In one embodiment, the IL-15 polypeptide comprises SEQ ID NO:166. In one embodiment, the IL-15 polypeptide consists of SEQ ID NO:166.

In one embodiment, the linker joining the IL-15Rα sushi domain polypeptide and the IL-15 polypeptide comprises SEQ ID NO:168. In one embodiment, the linker joining the IL-15Rα sushi domain polypeptide and the IL-15 polypeptide consists of SEQ ID NO:168.

In one embodiment, the fusion protein comprises SEQ ID NO:169.

In one aspect, provided is a fusion protein comprising an anti-B7-H4 antibody, or antigen-binding fragment thereof, wherein the antibody, or antigen-binding fragment thereof, comprises a heavy chain variable region and a light chain variable region, wherein each of the heavy chain and the light chain variable regions comprise a CDR1, CDR2, and CDR3.

In one embodiment, the fusion protein comprises an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO:73, CDR2L comprises SEQ ID NO:74, and CDR3L comprises SEQ ID NO:75.

Provided herein is a fusion protein comprising an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO:20, CDR2L comprises SEQ ID NO:21, and CDR3L comprises SEQ ID NO:22.

Provided herein is a fusion protein comprising an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO:66, CDR2L comprises SEQ ID NO:67, and CDR3L comprises SEQ ID NO:68.

Provided herein is a fusion protein comprising an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO:70, CDR2L comprises SEQ ID NO:71, and CDR3L comprises SEQ ID NO:72.

Provided herein is a fusion protein comprising an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO: 84, CDR2L comprises SEQ ID NO:85, and CDR3L comprises SEQ ID NO:86.

Provided herein is a fusion protein comprising an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO: 8, CDR2L comprises SEQ ID NO: 9, and CDR3L comprises SEQ ID NO:10.

Provided herein is a fusion protein comprising an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO: 77, CDR2L comprises SEQ ID NO: 78, and CDR3L comprises SEQ ID NO: 79.

Provided herein is a fusion protein comprising an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO: 81, CDR2L comprises SEQ ID NO:82, and CDR3L comprises SEQ ID NO:83.

Provided herein is a fusion protein comprising an anti-B7-H4 antibody, or antigen-binding fragment thereof, wherein the heavy chain variable region comprises SEQ ID NO: 15, or a sequence that is at least 90%, at least 95%, at least 96%, at least 97%, at least 98%, or at least 99% identical to SEQ ID NO: 15; and wherein the light chain variable region comprises SEQ ID NO: x, or a sequence that is at least 90%, at least 95%, at least 96%, at least 97%, at least 98%, or at least 99% identical to SEQ ID NO: 19.

Provided herein is a fusion protein comprising an anti-B7-H4 antibody, or antigen-binding fragment thereof, wherein the heavy chain variable region comprises SEQ ID NO: 15, or a sequence that is at least 90%, at least 95%, at least 96%, at least 97%, at least 98%, or at least 99% identical to SEQ ID NO: 15; and wherein the light chain variable region comprises SEQ ID NO: 65, or a sequence that is at least 90%, at least 95%, at least 96%, at least 97%, at least 98%, or at least 99% identical to SEQ ID NO: 65.

Provided herein is a fusion protein comprising an anti-B7-H4 antibody, or antigen-binding fragment thereof, wherein the heavy chain variable region comprises SEQ ID NO: 15, or a sequence that is at least 90%, at least 95%, at least 96%, at least 97%, at least 98%, or at least 99% identical to SEQ ID NO: 15; and wherein the light chain variable region comprises SEQ ID NO: 69, or a sequence that is at least 90%, at least 95%, at least 96%, at least 97%, at least 98%, or at least 99% identical to SEQ ID NO: 69.

Provided herein is a fusion protein comprising an anti-B7-H4 antibody, or antigen-binding fragment thereof, wherein the heavy chain variable region comprises SEQ ID NO: 3, or a sequence that is at least 90%, at least 95%, at least 96%, at least 97%, at least 98%, or at least 99% identical to SEQ ID NO: 3 and wherein the light chain variable region comprises SEQ ID NO: 7, or a sequence that is at least 90%, at least 95%, at least 96%, at least 97%, at least 98%, or at least 99% identical to SEQ ID NO: 7.

Provided herein is a fusion protein comprising an anti-B7-H4 antibody, or antigen-binding fragment thereof, wherein the heavy chain variable region comprises SEQ ID NO: 3, or a sequence that is at least 90%, at least 95%, at least 96%, at least 97%, at least 98%, or at least 99% identical to SEQ ID NO: 3 and wherein the light chain variable region comprises SEQ ID NO: 76, or a sequence that is at least 90%, at least 95%, at least 96%, at least 97%, at least 98%, or at least 99% identical to SEQ ID NO: 76.

Provided herein is a fusion protein comprising an anti-B7-H4 antibody, or antigen-binding fragment thereof, wherein the heavy chain variable region comprises SEQ ID NO: 3, or a sequence that is at least 90%, at least 95%, at least 96%, at least 97%, at least 98%, or at least 99% identical to SEQ ID NO: 3 and wherein the light chain variable region comprises SEQ ID NO: 80, or a sequence that is at least 90%, at least 95%, at least 96%, at least 97%, at least 98%, or at least 99% identical to SEQ ID NO: 80.

Provided herein is a fusion protein comprising an anti-B7-H4 antibody, or antigen-binding fragment thereof, wherein the heavy chain variable region comprises SEQ ID NO: 15 and wherein the light chain variable region comprises SEQ ID NO: 19.

Provided herein is a fusion protein comprising an anti-B7-H4 antibody, or antigen-binding fragment thereof, wherein the heavy chain variable region comprises SEQ ID NO: 15 and wherein the light chain variable region comprises SEQ ID NO: 65.

Provided herein is a fusion protein comprising an anti-B7-H4 antibody, or antigen-binding fragment thereof, wherein the heavy chain variable region comprises SEQ ID NO: 15 and wherein the light chain variable region comprises SEQ ID NO: 69.

Provided herein is a fusion protein comprising an anti-B7-H4 antibody, or antigen-binding fragment thereof, wherein the heavy chain variable region comprises SEQ ID NO: 3 and wherein the light chain variable region comprises SEQ ID NO: 7.

Provided herein is a fusion protein comprising an anti-B7-H4 antibody, or antigen-binding fragment thereof, wherein the heavy chain variable region comprises SEQ ID NO: 3 and wherein the light chain variable region comprises SEQ ID NO: 76.

Provided herein is a fusion protein comprising an anti-B7-H4 antibody, or antigen-binding fragment thereof, wherein the heavy chain variable region comprises SEQ ID NO: 3 and wherein the light chain variable region comprises SEQ ID NO: 80.

In one aspect, provided is an anti-B7-H4 antibody, or antigen-binding fragment thereof, wherein the antibody, or antigen-binding fragment thereof, comprises a heavy chain variable region and a light chain variable region, wherein each of the heavy chain and the light chain variable regions comprise a CDR1, CDR2, and CDR3.

Provided herein is a fusion protein comprising an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:111, CDR2L comprises SEQ ID NO:112, and CDR3L comprises SEQ ID NO:113.

Provided herein is a fusion protein comprising an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:50, CDR2L comprises SEQ ID NO:51, and CDR3L comprises SEQ ID NO:52.

Provided herein is a fusion protein comprising an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:88, CDR2L comprises SEQ ID NO:89, and CDR3L comprises SEQ ID NO:90.

Provided herein is a fusion protein comprising an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:92, CDR2L comprises SEQ ID NO:93, and CDR3L comprises SEQ ID NO:94.

Provided herein is a fusion protein comprising an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:96, CDR2L comprises SEQ ID NO:97, and CDR3L comprises SEQ ID NO:98.

Provided herein is a fusion protein comprising an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:100, CDR2L comprises SEQ ID NO:101, and CDR3L comprises SEQ ID NO:102.

Provided herein is a fusion protein comprising an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:104, CDR2L comprises SEQ ID NO:105, and CDR3L comprises SEQ ID NO:106.

Provided herein is a fusion protein comprising an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:108, CDR2L comprises SEQ ID NO:109, and CDR3L comprises SEQ ID NO:110.

Provided herein is a fusion protein comprising an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:49, or a sequence that is at least 90%, at least 95%, at least 96%, at least 97%, at least 98%, or at least 99% identical to SEQ ID NO:49.

Provided herein is a fusion protein comprising an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:87, or a sequence that is at least 90%, at least 95%, at least 96%, at least 97%, at least 98%, or at least 99% identical to SEQ ID NO:87.

Provided herein is a fusion protein comprising an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:91, or a sequence that is at least 90%, at least 95%, at least 96%, at least 97%, at least 98%, or at least 99% identical to SEQ ID NO:91.

Provided herein is a fusion protein comprising an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:95, or a sequence that is at least 90%, at least 95%, at least 96%, at least 97%, at least 98%, or at least 99% identical to SEQ ID NO:95.

Provided herein is a fusion protein comprising an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:99, or a sequence that is at least 90%, at least 95%, at least 96%, at least 97%, at least 98%, or at least 99% identical to SEQ ID NO:99.

Provided herein is a fusion protein comprising an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:103, or a sequence that is at least 90%, at least 95%, at least 96%, at least 97%, at least 98%, or at least 99% identical to SEQ ID NO:103.

Provided herein is a fusion protein comprising an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:107, or a sequence that is at least 90%, at least 95%, at least 96%, at least 97%, at least 98%, or at least 99% identical to SEQ ID NO:107.

Provided herein is a fusion protein comprising an anti-B7-H4 antibody, or antigen-binding fragment thereof, wherein the heavy chain variable region comprises SEQ ID NO: 45 and wherein the light chain variable region comprises SEQ ID NO:49.

Provided herein is a fusion protein comprising an anti-B7-H4 antibody, or antigen-binding fragment thereof, wherein the heavy chain variable region comprises SEQ ID NO: 45 and wherein the light chain variable region comprises SEQ ID NO:87.

Provided herein is a fusion protein comprising an anti-B7-H4 antibody, or antigen-binding fragment thereof, wherein the heavy chain variable region comprises SEQ ID NO: 45 and wherein the light chain variable region comprises SEQ ID NO:91.

Provided herein is a fusion protein comprising an anti-B7-H4 antibody, or antigen-binding fragment thereof, wherein the heavy chain variable region comprises SEQ ID NO: 45 and wherein the light chain variable region comprises SEQ ID NO:95.

Provided herein is a fusion protein comprising an anti-B7-H4 antibody, or antigen-binding fragment thereof, wherein the heavy chain variable region comprises SEQ ID NO: 45 and wherein the light chain variable region comprises SEQ ID NO:99.

Provided herein is a fusion protein comprising an anti-B7-H4 antibody, or antigen-binding fragment thereof, wherein the heavy chain variable region comprises SEQ ID NO: 45 and wherein the light chain variable region comprises SEQ ID NO:103.

Provided herein is a fusion protein comprising an anti-B7-H4 antibody, or antigen-binding fragment thereof, wherein the heavy chain variable region comprises SEQ ID NO: 45 and wherein the light chain variable region comprises SEQ ID NO:107.

In one aspect, provided is an anti-B7-H4 antibody, or antigen-binding fragment thereof, wherein the antibody, or antigen-binding fragment thereof, comprises a heavy chain variable region and a light chain variable region, wherein each of the heavy chain and the light chain variable regions comprise a CDR1, CDR2, and CDR3.

Provided herein is a fusion protein comprising an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:134, CDR2L comprises SEQ ID NO:135, and CDR3L comprises SEQ ID NO:136.

Provided herein is a fusion protein comprising an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:62, CDR2L comprises SEQ ID NO:63, and CDR3L comprises SEQ ID NO:64.

Provided herein is a fusion protein comprising an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:115, CDR2L comprises SEQ ID NO:116, and CDR3L comprises SEQ ID NO:117.

Provided herein is a fusion protein comprising an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:119, CDR2L comprises SEQ ID NO:120, and CDR3L comprises SEQ ID NO:121.

Provided herein is a fusion protein comprising an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:123, CDR2L comprises SEQ ID NO:124, and CDR3L comprises SEQ ID NO:125.

Provided herein is a fusion protein comprising an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:127, CDR2L comprises SEQ ID NO:128, and CDR3L comprises SEQ ID NO:129.

Provided herein is a fusion protein comprising an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:131, CDR2L comprises SEQ ID NO:132, and CDR3L comprises SEQ ID NO:133.

Provided herein is a fusion protein comprising an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:61, or a sequence that is at least 90%, at least 95%, at least 96%, at least 97%, at least 98%, or at least 99% identical to SEQ ID NO:61.

Provided herein is a fusion protein comprising an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:114, or a sequence that is at least 90%, at least 95%, at least 96%, at least 97%, at least 98%, or at least 99% identical to SEQ ID NO:114.

Provided herein is a fusion protein comprising an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:118, or a sequence that is at least 90%, at least 95%, at least 96%, at least 97%, at least 98%, or at least 99% identical to SEQ ID NO:118.

Provided herein is a fusion protein comprising an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:122, or a sequence that is at least 90%, at least 95%, at least 96%, at least 97%, at least 98%, or at least 99% identical to SEQ ID NO:122.

Provided herein is a fusion protein comprising an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:126, or a sequence that is at least 90%, at least 95%, at least 96%, at least 97%, at least 98%, or at least 99% identical to SEQ ID NO:126. or

Provided herein is a fusion protein comprising an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:130, or a sequence that is at least 90%, at least 95%, at least 96%, at least 97%, at least 98%, or at least 99% identical to SEQ ID NO:130.

Provided herein is a fusion protein comprising an anti-B7-H4 antibody, or antigen-binding fragment thereof, wherein the heavy chain variable region comprises SEQ ID NO:57 and wherein the light chain variable region comprises SEQ ID NO:61.

Provided herein is a fusion protein comprising an anti-B7-H4 antibody, or antigen-binding fragment thereof, wherein the heavy chain variable region comprises SEQ ID NO:57 and wherein the light chain variable region comprises SEQ ID NO:114.

Provided herein is a fusion protein comprising an anti-B7-H4 antibody, or antigen-binding fragment thereof, wherein the heavy chain variable region comprises SEQ ID NO:57 and wherein the light chain variable region comprises SEQ ID NO:118.

Provided herein is a fusion protein comprising an anti-B7-H4 antibody, or antigen-binding fragment thereof, wherein the heavy chain variable region comprises SEQ ID NO:57 and wherein the light chain variable region comprises SEQ ID NO:122.

Provided herein is a fusion protein comprising an anti-B7-H4 antibody, or antigen-binding fragment thereof, wherein the heavy chain variable region comprises SEQ ID NO:57 and wherein the light chain variable region comprises SEQ ID NO:126.

Provided herein is a fusion protein comprising an anti-B7-H4 antibody, or antigen-binding fragment thereof, wherein the heavy chain variable region comprises SEQ ID NO:57 and wherein the light chain variable region comprises SEQ ID NO:130.

In one embodiment, provided is a fusion protein comprising
(a) a first chain comprising from N- to C-terminus:
   a. a variable heavy chain of an anti-B7-H4 antibody, or antigen-binding fragment thereof, wherein the variable heavy chain comprises a CDR1H, CDR2H, and CDR3H, wherein CDR1H comprises SEQ ID NO:16, CDR2H comprises SEQ ID NO:17, CDR3H comprises SEQ ID NO:18,
   b. a constant heavy chain,
   c. an IL-15Rα sushi domain polypeptide comprising SEQ ID NO:167, or an amino acid sequence that at least 80%, at least 85%, at least 90%, at least 95%, at least 96%, at least 97%, at least 98%, or at least 99% identical to SEQ ID NO:167,
   d. a linker joining the IL-15Rα sushi domain polypeptide and an IL-15 polypeptide, and
   e. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166, and
(b) a second chain comprising from N- to C-terminus:
   a. a variable light chain of an anti-B7-H4 antibody, or antigen-binding fragment thereof, wherein the variable light chain comprises a CDR1L, CDR2L, and CDR3L, wherein CDR1L comprises SEQ ID NO:70, CDR2L comprises SEQ ID NO:71, and CDR3L comprises SEQ ID NO:72, and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof, wherein the variable heavy chain comprises a CDR1H, CDR2H, and CDR3H, wherein CDR1H comprises SEQ ID NO:16, CDR2H comprises SEQ ID NO:17, CDR3H comprises SEQ ID NO:18,
   b. a constant heavy chain,
   c. a linker joining the constant heavy chain and an IL-15Rα sushi domain polypeptide,
   d. the IL-15Rα sushi domain polypeptide, wherein the IL-15Rα sushi domain polypeptide comprises SEQ ID NO:167, or an amino acid sequence that at least 80%, at least 85%, at least 90%, at least 95%, at least 96%, at least 97%, at least 98%, or at least 99% identical to SEQ ID NO:167,
   e. a linker joining the IL-15Rα sushi domain polypeptide and an IL-15 polypeptide, and
   f. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable heavy chain of an anti-B7-H4 antibody, or antigen-binding fragment thereof, wherein the variable heavy chain comprises a CDR1H, CDR2H, and CDR3H, wherein CDR1H comprises SEQ ID NO:16, CDR2H comprises SEQ ID NO:17, CDR3H comprises SEQ ID NO:18; and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof, wherein the variable heavy chain comprises a CDR1H, CDR2H, and CDR3H, wherein CDR1H comprises SEQ ID NO:46, CDR2H comprises SEQ ID NO:47, CDR3H comprises SEQ ID NO:48,
   b. a constant heavy chain,
   c. an IL-15Rα sushi domain polypeptide comprising SEQ ID NO:167, or an amino acid sequence that at least 80%, at least 85%, at least 90%, at least 95%, at least 96%, at least 97%, at least 98%, or at least 99% identical to SEQ ID NO:167,
   d. a linker joining the IL-15Rα sushi domain polypeptide and an IL-15 polypeptide, and
   e. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable light chain of an anti-B7-H4 antibody, or antigen-binding fragment thereof, wherein the variable light chain comprises a CDR1L, CDR2L, and CDR3L, wherein CDR1L comprises SEQ ID NO:108, CDR2L comprises SEQ ID NO:109, and CDR3L comprises SEQ ID NO:110, and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof, wherein the variable heavy chain comprises a CDR1H, CDR2H, and CDR3H, wherein CDR1H comprises SEQ ID NO:46, CDR2H comprises SEQ ID NO:47, CDR3H comprises SEQ ID NO:48,
   b. a constant heavy chain,
   c. a linker joining the constant heavy chain and an IL-15Rα sushi domain polypeptide,
   d. the IL-15Rα sushi domain polypeptide, wherein the IL-15Rα sushi domain polypeptide comprises SEQ ID NO:167, or an amino acid sequence that at least 80%, at least 85%, at least 90%, at least 95%, at least 96%, at least 97%, at least 98%, or at least 99% identical to SEQ ID NO:167,
   e. a linker joining the IL-15Rα sushi domain polypeptide and an IL-15 polypeptide, and
   f. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable light chain of an anti-B7-H4 antibody, or antigen-binding fragment thereof, wherein the variable light chain comprises a CDR1L, CDR2L, and CDR3L, wherein CDR1L comprises SEQ ID NO:108, CDR2L comprises SEQ ID NO:109, and CDR3L comprises SEQ ID NO:110, and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof, wherein the variable heavy chain comprises a CDR1H, CDR2H, and CDR3H, wherein CDR1H comprises SEQ ID NO:58, CDR2H comprises SEQ ID NO:59, CDR3H comprises SEQ ID NO:60,
   b. a constant heavy chain,
   c. an IL-15Rα sushi domain polypeptide comprising SEQ ID NO:167, or an amino acid sequence that at least 80%, at least 85%, at least 90%, at least 95%, at least 96%, at least 97%, at least 98%, or at least 99% identical to SEQ ID NO:167,
   d. a linker joining the IL-15Rα sushi domain polypeptide and an IL-15 polypeptide, and
   e. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable light chain of an anti-B7-H4 antibody, or antigen-binding fragment thereof, wherein the variable light chain comprises a CDR1L, CDR2L, and CDR3L, wherein CDR1L comprises SEQ ID NO:119, CDR2L comprises SEQ ID NO:120, and CDR3L comprises SEQ ID NO:121, and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof, wherein the variable heavy chain comprises a CDR1H, CDR2H, and CDR3H, wherein CDR1H comprises SEQ ID NO:58, CDR2H comprises SEQ ID NO:59, CDR3H comprises SEQ ID NO:60,
   b. a constant heavy chain,
   c. a linker joining the constant heavy chain and an IL-15Rα sushi domain polypeptide,
   d. the IL-15Rα sushi domain polypeptide, wherein the IL-15Rα sushi domain polypeptide comprises SEQ ID NO:167, or an amino acid sequence that at least 80%, at least 85%, at least 90%, at least 95%, at least 96%, at least 97%, at least 98%, or at least 99% identical to SEQ ID NO:167,
   e. a linker joining the IL-15Rα sushi domain polypeptide and an IL-15 polypeptide,
   f. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable light chain of an anti-B7-H4 antibody, or antigen-binding fragment thereof, wherein the variable light chain comprises a CDR1L, CDR2L, and CDR3L, wherein CDR1L comprises SEQ ID NO:119, CDR2L comprises SEQ ID NO:120, and CDR3L comprises SEQ ID NO:121, and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof, comprising SEQ ID NO: 15 or an amino acid sequence that at least 80%, at least 85%, at least 90%, at least 95%, at least 96%, at least 97%, at least 98%, or at least 99% identical to SEQ ID NO: 15,
   b. a constant heavy chain,
   c. an IL-15Rα sushi domain polypeptide comprising SEQ ID NO:167, or an amino acid sequence that at least 80%, at least 85%, at least 90%, at least 95%, at least 96%, at least 97%, at least 98%, or at least 99% identical to SEQ ID NO:167,
   d. a linker joining the IL-15Rα sushi domain polypeptide and an IL-15 polypeptide, and
   e. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable light chain of an anti-B7-H4 antibody, or antigen-binding fragment thereof, comprising SEQ ID NO: 69 or an amino acid sequence that at least 80%, at least 85%, at least 90%, at least 95%, at least 96%, at least 97%, at least 98%, or at least 99% identical to SEQ ID NO: 69, and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof, comprising SEQ ID NO: 15 or an amino acid sequence that at least 80%, at least 85%, at least 90%, at least 95%, at least 96%, at least 97%, at least 98%, or at least 99% identical to SEQ ID NO: 15,
   b. a constant heavy chain,
   c. a linker joining the constant heavy chain and an IL-15Rα sushi domain polypeptide,
   d. the IL-15Rα sushi domain polypeptide, wherein the IL-15Rα sushi domain polypeptide comprises SEQ ID NO:167, or an amino acid sequence that at least 80%, at least 85%, at least 90%, at least 95%, at least 96%, at least 97%, at least 98%, or at least 99% identical to SEQ ID NO:167,
   e. a linker joining the IL-15Rα sushi domain polypeptide and an IL-15 polypeptide, and
   f. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable light chain of an anti-B7-H4 antibody, or antigen-binding fragment thereof, comprising SEQ ID NO: 69 or an amino acid sequence that at least 80%, at least 85%, at least 90%, at least 95%, at least 96%, at least 97%, at least 98%, or at least 99% identical to SEQ ID NO:69, and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof, comprising SEQ ID NO: 45 or an amino acid sequence that at least 80%, at least 85%, at least 90%, at least 95%, at least 96%, at least 97%, at least 98%, or at least 99% identical to SEQ ID NO:45,
   b. a constant heavy chain,
   c. an IL-15Rα sushi domain polypeptide comprising SEQ ID NO:167, or an amino acid sequence that at least 80%, at least 85%, at least 90%, at least 95%, at least 96%, at least 97%, at least 98%, or at least 99% identical to SEQ ID NO:167,
   d. a linker joining the IL-15Rα sushi domain polypeptide and an IL-15 polypeptide, and
   e. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable heavy chain of an anti-B7-H4 antibody, or antigen-binding fragment thereof, comprising SEQ ID NO: 107 or an amino acid sequence that at least 80%, at least 85%, at least 90%, at least 95%, at least 96%, at least 97%, at least 98%, or at least 99% identical to SEQ ID NO:107, and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof, comprising SEQ ID NO: 45 or an amino acid sequence that at least 80%, at least 85%, at least 90%, at least 95%, at least 96%, at least 97%, at least 98%, or at least 99% identical to SEQ ID NO:45,
   b. a constant heavy chain,
   c. a linker joining the constant heavy chain and an IL-15Rα sushi domain polypeptide,
   d. the IL-15Rα sushi domain polypeptide, wherein the IL-15Rα sushi domain polypeptide comprises SEQ ID NO:167, or an amino acid sequence that at least 80%, at least 85%, at least 90%, at least 95%, at least 96%, at least 97%, at least 98%, or at least 99% identical to SEQ ID NO:167,
   e. a linker joining the IL-15Rα sushi domain polypeptide and an IL-15 polypeptide, and
   f. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable heavy chain of an anti-B7-H4 antibody, or antigen-binding fragment thereof, comprising SEQ ID NO: 107 or an amino acid sequence that at least 80%, at least 85%, at least 90%, at least 95%, at least 96%, at least 97%, at least 98%, or at least 99% identical to SEQ ID NO:107, and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof, comprising SEQ ID NO: 57 or an amino acid sequence that at least 80%, at least 85%, at least 90%, at least 95%, at least 96%, at least 97%, at least 98%, or at least 99% identical to SEQ ID NO:57,
   b. a constant heavy chain,
   c. an IL-15Rα sushi domain polypeptide comprising SEQ ID NO:167, or an amino acid sequence that at least 80%, at least 85%, at least 90%, at least 95%, at least 96%, at least 97%, at least 98%, or at least 99% identical to SEQ ID NO:167,
   d. a linker joining the IL-15Rα sushi domain polypeptide and an IL-15 polypeptide, and
   e. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable heavy chain of an anti-B7-H4 antibody, or antigen-binding fragment thereof, comprising SEQ ID NO: 118 or an amino acid sequence that at least 80%, at least 85%, at least 90%, at least 95%, at least 96%, at least 97%, at least 98%, or at least 99% identical to SEQ ID NO:118, and
   b. a constant light chain;
optionally wherein the constant heavy chain comprises one or modifications causing heterodimerization.

In one embodiment, provided is a fusion protein comprising
(a) a first chain comprising from N- to C-terminus:
   a. a variable heavy chain of an anti-B7-H4 antibody, or antigen-binding fragment thereof, comprising SEQ ID NO: 57 or an amino acid sequence that at least 80%, at least 85%, at least 90%, at least 95%, at least 96%, at least 97%, at least 98%, or at least 99% identical to SEQ ID NO:57,
   b. a constant heavy chain,
   c. a linker joining the constant heavy chain and an IL-15Rα sushi domain polypeptide,
   d. the IL-15Rα sushi domain polypeptide, wherein the IL-15Rα sushi domain polypeptide comprises SEQ ID NO:167, or an amino acid sequence that at least 80%, at least 85%, at least 90%, at least 95%, at least 96%, at least 97%, at least 98%, or at least 99% identical to SEQ ID NO:167,
   e. a linker joining the IL-15Rα sushi domain polypeptide and an IL-15 polypeptide, and
   f. the IL-15 polypeptide, wherein the IL-15 polypeptide comprises SEQ ID NO:166, or an amino acid sequence that is at least 80%, at least 85%, at least 90%, at least 95%, at least 96%, at least 97%, at least 98%, or at least 99% identical to SEQ ID NO:166; and
(b) a second chain comprising from N- to C-terminus:
   a. a variable heavy chain of an anti-B7-H4 antibody, or antigen-binding fragment thereof, comprising SEQ ID NO: 118 or an amino acid sequence that at least 80%, at least 85%, at least 90%, at least 95%, at least 96%, at least 97%, at least 98%, or at least 99% identical to SEQ ID NO:118, and
   b. a constant light chain;
optionally wherein the constant heavy chain comprises one or modifications causing heterodimerization.

In one embodiment, provided is a fusion protein comprising:
(a) a light chain comprising SEQ ID NO:69 or an amino acid sequence that is at least 80%, at least 85%, at least 90%, at least 95%, at least 96%, at least 97%, at least 98%, or at least 99% identical to SEQ ID NO:69 and/or SEQ ID NO:145 or an amino acid sequence that is at least 80%, at least 85%, at least 90%, at least 95%, at least 96%, at least 97%, at least 98%, or at least 99% identical to SEQ ID NO:145;
(b) a first heavy chain comprising SEQ ID NO:150 or an amino acid sequence that is at least 80%, at least 85%, at least 90%, at least 95%, at least 96%, at least 97%, at least 98%, or at least 99% identical to SEQ ID NO:150; and
(c) a second heavy chain comprising SEQ ID NO:157 or an amino acid sequence that is at least 80%, at least 85%, at least 90%, at least 95%, at least 96%, at least 97%, at least 98%, or at least 99% identical to SEQ ID NO:157.

In one embodiment, provided is a fusion protein comprising:
(a) a light chain comprising SEQ ID NO:69 or an amino acid sequence that is at least 80%, at least 85%, at least 90%, at least 95%, at least 96%, at least 97%, at least 98%, or at least 99% identical to SEQ ID NO:69 and/or SEQ ID NO:145 or an amino acid sequence that is at least 80%, at least 85%, at least 90%, at least 95%, at least 96%, at least 97%, at least 98%, or at least 99% identical to SEQ ID NO:145;
(b) a first heavy chain comprising SEQ ID NO:151 or an amino acid sequence that is at least 80%, at least 85%, at least 90%, at least 95%, at least 96%, at least 97%, at least 98%, or at least 99% identical to SEQ ID NO:151; and
(c) a second heavy chain comprising SEQ ID NO:158 or an amino acid sequence that is at least 80%, at least 85%, at least 90%, at least 95%, at least 96%, at least 97%, at least 98%, or at least 99% identical to SEQ ID NO:158.

In one embodiment, provided is a fusion protein comprising:
(a) a light chain comprising SEQ ID NO:118 or an amino acid sequence that is at least 80%, at least 85%, at least 90%, at least 95%, at least 96%, at least 97%, at least 98%, or at least 99% identical to SEQ ID NO:118 and/or SEQ ID NO:144 or an amino acid sequence that is at least 80%, at least 85%, at least 90%, at least 95%, at least 96%, at least 97%, at least 98%, or at least 99% identical to SEQ ID NO:144;
(b) a first heavy chain comprising SEQ ID NO:148 or an amino acid sequence that is at least 80%, at least 85%, at least 90%, at least 95%, at least 96%, at least 97%, at least 98%, or at least 99% identical to SEQ ID NO:148; and
(c) a second heavy chain comprising SEQ ID NO:155 or an amino acid sequence that is at least 80%, at least 85%, at least 90%, at least 95%, at least 96%, at least 97%, at least 98%, or at least 99% identical to SEQ ID NO:155.

In one embodiment, provided is a fusion protein comprising:
(a) a light chain comprising SEQ ID NO:118 or an amino acid sequence that is at least 80%, at least 85%, at least 90%, at least 95%, at least 96%, at least 97%, at least 98%, or at least 99% identical to SEQ ID NO:118 and/or SEQ ID NO:144 or an amino acid sequence that is at least 80%, at least 85%, at least 90%, at least 95%, at least 96%, at least 97%, at least 98%, or at least 99% identical to SEQ ID NO:144;
(b) a first heavy chain comprising SEQ ID NO:149 or an amino acid sequence that is at least 80%, at least 85%, at least 90%, at least 95%, at least 96%, at least 97%, at least 98%, or at least 99% identical to SEQ ID NO:149; and
(c) a second heavy chain comprising SEQ ID NO:156 or an amino acid sequence that is at least 80%, at least 85%, at least 90%, at least 95%, at least 96%, at least 97%, at least 98%, or at least 99% identical to SEQ ID NO:156.

In one embodiment, provided is a fusion protein comprising:
(a) a light chain comprising SEQ ID NO:69 and/or SEQ ID NO: 145;
(b) a first heavy chain comprising SEQ ID NO:150; and
(c) a second heavy chain comprising SEQ ID NO:157.

In one embodiment, provided is a fusion protein comprising:
(a) a light chain comprising SEQ ID NO:69 and/or SEQ ID NO: 145;
(b) a first heavy chain comprising SEQ ID NO:151; and
(c) a second heavy chain comprising SEQ ID NO:158.

In one embodiment, provided is a fusion protein comprising:
(a) a light chain comprising SEQ ID NO:118 and/or SEQ ID NO: 144;
(b) a first heavy chain comprising SEQ ID NO:148; and
(c) a second heavy chain comprising SEQ ID NO:155.

In one embodiment, provided is a fusion protein comprising:
(a) a light chain comprising SEQ ID NO:118 and/or SEQ ID NO: 144;
(b) a first heavy chain comprising SEQ ID NO:149; and
(c) a second heavy chain comprising SEQ ID NO:156.

In one embodiment, provided is a fusion protein comprising:
(a) a light chain comprising SEQ ID NO:145;
(b) a first heavy chain comprising SEQ ID NO:150; and
(c) a second heavy chain comprising SEQ ID NO:157.

Provided herein is a fusion protein comprising one or more sequences disclosed in Tables 13, 21, or 22.

Antibodies and Antigen-Binding Fragments Thereof

The term antibody is used here in the broadest sense and includes monoclonal antibodies (including full length or intact monoclonal antibodies), polyclonal antibodies, bispecific antibodies, humanized antibodies, single chain antibodies, chimeric antibodies, synthetic antibodies, recombinant antibodies, hybrid antibodies, mutagenized antibodies and grafted antibodies (grafted antibodies), bispecific antibodies, a specific antibody portion (e.g., a domain antibody), as well as any antigen-binding portion thereof that competes with an intact antibody for specific binding, an antigen-binding portion thereof (e.g., paratopes, CDRs), and any other modified conformations of the immunoglobulin molecule comprising the antigen recognition site so long as they exhibit the desired biological activity and specificity. Accordingly, an antibody is an immunoglobulin molecule or fragment or derivative thereof including any polypeptide comprising an antigen-binding site, capable of specifically binding to a target through at least one antigen recognition site located in the variable region of the immunoglobulin molecule. The disclosed antibody can be murine, rat, human, or any other origin (including chimeric or humanized antibodies).

In certain embodiments, the framework regions of the antibody (or antigen-binding fragment thereof) may be identical to human germline sequences or may be naturally or artificially modified.

In one preferred embodiment, the disclosed antibody structures belong to the IgG class of immunoglobulin molecules. A standard IgG immunoglobulin molecule comprises two identical light chain polypeptides, and two identical heavy chain polypeptides. The molecular weight of the light chain polypeptide is around 23,000 Daltons and the molecular weight of the heavy chain polypeptide varies between 53,000-70,000 Daltons. The four chains are typically joined by disulfide bonds in a “Y” configuration.

Two heavy chains (HC) and two light chains (LC) of an immunoglobulin molecule are covalently bonded to each other, and the end portions of the two heavy chains are bonded to each other by covalent disulfide linkages or non-covalent linkages when the immunoglobulins are generated by either hybridomas, B cells, or genetically engineered host cells. The light and heavy chains both contain regions of structural and functional homology. The term “variable” and “constant” are used functionally. Each heavy chain is comprised of a heavy chain variable region (“HCVR” or “VH”) and a heavy chain constant region (comprised of domains CH1, CH2, and CH3). Each light chain is comprised of a light chain variable region (“LCVR or “VL”) and a light chain constant region (CL). The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

The variable region allows the antibody to recognize and specifically bind epitopes located on antigens. The variable domains of both the light (VL) and heavy (VH) chain portions determine antigen recognition and specificity. The antigen-binding site of an antibody is comprised of the VL domain and VH domain, or a subset of the CDRs. More specifically, the antigen-binding site is defined by one, two, or three CDRs on each of the VH and VL chains (i.e., CDR-H1, CDR-H2, CDR-H3, CDR-L1, CDR-L2, and CDR-L3). Conversely, the constant domains of the light chain (CL) and the heavy chain (CH1, CH2, or CH3) confer biological properties such as secretion, transplacental mobility, Fc receptor binding, complement binding, and the like. By convention, the numbering of the constant region domains increases as they become more distal from the antigen-binding site or amino-terminus of the antibody.

As used herein, the term “Complementarity Determining Regions” (CDRs) refers to portions of an antibody variable domain that are (typically) involved in antigen binding. Each variable region has three non-consecutive CDRs, known as CDR1, CDR2, and CDR3. The CDRs are separated by structurally conserved regions called framework regions (FR-1, -2, -3, and -4) that form a “core” 3-sheet structure displaying these loops on the surface of the variable domain. The six CDRs present in each antigen-binding domain are short, non-contiguous sequences of amino acids that are specifically positioned to form the antigen-binding domain as the antibody assumes its three-dimensional configuration in an aqueous environment. The length and composition of the CDR sequences are highly variable, especially in the CDR3. The remainder of the amino acids located in the antigen-binding domains or the “framework” regions, show less inter-molecular variability. The antigen-binding domain formed by the positioned CDRs defines a surface complementary to the epitope on the immunoreactive antigen. This complementary surface promotes the non-covalent binding of the antibody to its cognate epitope. Each CDR can comprise amino acid residues from a CDR as defined by e.g., Kabat (i.e., about residues 24-34 (L1), 50-56 (L2) and 89-97 (L3) in the light chain variable domain and 31-35 (H1), 50-65 (H2) and 95-102 (H3) in the heavy chain variable domain (Kabat et al., Sequences of Proteins of Immunological Interest, 5th Ed. Public Health Service, National Institutes of Health, Bethesda, Md. (1987, 1991)). Each CDR can also comprise amino acid residues from a “hypervariable loop” (i.e., about residues 26-32 (LI), 50-52 (L2) and 91-96 (L3) in the light chain variable domain and 26-32 (H1), 53-55 (H2) and 96-101 (H3) in the heavy chain variable domain (Chothia & Lesk 196 J. Mol. Biol. 901 (1987)). In some instances, a CDR can include amino acids from both a CDR region defined according to Kabat and a hypervariable loop. The Kabat numbering may not always correspond to the linear numbering on the amino acid residues due to a shortening of, or insertion into, a structural component, whether framework or CDR, of the basic variable domain structure. The correct Kabat numbering of residues may be determined for a given antibody, or antigen-binding fragment thereof, by alignment of residues of homology in the sequence of the antibody, or antigen-binding fragment thereof, with a “standard” Kabat numbered sequence or be defined according to ImMunoGeneTics (IMGT) system (Lefranc, M.-P. et al., Dev. Comp. Immunol., 27, 55-77 (2003)).

As used herein, the term “antigen-binding portion” or “antigen-binding fragment” may be a fragment comprising a Fab, Fab′, F(ab′)2, Fd, Fv, domain antibodies (dAbs such as shark and camel antibodies), ScFv, a maxibody, a minibody, a nanobody, an intrabody, a diabody, a triabody, a tetrabody, a v-NAR and a bis-scFv, or a polypeptide that contain at least certain portions of an immunoglobulin sufficient to confer specific antigen-binding to the polypeptide.

The antibody may be any class of antibody, such as IgG, IgA, or IgM (or a subclass thereof), and the antibody need not be of any particular class, and any of the immunoglobulin molecules comprising the antigen recognition site of the required specificity, other modified configurations (including glycosylation variants of antibodies, amino acid sequence variants of antibodies, and covalently modified antibodies) can be encompassed. Modified versions of each of these classes and isotypes are known to a person skilled in the art, accordingly, are within the scope of the instant disclosure.

In some embodiments of the aspects described herein, the anti-B7-H4 antibody fragment is a Fab fragment, which comprises or consist essentially of a variable (VL) and constant (CL) domain of the light chain and a variable domain (VH) and the first constant domain (CH1) of the heavy chain.

In some embodiments of the aspects described herein, the anti-B7-H4 antibody fragment is a Fab′ fragment, which refers to a Fab fragment having one or more cysteine residues at the C-terminus of the CH1 domain.

In some embodiments of the aspects described herein, the anti-B7-H4 antibody fragment is an Fd fragment comprising or consisting essentially of VH and CH1 domains.

In some embodiments of the aspects described herein, the anti-B7-H4 antibody fragment is an Fd′ fragment comprising VH and CH1 domains and one or more cysteine residues at the C-terminus of the CH1 domain.

Single-chain Fv or scFv antibody fragments comprise or consist essentially of the VH and VL domains of an antibody, such that these domains are present in a single polypeptide chain. Generally, an Fv polypeptide further comprises a polypeptide linker between the VH and VL domains, which allows the scFv to form the desired structure for antigen-binding. Accordingly, in some embodiments of the aspects described herein, the anti-B7-H4 antibody fragment is a Fv fragment comprising or consisting essentially of the VL and VH domains of a single arm of an antibody.

In some embodiments of the aspects described herein, the anti-B7-H4 antibody fragment is a diabody comprising two antigen-binding sites, comprising a heavy chain variable domain (VH) connected to a light chain variable domain (VL) in the same polypeptide chain.

In some embodiments of the aspects described herein, the anti-B7-H4 antibody fragment is a dAb fragment comprising or consisting essentially of a VH domain.

In some embodiments of the aspects described herein, the anti-B7-H4 antibody fragment is a F(ab′)2 fragment, which comprises a bivalent fragment comprising two Fab′ fragments linked by a disulfide bridge at the hinge region.

In some embodiments of the aspects described herein, the anti-B7-H4 antibody fragment is a linear antibody comprising a pair of tandem Fd segments (VH-CH1-VH-CH1) which, together with complementary light chain polypeptides, form a pair of antigen-binding regions.

A person skilled in the arts can use various techniques that have been developed and are available for the production of antibody fragments. Traditionally, these fragments were derived via proteolytic digestion of intact antibodies. However, F(ab′)2 fragments can be isolated directly from recombinant host cell culture. Other techniques for the production of antibody fragments will be apparent to the skilled practitioner. In other embodiments, the antibody fragment of choice is a single chain Fv fragment (scFv). See, for example, WO 93/16185. Alternatively, these fragments can also be produced directly by recombinant host cells. For example, antibody fragments can be isolated from the antibody phage libraries discussed herein. In another approach, Fab′-SH fragments can be directly recovered from E. coli and chemically coupled to form F(ab′)2 fragments (Carter et al., 1992).

In one embodiment, the antibody is a bispecific antibody comprising a complementary region that binds B7-H4.

Contemplated antibodies or antigen-binding fragments may have all types of constant regions, including IgM, IgG, IgD, and IgE, and any isotype, including IgG1, IgG2, IgG3, and IgG4. In one embodiment, the isotype is human IgG1. In another embodiment, the human isotype IgG4 is used. Light chain constant regions can be X or K. The antibody, or antigen-binding fragment thereof, may comprise sequences from more than one class or isotype.

The disclosure describes antibodies that bind to B7-H4, and antigen-binding fragments thereof, that bind to B7-H4, as well as fusion proteins comprising such anti-B7-H4 antibodies, or antigen-binding fragments thereof. The term “B7-H4” refers to V-set domain-containing T-cell activation inhibitor 1 (VTCN1), also known as B7 homolog 4, B7h.5, immune costimulatory protein B7-H4, protein B7S1, or T-cell costimulatory molecule B7x). The sequence of human B7-H4 is provided in SEQ ID NO:161. The sequence of murine B7-H4 is provided in SEQ ID NO:162. The term B7-H4 encompasses recombinant B7-H4 and/or a fragment thereof. The term also includes B7-H4, or a fragment thereof coupled to, for example, mouse or human Fe, histidine tag, and/or a signal sequence. The term may further encompass a fusion protein comprising B7-H4.

In embodiments, the anti-B7-H4 antibody or antigen binding fragment thereof, or the fusion protein comprising an anti-B7-H4 antibody, or antigen-binding fragments thereof, specifically binds to B7-H4 and antagonizes B7-H4 mediated immune suppression. The anti-B7-H4 antibodies, and antigen-binding fragments thereof, (and the fusion proteins comprising such anti-B7-H4 antibodies and antigen-binding fragments thereof) disclosed herein may interrupt, inhibit, or reduce B7-H4 biological activity including downstream events mediated by B7-H4. The anti-B7-H4 antibodies, and antigen-binding fragments thereof, (and the fusion proteins comprising such anti-B7-H4 antibodies and antigen-binding fragments thereof) disclosed herein may exhibit any one or more of the following features: (a) binding to B7-H4 and blocking of downstream signaling events; (b) blocking binding of B7-H4 to its cognate ligand; (c) increasing T cell proliferation; (d) upregulating the T cell-mediated immune response; (e) stimulating cytokine secretion; (f) reducing inhibitory signal transduction through B7-H4; and/or (g) killing tumor cells. The anti-B7-H4 antibodies, and antigen-binding fragments thereof, (and the fusion proteins comprising such anti-B7-H4 antibodies and antigen-binding fragments thereof) disclosed herein exhibit potent binding and inhibitory activities and are useful for therapeutic and diagnostics uses.

In one aspect, the disclosure provides antibodies, and antigen-binding fragments thereof, (and fusion proteins comprising such anti-B7-H4 antibodies and antigen-binding fragments thereof), that bind to B7-H4. In certain embodiments, the disclosure provides bispecific antibodies and binding proteins that bind specifically to B7-H4 and at least one other molecule.

In one aspect, provided is an anti-B7-H4 antibody, or antigen-binding fragment thereof, wherein the antibody, or antigen-binding fragment thereof, comprises a heavy chain variable region and a light chain variable region, wherein each of the heavy chain and the light chain variable regions comprise a CDR1, CDR2, and CDR3.

Provided herein is an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO:73, CDR2L comprises SEQ ID NO:74, and CDR3L comprises SEQ ID NO:75.

Provided herein is an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO:20, CDR2L comprises SEQ ID NO:21, and CDR3L comprises SEQ ID NO:22.

Provided herein is an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO:66, CDR2L comprises SEQ ID NO:67, and CDR3L comprises SEQ ID NO:68.

Provided herein is an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO:70, CDR2L comprises SEQ ID NO:71, and CDR3L comprises SEQ ID NO:72.

Provided herein is an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO: 84, CDR2L comprises SEQ ID NO:85, and CDR3L comprises SEQ ID NO:86.

Provided herein is an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO: 8, CDR2L comprises SEQ ID NO: 9, and CDR3L comprises SEQ ID NO:10.

Provided herein is an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO: 77, CDR2L comprises SEQ ID NO: 78, and CDR3L comprises SEQ ID NO: 79.

Provided herein is an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO: 81, CDR2L comprises SEQ ID NO:82, and CDR3L comprises SEQ ID NO:83.

Provided herein is an anti-B7-H4 antibody, or antigen-binding fragment thereof, wherein CDR1H comprises SEQ ID NO:16, CDR2H comprises SEQ ID NO:17, CDR3H comprises SEQ ID NO:18, CDR1L comprises SEQ ID NO:70, CDR2L comprises SEQ ID NO:71, and CDR3L comprises SEQ ID NO:72.

Provided herein is an anti-B7-H4 antibody, or antigen-binding fragment thereof, wherein CDR1H comprises SEQ ID NO:172, CDR2H comprises SEQ ID NO:173, CDR3H comprises SEQ ID NO:174, CDR1L comprises SEQ ID NO:175, CDR2L comprises SEQ ID NO:176, and CDR3L comprises SEQ ID NO:177.

Provided herein is an anti-B7-H4 antibody, or antigen-binding fragment thereof, wherein CDR1H comprises SEQ ID NO:178, CDR2H comprises SEQ ID NO:179, CDR3H comprises SEQ ID NO:180, CDR1L comprises SEQ ID NO:181, CDR2L comprises SEQ ID NO:182, and CDR3L comprises SEQ ID NO:183.

Provided herein is an anti-B7-H4 antibody, or antigen-binding fragment thereof, wherein CDR1H comprises SEQ ID NO:184, CDR2H comprises SEQ ID NO:185, CDR3H comprises SEQ ID NO:186, CDR1L comprises SEQ ID NO:187, CDR2L comprises SEQ ID NO:188, and CDR3L comprises SEQ ID NO:189.

Provided herein is an anti-B7-H4 antibody, or antigen-binding fragment thereof, wherein the heavy chain variable region comprises SEQ ID NO: 15, or a sequence that is at least 90%, at least 95%, at least 96%, at least 97%, at least 98%, or at least 99% identical to SEQ ID NO: 15; and wherein the light chain variable region comprises SEQ ID NO: x, or a sequence that is at least 90%, at least 95%, at least 96%, at least 97%, at least 98%, or at least 99% identical to SEQ ID NO: 19.

Provided herein is an anti-B7-H4 antibody, or antigen-binding fragment thereof, wherein the heavy chain variable region comprises SEQ ID NO: 15, or a sequence that is at least 90%, at least 95%, at least 96%, at least 97%, at least 98%, or at least 99% identical to SEQ ID NO: 15; and wherein the light chain variable region comprises SEQ ID NO: 65, or a sequence that is at least 90%, at least 95%, at least 96%, at least 97%, at least 98%, or at least 99% identical to SEQ ID NO: 65.

Provided herein is an anti-B7-H4 antibody, or antigen-binding fragment thereof, wherein the heavy chain variable region comprises SEQ ID NO: 15, or a sequence that is at least 90%, at least 95%, at least 96%, at least 97%, at least 98%, or at least 99% identical to SEQ ID NO: 15; and wherein the light chain variable region comprises SEQ ID NO: 69, or a sequence that is at least 90%, at least 95%, at least 96%, at least 97%, at least 98%, or at least 99% identical to SEQ ID NO: 69.

Provided herein is an anti-B7-H4 antibody, or antigen-binding fragment thereof, wherein the heavy chain variable region comprises SEQ ID NO: 3, or a sequence that is at least 90%, at least 95%, at least 96%, at least 97%, at least 98%, or at least 99% identical to SEQ ID NO: 3 and wherein the light chain variable region comprises SEQ ID NO: 7, or a sequence that is at least 90%, at least 95%, at least 96%, at least 97%, at least 98%, or at least 99% identical to SEQ ID NO: 7.

Provided herein is an anti-B7-H4 antibody, or antigen-binding fragment thereof, wherein the heavy chain variable region comprises SEQ ID NO: 3, or a sequence that is at least 90%, at least 95%, at least 96%, at least 97%, at least 98%, or at least 99% identical to SEQ ID NO: 3 and wherein the light chain variable region comprises SEQ ID NO: 76, or a sequence that is at least 90%, at least 95%, at least 96%, at least 97%, at least 98%, or at least 99% identical to SEQ ID NO: 76.

Provided herein is an anti-B7-H4 antibody, or antigen-binding fragment thereof, wherein the heavy chain variable region comprises SEQ ID NO: 3, or a sequence that is at least 90%, at least 95%, at least 96%, at least 97%, at least 98%, or at least 99% identical to SEQ ID NO: 3 and wherein the light chain variable region comprises SEQ ID NO: 80, or a sequence that is at least 90%, at least 95%, at least 96%, at least 97%, at least 98%, or at least 99% identical to SEQ ID NO: 80.

Provided herein is an anti-B7-H4 antibody, or antigen-binding fragment thereof, wherein the heavy chain variable region comprises SEQ ID NO: 15 and wherein the light chain variable region comprises SEQ ID NO: 19.

Provided herein is an anti-B7-H4 antibody, or antigen-binding fragment thereof, wherein the heavy chain variable region comprises SEQ ID NO: 15 and wherein the light chain variable region comprises SEQ ID NO: 65.

Provided herein is an anti-B7-H4 antibody, or antigen-binding fragment thereof, wherein the heavy chain variable region comprises SEQ ID NO: 15 and wherein the light chain variable region comprises SEQ ID NO: 69.

Provided herein is an anti-B7-H4 antibody, or antigen-binding fragment thereof, wherein the heavy chain variable region comprises SEQ ID NO: 3 and wherein the light chain variable region comprises SEQ ID NO: 7.

Provided herein is an anti-B7-H4 antibody, or antigen-binding fragment thereof, wherein the heavy chain variable region comprises SEQ ID NO: 3 and wherein the light chain variable region comprises SEQ ID NO: 76.

Provided herein is an anti-B7-H4 antibody, or antigen-binding fragment thereof, wherein the heavy chain variable region comprises SEQ ID NO: 3 and wherein the light chain variable region comprises SEQ ID NO: 80.

In one aspect, provided is an anti-B7-H4 antibody, or antigen-binding fragment thereof, wherein the antibody, or antigen-binding fragment thereof, comprises a heavy chain variable region and a light chain variable region, wherein each of the heavy chain and the light chain variable regions comprise a CDR1, CDR2, and CDR3.

Provided herein is an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:111, CDR2L comprises SEQ ID NO: 112, and CDR3L comprises SEQ ID NO: 113.

Provided herein is an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:50, CDR2L comprises SEQ ID NO:51, and CDR3L comprises SEQ ID NO:52.

Provided herein is an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:88, CDR2L comprises SEQ ID NO:89, and CDR3L comprises SEQ ID NO:90.

Provided herein is an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:92, CDR2L comprises SEQ ID NO:93, and CDR3L comprises SEQ ID NO:94.

Provided herein is an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:96, CDR2L comprises SEQ ID NO:97, and CDR3L comprises SEQ ID NO:98.

Provided herein is an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:100, CDR2L comprises SEQ ID NO:101, and CDR3L comprises SEQ ID NO: 102.

Provided herein is an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:104, CDR2L comprises SEQ ID NO:105, and CDR3L comprises SEQ ID NO:106.

Provided herein is an anti-B7-H4 antibody, or antigen-binding fragment thereof, wherein the CDR1H comprises SEQ ID NO:46, CDR2H comprises SEQ ID NO:47, CDR3H comprises SEQ ID NO:48, CDR1L comprises SEQ ID NO:108, CDR2L comprises SEQ ID NO:109, and CDR3L comprises SEQ ID NO:110.

Provided herein is an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:49, or a sequence that is at least 90%, at least 95%, at least 96%, at least 97%, at least 98%, or at least 99% identical to SEQ ID NO:49.

Provided herein is an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:87, or a sequence that is at least 90%, at least 95%, at least 96%, at least 97%, at least 98%, or at least 99% identical to SEQ ID NO:87.

Provided herein is an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:91, or a sequence that is at least 90%, at least 95%, at least 96%, at least 97%, at least 98%, or at least 99% identical to SEQ ID NO:91.

Provided herein is an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:95, or a sequence that is at least 90%, at least 95%, at least 96%, at least 97%, at least 98%, or at least 99% identical to SEQ ID NO:95.

Provided herein is an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:99, or a sequence that is at least 90%, at least 95%, at least 96%, at least 97%, at least 98%, or at least 99% identical to SEQ ID NO:99.

Provided herein is an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:103, or a sequence that is at least 90%, at least 95%, at least 96%, at least 97%, at least 98%, or at least 99% identical to SEQ ID NO:103.

Provided herein is an anti-B7-H4 antibody, or antigen-binding fragment thereof, wherein the heavy chain variable region comprises SEQ ID NO:45, or a sequence that is at least 90%, at least 95%, at least 96%, at least 97%, at least 98%, or at least 99% identical to SEQ ID NO:45 and wherein the light chain variable region comprises SEQ ID NO:107, or a sequence that is at least 90%, at least 95%, at least 96%, at least 97%, at least 98%, or at least 99% identical to SEQ ID NO:107.

Provided herein is an anti-B7-H4 antibody, or antigen-binding fragment thereof, wherein the heavy chain variable region comprises SEQ ID NO: 45 and wherein the light chain variable region comprises SEQ ID NO:49.

Provided herein is an anti-B7-H4 antibody, or antigen-binding fragment thereof, wherein the heavy chain variable region comprises SEQ ID NO: 45 and wherein the light chain variable region comprises SEQ ID NO:87.

Provided herein is an anti-B7-H4 antibody, or antigen-binding fragment thereof, wherein the heavy chain variable region comprises SEQ ID NO: 45 and wherein the light chain variable region comprises SEQ ID NO:91.

Provided herein is an anti-B7-H4 antibody, or antigen-binding fragment thereof, wherein the heavy chain variable region comprises SEQ ID NO: 45 and wherein the light chain variable region comprises SEQ ID NO:95.

Provided herein is an anti-B7-H4 antibody, or antigen-binding fragment thereof, wherein the heavy chain variable region comprises SEQ ID NO: 45 and wherein the light chain variable region comprises SEQ ID NO:99.

Provided herein is an anti-B7-H4 antibody, or antigen-binding fragment thereof, wherein the heavy chain variable region comprises SEQ ID NO: 45 and wherein the light chain variable region comprises SEQ ID NO:103.

Provided herein is an anti-B7-H4 antibody, or antigen-binding fragment thereof, wherein the heavy chain variable region comprises SEQ ID NO: 45 and wherein the light chain variable region comprises SEQ ID NO:107.

In one aspect, provided is an anti-B7-H4 antibody, or antigen-binding fragment thereof, wherein the antibody, or antigen-binding fragment thereof, comprises a heavy chain variable region and a light chain variable region, wherein each of the heavy chain and the light chain variable regions comprise a CDR1, CDR2, and CDR3.

Provided herein is an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:134, CDR2L comprises SEQ ID NO:135, and CDR3L comprises SEQ ID NO: 136.

Provided herein is an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:62, CDR2L comprises SEQ ID NO:63, and CDR3L comprises SEQ ID NO:64.

Provided herein is an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:115, CDR2L comprises SEQ ID NO:116, and CDR3L comprises SEQ ID NO: 117.

Provided herein is an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:119, CDR2L comprises SEQ ID NO:120, and CDR3L comprises SEQ ID NO: 121.

Provided herein is an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:123, CDR2L comprises SEQ ID NO:124, and CDR3L comprises SEQ ID NO:125.

Provided herein is an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:127, CDR2L comprises SEQ ID NO:128, and CDR3L comprises SEQ ID NO:129.

Provided herein is an anti-B7-H4 antibody, or antigen-binding fragment thereof, wherein CDR1H comprises SEQ ID NO:58, CDR2H comprises SEQ ID NO:59, CDR3H comprises SEQ ID NO:60, and wherein CDR1L comprises SEQ ID NO:131, CDR2L comprises SEQ ID NO:132, and CDR3L comprises SEQ ID NO:133.

Provided herein is an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:61, or a sequence that is at least 90%, at least 95%, at least 96%, at least 97%, at least 98%, or at least 99% identical to SEQ ID NO:61.

Provided herein is an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:114, or a sequence that is at least 90%, at least 95%, at least 96%, at least 97%, at least 98%, or at least 99% identical to SEQ ID NO:114.

Provided herein is an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:118, or a sequence that is at least 90%, at least 95%, at least 96%, at least 97%, at least 98%, or at least 99% identical to SEQ ID NO:118.

Provided herein is an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:122, or a sequence that is at least 90%, at least 95%, at least 96%, at least 97%, at least 98%, or at least 99% identical to SEQ ID NO:122.

Provided herein is an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:126, or a sequence that is at least 90%, at least 95%, at least 96%, at least 97%, at least 98%, or at least 99% identical to SEQ ID NO:126. or

Provided herein is an anti-B7-H4 antibody, or antigen-binding fragment thereof, wherein the heavy chain variable region comprises SEQ ID NO:57, or a sequence that is at least 90%, at least 95%, at least 96%, at least 97%, at least 98%, or at least 99% identical to SEQ ID NO:57 and wherein the light chain variable region comprises SEQ ID NO:130, or a sequence that is at least 90%, at least 95%, at least 96%, at least 97%, at least 98%, or at least 99% identical to SEQ ID NO:130.

Provided herein is an anti-B7-H4 antibody, or antigen-binding fragment thereof, wherein the heavy chain variable region comprises SEQ ID NO:57 and wherein the light chain variable region comprises SEQ ID NO:61.

Provided herein is an anti-B7-H4 antibody, or antigen-binding fragment thereof, wherein the heavy chain variable region comprises SEQ ID NO:57 and wherein the light chain variable region comprises SEQ ID NO:114.

Provided herein is an anti-B7-H4 antibody, or antigen-binding fragment thereof, wherein the heavy chain variable region comprises SEQ ID NO:57 and wherein the light chain variable region comprises SEQ ID NO:118.

Provided herein is an anti-B7-H4 antibody, or antigen-binding fragment thereof, wherein the heavy chain variable region comprises SEQ ID NO:57 and wherein the light chain variable region comprises SEQ ID NO:122.

Provided herein is an anti-B7-H4 antibody, or antigen-binding fragment thereof, wherein the heavy chain variable region comprises SEQ ID NO:57 and wherein the light chain variable region comprises SEQ ID NO:126.

Provided herein is an anti-B7-H4 antibody, or antigen-binding fragment thereof, wherein the heavy chain variable region comprises SEQ ID NO:57 and wherein the light chain variable region comprises SEQ ID NO:130.

Tables 5, 7, 9, 16-20 and 24 show the heavy and light chain variable regions and associated CDR of antibodies disclosed herein as does the accompanying sequence listing, which is incorporated by reference in its entirety. As shown in the Examples below, light chain shuffling of anti-B7-H4 antibodies led to the identification of antibody variants that conferred substantial improvements in B7-H4 binding affinity. Analysis of the variants revealed certain CDR positions at which amino acids remained relatively unchanged among the light chain shuffled antibodies and other CDR positions at which variation could be introduced without abolishing B7-H4 binding.

The anti-B7-H4 antibodies, and antigen-binding fragments thereof, disclosed herein and the fusion proteins disclosed herein can have one or more amino acid substitutions, deletions, insertions, and/or additions. In some embodiments, one or more CDR residues or more or more of the framework residues of the anti-B7-H4 antibodies or antigen-binding fragments (or the fusion proteins comprising the anti-B7-H4 antibodies or antigen-binding fragments) disclosed herein have been changed by amino acid substitution, deletion, insertion, and/or addition. Amino acid substitutions can be conservative or non-conservative substitutions. The present disclosure also includes anti-B7-H4 antibodies, and antigen-binding fragments thereof, (and fusion proteins comprising the anti-B7-H4 antibodies or antigen-binding fragments thereof), which are derived from the amino acid sequences disclosed herein, wherein one or more amino acids within one or more framework and/or CDR regions are mutated to the corresponding residue(s) of the germline sequence from which the antibody was derived, or to the corresponding residue(s) of another human germline sequence, or to a conservative amino acid substitution of the corresponding germline residue(s) (such sequence changes are referred to herein collectively as “germline mutations”). In certain embodiments, the anti-B7-H4 antibodies or binding fragments thereof (or the fusion proteins comprising the anti-B7-H4 antibodies or antigen-binding fragments) comprise one or more CDRs, or one or more variable domains with an amino acid sequence at least 85% at least 90%, at least 95%, at least 97%, at least 98%, or at least 99%, identical to the CDR and/or variable domain sequences set forth in Tables 5, 7, 9, 16-20, or 24.

Also provided herein variable heavy chain and variable light chain sequences (as well as pairings thereof) that are similar, but not identical to the variable heavy chain and variable light chains disclosed in Tables 16-20 (and pairings thereof). It will be evident that any of the frameworks described herein can be utilized in combination with any of the CDRs and CDR motifs described herein. In some embodiments, the anti-B7-H4 antibody, or antigen-binding fragment thereof, utilizes a framework described in Tables 16-20.

Also provided herein is a chimeric antigen receptor (CAR) comprising one, two, three, four, five, or six CDRs of the anti-B7-H4 antibodies and antigen binding fragments disclosed herein. Also provided herein is CAR comprising the six CDRs of any one of the anti-B7-H4 antibodies and antigen binding fragments disclosed herein.

Disclosed is an immune cell expressing a CAR comprising one, two, three, four, five, or six CDRs of the anti-B7-H4 antibodies and antigen binding fragments disclosed herein. Disclosed is an immune cell expressing a CAR comprising the six CDRs of any one of the anti-B7-H4 antibodies and antigen binding fragments disclosed herein. In some embodiments, the immune cell is a T cell.

“Identity” refers to the number or percentage of identical positions shared by two amino acid or nucleic acid sequences in optimally aligned sequences after considering number of gaps and the length of each gap that were needed for the optimal alignment. “Substantially identical” means an amino acid sequence, which differs from the original sequence only by conservative amino acid substitutions, which do not destroy the function of the protein.

Also disclosed herein are anti-B7-H4 antibodies or antigen-binding fragments thereof or fusion proteins comprising an amino acid sequence that is at least 80%, or at least 85%, or at least 90%, or at least 95%, or at least 98%, or at least 99% identical to an amino acid sequence disclosed herein. Methods and computer programs for determining sequence similarity are publicly available, including, but not limited to, the GCG program package (Devereux et al., Nucleic Acids Research 12: 387, 1984), BLASTP, BLASTN, FASTA (Altschul et al., J. Mol. Biol. 215:403 (1990), and the ALIGN program (version 2.0). The Smith Waterman algorithm may also be used to determine similarity. The BLAST program is publicly available from NCBI and other sources (BLAST Manual, Altschul, et al., NCBI NLM NIH, Bethesda, Md. 20894; BLAST 2.0 at http://www.ncbi.nlm.nih.gov/blast/). In comparing sequences, these methods account for various substitutions, deletions, and other modifications.

In some embodiments of the aspects described herein, amino acid sequence modification(s) of the antibodies or antigen-binding fragments thereof that bind to B7-H4 or the fusion proteins that bind to B7-H4 described herein are contemplated. Amino acid sequence variants of the anti-B7-H4 antibody, or antigen-binding fragment thereof, or the fusion proteins are prepared by introducing appropriate nucleotide changes into the nucleic acid encoding the anti-B7-H4 antibody, or antigen-binding fragment thereof, or the fusion protein, or by peptide synthesis. Such modifications include, for example, deletions from, and/or insertions into and/or substitutions of, residues within the amino acid sequences of the antibody or antigen-binding fragment thereof. Any combination of deletion, insertion, and substitution is made to arrive at the final construct, if the final construct possesses the desired characteristics, e.g., binding specificity, inhibition of biological activity.

Amino acid substitutions can be made, in some cases, by selecting substitutions that do not differ significantly in their effect on maintaining (a) the structure of the peptide backbone in the area of the substitution, (b) the charge or hydrophobicity of the molecule at the target sit; or (c) the bulk of the side chain (conservative amino acid substitution variant. These variants have at least one amino acid residue in the antibody, or antigen-binding fragment thereof, or fusion protein replaced by a different residue that has similar side chain properties. Amino acids can be grouped according to similarities in the properties of their side chains (see Lehninger, BIOCHEMISTRY (2nd ed., Worth Publishers, New York, 1975):
(1) non-polar: Ala (A), Val (V), Leu (L), Ile (I), Pro (P), Phe (F), Trp (W), Met (M);
(2) uncharged polar: Gly (G), Ser (S), Thr (T), Cys (C), Tyr (Y), Asn (N), Gln (Q);
(3) acidic: Asp (D), Glu (E);
(4) basic: Lys (K), Arg (R), His (H).

As such, a non-limiting example for a conservative amino acid substitution is one that replaces a non-polar amino acid with another non-polar amino acid.

Alternatively, naturally occurring residues can be divided into groups based on common side-chain properties:
(1) hydrophobic: Ala (A), Val (V), Leu (L), Ile (I), Met (M);
(2) neutral hydrophilic: Ser (S), Thr (T), Cys (C), Asn (N), Gln (Q);
(3) acidic: Asp (D), Glu (E);
(4) basic: Lys (K), Arg (R), His (H);
(5) residues that influence chain orientation: Gly (G), Pro (P);
(6) aromatic: Phe (F), Trp (W), Tyr (Y).

Substitutions made within these groups can be considered conservative substitutions. Examples of non-limiting substitutions include, substitution of valine for alanine, lysine for arginine, glutamine for asparagine, glutamic acid for aspartic acid, serine for cysteine, asparagine for glutamine, aspartic acid for glutamic acid, proline for glycine, arginine for histidine, leucine for isoleucine, isoleucine for leucine, arginine for lysine, leucine for methionine, leucine for phenylalanine, glycine for proline, threonine for serine, serine for threonine, tyrosine for tryptophan, phenylalanine for tyrosine, and/or leucine for valine.

Further contemplated are amino acid sequence insertions, which can include amino- and/or carboxyl-terminal fusions ranging in length from one residue to polypeptides containing a hundred or more residues, as well as intrasequence insertions of single or multiple amino acid residues.

Examples of terminal fusions include an antibody, or antigen-binding fragment thereof, with an N-terminal methionyl residue or an antibody, or antigen-binding fragment thereof, fused to a cytotoxic polypeptide (or fusion proteins comprising such antibody or antigen-binding fragment thereof). Other examples of terminal fusions of the antibody, or antigen-binding fragment thereof, include the fusion to the N- or C-terminus of the antibody, or antigen-binding fragment thereof, to an enzyme or a polypeptide which increases the serum half-life of the antibody or antigen-binding fragment thereof, such as, for example, biotin (or fusion proteins comprising such antibody or antigen-binding fragment thereof).

In some embodiments, anti-B7-H4 antibody, or antigen-binding fragment thereof, (or a fusion protein comprising such an antibody or antigen-binding fragment thereof), comprises a modification (including but not limited to an amino acid modification or substitution) that increases affinity of the anti-B7-H4 antibody, or antigen-binding fragment thereof, (or a fusion protein comprising such an antibody or antigen-binding fragment thereof) to FcRn. In some embodiments, the anti-B7-H4 antibody, or antigen-binding fragment thereof, (or a fusion protein comprising such an antibody or antigen-binding fragment thereof) comprises an M428L amino acid substitution (Kabat EU index numbering). In some embodiments, the anti-B7-H4 antibody, or antigen-binding fragment thereof, (or a fusion protein comprising such an antibody or antigen-binding fragment thereof) comprises an N434S amino acid substitution (Kabat EU index numbering). In some embodiments, the anti-B7-H4 antibody, or antigen-binding fragment thereof, (or a fusion protein comprising such an antibody or antigen-binding fragment thereof) comprises an M428L and N434S amino acid substitution (Kabat EU index numbering).

Any cysteine residue not involved in maintaining the proper conformation of the antibodies or antigen-binding fragments thereof that bind to B7-H4 also can be substituted, for example with a serine or an alanine, to improve the oxidative stability of the molecule and prevent aberrant crosslinking.

Conversely, cysteine bond(s) can be added to the anti-B7-H4 antibody, or antigen-binding fragment thereof, to improve its stability (particularly where the anti-B7-H4 antibody, or antigen-binding fragment thereof, is an antibody fragment such as an Fv fragment).

In some embodiments, the anti-B7-H4 antibodies, or antigen-binding fragments thereof described herein (or the fusion protein comprising the anti-B7-H4 antibodies, or antigen-binding fragments thereof described herein) have amino acid alterations that alter the original glycosylation pattern of the anti-B7-H4 antibody or antigen-binding fragment thereof. By “altering the original glycosylation pattern” is meant deleting one or more carbohydrate moieties found in the antibody or antigen-binding fragment thereof, and/or adding one or more glycosylation sites that are not present in the antibody or antigen-binding fragment thereof. Glycosylation of antibodies is typically either N-linked or O-linked. N-linked refers to the attachment of the carbohydrate moiety to the side chain of an asparagine residue. The tripeptide sequences asparagine-X-serine and asparagine-X-threonine, wherein X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine can also be used. The alteration can also be made by the addition of, or substitution by, one or more serine or threonine residues to the sequence of the original antibody, or antigen-binding fragment thereof, (for O-linked glycosylation sites).

In some embodiments, the anti-B7-H4 antibodies, or antigen-binding fragments thereof. described herein (or the fusion protein comprising the anti-B7-H4 antibodies, or antigen-binding fragments thereof described herein) may include a modification, including, but not limited to glycosylation, acetylation, pegylation, phosphorylation, phosphorylation, amidation, derivatization by known protecting/blocking groups, proteolytic cleavage, linkage to a cellular ligand or other protein, etc. The process of making chemical modifications is known in the art, and may include, but are not limited to, specific chemical cleavage, acetylation, formylation, metabolic synthesis of tunicamycin, etc. Additionally, the molecules may contain one or more non-classical amino acids.

The anti-B7-H4 antibodies, and antigen-binding fragments thereof, disclosed herein may include anti-B7-H4 antibodies, and antigen-binding fragments thereof, whose binding characteristics have been altered by direct mutation, affinity maturation, phage display, or chain shuffling. The affinity and specificity may be altered by mutating CDRs and screening for CDRs with desired characteristics. Methods of mutagenesis are known to one of skill in the art.

Antibody and Fusion Protein Binding

Also provided herein are (i) anti-B7-H4 antibodies, and antigen-binding fragments thereof, and (ii) fusion proteins comprising anti-B7-H4 antibodies, or antigen-binding fragments thereof, that bind to the same epitope on B7-H4 as one of the anti-B7-H4 antibodies or antigen-binding fragments thereof disclosed herein.

In some embodiments, the (i) anti-B7-H4 antibody, and antigen-binding fragment thereof, or (ii) fusion protein comprising an anti-B7-H4 antibody, or antigen-binding fragment thereof, binds to the IgV domain of B7-H4. The IgV domain is related to the variable domain of an immunoglobulin. The IgC domain of human and murine B7-H4 is indicated in Table 23 (see SEQ ID NOs:162 and 162).

In some embodiments, the (i) anti-B7-H4 antibody, and antigen-binding fragment thereof, or (ii) fusion protein comprising an anti-B7-H4 antibody, or antigen-binding fragment thereof, binds to the IgC domain of B7-H4. The IgC domain is related to the constant domain of an immunoglobulin. The IgC domain of human and murine B7-H4 is indicated in Table 23 (see SEQ ID NOs:162 and 162).

In some embodiments, the (i) anti-B7-H4 antibody, and antigen-binding fragment thereof, or (ii) fusion protein comprising an anti-B7-H4 antibody, or antigen-binding fragment thereof, binds to the IgV domain and the IgC domain of B7-H4.

In some embodiments, the (i) anti-B7-H4 antibody, and antigen-binding fragment thereof, or (ii) fusion protein comprising an anti-B7-H4 antibody, or antigen-binding fragment thereof, binds to murine B7-H4 or a domain thereof. In some embodiments, the (i) anti-B7-H4 antibody, and antigen-binding fragment thereof, or (ii) fusion protein comprising an anti-B7-H4 antibody, or antigen-binding fragment thereof, binds to human B7-H4 or a domain thereof. In some embodiments, the (i) anti-B7-H4 antibody, and antigen-binding fragment thereof, or (ii) fusion protein comprising an anti-B7-H4 antibody, or antigen-binding fragment thereof, binds to murine and human B7-H4 or a domain thereof.

As used herein, “binding” of an antibody or antigen-binding fragment thereof, or fusion protein comprising an antibody or antigen-binding fragment thereof, to B7-H4, an epitope on B7-H4, or, in certain embodiments described below, particular residues on B7-H4, includes the selective interaction of the antibody, or antigen-binding fragment thereof, with B7-H4. Binding therefore includes, e.g., primary and secondary interactions including hydrogen bonds, ionic interactions, salt bridges, as well as hydrophilic and hydrophobic interactions.

In certain embodiments, the anti-B7-H4 antibodies or antigen-binding fragments thereof (or the fusion proteins comprising such anti-B7-H4 antibodies or antigen-binding fragments thereof) described herein bind to B7-H4 with an equilibrium constant for the dissociation (K_{D}) of 10⁻² to 10⁻¹³ mol/l, 10⁻³ to 10⁻¹³ mol/l, 10⁻⁴ to 10⁻¹³ mol/l, 10⁻⁵ to 10⁻¹³ mol/l, 10⁻⁶ to 10⁻¹³ mol/l, 10⁻⁷ to 10⁻¹³ mol/l, 10⁻¹ to 10⁻¹³ mol/l, 10⁻⁹ to 10⁻¹³ mol/l, 10⁻¹⁰ to 10⁻¹³ mol/l, 10¹² to 10⁻¹³ mol/l. In some embodiments, the anti-B7-H4 antibodies or antigen-binding fragments thereof (or the fusion proteins comprising such anti-B7-H4 antibodies or antigen-binding fragments thereof) described herein bind to B7-H4 with a K_{D} of 10⁻² to 10⁻⁹ mol/l, 10⁻³ to 10⁻⁹ mol/l, 10⁻⁴ to 10⁻⁹ mol/l, 10⁻⁵ to 10⁻⁹ mol/l, 10⁻⁶ to 10⁻⁹ mol/l, 10⁻⁷ to 10⁻⁹ mol/l, 10⁻⁸ to 10⁻⁹ mol/l, 10⁻¹⁰ 10⁻¹¹ mol/l, 10⁻¹¹ 10⁻¹² mol/l, 10⁻¹² 10⁻¹³ mol/l, or 10⁻¹² 10⁻¹⁴ mol/l. In one embodiment, the anti-B7-H4 antibodies or antigen-binding fragments thereof (or the fusion proteins comprising such anti-B7-H4 antibodies or antigen-binding fragments thereof) described herein bind to B7-H4 with a K_{D} of 10⁻⁹ to 10⁻¹³ mol/l.

As used herein, “affinity,” represented by the K_{D} of an antigen with an antigen-binding protein, is a measure of the binding strength between an antigenic determinant and an antigen-binding site on the antigen-binding protein, such as an antibody or antibody fragment thereof. The value of K_{D} is inversely proportional to the binding strength between an antigenic determinant and the antigen-binding molecule. Alternatively, the affinity can also be expressed as the association constant (KA), which is 1/K_{D}). Affinity can be determined in a manner known per se, depending on the specific antigen of interest by a person skilled in the art.

The term “specificity” herein refers to the ability of an antibody or antigen-binding fragment thereof, such as an anti-B7-H4 antibody or antigen-binding fragment thereof, to recognize an epitope within B7-H4, while only having little or no detectable reactivity with other portions of B7-H4. Specificity can be relatively determined by competition assays or by epitope identification/characterization techniques described herein or their equivalents known in the art.

The term “epitope” herein refers to the specific target to which an antibody binds. Epitopes can be formed both by a contiguous stretch of amino acids (continuous epitopes) and by three-dimensional arrangement of amino acid residues that exists only when the target protein is folded in a particular conformation (discontinuous epitopes.) In general, an epitope comprises at least 3 amino acids, at least 4, at least 5, or about 7-10 amino acids.

Disclosed herein are anti-B7-H4 antibodies, and antigen-binding fragments thereof, as well as fusion proteins comprising an anti-B7-H4 antibody, or antigen-binding fragment thereof, that specifically bind to the same epitope as anti-B7-H4 antibody 38B2. Also disclosed herein are anti-B7-H4 antibodies, and antigen-binding fragments thereof, as well as fusion proteins comprising an anti-B7-H4 antibody, or antigen-binding fragment thereof, that bind to the same epitope as anti-B7-H4 antibody 31B1.

As used herein, a “blocking” antibody or an antibody “antagonist” is one that inhibits or reduces the biological activity of the antigen to which it binds. For example, in some embodiments, an anti-B7-H4 antagonist antibody, or antigen-binding fragment thereof, binds B7-H4 and inhibits activity of B7-H4 and/or binding of B7-H4 to its binding partner(s). Inhibition of activity and inhibition of binding includes partial inhibition. Methods for the identification of B7-H4 antibodies that block B7-H4 interactions are described herein and are known to the ones skilled in the art. For instance, competing, cross-blocking, and cross-blocked antibodies can be identified using any suitable method known in the art, including competition ELISAs or BIACORE® assays where binding of the competing or cross-blocking antibody to human B7-H4 prevents the binding of an antibody disclosed herein or vice versa.

In certain embodiments, not all CDRs are directly involved in binding to the antigen. In one embodiment, four out of six CDRs of the anti-B7-H4 antibody, or antigen-binding fragment thereof, make contact with the antigen. In one embodiment, five out of six CDRs of the anti-B7-H4 antibody, or antigen-binding fragment thereof, make contact with the antigen. In one embodiment, six out of six CDRs of the anti-B7-H4 antibody, or antigen-binding fragment thereof, make contact with the antigen.

The terms “selective” and “selectivity” herein refer to the preferential binding of an antibody, or antigen-binding fragment thereof, (i.e., a B7-H4 antibody or antigen-binding fragment thereof), for a particular region, target, or peptide; typically a region or epitope in B7-H4, as opposed to one or more other biological molecules, including other B7-H4 family members.

In one aspect, provided are anti-B7-H4 antibodies, and antigen-binding fragments thereof, that specifically bind to at least part of the binding site on B7-H4, thereby blocking B7-H4 interactions with the one or more B7-H4 ligands.

In certain embodiments, the anti-B7-H4 antibody, or antigen-binding fragment thereof, according to the disclosure comprises an Fc domain, composed of a first and a second subunit. The Fc domain of an antibody consists of a pair of polypeptide chains comprising heavy chain domains of an immunoglobulin molecule. The two subunits of the Fc domain form a stable association. In embodiments, the two subunits of the Fc domain are identical. In alternative embodiments, the two subunits of the Fc domain are non-identical. In embodiments, one subunit of the Fc domain may be fused with an immunoconjugate molecule. In embodiments, the Fc domain of the antibody may be an IgG Fc domain, an IgG₁ Fc domain, an IgG₂Fc domain, an IgG₃ Fc domain, an IgG₄ Fc domain. In a further particular embodiment, the Fc domain is a human Fc domain.

Fc Domain Modifications Promoting Heterodimerization

Further contemplated are modifications in the Fc domain of the disclosed anti-B7-H4 antibodies, or antigen-binding fragments, thereof promoting dimerization. A modification can be an amino acid substitution, insertion, deleting, or any other chemical or physical modification. In embodiments, the Fc domain of the anti-B7-H4 antibody, or antigen-binding fragment thereof, comprises a modification promoting the association of the first and the second subunit of the Fc domain. In one embodiment, said modification is in the CH3 domain of the Fc domain. In a specific embodiment, said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain.

Knob-into-hole modifications are a “protuberance-into-cavity” strategy, which serves to engineer an interface between a first and second polypeptide for hetero-oligomerization. “Protuberances” (i.e., the knobs) are constructed by replacing small amino acid side chains from the interface of the first polypeptide with larger side chains. Compensatory “cavities” (i.e., holes) of identical or similar size to the protuberances are optionally created on the interface of the second polypeptide by replacing large amino acid side chains with smaller ones. In particular embodiment, an amino acid residue in the CH3 domain of the first Fc subunit is replaced with an amino acid residue having a larger side chain volume, thereby generating a knob within the CH3 domain of the first Fc subunit which is positionable in a hole present within the CH3 domain of the second Fc subunit, generated by replacing one amino acid residue with an amino acid residue having a smaller side chain volume in the CH3 domain of the second Fc subunit. Preferably said amino acid residue having a larger side chain volume is selected from the group consisting of cysteine (C), valine (V), alanine (A), phenylalanine (F), tyrosine (Y), leucine (L), lysine (K), Proline (P), glutamic acid (E), and tryptophan (W). Preferably said amino acid residue having a smaller side chain volume is selected from the group consisting of alanine (A), serine (S), threonine (T), arginine (R), tryptophan (W), cysteine (C), lysine (L), glutamic acid (E), aspartic acid (D), and valine (V).

The mutations corresponding to the knob and the hole can be made by altering the nucleic acid encoding the polypeptides, e.g., by site-specific mutagenesis, or by peptide synthesis.

In fusion protein embodiments, in which only one heavy chain is linked to IL-15/IL-15Rα sushi, the Fc domains of the anti-B7-H4 antibody, or antigen-binding fragment thereof, may comprise one or more amino acid substitutions promoting heterodimer formation (i.e., the association the heavy chain fusion with a heavy chain lacking the fusion). In embodiments, an amino acid residue in the CH3 domain of one heavy chain (CH3-1) comprises an amino acid substitution replacing an amino acid with an amino acid residue having a larger side chain volume, thereby generating a “knob” within the CH3 domain, which is positionable in a “hole” present within the CH3 domain of the other heavy chain (CH3-2), generated by replacing an amino acid residue with an amino acid residue having a smaller side chain volume.

Provided herein is an anti-B7-H4 antibody, or antigen-binding fragment thereof, (or a fusion protein comprising such antibody, or antigen-binding fragment thereof), wherein:
(a) wherein the first heavy chain constant region comprises one or more amino acid substitutions selected from the group consisting of S354C, T366W, Y349C, T366S, L368A, and Y407V (Kabat EU index numbering) and wherein the second heavy chain constant region comprises one or more amino acid substitutions selected from the group consisting of S354C, T366W, Y349C, T366S, L368A, and Y407V (Kabat EU index numbering);
(b) the first heavy chain constant region comprises one or more amino acid substitutions selected from the group consisting of S354C and T366W (Kabat EU index numbering) and the second heavy chain constant region comprises one or more amino acid substitutions selected from the group consisting of Y349C, T366S, L368A, and Y407V (Kabat EU index numbering);
(c) the second heavy chain constant region comprises one or more amino acid substitutions selected from the group consisting of S354C and T366W (Kabat EU index numbering) and the first heavy chain constant region comprises one or more amino acid substitutions selected from the group consisting of Y349C, T366S, L368A, and Y407V (Kabat EU index numbering);
(d) the first heavy chain constant region comprises amino acid substitutions S354C and T366W (Kabat EU index numbering) and the second heavy chain constant region comprises amino acid substitutions Y349C, T366S, L368A, and Y407V (Kabat EU index numbering); or
(e) the second heavy chain constant region comprises amino acid substitutions S354C and T366W (Kabat EU index numbering) and the first heavy chain constant region comprises amino acid substitutions Y349C, T366S, L368A, and Y407V (Kabat EU index numbering).

Provided herein is an anti-B7-H4 antibody, or antigen-binding fragment thereof, (or a fusion protein comprising such an antibody or antigen-binding fragment thereof), wherein the antibody, or antigen-binding fragment thereof, comprises a heavy chain comprising a sequence selected from the group consisting of SEQ ID NOS:147-160 an/or a portion of comprising a sequence selected from the group consisting of SEQ ID NOS:147-160.

Linkers

In embodiments, the fusion proteins provided herein may comprise one or more linkers joining components of the fusion proteins disclosed herein. A linker may be located (i) between the IL-15 polypeptide and the IL15Rα polypeptide comprising the sushi domain; (ii) between a heavy chain of the anti-B7-H4 antibody, or antigen-binding fragment thereof, and the IL-15 polypeptide or the IL15Rα polypeptide comprising the sushi domain; or (iii) both. In embodiments of the disclosure, the IL-15 polypeptide and the IL15Rα polypeptide comprising the sushi domain are joined or linked by a first linker amino acid sequence. In embodiments, the IL15Rα polypeptide is linked to an antibody, or antigen binding fragment thereof, described herein by a second linker amino acid sequence. The first and second linkers may have the same or different amino acid sequences.

The linker amino acids sequences described herein may be of a length sufficient to ensure that the fusion protein forms proper secondary and tertiary structures. The length of the linker amino may be between 5 to 40 amino acids, preferably 10 to 40 amino acids, more preferably 15 to 40 amino acids, still more preferably 20 to 40 amino acids, most preferably 25 to 35 amino acids.

Preferably, the linker sequences comprise near neutral amino acids selected in the group comprising Gly (G), Asn (N), Ser (S), Thr (T), Ala (A), Leu (L), and Gln (Q), most preferably in amino acids selected from the group comprising Gly (G), Asn (N), and Ser (S). Preferably, the linker sequences are glycine- and serine-rich, and in some embodiments, the linker contains only serine and glycine residues.

In some embodiments, the linker comprises a portion of the IL15Rα polypeptide outside of the sushi domain, including, but not limited to the sequence of SEQ ID NO:171 (IRDPALVHQRPAPP). In some embodiments, the linker comprises SEQ ID NO:168 (SGGSGGGGSGGGSGGGGSLQ). In some embodiments, the linker comprises SEQ ID NO:171 and SEQ ID NO:168. In some embodiments, the linker comprises SEQ ID NO:170.

In embodiments, the heavy and light chains of the anti-B7-H4 antibody, or antigen-binding fragment thereof, disclosed herein may be connected into a single polypeptide chain (a “single-chain Fv” or “scFv”) using a third linker that allows the VR and VL domains to associate to form an antigen-binding site. The amino acid sequence of the linkers may be the same or different.

In one embodiment, an IL-15 polypeptide or an IL-15 derivative is covalently linked to an IL-15Rα sushi polypeptide or an IL-15Rα sushi derivative by a linker.

In some embodiments, an IL-15 polypeptide or an IL-15 derivative is covalently linked to an IL-15Rα sushi polypeptide or a IL-15Rα sushi derivative by a first linker and either the IL-15 polypeptide or IL-15 derivative or the IL-15Rα sushi polypeptide or IL-15Rα sushi derivative is covalently linked to an anti-B7-H4 antibody, or antigen-binding fragment thereof, by a second linker. In some embodiments, the amino acid sequences of the first and the second linker are identical. In other embodiments, the amino acid sequences of the first and the second linker are different.

Conjugates

The anti-B7-H4 antibodies, antigen-binding fragments thereof, and the fusion proteins disclosed herein may further comprise one or more functional moieties. Examples of useful functional moieties include, but are not limited to, a blocking moiety, a detectable moiety, a diagnostic moiety, a targeting, and a therapeutic moiety.

A blocking moiety may include moieties of sufficient steric bulk and/or charge such that reduced glycosylation occurs, for example, by blocking the ability of a glycosidase to glycosylate the antibody or antigen-binding fragment thereof. Preferred blocking moieties include cysteine adducts such as cysteine, mixed disulfide adducts, or disulfide linkages and PEG moieties such as polyethylene glycol (“PEG”), polypropylene glycol (“PPG”), polyoxyethylated glycerol (“POG”) and other polyoxyethylated polyols, polyvinyl alcohol (“PVA”) and other polyalkylene oxides, polyoxyethylated sorbitol, or polyoxyethylated glucose. PEG is a preferred moiety in biological applications for several reasons. PEGylating can improve pharmacokinetic performance of a molecule by increasing the molecule's apparent molecular weight. The increased apparent molecular weight reduces the rate of clearance from the body following subcutaneous or systemic administration. In many cases, pegylation can decrease antigenicity and immunogenicity. PEGylating can also increase the solubility of a biologically active molecule. Additionally, PEG typically is clear, colorless, odorless, soluble in water, stable to heat, inert to many chemical agents, does not hydrolyze, and is nontoxic, making it a preferable choice for biological applications.

The examples of detectable moieties that can be conjugated with the anti-B7-H4 antibody or the antigen-binding fragments or fusions disclosed herein may include fluorescent moieties or labels, imaging agents, radioisotopic moieties, radiopaque moieties, and the like, e.g., detectable labels such as biotin, fluorophores, chromophores, spin resonance probes, or radiolabels. Examples of fluorophores include fluorescent dyes (e.g., fluorescein, rhodamine, and the like) and other luminescent molecules (e.g., luminal). A fluorophore may be environmentally-sensitive such that its fluorescence changes if it is located close to one or more residues in the modified protein that undergo structural changes upon binding a substrate (e.g., dansyl probes). Exemplary radiolabels include small molecules containing atoms with one or more low sensitivity nuclei (¹³C, ¹⁵N, ²H, ¹²⁵I, ¹²³I, ⁹⁹Tc, ⁴³K, ⁵²Fe, ⁶⁷Ga, ⁶⁸Ga, ¹¹¹In and the like).

Diagnostic moieties include detectable moieties suitable for revealing the presence of a disease or disorder. Typically, a diagnostic moiety allows for determining the presence, absence, or levels of a molecule, for example, a target peptide, protein, or proteins, which are associated with a disease or disorder. Such diagnostics are also suitable for prognosing and/or diagnosing a disease or disorder and its progression.

Examples of therapeutic moieties include anti-inflammatory agents, anti-cancer agents, anti-neurodegenerative agents, anti-infective agents, or generally a therapeutic. The functional moiety may also have one or more of the above-mentioned functions. Exemplary therapeutic moieties may include an antibiotic, a second anti-B7-H4 antibody, or an antibody to another antigen such a tumor-specific antigen, an autoimmune tissue antigen, a virally-infected cell antigen, a Fc receptor, a T cell receptor, or a T cell co-inhibitor, or an immunotoxin, or any other therapeutic moiety useful for treating a disease or condition including cancer, autoimmune disease or chronic viral infection. Exemplary therapeutic moieties may also cytotoxin, radioactive agent, cytokine, interferon, target or reporter moiety, enzyme, toxin, peptide or therapeutic agent at any location along the molecule so long as it is able to bind its target. Examples of immunoconjugates include antibody drug conjugates and antibody-toxin fusion proteins. In certain embodiments, the antibody may be conjugated to an agent specific for a tumor cell or a virally infected cell.

A salvage receptor binding epitope as described, e.g., in U.S. Pat. No. 5,739,277 may also be attached to the antibody, or antigen-binding fragment thereof, (especially an antibody fragment) to increase the half-life of the antibodies or the antigen-binding fragments described herein. The term “salvage receptor binding epitope” may refer to an epitope of the Fc region of an IgG molecule (e.g., IgG1, IgG2, IgG3, or IgG4) that is responsible for increasing the in vivo serum half-life of the IgG molecule (e.g., Ghetie et al., 18 Ann. Rev. Immunol. 739 (2000).

Nucleic Acids

Also provided herein are nucleic acids encoding anti-B7-H4 antibodies, antigen-binding fragments thereof, and fusion proteins disclosed herein, as well as vectors, host cells, and expression systems. The term “nucleic acid” as used herein refers to a polymeric form of nucleotides of any length, either ribonucleotides or desoxyribonucleotides and includes but is not limited to, single-, double- or multi-stranded DNA or RNA, genomic DNA, cDNA, DNA-RNA hybrids, or a polymer comprising purine and pyrimidine bases, or other natural, chemically or biochemically modified, non-natural, or derivatized nucleotide bases. The nucleic acids encoding anti-B7-H4 antibodies, and antigen-binding fragments thereof, and fusion proteins disclosed herein may be, e.g., DNA, cDNA, RNA, synthetically produced DNA or RNA, or a recombinantly produced chimeric nucleic acid molecule comprising any of those polynucleotides either alone or in combination.

The term “vector” refers to vehicle comprising a nucleic acid molecule that is capable of transporting the nucleic acid molecule into a cell. A “vector” includes, but is not limited to, a viral vector, a plasmid, an RNA vector or a linear or circular DNA or RNA molecule, which may consist of a chromosomal, non-chromosomal, semi-synthetic or synthetic nucleic acids. In some embodiments, the employed vectors are those capable of autonomous replication (episomal vector) and/or expression of nucleic acids to which they are linked (expression vectors). A number of suitable vectors are known to those of skill in the art and are commercially available.

Provided herein is a nucleic acid or a set of nucleic acids encoding an antibody, or antigen-binding fragment thereof, disclosed in Tables 1, 5, 7, 9, 16-20 or 24. Provided herein is a nucleic acid or a set of nucleic acids encoding a fusion protein disclosed in Tables 13, 21, or 22.

Provided herein is a nucleic acid encoding an VH sequence disclosed in Tables 16-21. Provided herein is a nucleic acid encoding an VL sequence disclosed in Tables 16-21. Provided herein is a nucleic acid encoding an HC sequence disclosed in Tables 21-22. Provided herein is a nucleic acid encoding an LC sequence disclosed in Tables 21-22.

Provided herein is a vector or a set of vectors comprising one or more nucleic acid sequences disclosed herein.

Provided herein is a cell comprising a vector or a set of vectors disclosed herein. In some embodiments, the cell is an immune cell. In some embodiments, the cell is a mammalian cell. In some embodiments, the cell is a human cell. In some embodiments, the cell is an isolated cell.

Antibody and Fusion Protein Preparation and Expression Systems

The anti-B7-H4 antibodies, antigen-binding fragments, or the fusion proteins disclosed herein are typically produced by recombinant expression. Nucleic acids encoding light and heavy chain variable regions, optionally linked to constant regions, may be inserted into the same expression vectors. Alternatively, the nucleic acids encoding light and heavy chain variable regions, optionally linked to constant regions, are inserted into different expression vectors. The expression vector may further comprise one or more expression control sequences, which include, but are not limited to, promoters (e.g., homologous or heterologous promoters), signal sequences, enhancer elements, and transcription termination sequences. Preferably, the expression control sequences are eukaryotic promoter systems in vectors capable of transforming or transfecting eukaryotic host cells. Typically, the host is maintained under conditions suitable for high-level expression of the nucleotide sequences, and the collection and purification of the cross-reacting antibodies after the vector is incorporated into the appropriate host.

Commonly, expression vectors contain selection markers (e.g., ampicillin-resistance, hygromycin-resistance, tetracycline resistance or neomycin resistance) to permit detection of those cells transformed with the desired DNA sequences.

The host used to express the anti-B7-H4 antibodies, antigen-binding fragments thereof or the fusion proteins disclosed herein can be a prokaryotic or eukaryotic host. Examples of suitable hosts include bacterial or eukaryotic hosts, including yeast, insects, fungi, bird and mammalian cells either in vivo, or in situ, or host cells of mammalian, insect, bird, or yeast origin. The mammalian cell or tissue can be of human, primate, hamster, rabbit, rodent, cow, pig, sheep, horse, goat, dog, or cat origin, but any other mammalian cell may be used.

Examples of bacterial hosts that can be used to express the antibodies, antigen-binding fragments or the fusion protein disclosed herein can be E. coli, bacilli, such as Bacillus subtilis, and other enterobacteriaceae, such as Salmonella, Serratia, and various Pseudomonas species.

Yeasts may also be used as hosts for expressing the express the antibodies, antigen-binding fragments or the fusion protein disclosed herein. Saccharomyces and Pichia are exemplary yeast hosts, with suitable vectors having expression control sequences (e.g., promoters), an origin of replication, termination sequences, and the like as desired. Typical promoters include 3-phosphoglycerate kinase and other glycolytic enzymes. Inducible yeast promoters include, among others, promoters from alcohol dehydrogenase, isocytochrome C, and enzymes responsible for methanol, maltose, and galactose utilization.

Mammalian cells in culture may also be used as host cells for expressing the antibodies, antigen-binding fragments or the fusion proteins disclosed herein. Examples of suitable host cell lines capable of secreting heterologous proteins (e.g., intact immunoglobulins) which are well known in the art, include CHO cell lines, various COS cell lines, HeLa cells, 293 cells, myeloma cell lines, transformed B-cells, and hybridomas. Expression vectors for these cells can include expression control sequences, such as an origin of replication, a promoter, an enhancer and necessary processing information sites such as ribosome binding site, RNA splice site and/or transcriptional terminator sequences. Examples of expression control sequences include SV40, adenovirus, bovine papilloma virus, cytomegalovirus and the like.

The anti-B7-H4 antibodies, antigen-binding fragments thereof, and the fusion proteins disclosed herein can be expressed using a single expression construct or vector or multiple expression constructs or vectors (e.g., two or three expression constructs). When the antibody heavy and light chains are cloned on separate expression vectors, the vectors are co-transfected to obtain expression and assembly of intact immunoglobulins. Once expressed, the whole antibodies, their dimers, individual light and heavy chains, or other immunoglobulin forms disclosed herein can be purified according to standard procedures of the art, including ammonium sulfate precipitation, affinity columns, column chromatography, HPLC purification, gel electrophoresis, and the like (see generally Scopes, Protein Purification (Springer-Verlag, N.Y., (1982)). Substantially pure immunoglobulins of at least about 90 to 95% homogeneity are preferred, and 98 to 99% or more homogeneity most preferred, for pharmaceutical uses.

The disclosed anti-B7-H4 antibodies, antigen-binding fragments, and the fusion proteins can be made by any method known in the art. General techniques for generating human or mouse antibodies or fusion molecules are known in the art.

Methods for Modulating B7-H4 Activity

In one aspect, provided are methods of using the anti-B7-H4 antibodies, antigen-binding fragments thereof, and fusion proteins described herein for decreasing the interaction between B7-H4 and its cognate ligand. Such decrease may include internalization of the receptor following binding of the antibody or a reduction in expression of the receptor on the target cell.

In one aspect, provided are methods of using the anti-B7-H4 antibodies, antigen-binding fragments thereof, and fusion proteins described herein for decreasing signal transduction that occurs as a consequence of B7-H4 −cognate receptor binding. Such decrease may be partial (i.e., attenuating, but not abolishing, the activity of B7-H4) or it may completely abolish such activity (e.g., neutralize the ability of B7-H4 to mediate signal transduction). Such decrease may include internalization of the receptor following binding of the antibody or a reduction in expression of the receptor on the target cell.

In one aspect, provided are methods of using the anti-B7-H4 antibodies, antigen-binding fragments thereof, and fusion proteins described herein for reducing immunosuppression, e.g., T cell tolerance. Immunosuppression can be mediated by immune inhibitory receptors expressed on the surface of an immune cell, and their interactions with their ligands. Methods of measuring T cell activity are known in the art. By way of non-limiting example, T cell tolerance can be induced by contacting T cells with recall antigen, anti-CD3 in the absence of co-stimulation, and/or ionomycin. Levels of, e.g., IL-27, LDH-A, RAB10, and/or ZAP70 (both intracellular or secreted) can be monitored, for example, to determine the extent of T cell tolerogenesis (with levels of IL-2, interferon-7 and TNF correlating with increased T cell tolerance).

In one aspect, provided are methods of using the anti-B7-H4 antibodies, antigen-binding fragments thereof, and fusion proteins described herein for enhancing T cell expansion, activation, and/or proliferation. Provided are methods of using the anti-B7-H4 antibodies, antigen-binding fragments thereof, and fusion proteins described herein for enhancing CD8+ T cell proliferation. Provided are methods of using the anti-B7-H4 antibodies, antigen-binding fragments thereof, and fusion proteins described herein for enhancing CD4+ T cell proliferation.

In one aspect, provided are methods of using the anti-B7-H4 antibodies, antigen-binding fragments thereof, and fusion proteins described herein for depleting B7-H4-expressing tumor cells or tumor-associated macrophages (TAMs) in a recipient human or in human tissue (in situ or ex vivo). Depletion of B7-H4 positive tumor cells or TAMs can be monitored by immunohistochemistry (IHC) of tumor tissues using the disclosed anti-B7-H4 antibodies (or another tumor-specific or TAM-specific marker), or a reduction in B7-H4 mRNA levels by PCR, in-situ hybridization or another other method known to one skilled in the art.

In one aspect, provided are methods of using the anti-B7-H4 antibodies, antigen-binding fragments thereof, and fusion proteins described herein for inducing antibody dependent cell mediated cytotoxicity (ADCC) in a B7-H4-expressing cell in a subject in need thereof.

By “reducing” (or “decreasing”) is meant the ability to cause an overall decrease of about 20% or greater, 30% or greater, 40% or greater, 45% or greater, 50% or greater, of 55% or greater, of 60% or greater, of 65% or greater, of 70% or greater, or 75% or greater, 80% or greater, 85% or greater, 90% or greater, or 95% or greater, as compared to a control that is not treated.

Cells targeted by the anti-B7-H4 antibodies, antigen-binding fragments thereof, and fusion proteins described herein include, but are not limited to, cells expressing B7-H4, for example, transformed cells that typically do not express B7-H4 when they are not transformed, or cells characterized by increased expression compared to control cells. Preferred target cells include cancer cells and tumor associated macrophages.

Methods of Treatment

In one aspect, the disclosure provides anti-B7-H4 antibodies, antigen-binding fragments thereof, and fusion proteins that are useful for the treatment of subjects in need thereof.

In the methods described herein, a therapeutically effective amount of an anti-B7-H4 antibody, antigen-binding fragment thereof, or fusion protein disclosed herein is administered to a mammal in need thereof. The term “mammal” as used herein includes, but is not limited to, humans, laboratory animals, domestic pets, and farm animals. Preferably, the mammal is a human. “Therapeutically effective amount” as described herein refers to an amount of an anti-B7-H4 antibody, antigen-binding fragment thereof, or fusion protein which, when administered to a mammal, is effective in producing the desired therapeutic effect.

In embodiments, the “subject” is a mammal. In embodiment, the subject is a non-primate (e.g., cows, pigs, horses, cats, dogs, rats, mouse, etc.) or a primate (e.g., monkey and human). Individuals and patients are also subjects herein.

The terms “treat,” “treated,” “treating,” or “treatment” as used herein refer to therapeutic treatment, wherein the object is to slow down (lessen) an undesired physiological condition, disorder, or disease, or to obtain beneficial or desired clinical results. For the purposes of this disclosure, beneficial or desired clinical results include, but are not limited to, alleviation of symptoms; diminishment of the extent of the condition, disorder or disease; stabilization (i.e., not worsening) of the state of the condition, disorder or disease; delay in onset or slowing of the progression of the condition, disorder or disease; amelioration of one or more symptoms of the condition, disorder or disease state; and remission (whether partial or total). Treatment includes eliciting a clinically significant response without excessive levels of side effects. Treatment also includes prolonging survival as compared to expected survival if not receiving treatment.

The terms “prevent,” “prevention,” and the like refer to acting prior to overt disease or disorder onset, to prevent the disease or disorder from developing or to minimize the extent of the disease or disorder or slow its course of development.

Subjects likely to benefit from treatment with an anti-B7-H4 antibody will express the target B7-H4 protein, either on tumor or TAMs, and this can be assessed by IHC of tumor samples, FACs, in-situ hybridization or another other method known to one skilled in the art. Tin embodiments, the subject has upregulated expression of B7-H4.

Provided herein is a method of treating cancer in a subject in need thereof, the method comprising administering to the subject an effective amount of an anti-B7-H4 antibody, or antigen-binding fragment thereof, or a fusion protein disclosed herein. Provided herein is an anti-B7-H4 antibody, or antigen-binding fragment thereof, or a fusion protein for use in treating cancer in a subject in need thereof. Provided herein is the use of an anti-B7-H4 antibody, or antigen-binding fragment thereof, or a fusion protein for use in treating cancer in a subject in need thereof. Provided herein is an anti-B7-H4 antibody, or antigen-binding fragment thereof, or a fusion protein in the manufacture of a medicament for treating cancer in a subject in need thereof.

Provided herein is a method of reducing tumor growth in a subject in need thereof, the method comprising administering to the subject an effective amount of an anti-B7-H4 antibody, or antigen-binding fragment thereof, or a fusion protein disclosed herein. Provided herein is an anti-B7-H4 antibody, or antigen-binding fragment thereof, or a fusion protein for use in reducing tumor growth in a subject in need thereof. Provided herein is the use of an anti-B7-H4 antibody, or antigen-binding fragment thereof, or a fusion protein for use in reducing tumor growth in a subject in need thereof. Provided herein is an anti-B7-H4 antibody, or antigen-binding fragment thereof, or a fusion protein in the manufacture of a medicament for reducing tumor growth in a subject in need thereof.

Provided herein is a method of reducing tumor metastasis in a subject in need thereof, the method comprising administering to the subject an effective amount of an anti-B7-H4 antibody, or antigen-binding fragment thereof, or a fusion protein disclosed herein. Provided herein is an anti-B7-H4 antibody, or antigen-binding fragment thereof, or a fusion protein for use in reducing tumor metastasis in a subject in need thereof. Provided herein is the use of an anti-B7-H4 antibody, or antigen-binding fragment thereof, or a fusion protein for use in reducing tumor metastasis in a subject in need thereof. Provided herein is an anti-B7-H4 antibody, or antigen-binding fragment thereof, or a fusion protein in the manufacture of a medicament for reducing tumor metastasis in a subject in need thereof.

Cancers and related disorders that can be treated or prevented with the anti-B7-H4 antibodies, antigen-binding fragments thereof, and fusion proteins disclosed herein include, but are not limited to, the following: leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as, but not limited to, Hodgkin's disease, non-Hodgkin's disease; multiple myelomas such as, but not limited to, smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as, but not limited to, bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including, but not limited to, ocular melanoma such as iris melanoma, choroidal melanoma, and ciliary body melanoma, and retinoblastoma; vaginal cancers, including, but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including, but not limited to, squamous cell carcinoma, and adenocarcinoma; uterine cancers including, but not limited to, endometrial carcinoma and uterine sarcoma; ovarian cancers including, but not limited to, ovarian epithelial carcinoma, borderline tumor, germ cell tumor, and stromal tumor; esophageal cancers including, but not limited to, squamous cancer, adenocarcinoma, adenoid cystic carcinoma, mucoepidermoid carcinoma, adenosquamous carcinoma, sarcoma, melanoma, plasmacytoma, verrucous carcinoma, and oat cell (small cell) carcinoma; stomach cancers including, but not limited to, adenocarcinoma, fungating (polypoid), ulcerating, superficial spreading, diffusely spreading, malignant lymphoma, liposarcoma, fibrosarcoma, and carcinosarcoma; colon cancers; rectal cancers; liver cancers including, but not limited to, hepatocellular carcinoma and hepatoblastoma, gallbladder cancers including, but not limited to, adenocarcinoma; cholangiocarcinomas including, but not limited to, papillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including, but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including, but not limited to, adenocarcinoma, leiomyosarcoma, and rhabdomyosarcoma; penal cancers; oral cancers including, but not limited to, squamous cell carcinoma; basal cancers; salivary gland cancers including, but not limited to, adenocarcinoma, mucoepidermoid carcinoma, and adenoidcystic carcinoma; pharynx cancers including, but not limited to, squamous cell cancer, and verrucous; skin cancers including, but not limited to, basal cell carcinoma, squamous cell carcinoma and melanoma, superficial spreading melanoma, nodular melanoma, lentigo malignant melanoma, acral lentiginous melanoma; kidney cancers including, but not limited to, renal cell cancer, adenocarcinoma, hypernephroma, fibrosarcoma, transitional cell cancer (renal pelvis and/or uterer); Wilms' tumor; bladder cancers including, but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas.

Accordingly, the anti-B7-H4 antibodies, antigen-binding fragments thereof, and fusion proteins disclosed herein include are also useful in the treatment or prevention of a variety of cancers (particularly, ovarian, breast, prostate, gastric, renal, thyroid, and uterine cancer) or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosarcoma, rhabdomyosarcoma, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions disclosed herein. Such cancers can include, but are not limited to, follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or prevented by the disclosed methods and compositions in the ovary, bladder, breast, colon, lung, skin, pancreas, or uterus. In other specific embodiments, sarcoma, melanoma, or leukemia is treated or prevented by the methods and compositions provided.

Cancer cells acquire a characteristic set of functional capabilities during their development, albeit through various mechanisms. Such capabilities include evading apoptosis, self-sufficiency in growth signals, insensitivity to anti-growth signals, tissue invasion/metastasis, limitless explicative potential, and sustained angiogenesis. The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen (e.g., pan-carcinoma antigen (KS 1/4), ovarian carcinoma antigen (CA125), prostate specific antigen (PSA), carcinoembryonic antigen (CEA), CD19, CD20, HER2/neu, etc.).

Combination Therapies

The anti-B7-H4 antibodies, antigen-binding fragments thereof and fusion proteins disclosed herein may be advantageously combined with an additional therapeutic agent. Such additional agents include, but are not limited to, cytotoxic agents, chemotherapeutic agents, growth inhibitory agents, anti-inflammatory agents, anti-cancer agents, anti-neurodegenerative agents, immunosuppressive agents, and anti-infective agents. The administration of the anti-B7-H4 antibody, or antigen-binding fragment thereof, or the fusion protein and the additional therapeutic agent (or additional therapy) may be concurrently, consecutively or intermittently. The administration of the anti-B7-H4 antibody, or antigen-binding fragment thereof, or the fusion protein and the additional therapeutic agent (or additional therapy) may be separately or as a mixture. Further, the methods of treatment provided herein can relate to a treatment in combination with one or more therapies including but not limited to the group of antibody therapy, chemotherapy, cytokine therapy, dendritic cell therapy, gene therapy, hormone therapy, laser light therapy, and radiation therapy.

The anti-B7-H4 antibodies, antigen-binding fragments thereof, and the fusion proteins of the present disclosure may be combined synergistically with one or more anti-cancer drugs or therapy used to treat cancer, including, but not limited to, renal cell carcinoma, colorectal cancer, glioblastoma multiforme, squamous cell carcinoma of head and neck, non-small-cell lung cancer, colon cancer, ovarian cancer, adenocarcinoma, prostate cancer, glioma, and melanoma. Examples of such agents include but are not limited to with an antibody to PD-L1 (e.g., nivolumab), a LAG-3 inhibitor, a CTLA-4 inhibitor (e.g., ipilimumab), a TIM3 inhibitor, a BTLA inhibitor, a T IGIT inhibitor, a CD47 inhibitor, an antagonist of another T cell co-inhibitor or ligand (e.g., an antibody to CD-28, 2B4, LY108, LAI R 1, ICOS, CD1 60 or VISTA), an indoleamine-2,3-dioxygenase (IDO) inhibitor, a vascular endothelial growth factor (VEGF) antagonist (e.g., a “VEGF-Trap” such as aflibercept or other VEGF-inhibiting fusion protein as set forth in U.S. Pat. No. 7,087,411, or an anti-VEGF antibody, or antigen-binding fragment thereof, (e.g., bevacizumab, or ranibizumab) or a small molecule kinase inhibitor of VEGF receptor (e.g., sunitinib, sorafenib, or pazopanib)), an Ang2 inhibitor (e.g., nesvacumab), a transforming growth factor β (TGF3) inhibitor, an epidermal growth factor receptor (EGFR) inhibitor (e.g., erlotinib, cetuximab), an agonist to a costimulatory receptor (e.g., an agonist to glucocorticoid-induced TNFR-related protein), an antibody to a tumor-specific antigen (e.g., CA9, CA1 25, melanoma-associated antigen 3 (MAGE3), carcinoembryonic antigen (CEA), anti-viral drugs (e.g., zidovudine, lamivudine, abacavir, ribavirin, lopinavir, efavirenz, cobicistat, tenofovir, rilpivirine and corticosteroids), vimentin, tumor-M2-PK, prostate-specific antigen (PSA), mucin-1, MART-1, and CA1 9-9), a vaccine (e.g., Bacillus Calmette-Guerin, a cancer vaccine), an adjuvant to increase antigen presentation (e.g., granulocyte-macrophage colony stimulating factor), a bispecific antibody (e.g., CD3×CD20 bispecific antibody, PSMA×CD3 bispecific antibody), cancer vaccines (e.g., MAGE3, MUC1, EGFRv3, ALVAC-CEA), a cytotoxin, a chemotherapeutic agent (e.g., dacarbazine, temozolomide, cyclophosphamide, docetaxel, doxorubicin, daunorubicin, cisplatin, carboplatin, gemcitabine, methotrexate, mitoxantrone, oxaliplatin, paclitaxel, and vincristine), cyclophosphamide, radiotherapy, an IL-6R inhibitor (e.g., sarilumab), an IL-4R inhibitor (e.g., dupilumab), an IL-1 0 inhibitor, a cytokine such as IL-2, IL-7, IL-21, and IL-15, an antibody-drug conjugate (ADC) (e.g., anti-CD1 9-DM4 ADC, and anti-DS6-DM4 ADC), an anti-inflammatory drug (e.g., corticosteroids, and non-steroidal anti-inflammatory drugs), a dietary supplement such as anti-oxidants or any palliative care to treat cancer, radiation therapy, and/or an antibody to a Fc receptor on immune cells for the treatment of an autoimmune disease.

Methods of Administration

The therapeutic compositions comprising any of the anti-B7-H4 antibodies, antigen-binding fragments thereof or fusion proteins described herein may be administered to a subject in need thereof in any convenient manner including but not limited to by injection, transfusion, implantation or transplantation. The compositions described herein may be administered to a subject in need thereof subcutaneously, intradermally, intratumorally, intranodally, intramedullary, intramuscularly, intracranially, by intravenous or intralymphatic injection, or intraperitoneally. In one embodiment, the cell compositions of the present disclosure are preferably administered by intravenous injection.

In certain embodiments, the anti-B7-H4 antibody, antigen-binding fragment thereof or the fusion protein is administered to the mammal by intravenous infusion, i.e., introduction of the anti-B7-H4 antibody, antigen-binding fragment thereof or the fusion protein into the vein of a mammal over a certain period. In certain embodiments, the period is about 5 min, about 10 min, about 30 min, about 1 h, about 2 h, about 4 h, or about 8 h.

In some embodiments, the composition is administered directly to the tumor or the tumor microenvironment to enhance localization of the composition to the tumor site and reduce toxicity to non-target cells expressing B7-H4.

Administrative Regimens

The methods according to this aspect of the disclosure comprise sequentially administering to a subject multiple doses of an anti-B7-H4 antibody, antigen-binding fragment thereof, or a fusion protein of the disclosure. As used herein, “sequentially administering,” means that each dose of an anti-B7-H4 antibody, antigen-binding fragment thereof, or a fusion protein of the disclosure is administered to the subject at a different point in time, e.g., on different days separated by a predetermined interval (e.g., hours, days, weeks or months). The present disclosure includes methods, which comprise sequentially administering to the patient a single initial dose of an anti-B7-H4 antibody, antigen-binding fragment thereof, or a fusion protein of the disclosure, followed by one or more secondary doses of an anti-B7-H4 antibody, antigen-binding fragment thereof, or a fusion protein of the disclosure, and optionally followed by one or more tertiary doses of an anti-B7-H4 antibody, antigen-binding fragment thereof, or a fusion protein of the disclosure. An anti-B7-H4 antibody, antigen-binding fragment thereof, or a fusion protein of the disclosure may be administered at a dose between 0.01 mg/kg to 100 mg/kg.

In certain embodiments, a dose of a compound or a composition is administered to a subject every day, every other day, every couple of days, every third day, once a week, twice a week, three times a week, once every two weeks, or once a month. In other embodiments, two, three or four doses of a compound or a composition is administered to a subject every day, every couple of days, every third day, once a week, once every two weeks or once a month. In some embodiments, a dose(s) of a compound or a composition is administered for 2 days, 3 days, 5 days, 7 days, 14 days, 21 days, or 28 days. In certain embodiments, a dose of a compound or a composition is administered for 1 month, 1.5 months, 2 months, 2.5 months, 3 months, 4 months, 5 months, 6 months or more.

Pharmaceutical Compositions

In another aspect, provided are pharmaceutically acceptable compositions that comprise a therapeutically effective amount of an anti-B7-H4 antibody, antigen-binding fragment thereof, or a fusion protein of the disclosure formulated together with one or more pharmaceutically acceptable excipients.

The dosage of active agent(s) may vary, depending on the reason for use, the individual subject, and the mode of administration. The dosage may be adjusted based on the subject's weight, the age, and health of the subject, and tolerance for the compound(s) or composition.

The active agent and excipient(s) may be formulated into compositions and dosage forms according to methods known in the art. The pharmaceutical compositions provided herein may be specially formulated in solid or liquid form, including those adapted for parenteral administration, for example, by subcutaneous, intratumoral, intramuscular or intravenous injection as, for example, a sterile solution, or suspension.

Therapeutic compositions comprising anti-B7-H4 antibodies or antigen-binding fragments thereof, or fusion proteins thereof may formulated with one or more pharmaceutically-acceptable excipients, which can be a pharmaceutically-acceptable material, composition or vehicle, such as a liquid or solid filler, diluent, carrier, manufacturing aid (e.g., lubricant, talc magnesium, calcium or zinc stearate, or steric acid), solvent or encapsulating material, involved in carrying or transporting the therapeutic compound for administration to the subject, bulking agent, salt, surfactant and/or a preservative. Some examples of materials which can serve as pharmaceutically-acceptable excipients include: sugars, such as lactose, glucose and sucrose; starches, such as corn starch and potato starch; cellulose and its derivatives, such as sodium carboxymethyl cellulose, ethyl cellulose and cellulose acetate; gelatin; talc; waxes; oils, such as peanut oil, cottonseed oil, safflower oil, sesame oil, olive oil, corn oil and soybean oil; glycols, such as ethylene glycol and propylene glycol; polyols, such as glycerin, sorbitol, mannitol and polyethylene glycol; esters, such as ethyl oleate and ethyl laurate; agar; buffering agents; water; isotonic saline; pH buffered solutions; and other non-toxic compatible substances employed in pharmaceutical formulations.

A bulking agent as referred herein may be described as a compound added to increase the mass of a pharmaceutical composition and to contribute to the physical structure of the formulation in the lyophilized form. Examples of bulking agent may include but is not limited to Suitable mannitol, glycine, polyethylene glycol, and sorbitol.

The therapeutic composition may optionally include a surfactant. The use of a surfactant can reduce aggregation of the reconstituted protein and/or reduce the formation of particulates in the reconstituted formulation. Examples of suitable surfactants that might be used according to the present disclosure includes but is not limited to polysorbates (e.g. polysorbates 20 or 80); poloxamers (e.g. poloxamer 188); Triton; sodium dodecyl sulfate (SDS); sodium laurel sulfate; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and polyethyl glycol, polypropyl glycol, and copolymers of ethylene and propylene glycol (e.g. Pluronics, PF68, etc.).

A preservative may optionally be used in the therapeutic composition described herein. Suitable preservatives for use in the formulation provided herein include octadecyldimethylbenzyl ammonium chloride, hexamethonium chloride, benzalkonium chloride (a mixture of alkylbenzyl-dimethylammonium chlorides in which the alkyl groups are long-chain compounds), and benzethonium chloride. Other types of preservatives include aromatic alcohols such as phenol, butyl, and benzyl alcohol, alkyl parabens such as methyl or propyl paraben, catechol, resorcinol, cyclohexanol, 3-pentanol, and m-cresol.

The therapeutic composition described herein may have a varying concentration of the anti-B7-H4 antibody, antigen-binding fragment thereof, or fusion protein. For example, the compositions may comprise an anti-B7-H4 antibody, antigen-binding fragment thereof or fusion protein at 10 mg/ml to 200 mg/ml, 25 mg/ml to 130 mg/ml, 50 mg/ml to 125 mg/ml, 75 mg/ml to 110 mg/ml, or 80 mg/ml to 100 mg/ml. The compositions also may comprise an anti-B7-H4 antibody, antigen-binding fragment thereof or fusion protein at about 10 mg/ml, 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, or 150 mg/ml. In some embodiments, the therapeutic composition may be lyophilized and provided in a composition for reconstitution prior to administration.

Diagnostic Uses

The anti-B7-H4 antibodies, antigen-binding fragments thereof or the fusion proteins of the present disclosure may be used to detect and/or measure B7-H4 in a sample, e.g., for diagnostic purposes. The anti-B7-H4 antibodies, antigen-binding fragments thereof or the fusion proteins disclosed herein may be used in an assay to detect a disease or disorder such as cancer. Exemplary diagnostic assays for B7-H4 may comprise, e.g., contacting a sample, obtained from a patient, with an anti-B7-H4 antibody, antigen-binding fragment thereof, or a fusion protein of the disclosure, wherein the anti-B7-H4 antibody, antigen-binding fragment thereof, or the fusion protein is labeled with a detectable label or reporter molecule or used as a capture ligand to selectively isolate B7-H4 from patient samples or alternatively used in combination with a secondary antibody which is itself detectably labeled. The detectable label or reporter molecule can be a radioisotope, a fluorescent or chemiluminescent moiety such as fluorescein isothiocyanate, or rhodamine; or an enzyme such as alkaline phosphatase, β-galactosidase, horseradish peroxidase, or luciferase. Specific exemplary assays that can be used to detect or measure B7-H4 in a sample include enzyme linked immunosorbent assay (ELISA), radioimmunoassay (RIA), and fluorescence-activated cell sorting (FACS).

Kits of the present disclosure can include any combination of agents, compositions, components, reagents, administration devices, or mechanisms, or other entities provided herein. For instance, a kit of the present disclosure may include one or more anti-B7-H4 antibodies or antigen-binding fragments thereof or fusion proteins disclosed herein and one or more of a carrier composition, an administration device, and a combination therapy agent. Kits may further include a device to facilitate delivery such as syringe for injection or a tool that facilitates the delivery of therapeutic compositions to the subject in need thereof. Any of the kits provided herein can be included in a container, pack, or dispenser together with instructions for administration.

All other referenced patents and applications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

It is to be understood that this disclosure is not limited to the particular molecules, compositions, methodologies, or protocols described, as these may vary. Any methods and materials similar or equivalent to those described herein can be used in the practice or testing of embodiments of the present disclosure. It is further to be understood that the current disclosure in this specification includes all possible combinations of such particular features. For example, where a particular feature is disclosed in the context of a particular aspect or embodiment of the disclosure herein, or a particular claim, that feature can also be used, to the extent possible, in combination with and/or in the context of other particular aspects and embodiments of the disclosure, and in the disclosure generally.

Where reference is made herein to a method comprising two or more defined steps, the defined steps can be carried out in the order as listed, or in any order or simultaneously (except where the context excludes that possibility), and the method can include one or more other steps which are carried out before any of the defined steps, between two of the defined steps, or after all the defined steps (except where the context excludes those possibilities).

To facilitate a better understanding of the present disclosure, the following examples of specific embodiments are given. The following examples should not be read to limit or define the entire scope of the disclosure.

### EXAMPLES

### Example 1: Materials and Methods for Examples 1-21

Generation of Human/Mouse B7-H4-Fc, IgV Domain of Human/Mouse B7-H4, and IgC Domain of Human and Mouse B7-H4.

Genes for human B7-H4 (NCBI accession #: NP_078902, Phe29-Ala258) and mouse B7-H4 (NCBI accession #: Q7TSP5, Phe29-Pro258) were synthesized (IDT) and fused to a gene encoding the N-terminal of human IgG1 Fc (NCBI accession #: P01587, Pro100-Lys330). The genes encoding for the B7-H4-Fc fusion proteins were inserted to a mammalian expression vector such as pcDNA 3.1 (ThermoFisher) and transfected to the Expi293F cells (ThermoFisher). After incubating the cells in large capacity reach-In CO2 incubator (Thermo Scientific) at 30° C. for 4-5 days, the clear supernatant was loaded to MabSelect SuRe Protein A column. Purified protein was eluted was pH 3 glycine buffer. After neutralization with pH 7.5 Tris buffer, the protein was analyzed by SDS-PAGE or/and SE-HPLC. The same method was used to generate IgV domain of both human and mouse B7-H4-Fc (Phe29-Val157) and IgC domain of human B7-H4-Fc (Asp158-Ala258) or mouse B7-H4-Fc (Asp158-Pro258).

Antibody Identification from Superman 2.0 Phage Display Library

Human and mouse B7-H4-his were biotinylated by using the EZ-Link™ NHS-PEG4-Biotin (ThermoFisher). Purified phage from Superman 2.0 phage display library (Distributed Bio) was mixed with 100 μg to 0.5 μg of human/mouse B7-H4-Fc or biotinylated human/mouse B7-H4-his. After incubating in RT for 0.5 to 1 hour, the mixture of B7-H4/phage was captured by protein G (for Fc-fusion B7-H4) or strep (for biotinylated B7-H4) magnetic beads. The unbound phage was washed out with PBS-T and PBS and eluted with 100 mm Triethylamine (TEA). Eluted phage was neutralized with pH 7.5 Tris buffer and used for the infection electrocompetent ER2738 E. coli cells growing in log phase. After incubating in 30° C. overnight, the bacteria were harvested and used to inoculate to 2YT with glucose, tetracycline and carbenicillin. M13 helper phage was used to infect the log phase bacterial, and media was changed to 2YT with kanamycin and carbenicillin to continue to amplify the phage overnight. The next day, the phage were purified by PEG and re-suspended in PBS. Purified phages were ready for the next round panning. To monitor the panning, individual bacterial colonies were pick and grew to log phase. M13 helper phage was added to amplify the phage as described above. The crude phage mix was added to a B7-H4 coated plate. Binding was detected using an HRP-anti-M13 antibody. Generally, 3-4 rounds of panning were performed until the positive rate reached >50%.

The light chain shuffling method was used to improve the affinity of the lead. Briefly, the new phage library was constructed by paired the lead heavy chain with the pool of light chain from Superman 2.0 library. Higher affinity antibodies were identified in harsh conditions such as higher temperature, lower or high pH, and the presence of additional B7-H4 antigens in the washing buffers.

IG Conversion from scFv, Expression, and Purification

The genes encoding the variable domains light chain and the variable heavy chain were fused to genes encoding the constant domain of human IgG1 light chain or heavy chain, respectively. The entire protein-encoding sequences (encoding both variable and constant domains) were inserted into a mammalian expression vector such as pcDNA3.1 (ThermoFisher). The vector was transfected into an expression cell line such as HEK293, Chinese Hamster Ovary (CHO) cells, or al, 6-fucosyltransferase (FUT8) transfected CHO (enhancing the ADCC activity). The antibodies were purified using a Protein A column and SEC-HPLC to produce pure antibodies with monomer >95%.

Fusion Protein Generation

The Gene encoding the IL-15 receptor sushi domain (residues 33-93 of NCBI accession #: EAW86418.1) and mature IL-15 (residues 49-162 of NCBI accession #: P40933) were synthesized and fused to the 3′ end of the sequence encoding for a heavy chain of anti-B7-H4 antibody (resulting in a gene encoding a C-terminal fusion protein). Mutations of the heavy chains (S354C and T366W in one heavy chain, Y349C, T366S, L368A and Y407V in the other heavy chain) were introduced to stabilize the heterodimer of two heavy chains. Additional mutations (M428L and N434S) in the Fc region of the antibody were introduced to increase antibody binding to FcRn at pH 6, thus extending the serum half-life of the protein. The expression and purification of fusion antibodies was performed as described above.

Dose Response ELISA

About 1-2 μg/ml of human or mouse B7-H4-Fc (or his-tagged) was immobilized on ELISA plates (Immuno 2HB, Thermo scientific, cat #3455) at 4° C. overnight. After blocking the plates with 3% PBS+milk, serial diluted antibodies were added to the blocked plates and incubated at RT for 1-1.5 h. After washing the plates with PBS-T (0.05%), horse radish peroxidase (HRP)-conjugated antibody targeting the hFab was added to the plate. Signals were developed by adding 3,3′,5,5′-Tetramethylbenzidine (TMB (followed 1N of H₂SO₄ to stop the reaction. The OD at 450 nm was read by ELISA plate reader (Infinite M1000 from TECAN). GraphPad from Prism was used to plot OD450 vs. antibody concentration curve and to calculate the EC50 values.

Biacore Kinetics

Surface plasmon resonance experiments were performed using a Biacore T200 instrument equipped with CM5 sensor chips. The system was equilibrated in running buffer HBS-EP pH 7.4. The ligands were immobilized using amine-coupling chemistry. The surfaces of flow cells one through four were activated for 5 min using an amine coupling kit at a flow rate of 10 l/min. The ligands (human B7-H4-Fc, human B7-H4-hism and mouse B7-H4-Fc; at a concentration of 5 μg/ml in 10 mM sodium acetate, pH 5.0) were immobilized at a density of 50-100 RU on flow cell 2, 3, 4 respectively. Flow cell 1 was left blank to serve as a reference surface. All flow cell surfaces were blocked with a 5 min injection of 1 M ethanolamine, pH 8.0. To collect kinetic binding data, the respective analyte (diluted in 1×HBS-EP buffer, pH 7.4) was injected over the four flow cells at concentrations of 100 nM to 0.1 nM at a flow rate of 30 l/min and at a temperature of 25° C. The protein complex was allowed to associate and dissociate for 180 seconds and 1000 or 1200 seconds, respectively. The surfaces were regenerated with a 30 second injection of 1.5 M NaCl/0.05 M NaOH. The data were fit to a simple 1:1 interaction model using the Langmuir fit option available within the Biaevaluaton software

Generation of a CT26 Cell Line Expressing mB7-H4

Murine carcinoma CT26 cell line (CT26.CL25, cat #CRL-2639) was obtained from ATCC. Murine B7-H4 (Vtcn1) ORF cDNA in pReceiver-M68 vector (cat #EX-32401-M68) and its empty control vector pReceiver-M68 vector (cat #EX-NEG-M68) were purchased from GeneCopoeia. CT26 cells were transfected with purified plasmids using Lipofectamine® 2000 DNA transfection reagent. After 48-72 hours of transfection, transfected CT26 cell colonies were selected and cultured in puromycin-containing medium. mB7-H4 expression level of the selected clones was detected by staining PE anti-mouse B7-H4 antibody (cat #139406, Biolegend) using Guava® FLOW Cytometry easyCyte™ System.

Assessment of Antibody Fusion Protein Binding to Cell-Expressed B7-H4 by FACS

Cells were harvested, washed once, and then re-suspended to the concentration of 1×10⁶ cells/ml in ice cold FACS Buffer. 25 μl of cell suspension was added to each well of a 96-well round-bottom microliter plate. Serially diluted antibodies or fusion proteins were added to each well and incubated for 1 hour at 4° C. Cells were washed three times by centrifugation at 1500 rpm for 5 minutes, and then stained with secondary antibody (1:200 dilution) for 30 min at 4° C. in the dark. Cells were washed once, re-suspended in 100 μl PBS buffer and then analyzed using Guava Flow Cytometry EasyCyte system.

M-07e Cell Culture and Proliferation Assay

Human acute megakaryoblastic leukemia M-07e cell line were cultured in IMDM medium supplemented with 15% heat-inactivated FBS and 20% conditioned medium of cell line 5637. Serial dilutions of antibodies or fusion proteins were added in a 96-well plate, and then cells were seeded at 2.5×10⁴ per well in complete IMDM medium. Cells were incubated at 37° C. for 3 days and cell proliferation were detected using CellTiter-Glo® luminescent cell viability assay kit.

Proliferation of Human PBMC and T Cell Subtype Analysis

Human frozen PBMCs were thawed on the day of the experiment and were prepared in complete IMDM medium supplemented with 10% heat-inactivated FBS for proliferation assay immediately. Serial dilutions of antibodies were added in a 96-well plate and then cells were seeded at 1×10⁵ cells per well. Cells were cultured for 6 days at 37° C. and cell proliferation was detected using CellTiter-Glo® luminescent cell viability assay kit.

For T cell population analysis, cells were washed once and stained with fixable viability dye eFluor780 for 10 minutes at 4° C. After washing, cell were fixed, permeabilized with Foxp3 transcription factor staining buffer set, and then stained with anti-CD3-PE, anti-CD4-APC, anti-CD8α-PECy7 and anti-Ki67-FITC for 1 hour at 4° C. Proliferative CD4 (CD4+Ki67+) or CD8 (CD8+Ki67+) T cell population was analyzed and defined using Guava Flow Cytometry EasyCyte system.

IFNγ Cytokine Secretion by T cell

Human B7-H4 transfected cell line hB7-H4-CHO was seeded at 4×10⁴ cells per well into a 96-well U-bottom plate. Serially diluted antibodies were added into each well. A cell suspension of effector cells and human T cells (isolated from LeuPak using EasySep human T cell isolation kit) was prepared and seeded at a desired effector-to-target cell ratio (e.g., as 5:1) into each well. The co-culture was incubated at 37° C. for 96 hours and the medium was collected for an IFNγ cytokine ELISA (R&D).

Antibody Internalization Assay

Target cells (such as SK-BR-3 MDA-MB-468 cells) were seeded at 6,000 cells per well into 96-well flat-bottom plate and incubate for 3 hours to allow cells to settle on a level surface. An antibody/Incucyte FabFlour reagent mix (IncuCyte® Human Fabfluor-pH Red Antibody Labeling Dye) was added into each well to test in a final concentration of 2 μg/ml. The assay plate was repeatedly, scanning every hour using IncuCyte© live-cell analysis system. The images were analyzed by IncuCyte® software.

Assessment of Tumor Cell Killing Using an ADCC Reporter Bioassay from Promega

The ADCC Reporter Bioassay uses engineered effector cells. Assays were performed using an ADCC Reporter Bioassay Kit (ADCC Reporter Bioassay G7010 for the hIgG1). Briefly, target cancer cells were seeded at 1.5×10⁴ cells per well into 96-well flat-bottom plate. Next day, the growth medium was aspirated off, and ADCC assay buffer and antibody dilution series were added into each well. A cell suspension of ADCC Bioassay effector cells was prepared and seeded at a desired effector-to-target cell ratio (e.g., 5:1). After 6 hours incubation at 37° C., Bio-Glo™ Luciferase Assay Reagent was added into each well and incubated for 15 minutes, and luminescence signal was measured using a plate reader.

Tumor Cell Killing by hPBMCs (Immune Cell Killing Assay)

For the measurement of tumor cell killing by immune cell in the present of antibodies or fusion proteins, an SKB-R3/hPBMC co-culture model was set-up for analyzing antibody-dependent cell mediated cytotoxicity (ADCC) (short term analysis) or cytotoxic T cell killing (long term analysis). Target cancer cells, SKB-R3 cells, were labeled with IncuCyte© CytoLight Red Rapid Reagent and seeded at 5,000 cells per well into 96-well flat-bottom plate. The next day, the growth medium was aspirated off, and IncuCyte© Annexin V Green Dye and antibody dilutions were added into each well. A cell suspension of effector cells, hPBMCs, was prepared and seeded at a desired effector-to-target cell ratio (10:1 or 5:1). The assay plate was repeatedly scanned and the images were analyzed based the instruction using IncuCyte© live-cell analysis system and IncuCyte© software.

pSTAT5 Measurement Using HEK-Blue IL-2 Reporter Cells

HEK-Blue™ IL-2 cells were generated by stable transfection of the human embryonic kidney HEK 293 cell line with the human IL-2Rα, IL-2Rβ, and IL-2Rγ genes, along with the human JAK3 and STAT5 genes to obtain a fully functional IL-2 signaling pathway. In addition, a STAT5-inducible SEAP reporter gene was also introduced. Upon IL-2 stimulation, HEK-Blue™ IL-2 cells trigger the activation of STAT5 and the subsequent secretion of SEAP. The levels of STAT5-induced SEAP can be readily monitored using QUANTI-Blue™ Solution.

The HEK-Blue IL-2 cells were maintained and sub-cultured in complete DMEM medium (supplemented with 10% heat-inactivated FBS) with 1 μg/ml puromycin and 1×HEK-Blue CLR Selection. Cells were suspended at 2.8×10⁵/ml in complete DMEM medium for reporter assay. 20 μl of the serially diluted antibodies or fusion antibodies was added to each well of a flat-bottom 96-well plate first, and then 180 μl of cell suspension. The cells were incubated at 37° C. in a CO2 incubator for 20-24 h. The SEAP levels were detected at 630 nm following the instruction of QUANTI-Blue™ Solution.

Antibody Nomenclature

An antibody called simply “X” refers to an antibody comprising both a heavy variable chain called “X” and a light variable chain called “X.” For example, antibody 3F2 comprises a 3F2 light and a 3F2 heavy variable chain.

An antibody called “X/Y” comprises a heavy variable chain called “X” and a light variable chain “Y.” For example, antibody 3F2/50A10 comprises a 3F2 heavy variable chain and a 50A10 light variable chain.

Control Antibodies

The following antibodies were used as control: (1) B7-H4 IgV domain binding antibody FPA150 (Five Prime), see antibody “20502” in US publication US2019/0085080A1 (“control antibody 1”; binds to IgV domain of b7-H4, also referred to as FPA150) (2) B7-H4 IgC domain binding antibody 6H3 (Medimmune), see antibody “6H3” in U.S. patent U.S. Pat. No. 9,574,000 (“control antibody 2”; binds to IgC domain of b7-H4), and (3) DP47.

### Example 2: Generation of High Affinity Anti-B7-H4 Antibodies

Phage display was conducted as described above. First, panning on immobilized hB7-H4-Fc or Biotin-hB7-H4-his captured by strep beads was performed. See FIG. 1 (Step 1). In this step, the following antibodies were identified:

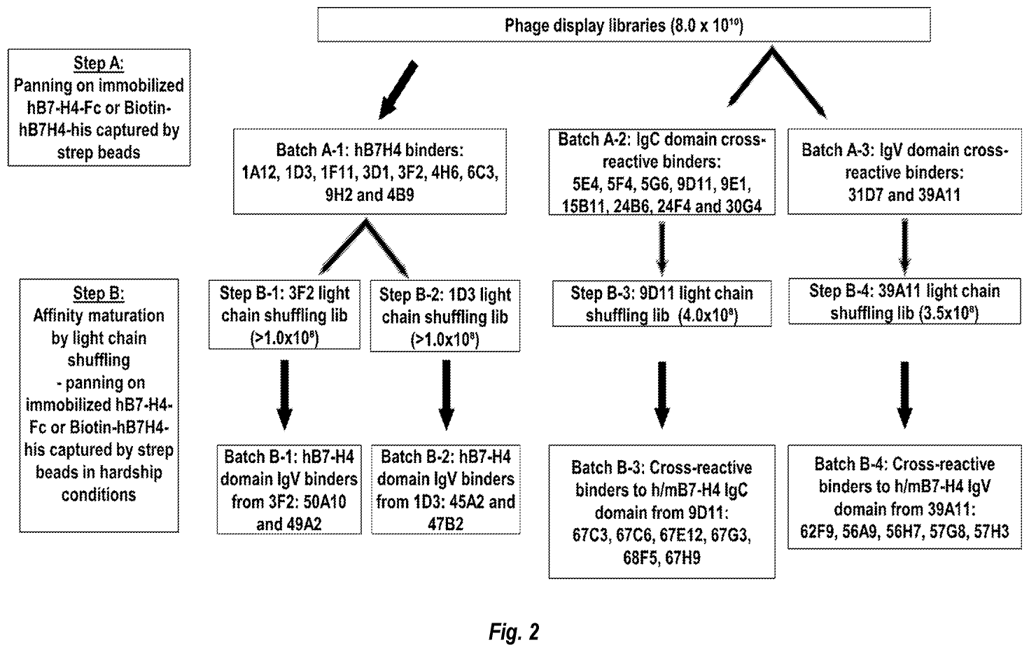

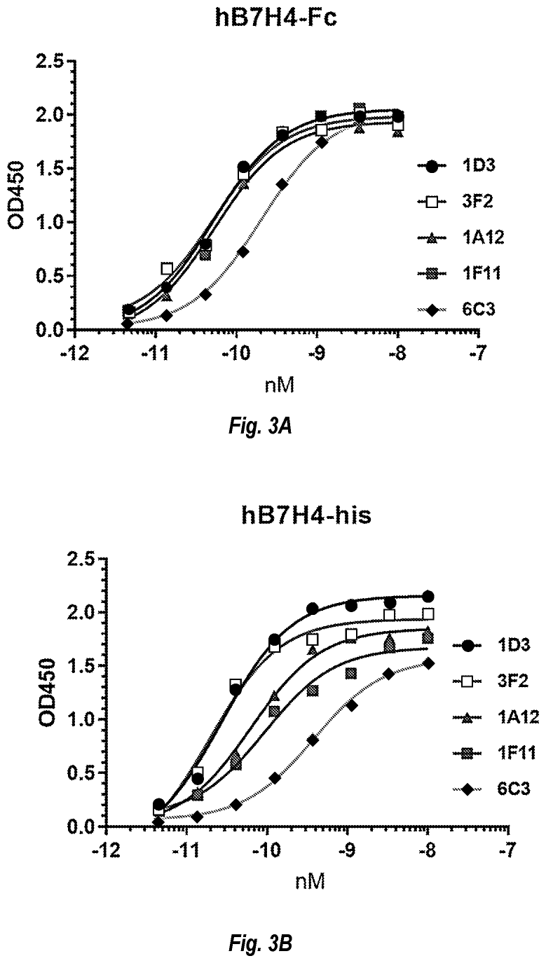

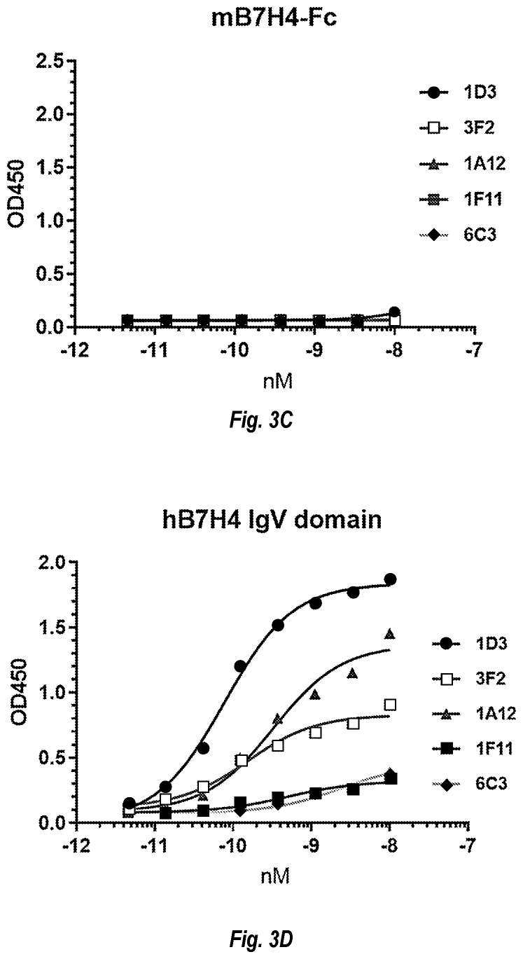

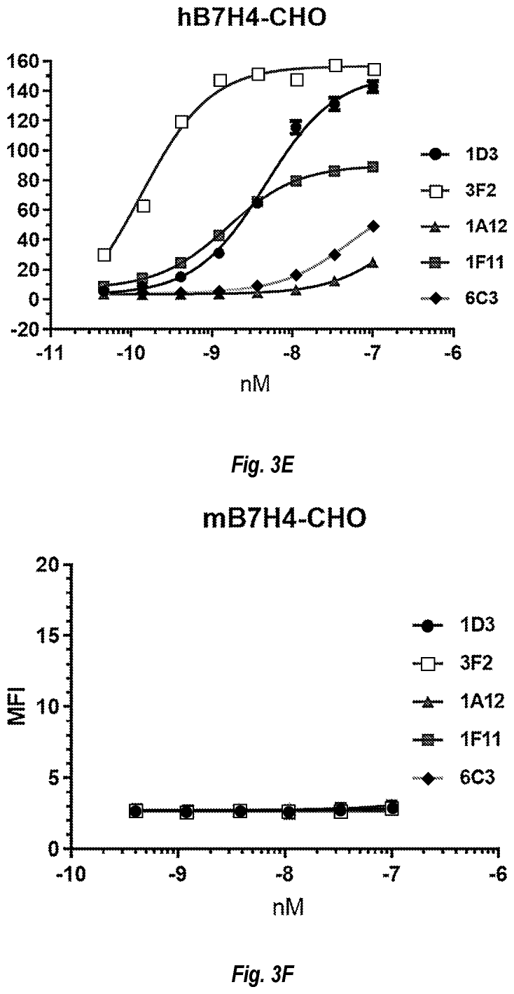

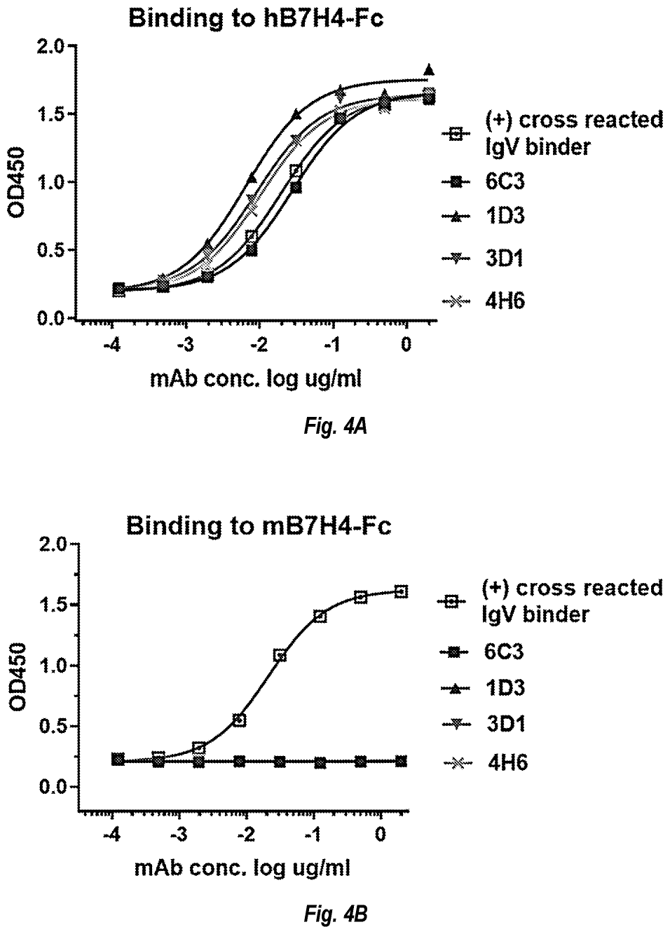

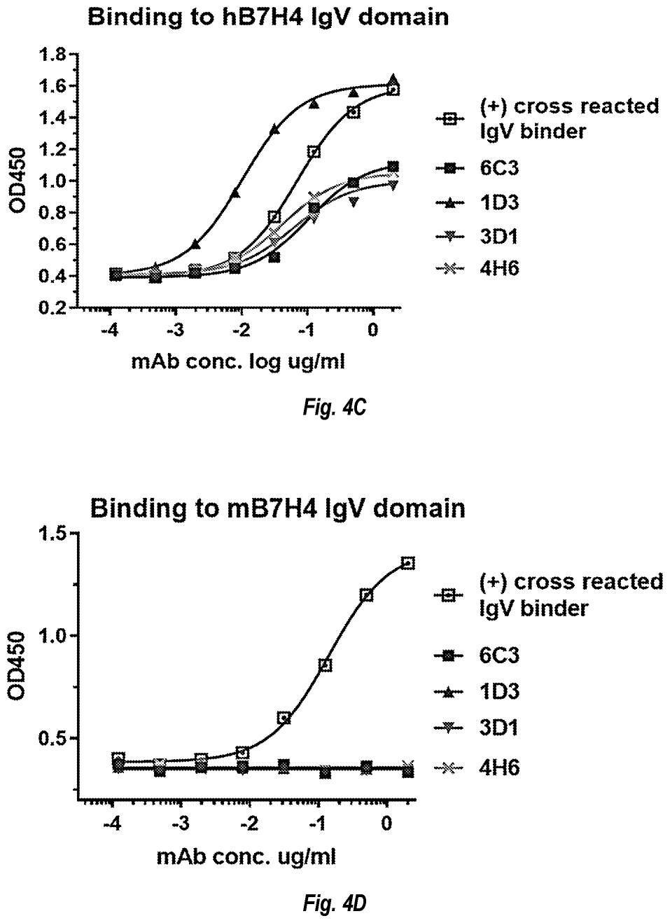

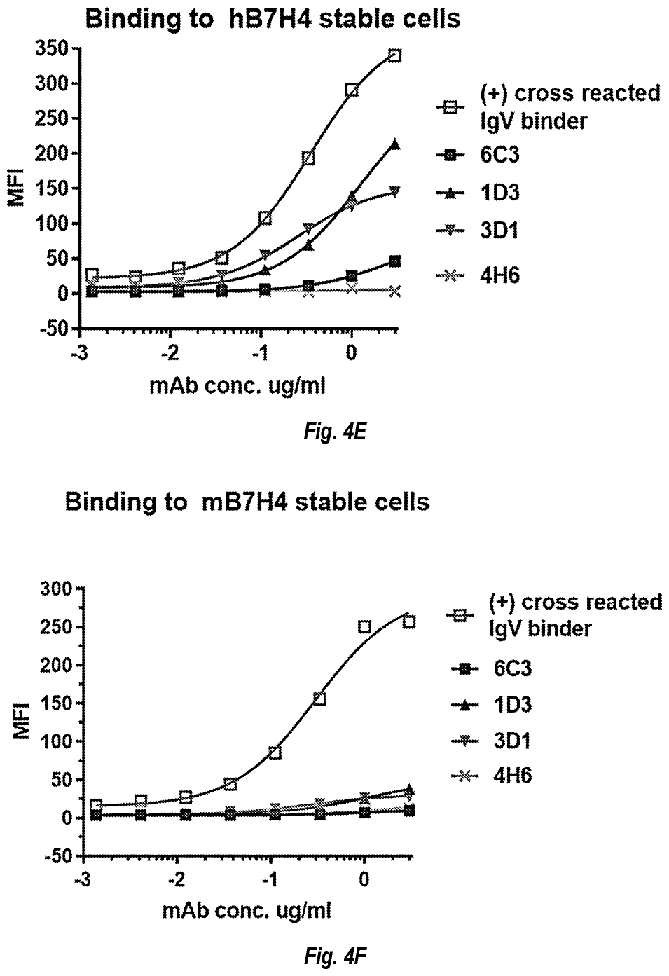

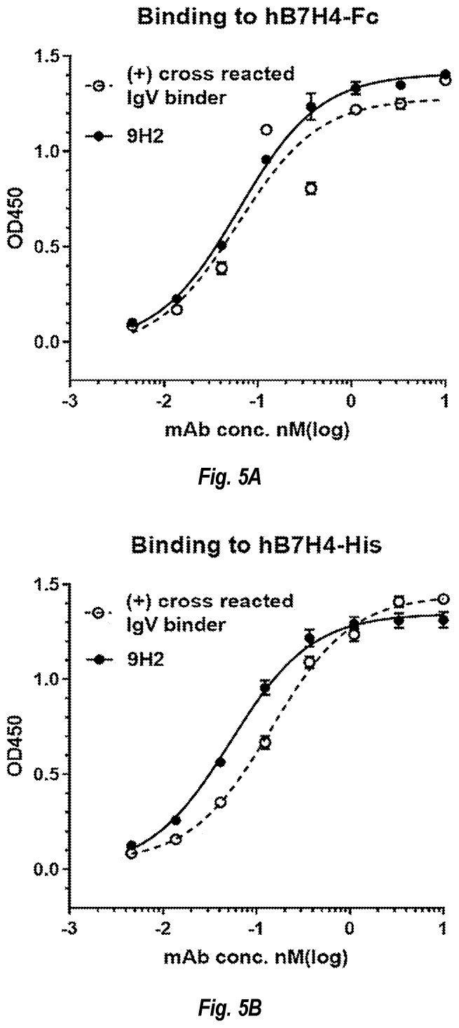

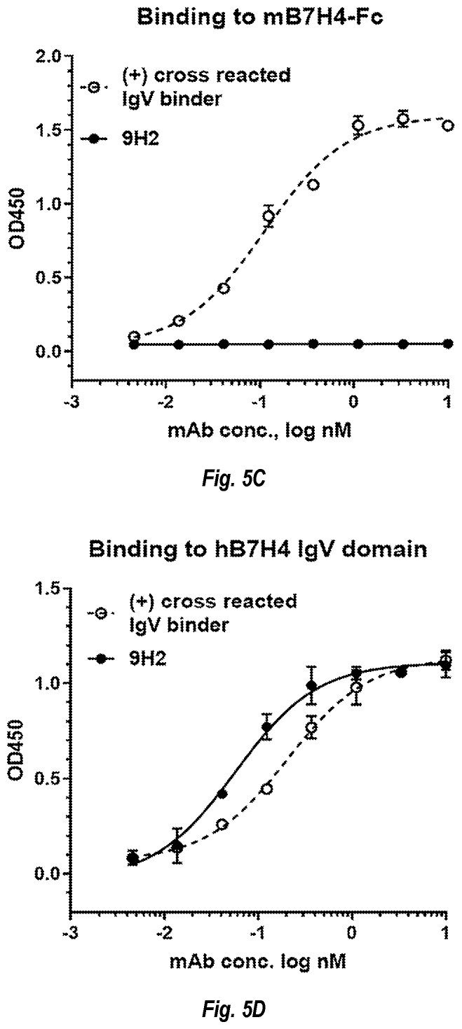

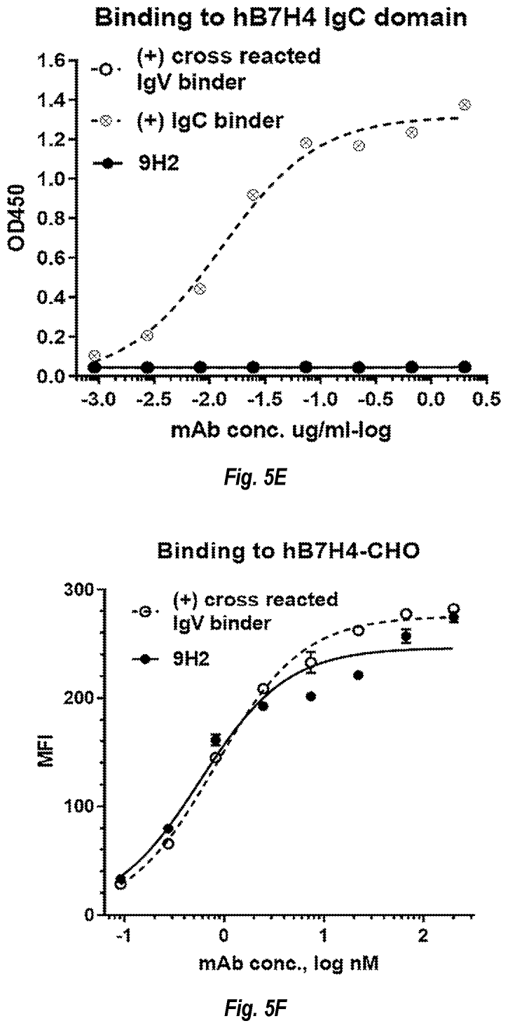

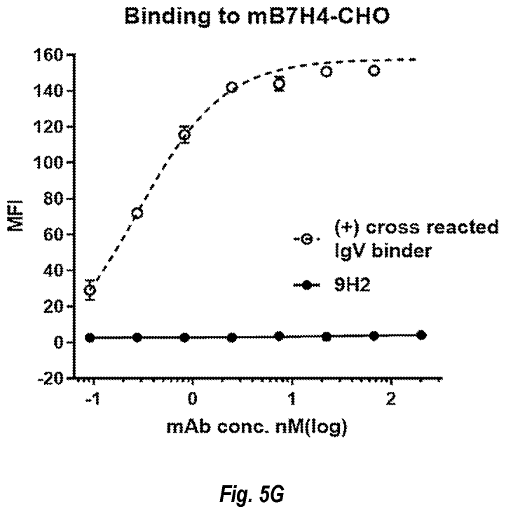

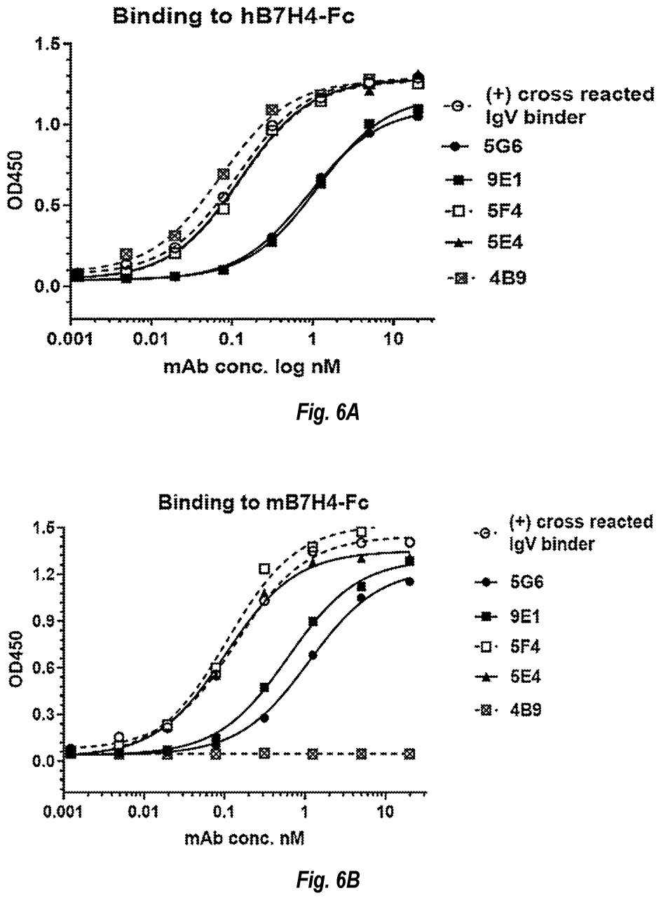

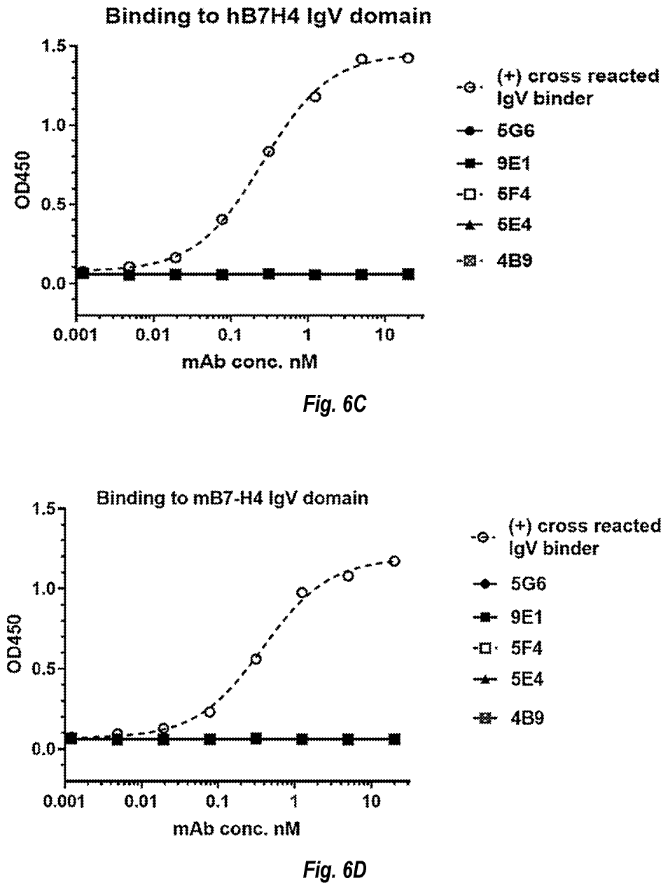

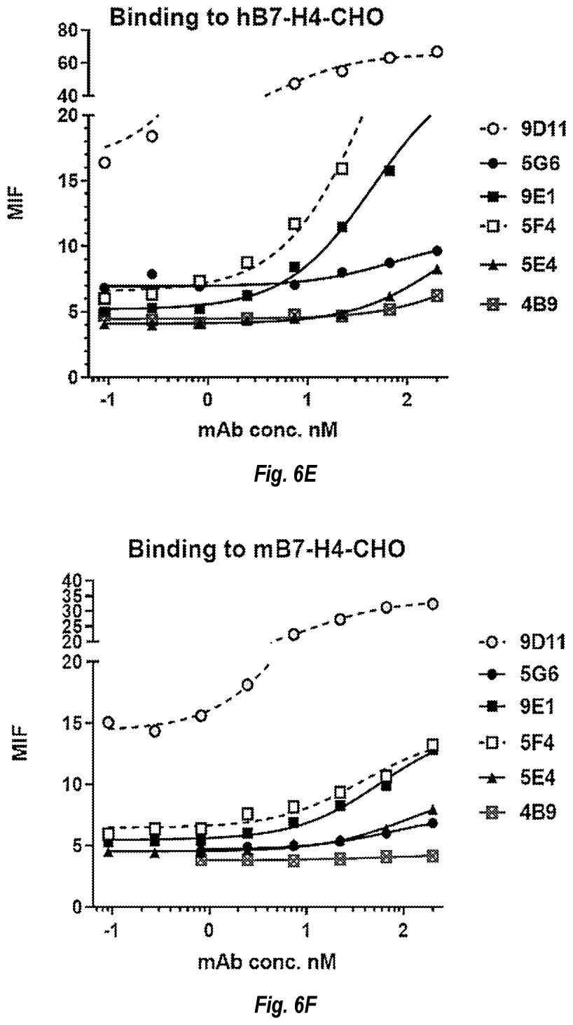

(1) Antibodies binding to hB7-H4: 1A12, 1D3, 1F11, 3D1, 3F2, 4H6, 6C3, 9H2, and 4B9 (FIG. 2 (Batch A-1)). Antibodies 1A12, 1D3, 1F11, 3D1, 3F2, 4H6, 6C3 and 9H2 bound to both soluble and cellular hB7-H4, specifically to the IgV domain of hB7-H4 (FIGS. 2-5). 4B9 bound to hB7-H4-Fc strongly but did not bind to mB7-H4. Binding of 4B9 to hB7-H4 required both IgV and IgC (FIG. 6).

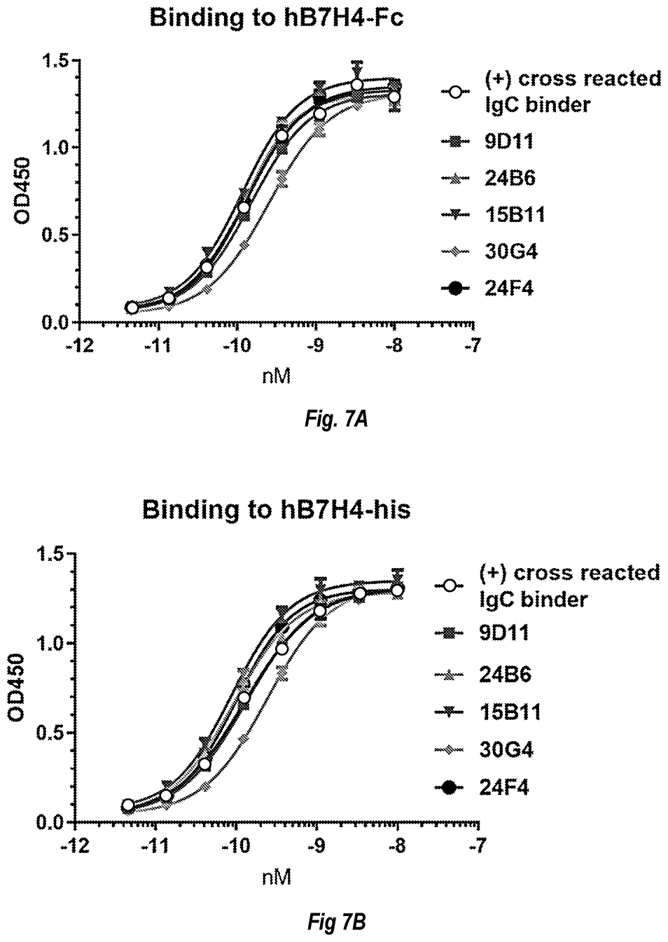

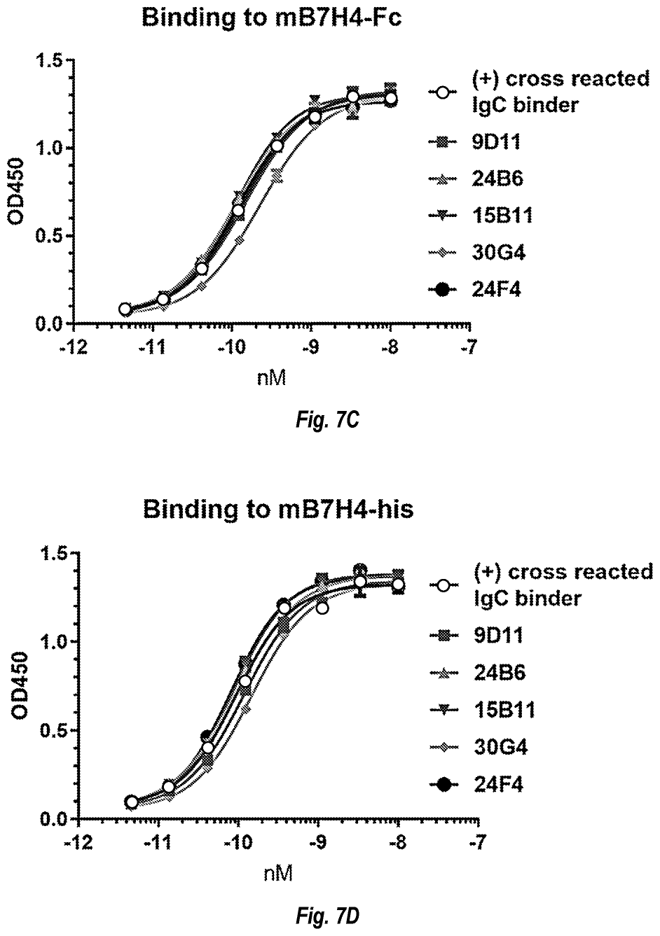

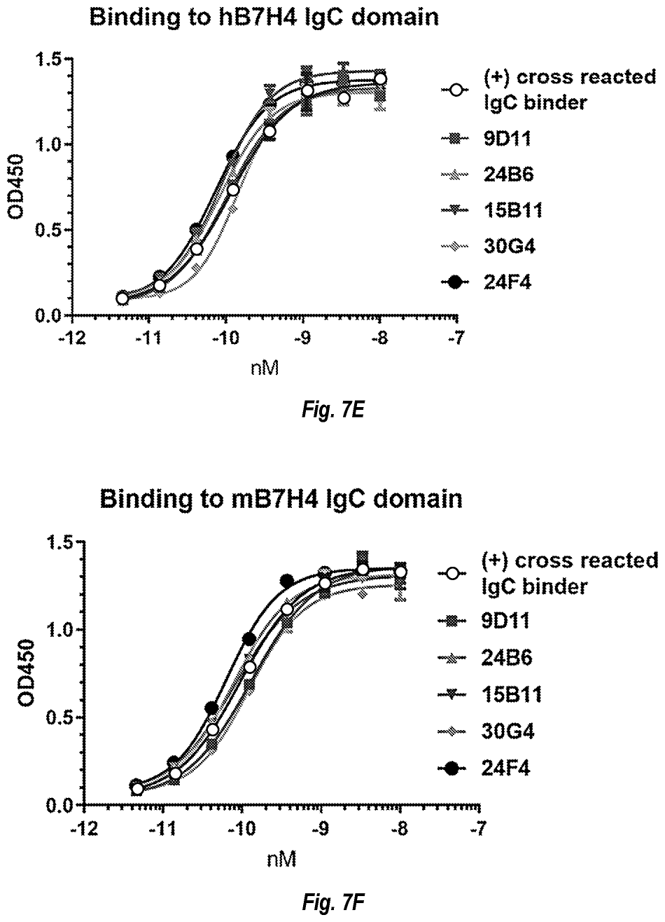

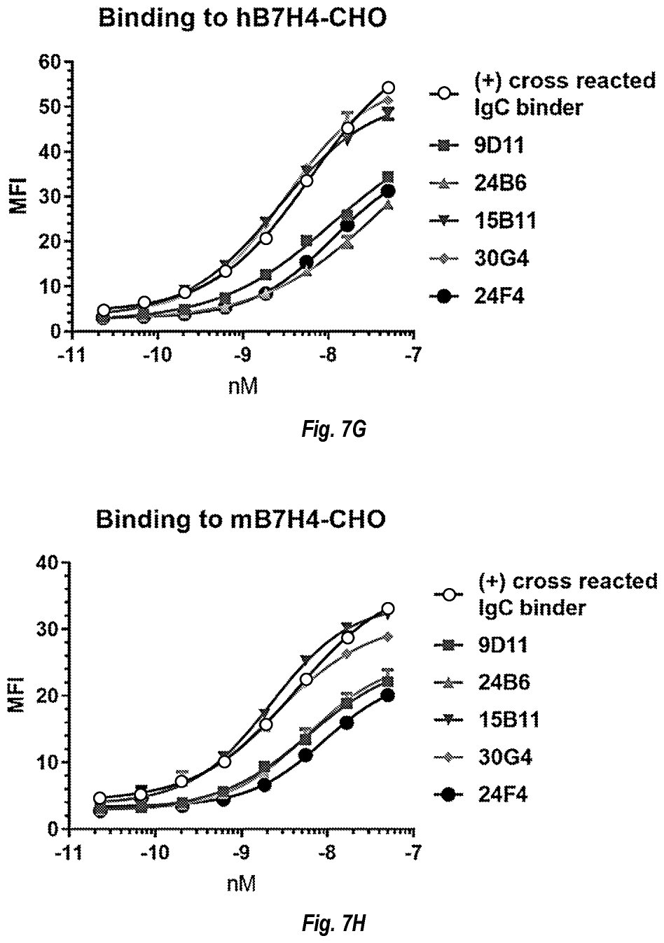

(2) Antibodies binding to IgC domain of human and murine hB7-H4: 5E4, 5F4, 5G6, 9111, 9E1, 15B11, 24B6, 24F4, and 30G4 (FIG. 2 (Batch A-2) and FIGS. 6-7).

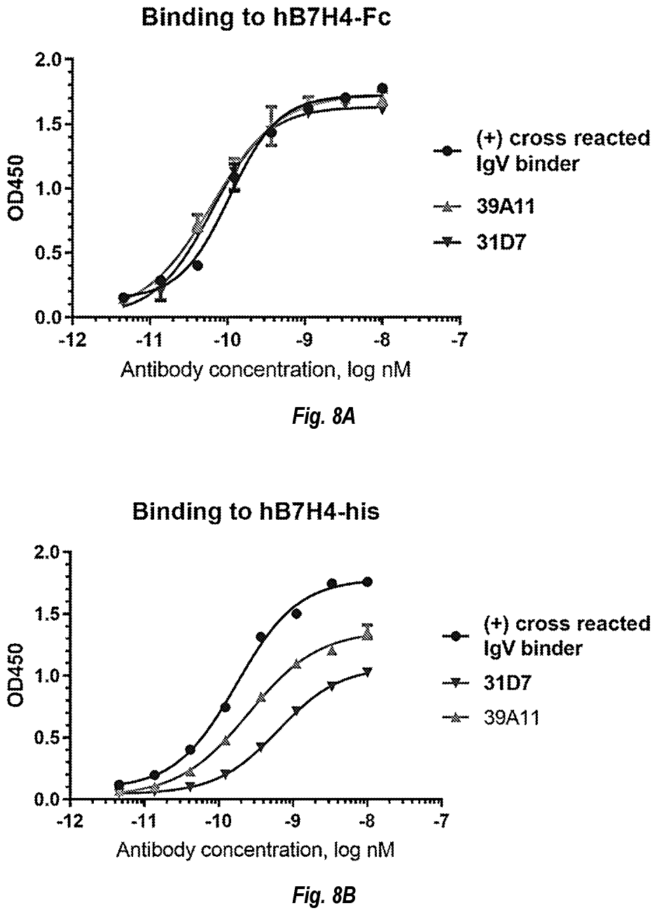

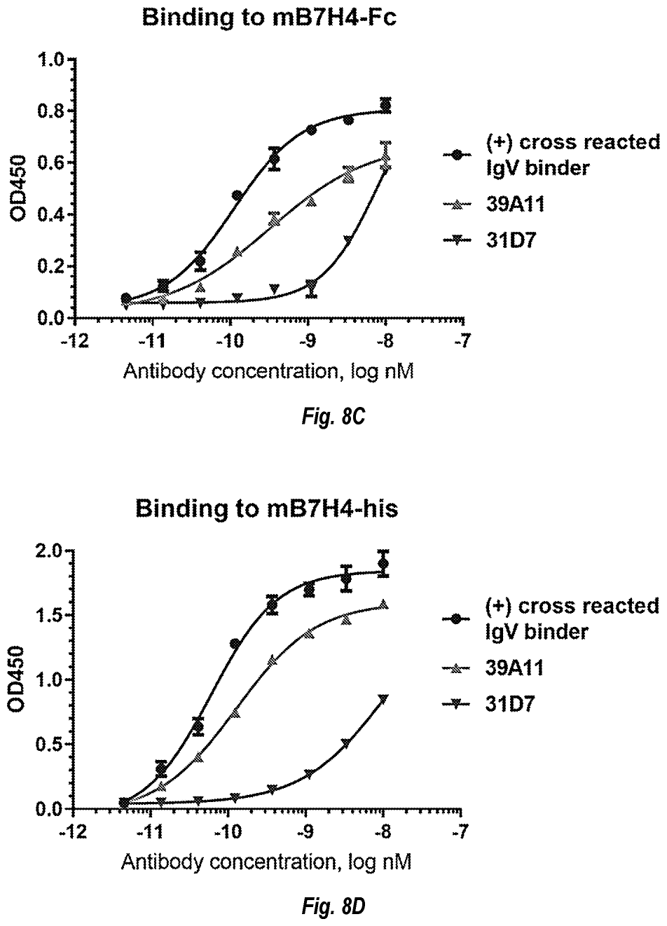

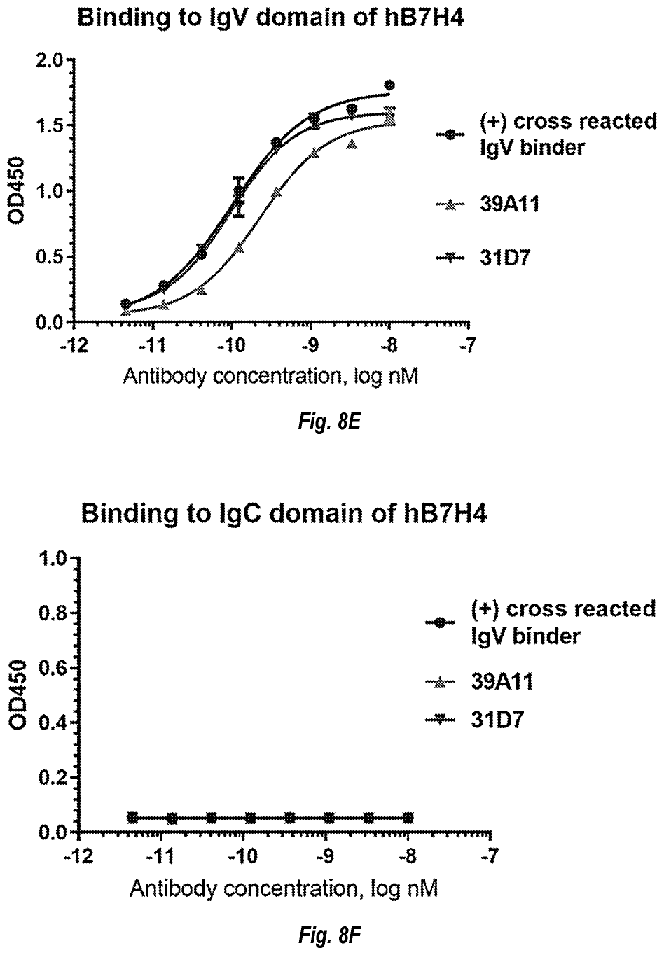

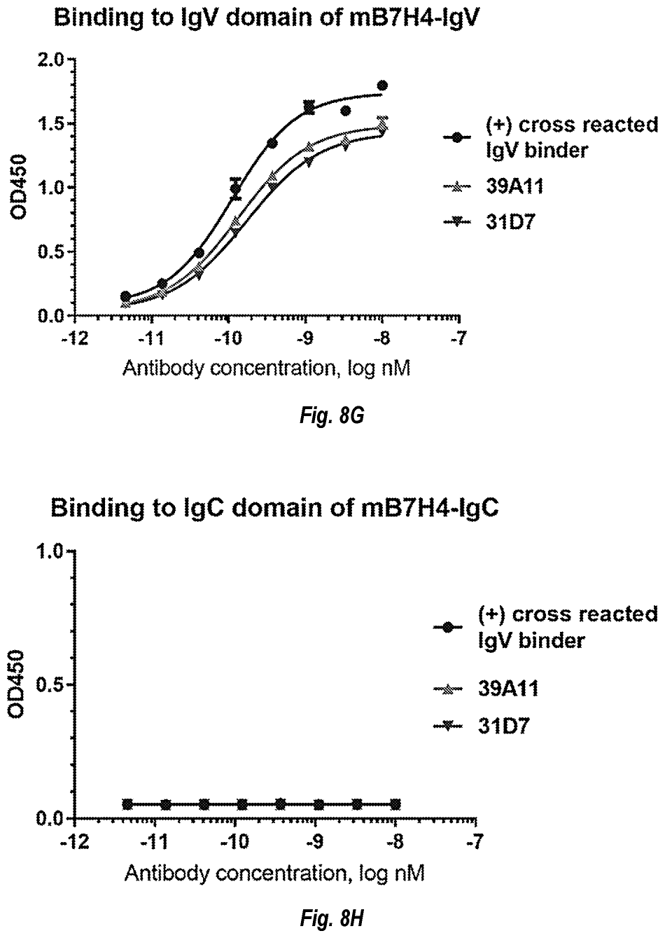

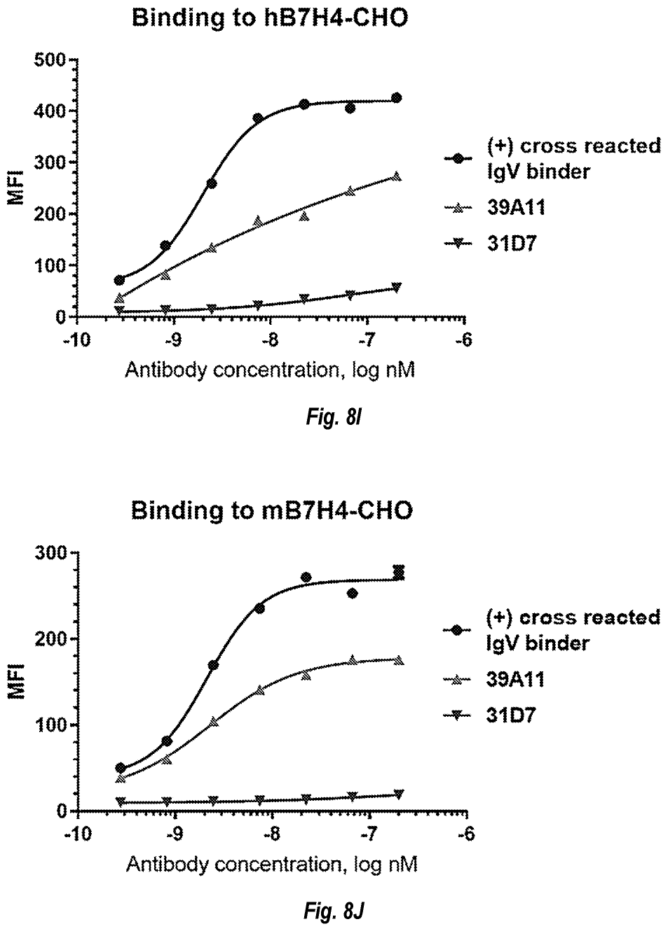

(3) Antibodies binding to IgV domain of human and murine hB7-H4: 317 and 39A11. See FIG. 1 (Batch A-3) and FIG. 8.

The specificities and specific binding sites of the isolated antibodies are summarized in Table 1.

TABLE 1
Specificities and specific binding sites
of isolated anti-B7-H4 antibodies.

| Antibody | | |
|---|---|---|
| [VH/VL] | Specificity | Binding site |
| 1A12/1A12 | Human hB7-H4 only | IgV domain of human B7-H4 |
| 1D3/1D3 | Human hB7-H4 only | IgV domain of human B7-H4 |
| 1F11/1F11 | Human hB7-H4 only | IgV domain of human B7-H4 |
| 3D1/3D1 | Human hB7-H4 only | IgV domain of human B7-H4 |
| 3F2/3F2 | Human hB7-H4 only | IgV domain of human B7-H4 |
| 4H6/4H6 | Human hB7-H4 only | IgV domain of human B7-H4 |
| 6C3/6C3 | Human hB7-H4 only | IgV domain of human B7-H4 |
| 9H2/9H2 | Human hB7-H4 only | IgV domain of human B7-H4 |
| 4B9/4B9 | Human hB7-H4 only | Both IgV and IgC domains |
| | | of human B7-H4 |
| 5E4/5E4 | Human and mouse B7-H4 | IgC domain of h/mB7-H4 |
| 5F4/5F4 | Human and mouse B7-H4 | IgC domain of h/mB7-H4 |
| 5G6/5G6 | Human and mouse B7-H4 | IgC domain of h/mB7-H4 |
| 9D11/9D11 | Human and mouse B7-H4 | IgC domain of h/mB7-H4 |
| 9E1/9E1 | Human and mouse B7-H4 | IgC domain of h/mB7-H4 |
| 15B11/ | Human and mouse B7-H4 | IgC domain of h/mB7-H4 |
| 15B11 | | |
| 24B6/24B6 | Human and mouse B7-H4 | IgC domain of h/mB7-H4 |
| 24F4/24F4 | Human and mouse B7-H4 | IgC domain of h/mB7-H4 |
| 30G4/30G4 | Human and mouse B7-H4 | IgC domain of h/mB7-H4 |
| 39A11/ | Human and mouse B7-H4 | IgV domain of h/mB7-H4 |
| 39A11 | | |
| 31D7/31D7 | Human and mouse B7-H4 | IgV domain of h/mB7-H4 |

### Example 3: Kinetics for the Binding of Isolated Anti-1B7-114 Antibodies to Soluble 1B7-114

The kinetics for binding of the different antibodies to their targets were examined using Biacore analysis. The results are summarized in Tables 2-4.

Antibodies 11D3 and 3F2 bound strongly to hB7-H4 and were chosen for affinity maturation (light chain shuffling) (FIG. 1 (Step B)). h/mB7-H4 cross-reactive antibodies 9D11 (IgC domain binding antibody) and 39A11 (IgV domain binding antibody) bound strongly to their targets and were also selected for the affinity maturation (light chain shuffling) (FIG. 1 (Step B)).

TABLE 2
Binding of indicated hB7-H4 IgV domain binding
antibodies to hB7-H4-Fc and hB7-H4-his.

| | Binding kinetics for hB7-H4-Fc | | | Binding kinetics for hB7-H4-his | | |
|---|---|---|---|---|---|---|
| Antibody | kₐ | k_{d} | K_{D} | kₐ | k_{d} | K_{D} |
| 1A12 | 1.41E+05 | 5.04E−04 | 3.57E−09 | 2.36E+05 | 5.83E−04 | 2.47E−09 |
| 1D3* | 9.02E+05 | 7.26E−04 | 8.05E−10 | 1.01E+06 | 5.58E−04 | 5.51E−10 |
| 1F11 | 1.75E+06 | 2.91E−03 | 1.66E−09 | 4.38E+06 | 4.88E−03 | 1.11E−09 |
| 3D1 | 1.12E+06 | 2.49E−03 | 2.23E−09 | 1.36E+05 | 3.88E−03 | 2.85E−08 |
| 3F2* | 1.12E+07 | 1.06E−03 | 9.51E−11 | 1.19E+08 | 1.01E−02 | 8.49E−11 |
| 4B9 | 4.10E+05 | 1.18E−03 | 2.89E−09 | 4.90E+04 | 2.02E−04 | 4.13E−09 |
| 4H6 | 5.51E+05 | 1.57E−03 | 2.86E−09 | 9.03E+05 | 3.61E−03 | 4.00E−09 |
| 6C3 | 1.29E+05 | 1.40E−03 | 1.09E−08 | 3.15E+05 | 1.99E−03 | 6.30E−09 |
| 9H2 | n/d | n/d | n/d | n/d | n/d | n/d |

No binding was observed for mB7-H4-Fc or mB7-H4-his.
n/a = not determined.
*Antibodies 1D3 and 3F2 were chosen for affinity maturation (light chain shuffling).

TABLE 3
Binding of anti-h/mB7-H4 antibodies to hB7-H4-Fc and hB7-H4-his.

| | Binding kinetics for hB7-H4-Fc | | | Binding kinetics for hB7-H4-his | | |
|---|---|---|---|---|---|---|
| Antibody | kₐ | k_{d} | K_{D} | kₐ | k_{d} | K_{D} |
| 5E4 | 3.87E+05 | 4.52E−03 | 1.17E−08 | 4.53E+05 | 4.55E−03 | 1.00E−08 |
| 5F4 | 1.92E+05 | 2.88E−03 | 1.50E−08 | 1.99E+05 | 3.29E−03 | 1.66E−08 |
| 5G6 | 1.87E+05 | 2.26E−03 | 1.21E−08 | 3.77E+05 | 1.96E−03 | 5.19E−09 |
| 9D11 | 3.74E+05 | 6.74E−04 | 1.80E−09 | 6.47E+05 | 5.49E−04 | 8.50E−10 |
| 9E1 | 2.72E+05 | 3.72E−03 | 1.37E−08 | 7.11E+05 | 5.62E−03 | 7.91E−09 |
| 15B11 | 1.84E+05 | 7.84E−04 | 4.26E−09 | 1.11E+05 | 5.73E−04 | 5.17E−09 |
| 24B6 | n/d | | | 1.49E+05 | 7.89E−04 | 5.30E−09 |
| 24F4 | | | | 2.68E+04 | 5.20E−03 | 1.94E−07 |
| 30G4 | | | | 1.15E+05 | 6.94E−04 | 6.05E−09 |
| 39A11* | 7.27E+05 | 6.74E−04 | 9.26E−10 | n/d | | |
| 31D7 | 8.97E+05 | 7.66E−04 | 8.54E−10 | | | |

n/a = not available.
Antibody 39A11 was chosen for affinity maturation (light chain shuffling).

TABLE 4
Binding of anti-h/mB7-H4 antibodies to mB7-H4-Fc and mB7-H4-his.

| | Binding kinetics for mB7-H4-Fc | | | Binding kinetics for mB7-H4-his | | |
|---|---|---|---|---|---|---|
| Antibody | kₐ | k_{d} | K_{D} | kₐ | k_{d} | K_{D} |
| 5E4 | 1.97E+05 | 2.79E−03 | 1.41E−08 | 1.17E+06 | 1.06E−02 | 9.09E−09 |
| 5F4 | 2.40E+05 | 2.86E−03 | 1.19E−08 | 2.56E+05 | 3.84E−03 | 1.50E−08 |
| 5G6 | 2.00E+05 | 2.61E−03 | 1.31E−08 | 2.86E+05 | 2.24E−03 | 7.83E−09 |
| 9D11 | 6.99E+05 | 4.49E−04 | 6.42E−10 | 1.85E+05 | 2.81E−04 | 1.52E−09 |
| 9E1 | 3.09E+05 | 3.28E−03 | 1.06E−08 | 2.81E+05 | 3.49E−03 | 1.24E−08 |
| 15B11 | 2.76E+05 | 9.80E−04 | 3.55E−09 | 1.18E+05 | 7.29E−04 | 6.17E−09 |
| 24B6 | n/d | | | 1.62E+05 | 5.52E−04 | 3.40E−09 |
| 24F4 | | | | 1.07E+05 | 3.14E−03 | 2.92E−08 |
| 30G4 | | | | 9.54E+04 | 6.09E−04 | 6.38E−09 |
| 39A11 | 7.30E+05 | 1.11E−03 | 1.53E−09 | n/d | | |
| 31D7 | 8.88E+05 | 1.43E−03 | 1.61E−09 | | | |

n/a = not determined.
Antibody 39A11 was chosen for affinity maturation (light chain shuffling).

### Example 4: Affinity Maturation of Anti-B7-H4 Antibodies 3F2 and 1D3 (hB7-H4 IgV Domain Binding Antibodies) by Light Chain Shuffling

Affinity maturation of 3F2 and 1D3 was performed by light chain shuffling. For this, the heavy chains of 3F2 or 1D3, respectively, were paired with a library comprising >1.0×10⁸ light chains (See FIG. 1 (Steps B-1 and B-2)). The resulting libraries were panned on immobilized hB7-H4-Fc or Biotin-hB7-H4-his captured by strep beads in hardship conditions as described in Example 1. The sequences for the isolated 3F2 derivatives and 1D3 derivatives are shown in Table 5.

Isolated 3F2 derivatives 3F2/50A10 and 3F2/49A2 had the same heavy chain sequence as 3F2, but a different light chain sequence than 3F2 (FIG. 1 (Batch B-1)). Like their parental antibody, the derivatives 3F2/50A10 and 3F2/49A2 only bound to IgV domain of hB7-H4. 3F2 derivatives 3F2/50A10 and 3F2/49A2 showed stronger binding to the cellular human B7-H4 than parental antibody 3F2 (Table 6).

Isolated 1D3 derivatives 1D3/47B2 and 1D3/45A2 had the same heavy chain sequence as 1D3, but a different light chain sequence than 1D3 (FIG. 1 (Batch B-2)). Like their parental antibody, the derivatives 1D3/47B2 and 1D3/45A2 only bound to IgV domain of hB7-H4. 1D3 derivatives 1D3/47B2 and 1D3/45A2 showed stronger binding to the cellular human B7-H4 than parental antibody 1D3 (Table 6).

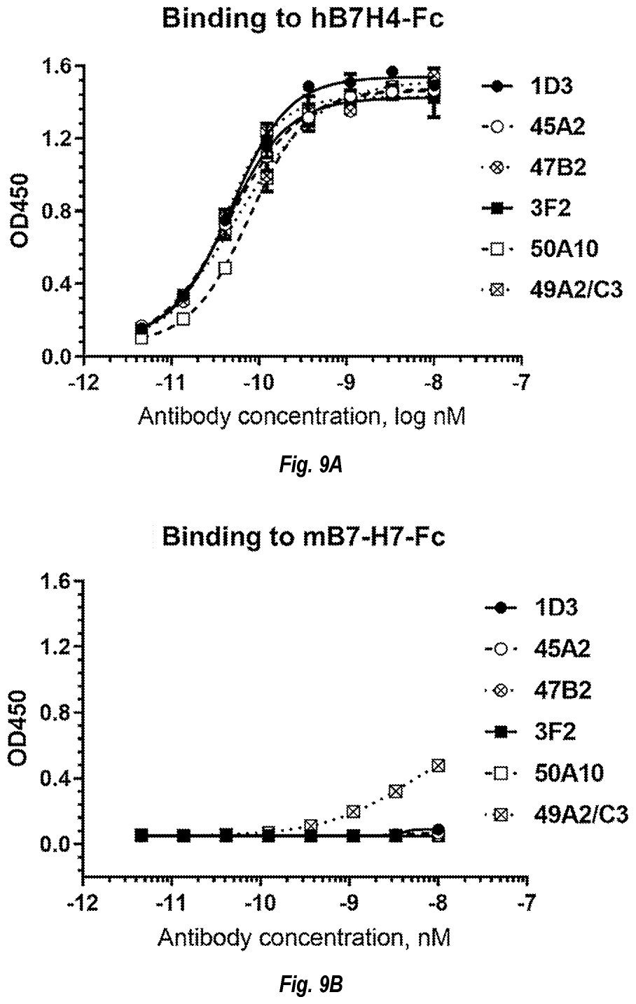

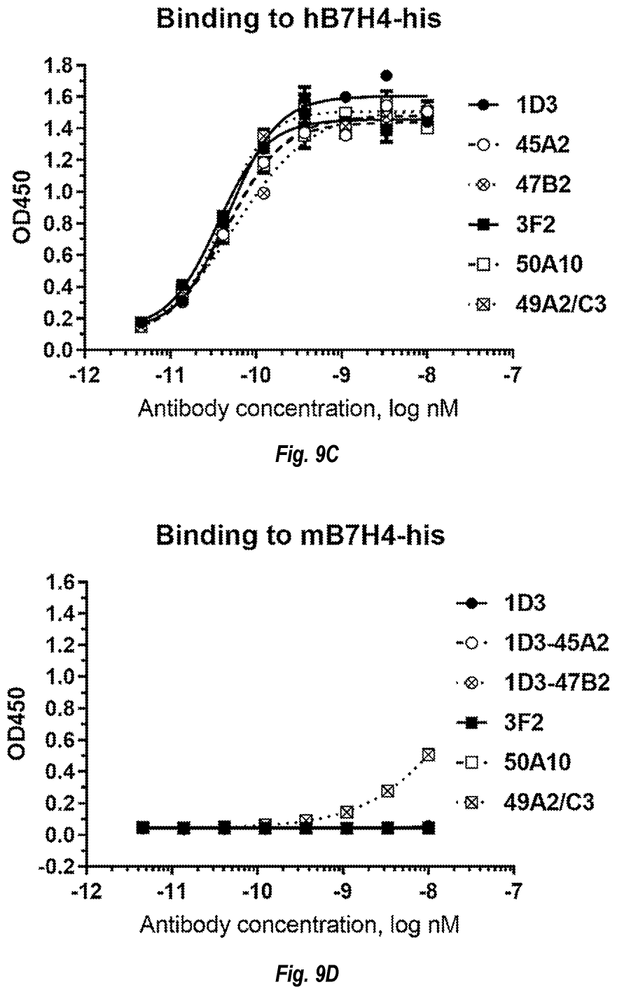

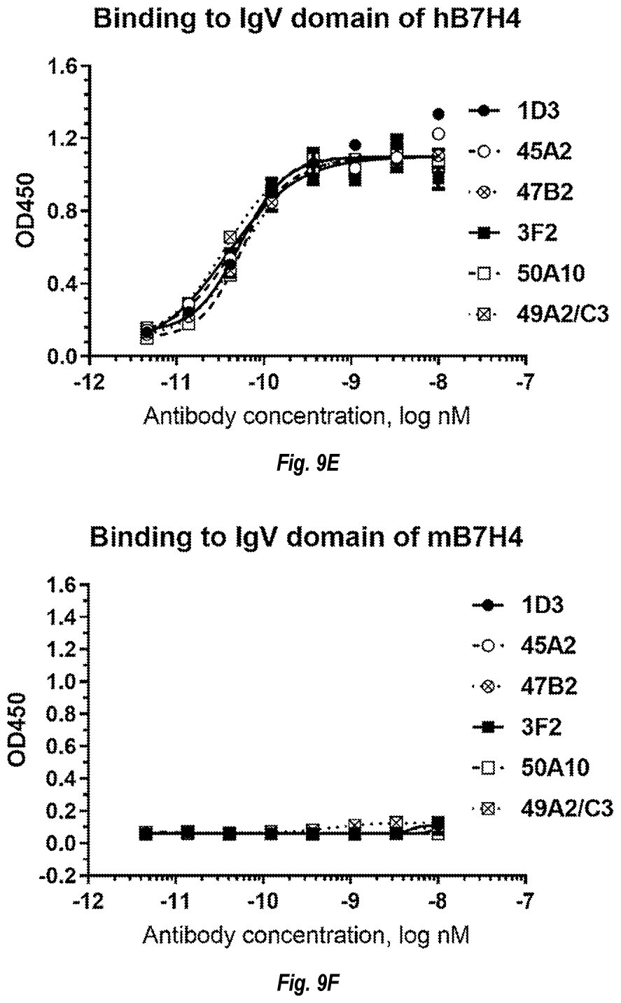

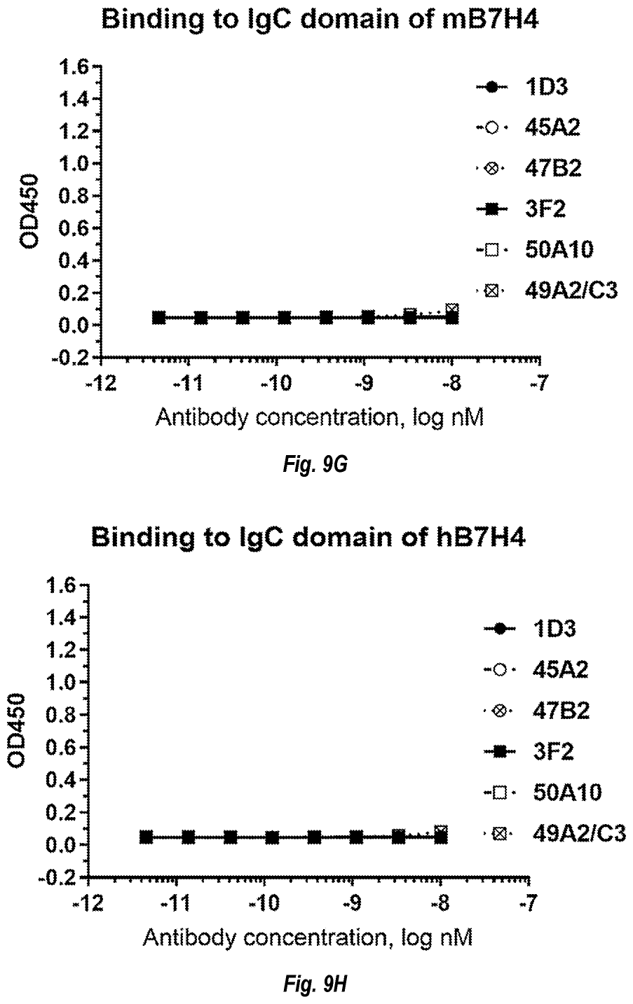

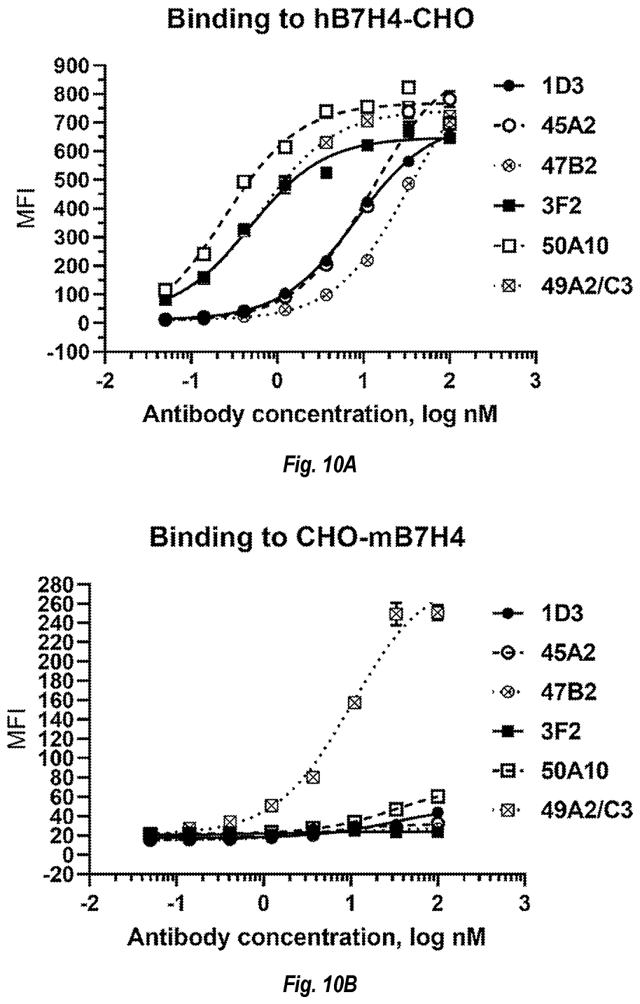

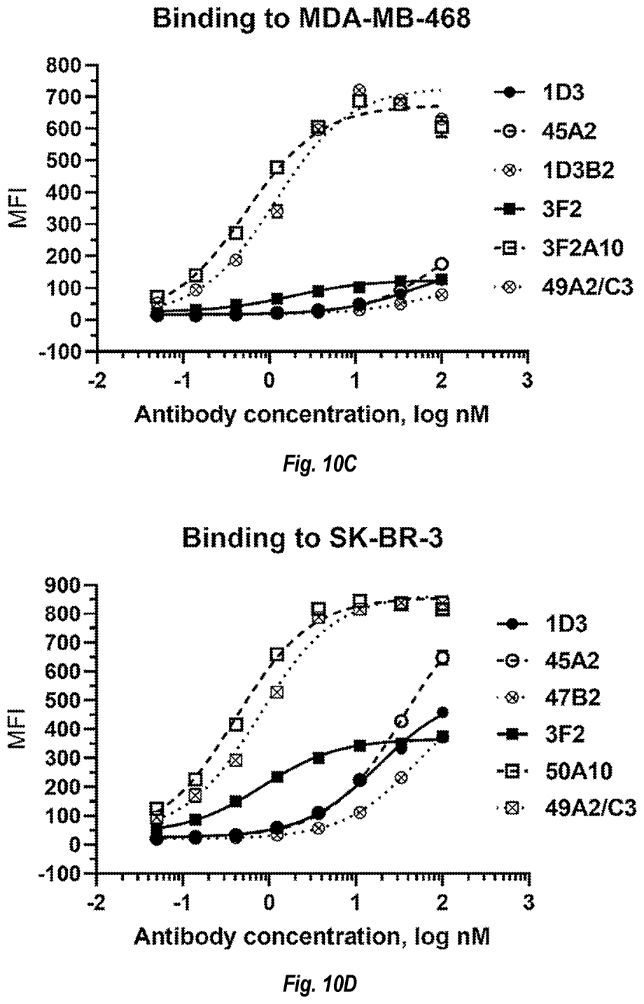

Binding of these antibodies to soluble and cellular B7-H4 is shown in FIGS. 9-10.

TABLE 5
Light chain CDR sequence for antibodies 3F2 and
1D3 and their respective derivatives.

| | SEQ | | SEQ | | SEQ | |
|---|---|---|---|---|---|---|
| Antibody VL | ID NO | LCDR1 | ID NO | LCDR2 | ID NO | LCDR3 |
| 3F2 | 20 | RASQSISSYLN | 21 | SSLQS | 22 | QQSYSTPLT |
| 50A10 | 70 | QASQDISNYIN | 71 | SRLQS | 72 | QQSYRSPFT |
| 49A2 | 66 | RASQNIDTYVN | 67 | SRLHT | 68 | QQSYTSPFT |
| 3F2 | 73 | X₁ASQX₂IX₃X₄YX₅N | 74 | SX₆LX₇X₈ | 75 | QQSYX₉X₁₀PX₁₁T |
| CDR | | Wherein: | | Wherein: | | Wherein: |
| motifs | | X₁ is R or Q | | X₆ is R or S | | X₉ is R, S, or T |
| | | X₂ is D, N, or S | | X₇ is Hor Q | | X₁₀ is S or T |
| | | X₃ is D or S | | X₈ is S or T | | X₁₁ is F or L |
| | | X₄ is N, S, or T | | | | |
| | | X₅ is I, L, or V | | | | |
| 1D3 | 8 | RASRSIYTWLA | 9 | STLQS | 10 | QQSYSTPYT |
| 47B2 | 81 | RASQTVYTWLA | 82 | TNLAT | 83 | QQSYSTSWT |
| 45A2 | 77 | RASQNIYTWLA | 78 | TNLPT | 79 | QQSYSTRWT |
| 1D3 | 84 | RASX₁₂X₁₃X₁₄YTWLA | 85 | X₁₅X₁₆LX₁₇X₁₈ | 86 | QQSYSTX₁₉X₂₀T |
| CDR | | Wherein: | | Wherein: | | Wherein: |
| motifs | | X₁₂ is Q or R | | X₁₅ is S or T | | X₁₉ is P, R, or S |
| | | X₁₃ is N, S, or T | | X₁₆ is N or T | | X₂₀ is Wor Y |
| | | X₁₄ is I or V | | X₁₇ is A, P, | | |
| | | | | or Q | | |
| | | | | X₁₈ is S or T | | |

TABLE 6
Binding properties of antibody 3F2 (hB7-H4 IgV domain binding
antibody) and its derivatives and of antibody 1D3 (specific
for the IgV domain of hB7-H4) and its derivatives.

| | EC50, nM | | | | | EC50, nM | | Binding | | |
|---|---|---|---|---|---|---|---|---|---|---|
| | SK- | | | | | mB7- | | kinetics for | | |
| Antibody | BR- | hB7- | Binding kinetics for hB7-H4 | | | H4/ | mB7- | mB7-H4 | | |
| (VH/VL) | 3 | H4 | Kₐ | K_{d} | K_{D} | CT26 | H4 | Kₐ | K_{d} | K_{D} |
| 3F2/3F2 | 0.84 | 0.048 | 1.59E+07 | 1.30E−03 | 8.19E−11 | No binding | | | | |
| 3F2/50A10 | 0.41 | 0.046 | 1.07E+07 | 4.13E−04 | 3.85E−11 | | | | | |
| 3F2/49A2 | 0.79 | 0.056 | 8.12E+06 | 1.22E−04 | 1.50E−11 | | | | | |
| 1D3/1D3 | 18.6 | 0.044 | 6.37E+05 | 4.94E−04 | 7.75E−10 | | | | | |
| 1D3/47B2 | 36.0 | 0.078 | 5.85E+05 | 1.22E−04 | 2.09E−10 | | | | | |
| 1D3/45A2 | 52.1 | 0.041 | 2.32E+05 | 7.36E−05 | 3.17E−10 | | | | | |

### Example 5: Affinity Maturation of Anti-B7-H14 Antibody 9D11 (h/mB7-H14 IgC Domain Binding Antibody) by Light Chain Shuffling

Affinity maturation of 9D11 was performed by light chain shuffling. For this, the heavy chain of 9D11 was paired with a library comprising >1.0×10⁸ light chains. See FIG. 1 (Step B-3). The resulting library was panned on immobilized hB7-H4-Fc or Biotin-hB7-H4-his captured by strep beads in hardship conditions as described in Example 1. See FIG. 1 (Step B). The sequences for the isolated 9D11 derivatives are shown in Table 7.

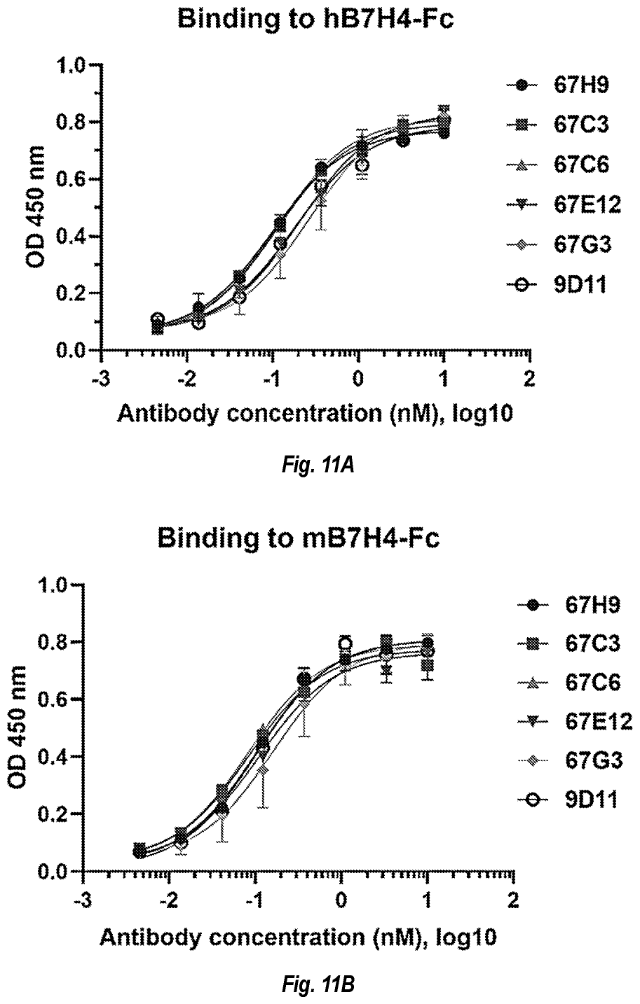

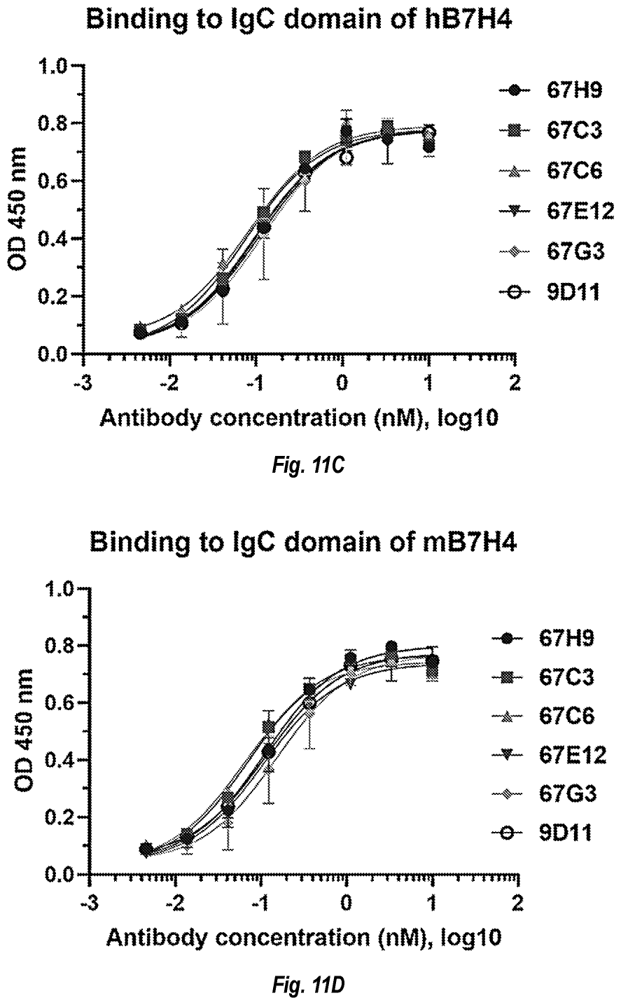

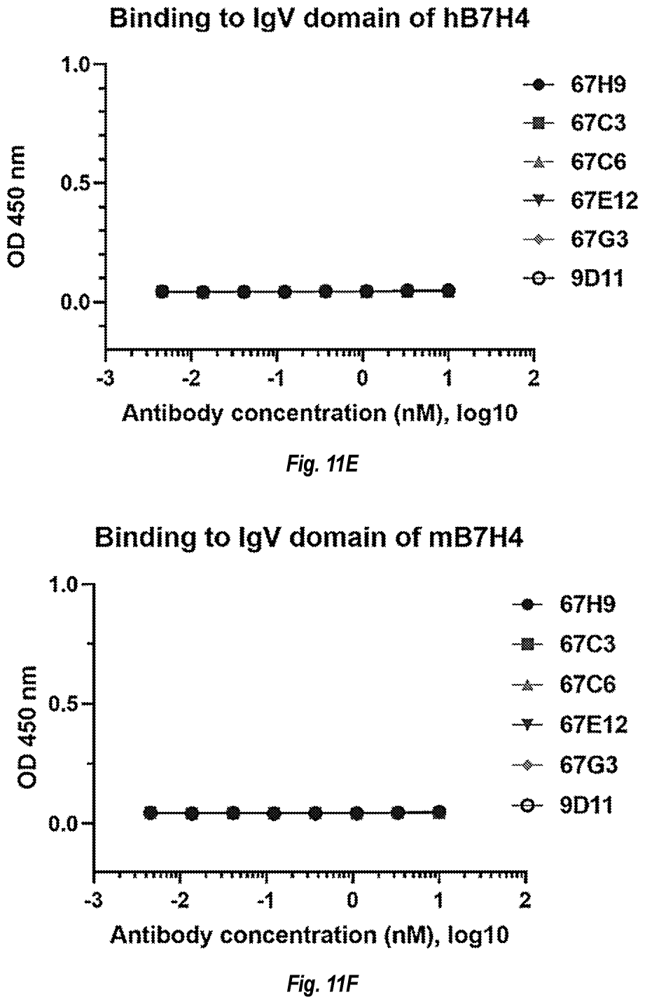

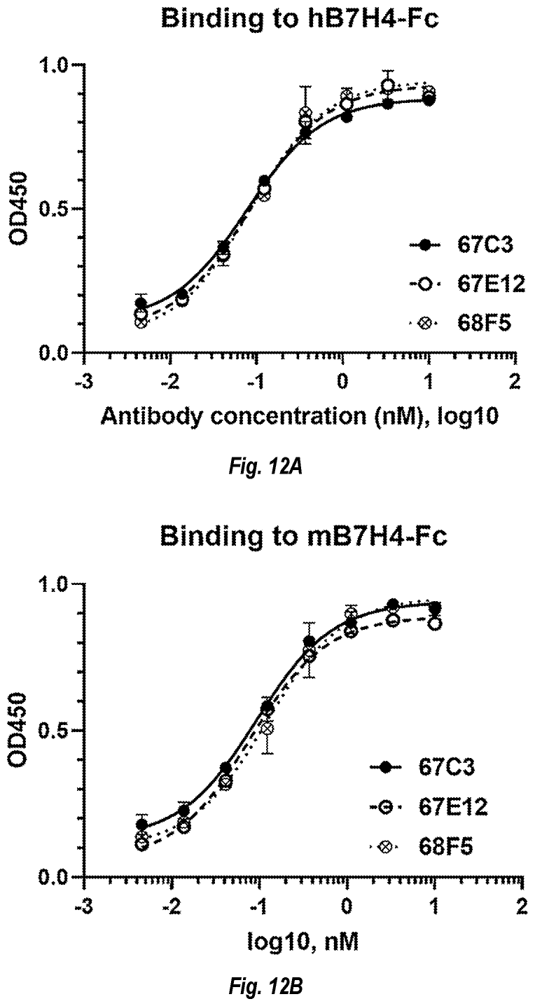

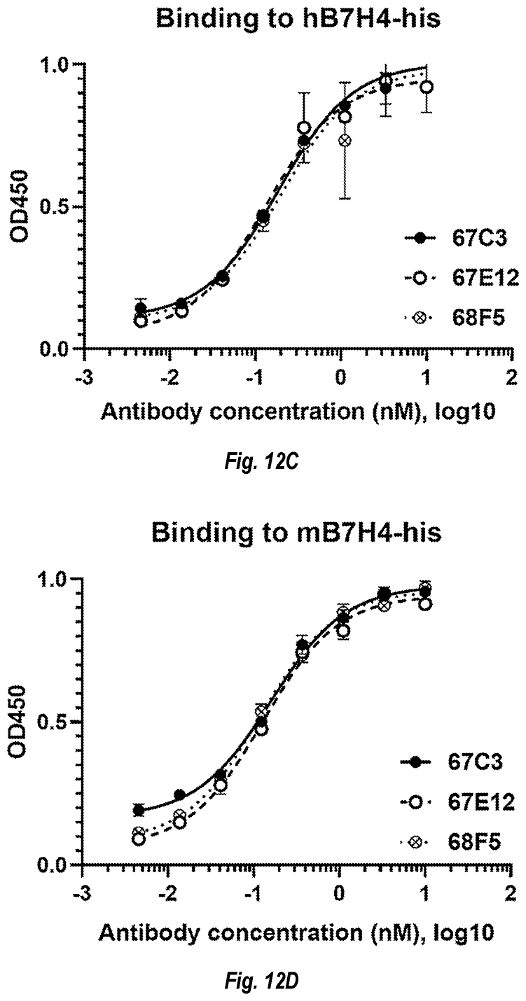

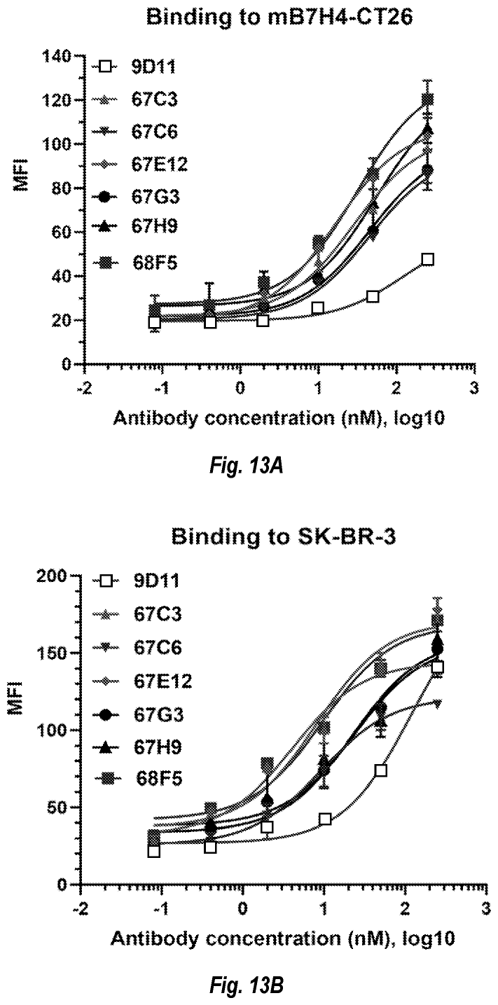

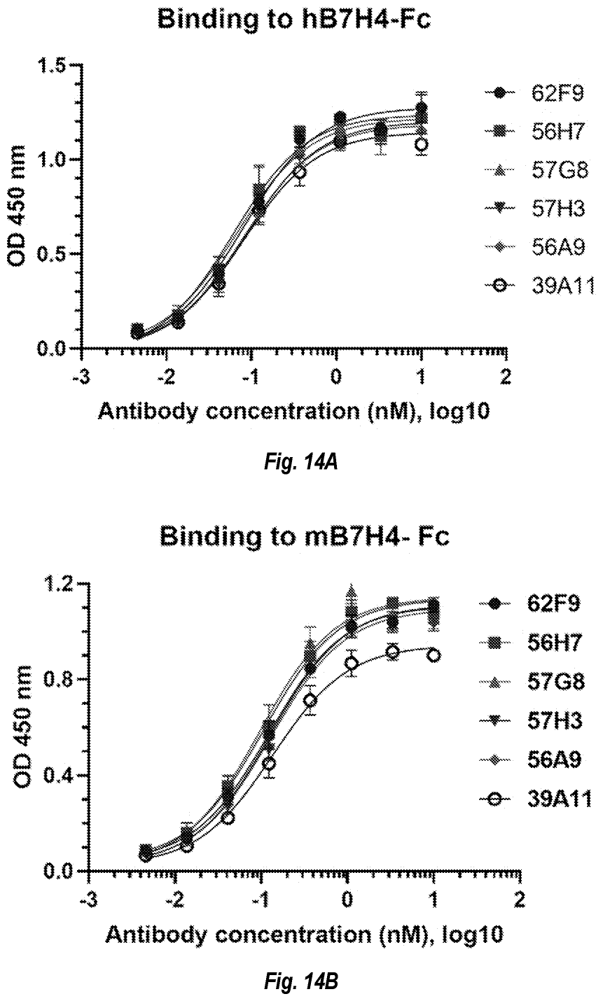

Isolated 9D11 derivatives 9D11/67E12, 9D11/67C3, 9D11/67C6, 9D11/67H9, 9D11/67G3, and 9D11/68F5 have the same heavy chain sequence as 9D11, but a different light chain sequence than 9D11 (FIG. 1 (Batch B-3)). Binding affinities for these antibodies are shown in Table 8, binding of these antibodies to soluble and cellular B7-H4 is shown in FIGS. 11-13.

TABLE 7
Light chain CDR sequences for antibody 9D11 and its derivatives.

| | SEQ | | SEQ | | SEQ | |
|---|---|---|---|---|---|---|
| | ID | | ID | | ID | |
| Antibody VL | NO | LCDR1 | NO | LCDR2 | NO | LCDR3 |
| 9D11 | 50 | KSSQSVLYSSNNKNYLA | 51 | STRES | 2 | QQYFSTPS |
| 67E12 | 88 | KSSQSVLYSSNNKNYLA | 89 | SKRVS | 90 | QQYFDSPT |
| 67C6 | 92 | KSSQSVLSSSNNKNYLA | 93 | STRQS | 94 | QQYYSDPT |
| 67C3 | 96 | KSSQSVLYSSNNKNYLA | 97 | STRAS | 98 | QQYYDTPT |
| 68F5 | 108 | KSSRSVLSRSNNKNYLA | 109 | STRQF | 110 | QQYYDTPT |
| 67G3 | 100 | KSSQSVLSSSNNKNYLA | 101 | STRQS | 102 | QQYYTSPT |
| 67H9 | 104 | RSSQSVLYSSNNKNYLA | 105 | SNRKS | 106 | QQYYSAPT |
| 9D11 | 111 | X₂₁SSX₂₂SVLX₂₃X₂₄SNNKNY | 112 | SX₂₅RX₂₆X₂₇ | 113 | QQYX₂₈X₂₉X₃₀PX₃₁ |
| CDR | | LA | | Wherein: | | Wherein: |
| motifs | | Wherein: | | X₂₅ is K, N, T | | X₂₈ is F or Y |
| | | X₂₁ is K or R | | X₂₆ is A, E, K, | | X₂₉ is D, S, or T |
| | | X₂₂ is Q or R | | Q, or V | | X₃₀ is A, D, S, or T |
| | | X₂₃ is S or Y | | X₂₇ is F or S | | X₃₁ is S or T |
| | | X₂₄ is R or S | | | | |

TABLE 8
Binding properties of antibody 9D11 (h/mB7-H4 IgC
domain binding antibodies) and its derivatives.

| Antibody | EC50, nM | | Binding kinetics for hB7-H4 | | |
|---|---|---|---|---|---|
| (VH/VL) | SK-BR-3 | hB7-H4 | Kₐ | K_{d} | K_{D} |
| 9D11/9D11 | 125.9 | 0.180 | 3.72E+05 | 2.22E−03 | 5.97E−09 |
| 9D11/67E12 | 8.08 | 0.203 | 1.04E+05 | 6.54E−04 | 6.30E−09 |
| 9D11/67C6 | 9.20 | 0.115 | 4.25E+04 | 6.36E−04 | 1.50E−08 |
| 9D11/67C3 | 4.06 | 0.112 | 3.45E+04 | 3.94E−04 | 1.14E−08 |
| 9D11/67G3 | 22.5 | 0.261 | 1.44E+05 | 1.53E−03 | 1.06E−08 |
| 9D11/68F5 | 9.53 | 0.778* | 1.18E+05 | 8.42E−04 | 7.15E−09 |
| 9D11/68H9 | 24.0 | 0.100 | 1.52E+05 | 1.08E−03 | 7.09E−09 |

| Antibody | EC50, nM | | Binding kinetics for mB7-H4 | | |
|---|---|---|---|---|---|
| (VH/VL) | mB7-H4/CT26 | mB7-H4 | Kₐ | K_{d} | K_{D} |
| 9D11/9D11 | 122.4 | 0.104 | 4.28E+05 | 2.05E−03 | 4.78E−09 |
| 9D11/67E12 | 16.7 | 0.110 | 1.65E+05 | 7.67E−04 | 4.66E−09 |
| 9D11/67C6 | 45.7 | 0.082 | 3.36E+04 | 9.41E−05 | 2.80E−09 |
| 9D11/67C3 | 28.2 | 0.086 | 3.11E+04 | 3.23E−04 | 1.04E−08 |
| 9D11/67G3 | 45.3 | 0.162 | 1.91E+05 | 1.61E−03 | 8.46E−09 |
| 9D11/68F5 | 31.8 | 0.145* | 1.52E+05 | 9.03E−04 | 5.96E−09 |
| 9D11/68H9 | 54.3 | 0.105 | 1.75E+05 | 1.06E−03 | 6.08E−09 |

### Example 6: Affinity Maturation of Anti-B7-H4 Antibody 39A11 (h/mB7-H4 IgV Domain Binding Antibody) by Light Chain Shuffling

Affinity maturation of 39A11 was performed by light chain shuffling. For this, the heavy chain of 39A11 was paired with a library comprising >1.0×10⁸ light chains. See FIG. 1 (Step B-4). The resulting library was panned on immobilized hB7-H4-Fc or Biotin-hB7-H4-his captured by strep beads in hardship conditions as described in Example 1 (FIG. 1 (Step B)). The sequences for the isolated 39A11 derivatives are shown in Table 9.

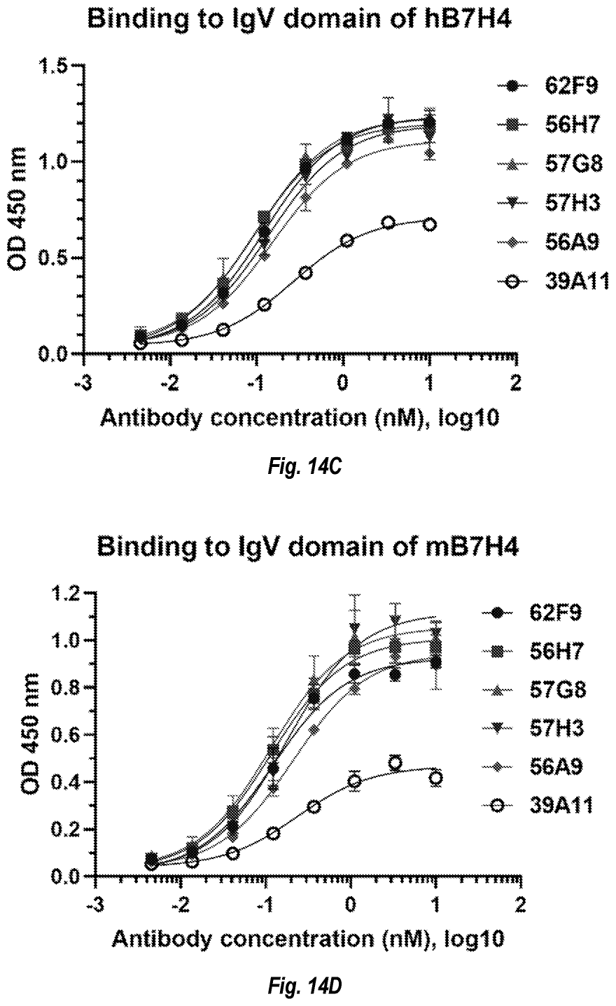

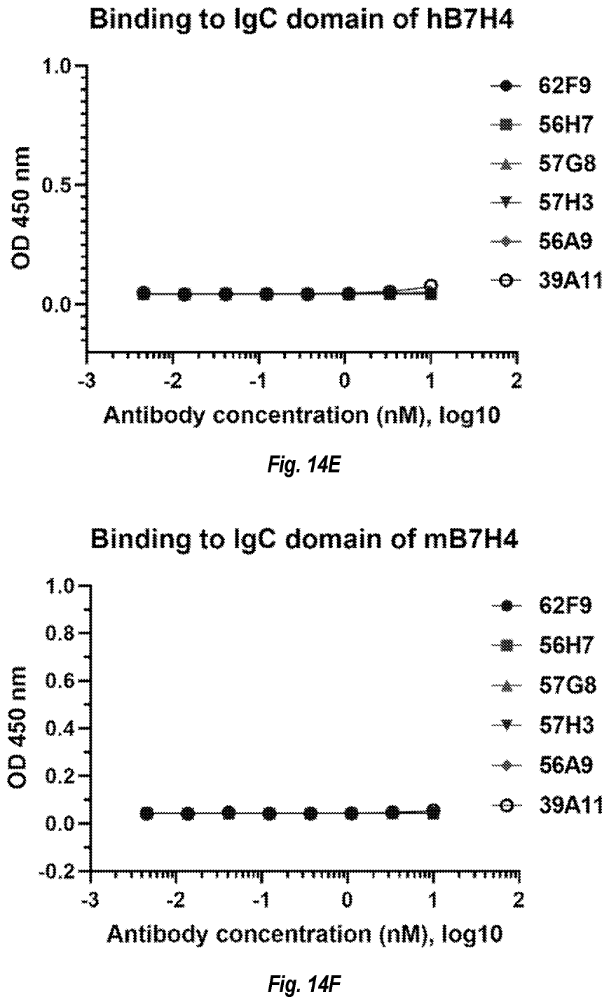

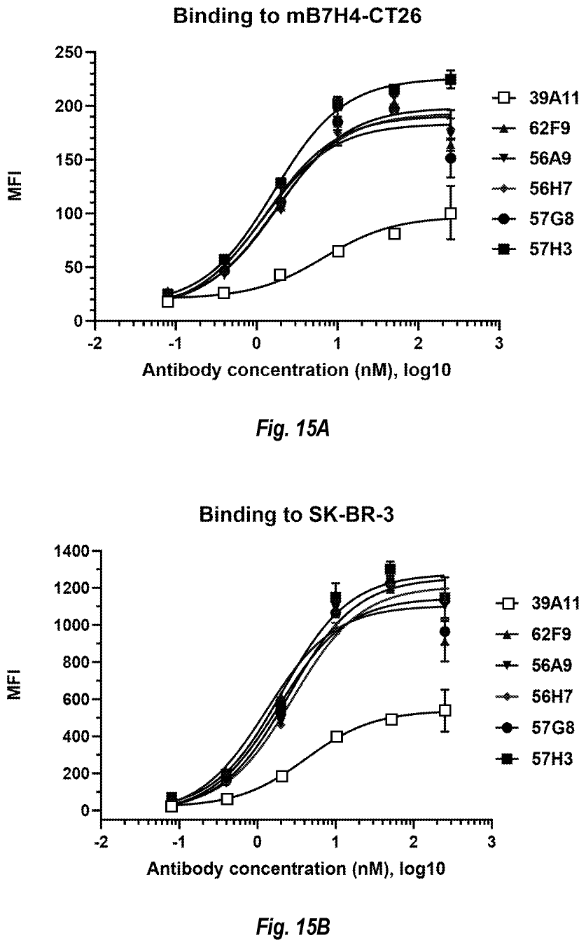

Isolated 39A11 derivatives 39A11/57H3, 39A11/57G8, 39A11/56A9, 39A11/56H7 and 39A11/62F9 have the same heavy chain sequence as 39A11, but a different light chain sequence than 39A11 (FIG. 1 (Batch B-4)). Like the parental 39A11, all derivatives only bound to IgV domain of human and mouse B7-H4. 39A11 derivatives generally showed stronger binding to the cellular human B7-H4 than parental antibody 39A11. Binding of these antibodies to soluble and cellular B7-H4 is shown in Table 10 and FIGS. 14-15.

TABLE 9
Light chain CDR sequence for antibody 39A11 and its derivatives.

| | SEQ | | SEQ | | SEQ | |
|---|---|---|---|---|---|---|
| Antibody | ID | | ID | | ID | |
| VL | NO | LCDR1 | NO | LCDR2 | NO | LCDR3 |
| 39A11 | 62 | RASQTIRSYLN | 63 | SHLQS | 64 | QQSYTTPYT |
| 57H3 | 115 | QASQDIRKYLN | 116 | STRES | 117 | QQYYSLPLT |
| 57G8 | 119 | RASQSIRSYLN | 120 | SSLQS | 121 | QQYYSTPLT |
| 56A9 | 123 | RASQSISSYLN | 124 | SIRES | 125 | QQYYTTPLT |
| 56H7 | 127 | RASQSISSYLN | 128 | SNLQS | 129 | QQYYTTPLT |
| 62F9 | 131 | RASQSVSSAVA | 132 | SIRES | 133 | QQYYSTPLT |
| CDR | 134 | X₃₂ASQX₃₃X₃₄X₃₅X₃₆X₃₇ | 135 | SX₄₀X₄₁X₄₂S | 136 | QQX₄₃YX₄₄X₄₅ |
| motif | | X₃₈X₃₉ | | Wherein: | | PX₄₆T |
| | | Wherein: | | X₄₀ is H, I, | | Wherein: |
| | | X₃₂ is Q or R | | N, S, or T | | X₄₃ is S or Y |
| | | X₃₃ is D, S, or T | | X₄₁ is L or | | X₄₄ is S or T |
| | | X₃₄ is I or V | | R | | X₄₅ is L or T |
| | | X₃₅ is R or S | | X₄₂ is E or | | X₄₆ is L or Y |
| | | X₃₆ is K or S | | Q | | |
| | | X₃₇ is A or Y | | | | |
| | | X₃₈ is L or V | | | | |
| | | X₃₉ is A or N | | | | |

TABLE 10
Binding properties of antibody 39A11 (h/mB7-H4 IgV
domain binding antibody) and its derivatives.

| Antibody | EC50, nM | | Binding kinetics for hB7-H4 | | |
|---|---|---|---|---|---|
| (VH/VL) | SK-BR-3 | hB7-H4 | Kₐ | K_{d} | K_{D} |
| 39A11/39A11 | 4.17 | 0.080 | 1.73E+06 | 1.81E−03 | 1.05E−09 |
| 39A11/57H3 | 2.07 | 0.086 | 6.16E+05 | 2.95E−04 | 4.78E−10 |
| 39A11/57G8 | 1.90 | 0.068 | 8.63E+05 | 2.61E−04 | 3.03E−10 |
| 39A11/56A9 | 2.53 | 0.081 | 5.73E+05 | 6.91E−04 | 1.21E−09 |
| 39A11/56H7 | 2.85 | 0.061 | 5.14E+05 | 4.00E−04 | 7.79E−10 |
| 39A11/62F9 | 1.31 | 0.079 | 7.33E+05 | 6.25E−04 | 8.53E−10 |

| Antibody | EC50, nM | | Binding kinetics for mB7-H4 | | |
|---|---|---|---|---|---|
| (VH/VL) | mB7-H4/CT26 | mB7-H4 | Kₐ | K_{d} | K_{D} |
| 39A11/39A11 | 6.73 | 0.136 | 5.21E+06 | 6.80E−02 | 1.30E−08 |
| 39A11/57H3 | 1.63 | 0.128 | 5.00E+05 | 6.22E−07 | 1.25E−12 |
| 39A11/57G8 | 1.22 | 0.096 | 9.69E+05 | 9.66E−05 | 9.98E−11 |
| 39A11/56A9 | 1.85 | 0.130 | 7.37E+05 | 1.02E−06 | 1.38E−12 |
| 39A11/56H7 | 1.67 | 0.107 | 6.30E+05 | 1.38E−07 | 2.19E−13 |
| 39A11/62F9 | 1.32 | 0.124 | 4.55E+05 | 8.41E−04 | 1.85E−09 |

Top three antibodies 3F2/50A10 (hB7-H4 IgV domain binding antibody), 39A11/57G8 (h/mB7-H4 IgV domain binding antibody), and 9D11/68F5 (h/mB7-H4 IgC domain binding antibody) from Examples 3-6 were chosen for further characterization.

### Example 7: Binding of Anti-B7-H14 Antibodies to Soluble and Cell-Expressed B7-H14

Binding of antibodies 3F2/50A10 (hB7-H4 IgV domain binding antibody), 39A11/57G8 (h/mB7-H4 IgV domain binding antibody), and 9D11/68F5 (h/mB7-H4 IgC domain binding antibody) to soluble B7-H4 or cell-expressed B7-H4 was determined by ELISA or flow cytometry, respectively.

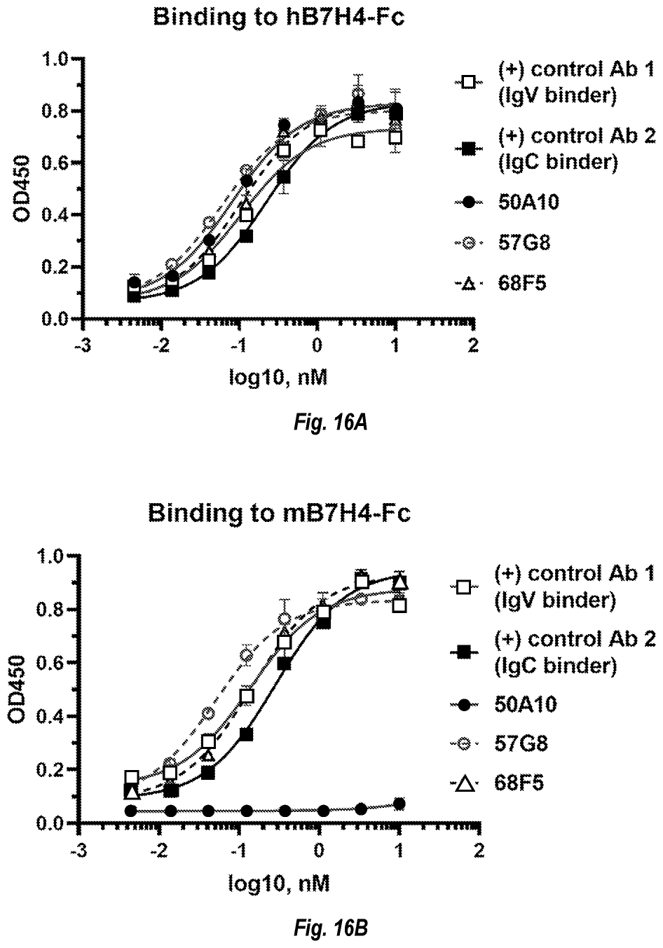

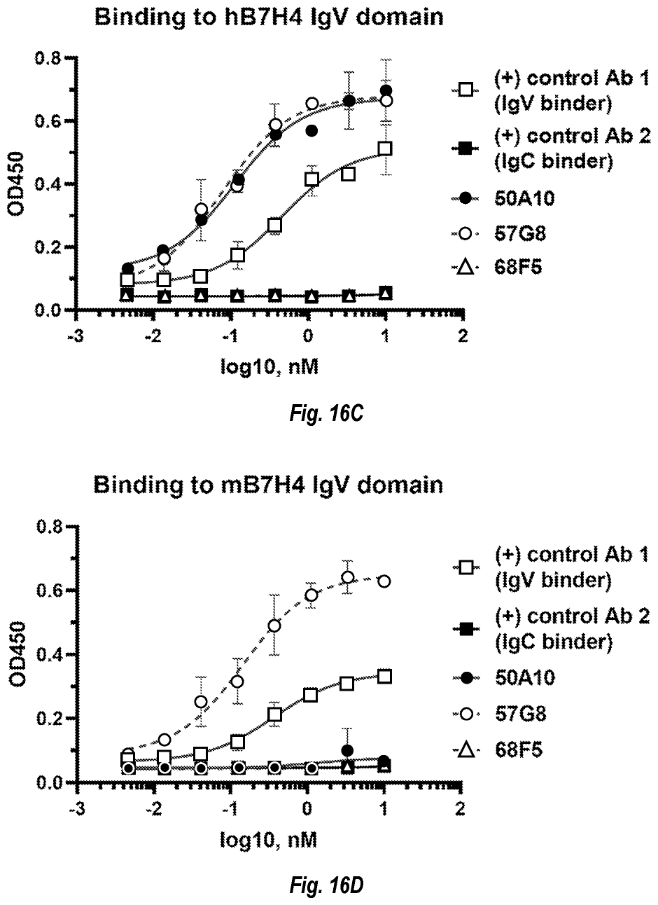

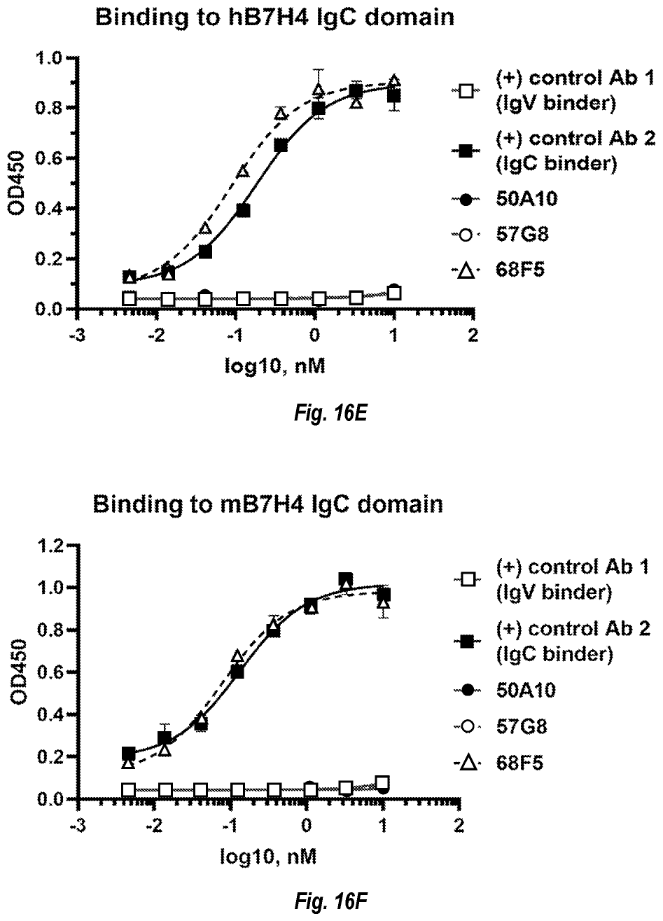

ELISA experiments demonstrated that both antibodies 3F2/50A10 and 39A11/57G8 bound to IgV domain of hB7-H4 strongly. Antibody 39A11 cross-reacted with mB7-H4. Antibody 9D11/68F5 bound to the IgC domain of human and mouse B7-H4 (FIG. 16).

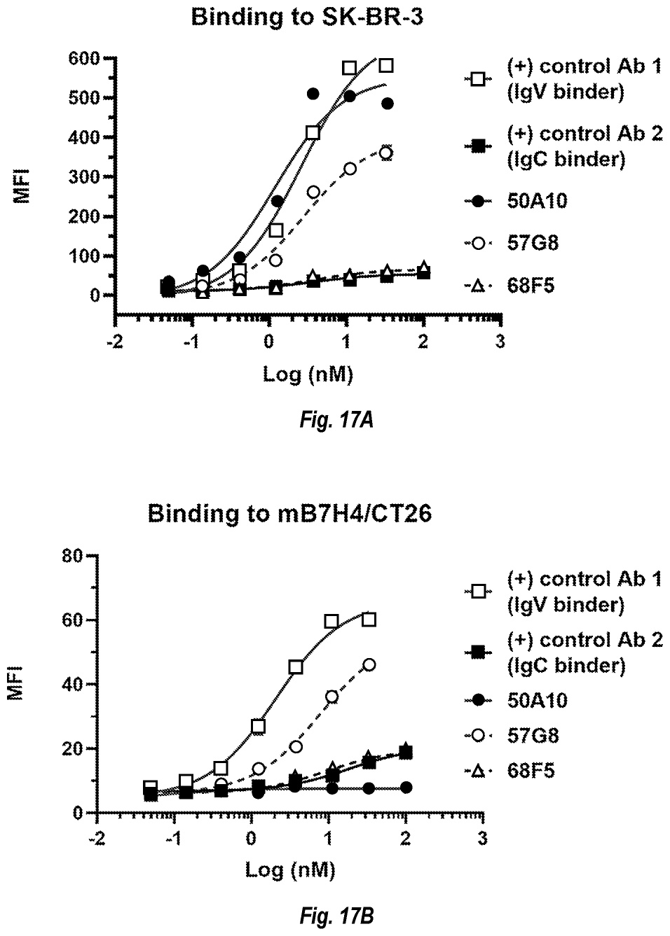

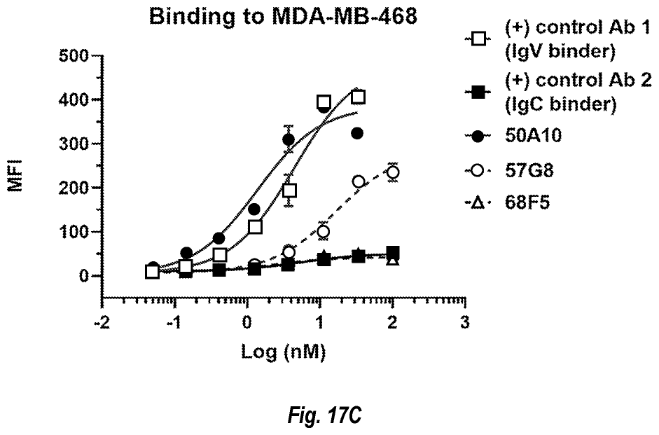

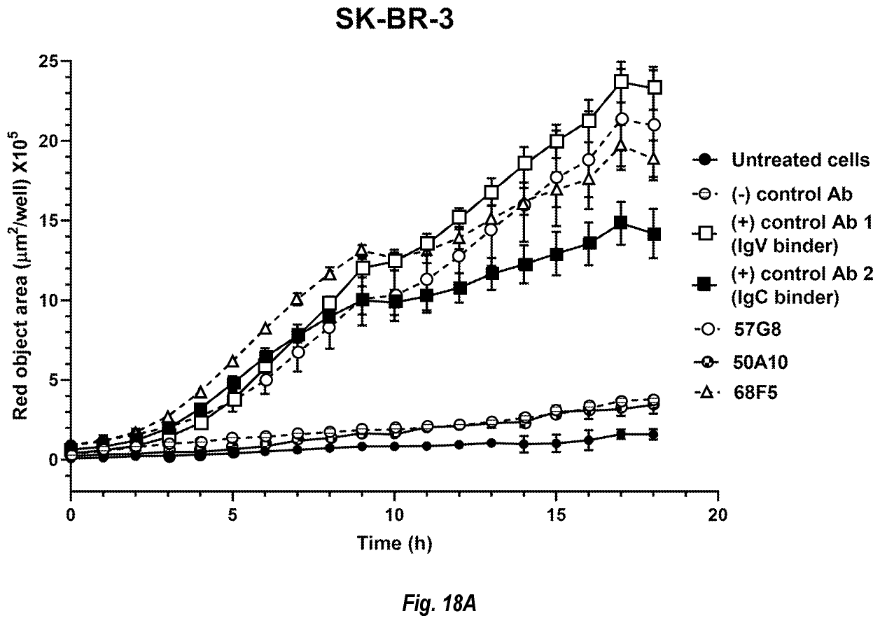

Flow cytometry experiments demonstrated that both antibodies 3F2/50A10 and 39A11/57G8 bound strongly to cellular hB7-H4 expressed on triple negative breast cancer cell line MDA-MB-468 and ovary cancer cell line SK-BR-3. Antibodies 39A11/57G8 and 9D11/68F5 also bound to cellular mB7-H4 expressed on the mB7-H4 transfected mouse colon cancer cell line CT26 (FIG. 17).

### Example 8: Anti-B7-H-4 Antibodies Cause B7-114 Internalization

Antibodies specific for cell surface antigens can induce antigen-mediated endocytosis of the antibody/antigen complex. To determine whether binding of the anti-B7-H-4 antibodies 3F2/50A10 (hB7-H4 IgV domain binding antibody), 39A11/57G8 (h/mB7-H4 IgV domain binding antibody) and 9D11/68F5 (h/mB7-H4 IgC domain binding antibody) to their target leads to internalization of the antibody/antigen complex, an IncuCyte® FabFluor antibody labeling reagent was used. FabFluor is an Fc-region targeting Fab fragment conjugated to a pH-sensitive fluorescent probe. A fluorogenic signal is observed as the Fab-Ab complex is internalized and processed via acidic (pH 4.5-5.5) lysosomes and endosomes.

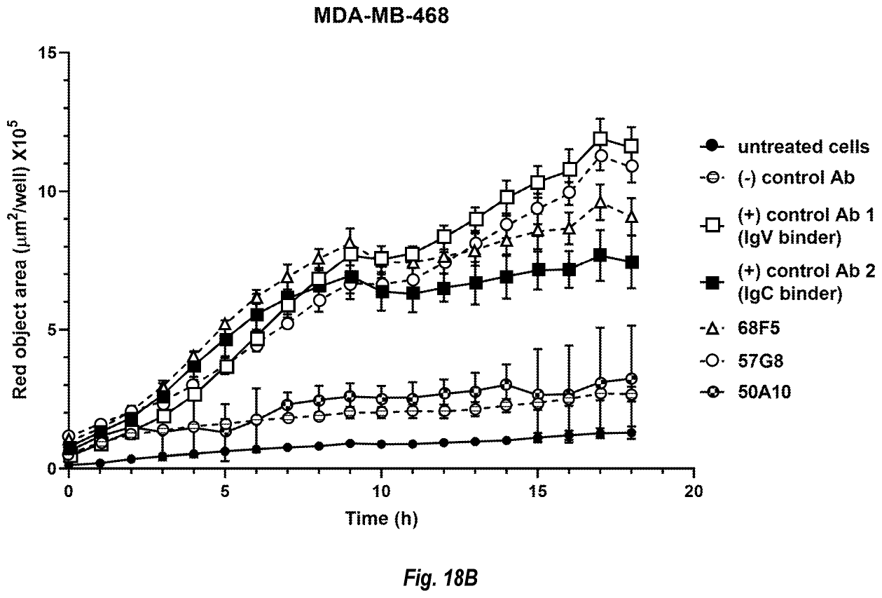

IgC domain antibody 9D111/68F5 and IgV domain antibody 39A11/57G8 triggered internalization by binding to B7-H4 (FIG. 18).

### Example 9: Anti-B7-H-4 Antibodies Restore T Cell Function

Binding of cell-expressed B7-H4 to its (unknown) cognate receptor on T cells inhibits T cell cytokine secretion function. Accordingly, it was tested whether anti-B7-H-4 antibodies 3F2/50A10 (hB7-H4 IgV domain binding antibody), 39A11/57G8 (h/mB7-H4 IgV domain binding antibody) and 9D11/68F5 (h/mB7-H4 IgC domain binding antibody) can restore T cell cytokine secretion function (e.g., IFNγ secretion) by disrupting the interaction of B7-H4 with its cognate receptor on T cells.

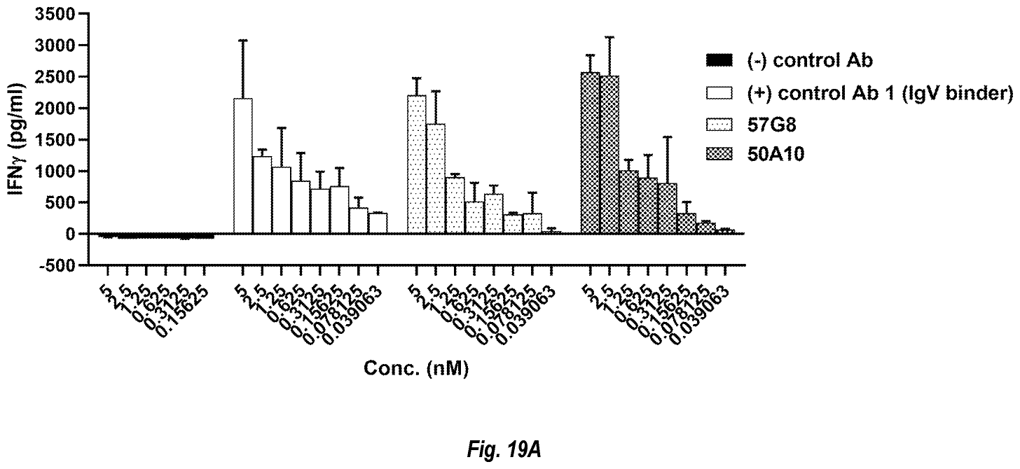

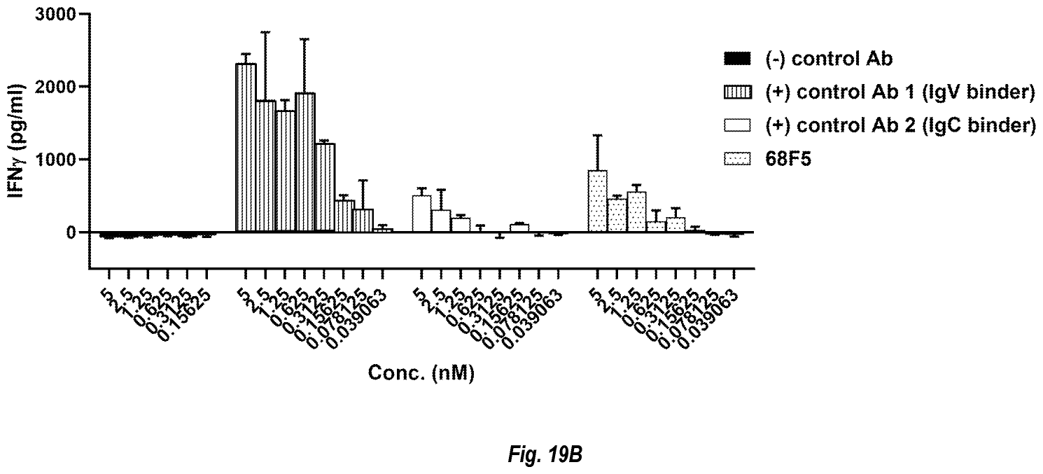

IgV-binding antibodies 39A11/57G8 and 3F2/50A10 showed high potency in restoring T cell function as measured by IFNγ secretion (FIG. 19).

### Example 10: Anti-B7-H-4 Antibodies Kill Tumor Cells

To determine the ability of anti-B7-H4 antibodies 3F2/50A10 (hB7-H4 IgV domain binding antibody), 39A11/57G8 (h/mB7-H4 IgV domain binding antibody) and 9D11/68F5 (h/mB7-H4 IgC domain binding antibody) to kill tumor cells, an antibody-dependent cell-mediated cytotoxicity (ADCC) bioassay was used (Promega G7010/G7018). The assay works as follows: When the FcγRIIIa receptor on the cell surface of effector cells (e.g., a natural killer cells) binds to the Fc effector portion of an antibody bound to its target on, for example, a cancer cell, cross-linking of the effector cell and the cancer cell occurs. This leads to activation of ADCC. The ADCC reporter assay employed in this experiment detects an early step in ADCC pathway activation: the activation of gene transcription through the NFAT (nuclear factor of activated T-cells) pathway in the effector cell. In addition, the assay uses engineered Jurkat cells stably expressing the V158 (high affinity) variant of the FcγRIIIa receptor and an NFAT response element driving expression of firefly luciferase as effector cells.

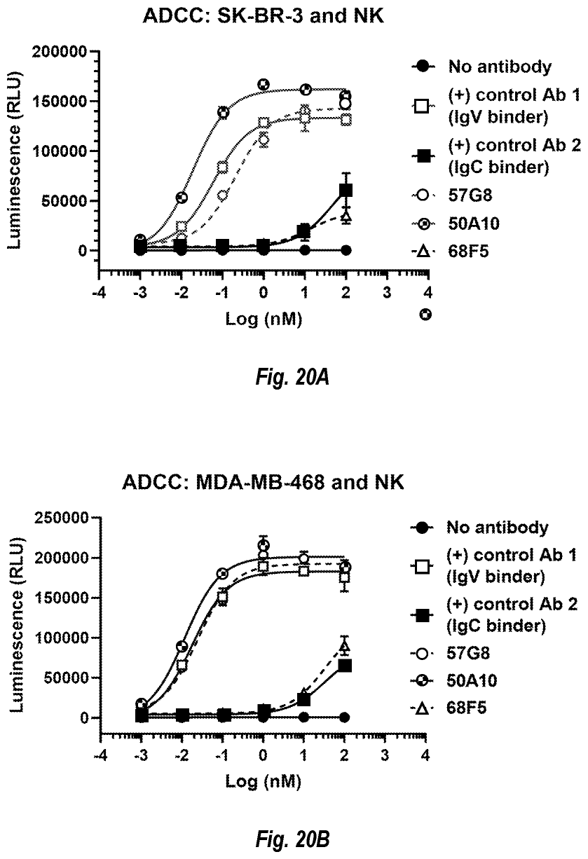

All three antibodies tested were effective in killing both SK-BR-3 and MDA-MB-468 cells (FIG. 20).

Tumor cell killing was also assessed using a IncuCyte® immune cell killing assay. For this, an SKBR3/hPBMC co-culture model was set-up for analyzing ADCC.

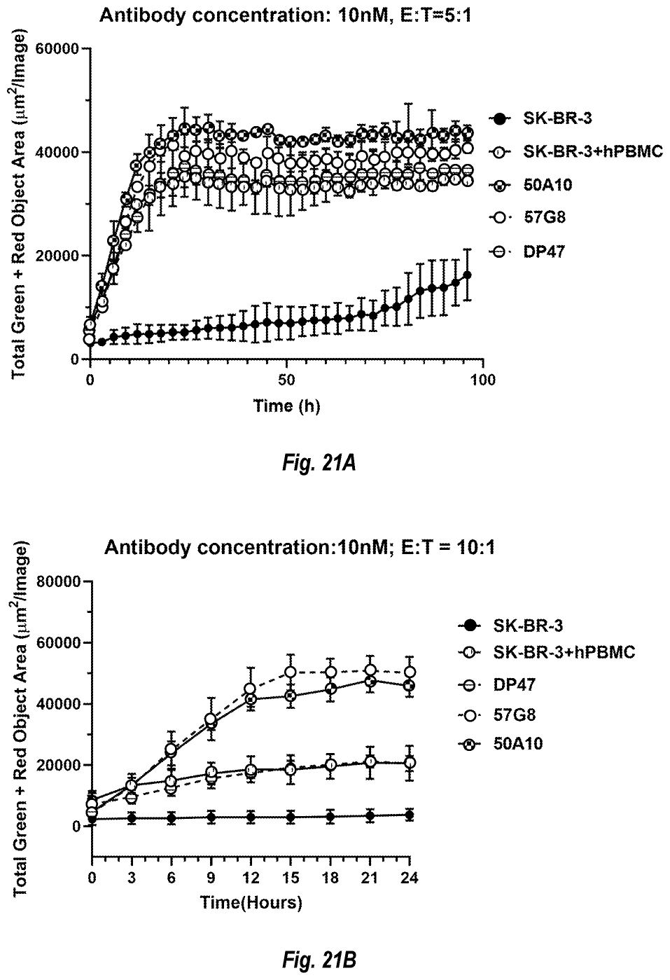

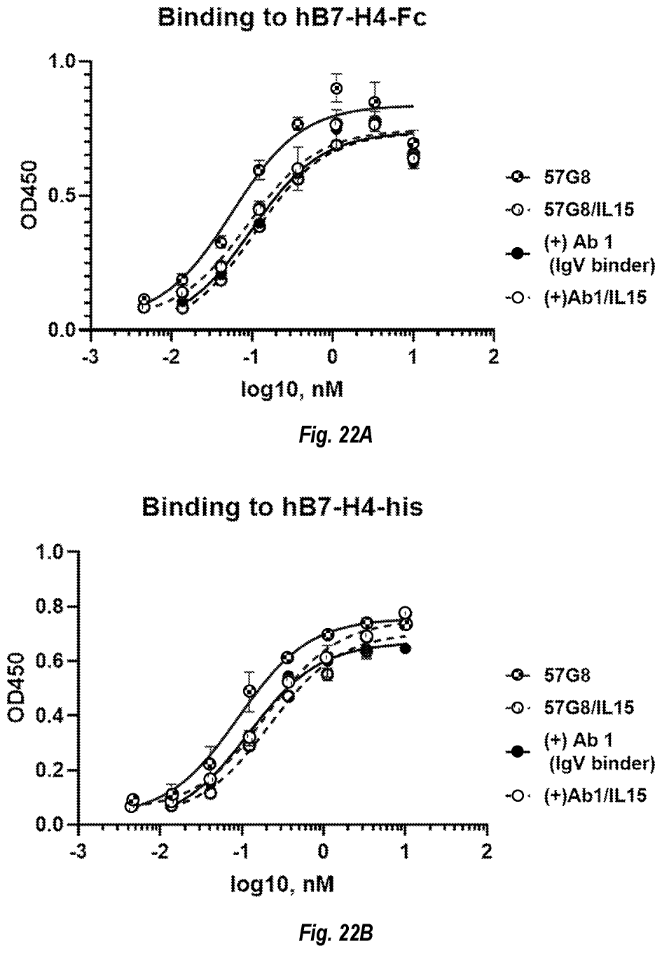

The IgV domain binding antibodies 3F2/50A10 and 39A11/57G8, but not negative control antibody DP47, were effective in killing SK-BR-3 cancer cells when mixed with hPBMCs (FIG. 21).

### Example 11: Binding Properties for Selected Anti-B7-H4 Antibodies

The kinetics to antigen binding were determined for anti-B7-H4 antibodies 3F2/50A10 (hB7-H4 IgV domain binding antibody), 39A11/57G8 (h/mB7-H4 IgV domain binding antibody) and 9D11/68F5 (h/mB7-H4 IgC domain binding antibody) (Tables 11 and 12). These experiments were conducted as a separate experiment from the Biacore analyses described above.

TABLE 11
Binding properties of antibody 39A11 and its derivatives. Ab1
was B7-H4 IgV domain binding antibody FPA150 (Five Prime).
Ab2 was B7-H4 IgC domain binding antibody 6H3 (Medimmune).

| Antibody | EC50, nM | | Kinetics for binding to hB7-H4 | | |
|---|---|---|---|---|---|
| (VH/VL) | SK-BR-3 | hB7-H4 | Kₐ | K_{d} | K_{D} |
| 3F2/50A10 | 1.18 | 0.082 | 8.67E+06 | 3.93E−04 | 4.54E−11 |
| 9D11/68F5 | 3.53 | 0.105 | 7.20E+04 | 2.13E−04 | 2.96E−09 |
| 39A11/57G8 | 2.82 | 0.060 | 9.18E+05 | 1.40E−04 | 1.52E−10 |
| (+) control Ab1 | 2.64 | 0.106 | 5.33E+05 | 2.66E−04 | 4.99E−10 |
| (+) control Ab2 | 3.37 | 0.222 | 2.04E+05 | 1.28E−04 | 6.26E−10 |

TABLE 12
Binding properties of antibody 39A11 and its derivatives. Ab1
was B7-H4 IgV domain binding antibody FPA150 (Five Prime).
Ab2 was B7-H4 IgC domain binding antibody 6H3 (Medimmune).

| Antibody | EC50, nM | | Kinetics for binding to mB7-H4 | | |
|---|---|---|---|---|---|
| (VH/VL) | mB7-H4/CT26 | mB7-H4 | Kₐ | K_{d} | K_{D} |
| 3F2/50A10 | ND | ND | 8.67E+06 | 3.93E−04 | 4.54E−11 |
| 9D11/68F5 | 9.41 | 0.144 | 7.20E+04 | 2.13E−04 | 2.96E−09 |
| 39A11/57G8 | 7.82 | 0.051 | 9.18E+05 | 1.40E−04 | 1.52E−10 |
| (+) control Ab1 | 2.06 | 0.139 | 5.33E+05 | 2.66E−04 | 4.99E−10 |
| (+) control Ab2 | 18.36 | 0.289 | 2.04E+05 | 1.28E−04 | 6.26E−10 |

### Example 12: Construction of Fusion Proteins Comprising Anti-B7-H4 Antibodies

Several heterodimeric, C-terminal fusion proteins were generated comprising anti-B7-H4 antibodies (Table 13). As illustrated in FIG. 1B, the fusion proteins comprised a first heavy chain (comprising from N- to C-terminus a variable region and a constant region), a second heavy chain (comprising from N- to C-terminus a variable region, constant region, an IL-15Ralpha sushi domain, and an IL-15), and two light chains. Heterodimierzation was achieved by introducing so-called knobs-into-holes (CW-CSAV)” mutation into the Fc region of the antibody, which further stabilize the heterodimer. The “LS” mutation (M428L/N434S) increases affinity to FcRn and lower the K_{off} rate at pH 6, thus leading to an extend serum half-life. Finally, the N65S mutation in IL-15 reduces the ability of IL-15 to stimulate lymphocyte proliferation.

TABLE 13
Structure of fusion proteins.

| Antibody | | | | | | | | | | |
|---|---|---|---|---|---|---|---|---|---|---|
| used in | | | HC1 | | HC2 | | | | LC | |
| fusion | | | | SEQ | | SEQ | | | | SEQ |
| protein | | | | ID | | ID | | | | ID |
| (VH/VL) | Antigen | Fusion protein | Name | NO | Name | NO | IL-15 | Constant Region Mutation | Name | NO |
| (+) control | IgV domain of | (+) Ab1/IL15 | 20502 | 147 | 20502 HC-2 | 154 | N65S | HC1: S354C, T366W; | 20502 | 143 |
| antibody 1 | h/m B7-H4 | | HC1 | | | | | HC2: Y349C, T366S, | LC | |
| (20502) | | | | | | | | L368A, Y407V | | |
| 39A11/57G8 | IgV domain of | 39A11/57G8-IL15 | 39A11 | 148 | 39A11-HC2 | 155 | N65S | HC1: S354C, T366W; | 57G8 | 144 |
| | h/m B7-H4 | (also called 57G8/IL15) | HC1 | | | | | HC2: Y349C, T366S, | LC | |
| | | | | | | | | L368A, Y407V | | |
| 39A11/57G8 | IgV domain of | 39A11/57G8-IL15-LS | 39A11 | 149 | 39A11- | 156 | N65S | HC1: S354C, T366W; | 57G8 | 144 |
| | h/m B7-H4 | (also called 57G8/IL15- | HC1-LS | | HC2-LS | | | M428L, N434S | LC | |
| | | LS) | | | | | | HC2: Y349C, T366S, | | |
| | | | | | | | | L368A, Y407V, M428L, | | |
| | | | | | | | | N434S | | |
| 3F2/50A10 | IgV domain of | 3F2/50A10-IL15 | 3F2 HC1 | 150 | 3F2 HC1 | 157 | N65S | HC1: S354C, T366W; | 50A10 | 145 |
| | hB7-H4 | (also called 50A10/IL15) | | | | | | HC2: Y349C, T366S, | LC | |
| | | | | | | | | L368A, Y407V | | |
| 3F2/50A10 | IgV domain of | 3F2/50A10-IL15-LS | 3F2 | 151 | 3F2 HC2- | 158 | N65S | HC1: S354C, T366W; | 50A10 | 145 |
| | hB7-H4 | (also called 50A10/IL15- | HC1-LS | | LS | | | M428L, N434S | LC | |
| | | LS) | | | | | | HC2: Y349C, T366S, | | |
| | | | | | | | | L368A, Y407V, M428L, | | |
| | | | | | | | | N434S | | |
| DP47 | Irrelevant | DP47/IL15 | DP47 | 152 | DP47 HC2 | 159 | N65S | HC1: S354C, T366W; | DP47 | 146 |
| | control antigen | | HC1 | | | | | HC2: Y349C, T366S, | LC | |
| | | | | | | | | L368A, Y407V | | |
| DP47 | Irrelevant | DP47/IL15-LS | DP47 | 153 | DP47 HC2- | 160 | N65S | HC1: S354C, T366W; | DP47 | 146 |
| | control antigen | | HC1-LS | | LS | | | M428L, N434S | LC | |
| | | | | | | | | HC2: Y349C, T366S, | | |
| | | | | | | | | L368A, Y407V, M428L, | | |
| | | | | | | | | N434S | | |

HC1 = heavy chain 1.
HC2 = heavy chain 2.
WT = wildtype.
Ab1 was B7-H4 IgV domain binding antibody FPA150 (Five Prime).

### Example 13: Binding of Fusion Proteins Comprising Anti-B7-H4 Antibodies to Soluble B7-H4

The binding of fusion proteins 57G8/IL15 and 50A10/IL15 to soluble B7-H4 was determined by Biacore and ELISA. The results of the Biocore analysis are shown in Table 14.

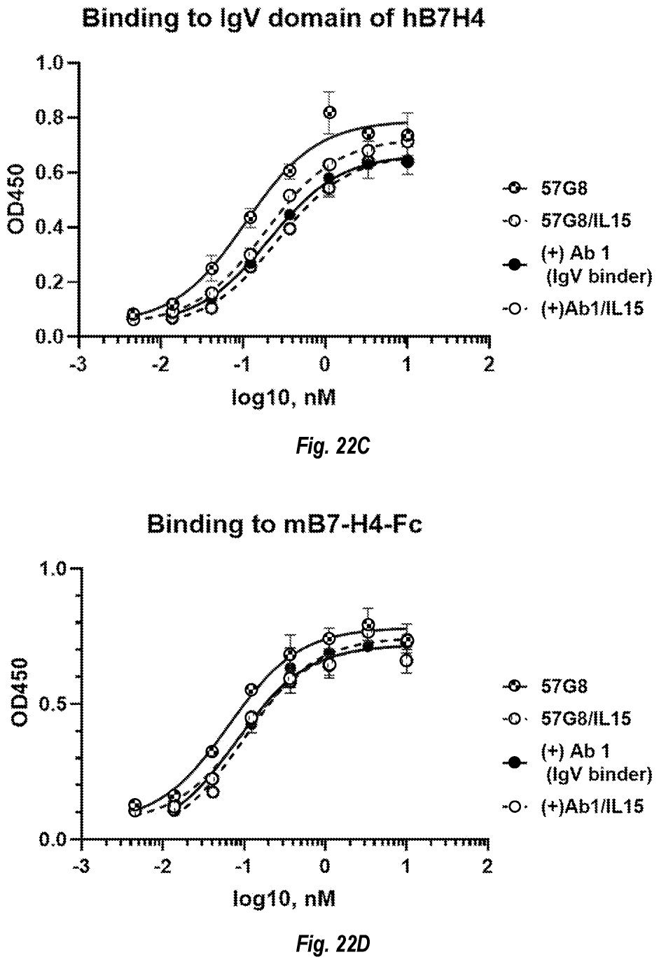

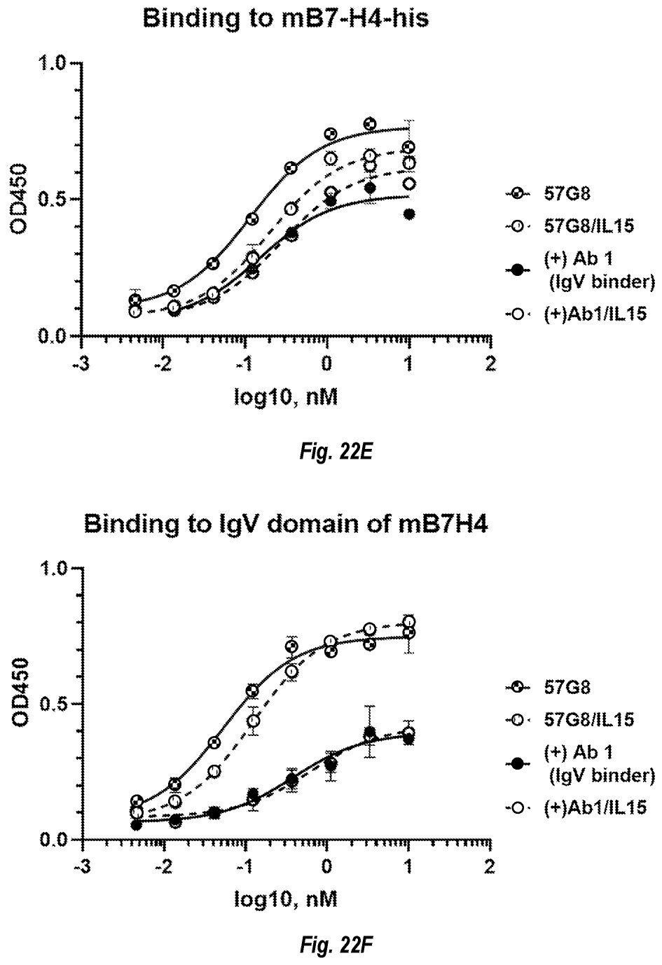

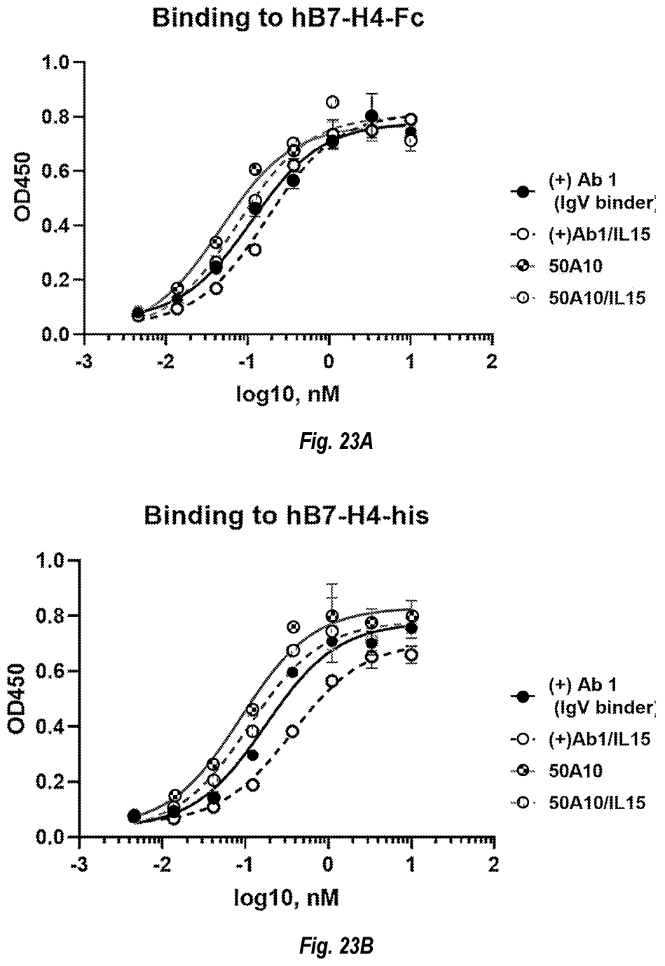

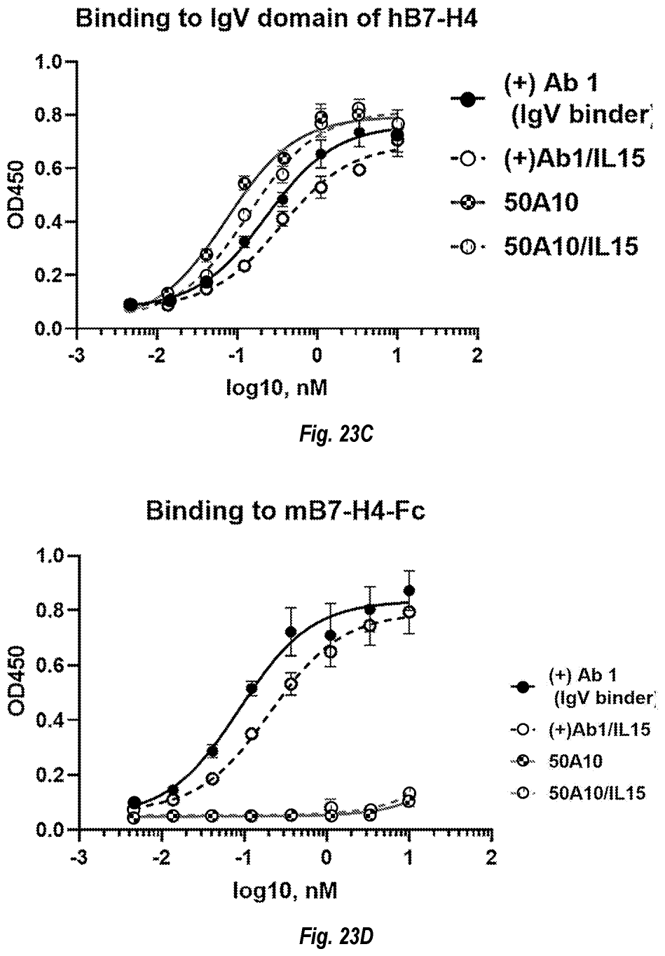

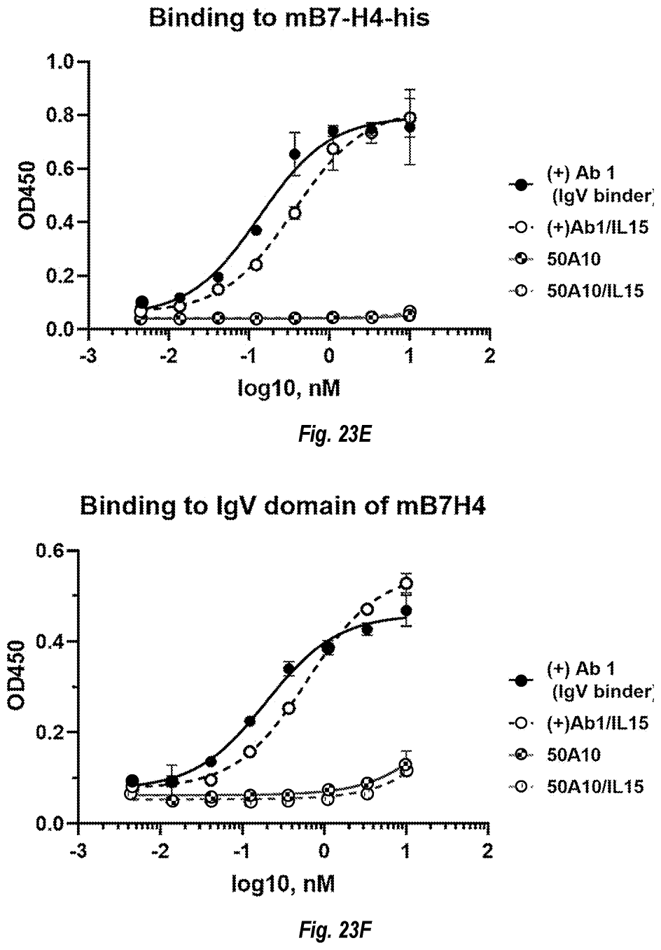

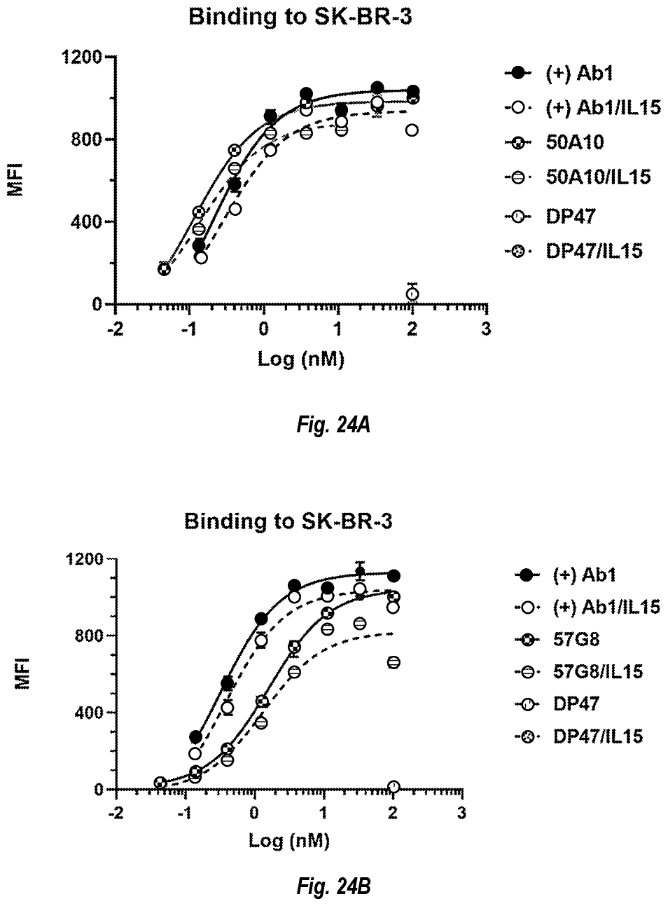

Like their parental antibody, fusion protein 50A10/IL15 only bound to hB7-H4 (IgV domain) and 57G8/IL15 bound to h/m B7-H4 (IgV domain) (see Table 14 and FIGS. 22 and 23). No significant difference was observed for binding to soluble B7-H4 between antibody and fusion antibody in both ELISA and Biacore.

TABLE 14
Binding affinity (K_{D} [M]) for fusion proteins comprising
anti-B7-H antibodies for binding to soluble B7-H4.

| | (+) | (+) | | | | |
|---|---|---|---|---|---|---|
| | Ab1 | Ab/IL15 | 39A11/57G8 | 57G8/IL15 | 3F2/50A10 | 50A10/IL15 |
| Antibody | ✓ | | ✓ | | ✓ | |
| Fusion | | ✓ | | ✓ | | ✓ |
| protein | | | | | | |
| hB7-H4 Fc | 4.50E−10 | 6.47E−10 | 1.07E−10 | 2.19E−10 | 4.33E−11 | 1.01E−10 |
| hB7-H4 his | 1.10E−10 | 1.80E−10 | 3.55E−11 | 4.54E−11 | 1.55E−11 | 2.59E−11 |

| | | | | | |
|---|---|---|---|---|---|
| mB7-H4 Fc | 5.09E−10 | 7.61E−10 | 5.18E−11 | 1.07E−10 | No binding |

(+) Ab1 = B7-H4 IgV domain binding antibody FPA150 (Five Prime).

### Example 14: Binding of Fusion Proteins Comprising Anti-B7-H4 Antibodies to Cell-Expressed B7-H4

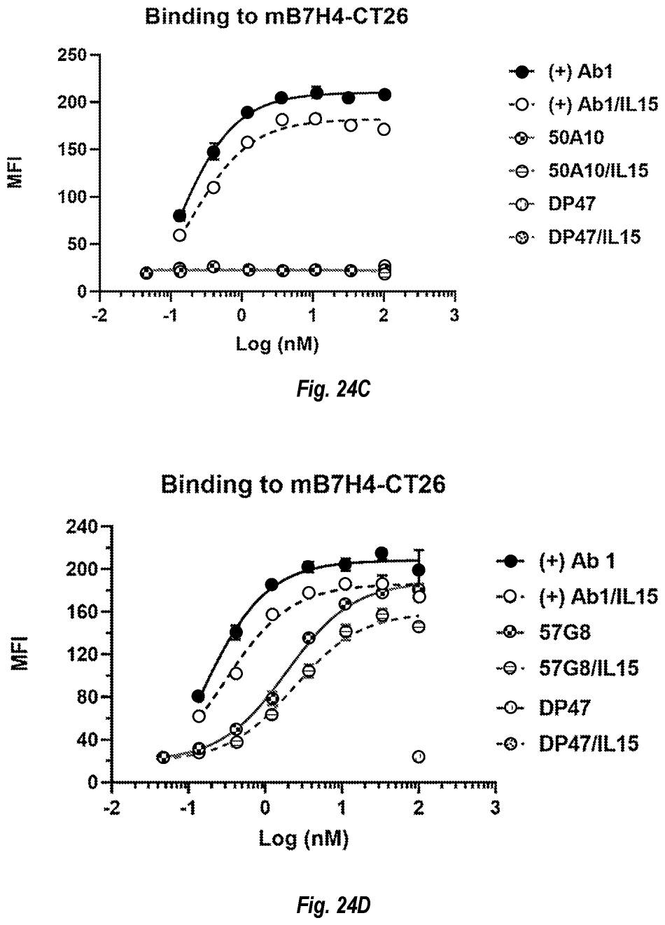

Binding of the fusion proteins to cell-expressed B7-H4 was assessed using FACS. Antibody 3F2/50A10 and fusion protein 3F2/50A10 bound to the human B7-H4 expressed on SK-BR-3 (FIGS. 24A and 24C). Antibody 39A11/57G8 and fusion protein 57G8/IL15 bound to both human and mouse B7-H4 expressed on the SK-BR-3 and mB7-H4-CT26, respectively (FIGS. 24B and 24D).

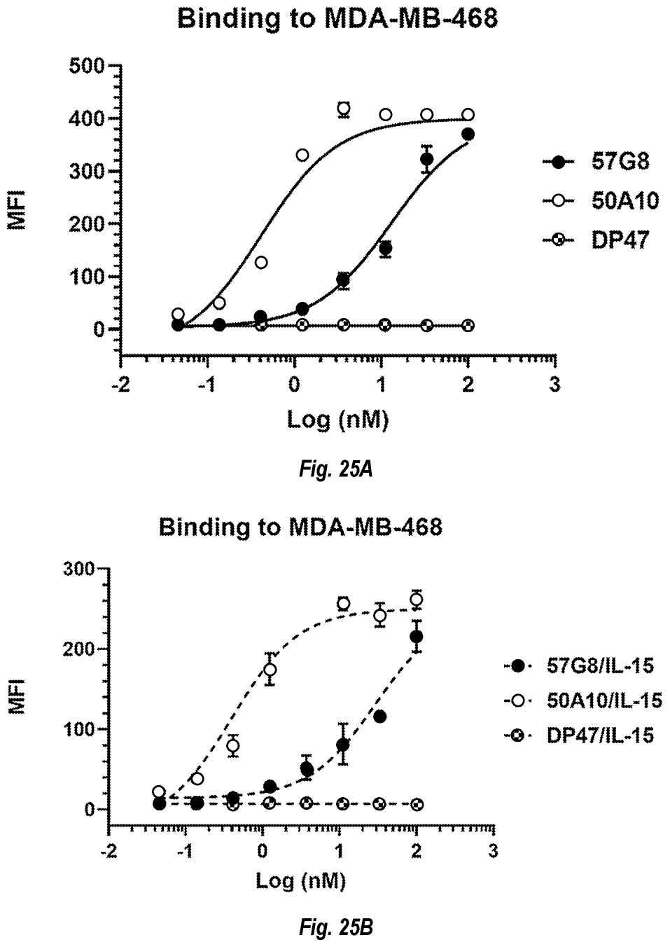

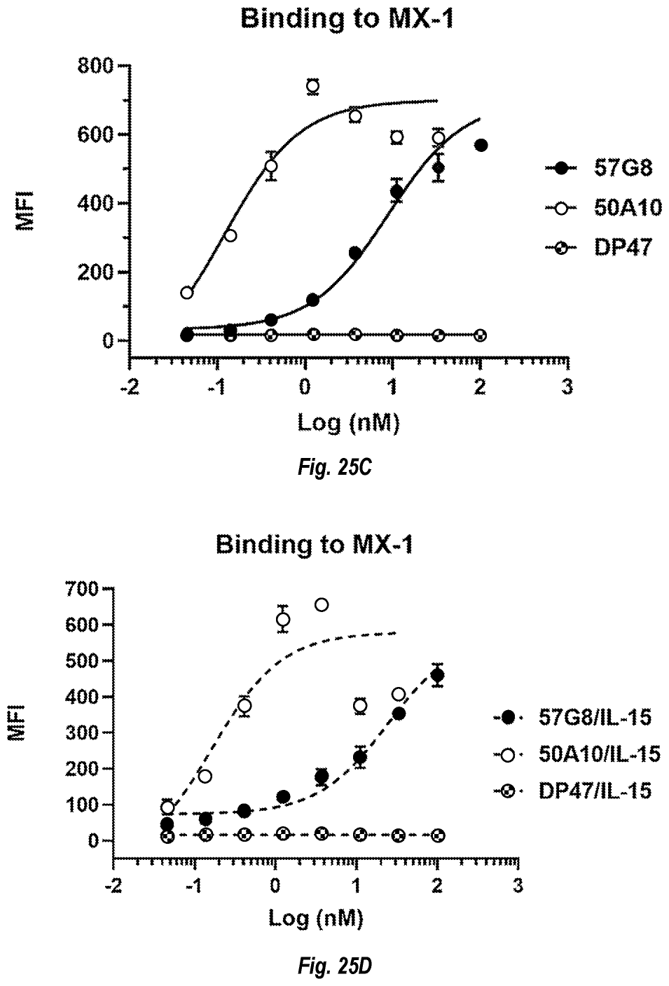

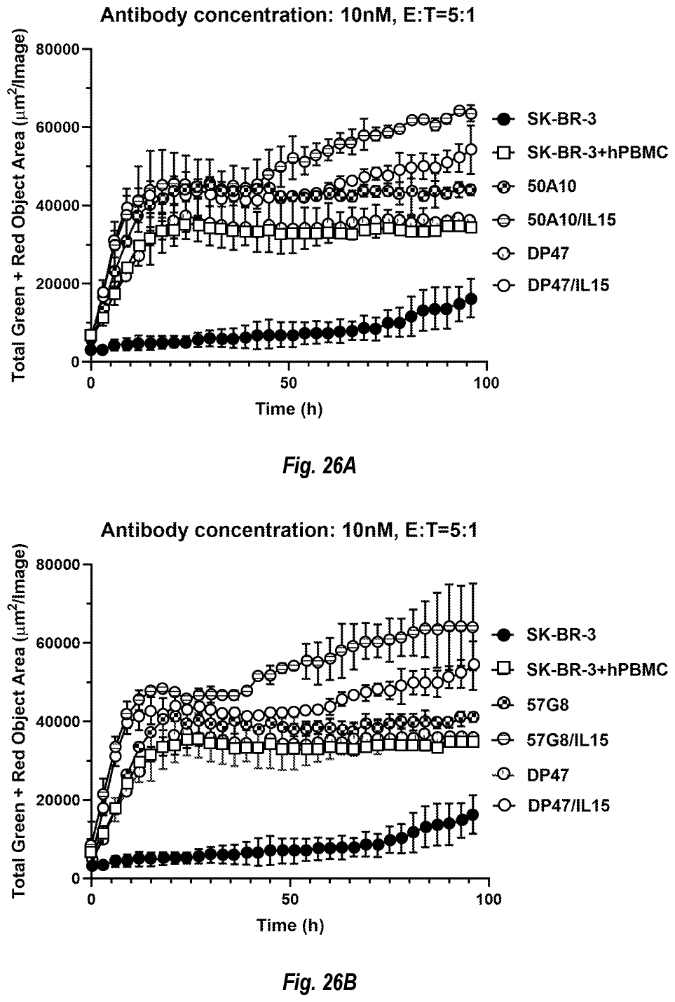

Both antibodies 39A11/57G8 and 3F2/50A10 bound to B7-H4 expressed on the triple negative breast cancer cell line MDA-MB-468 and the triple negative breast cancer cell line MX-1 (FIG. 25 and Table 15). IL-15 fusion protein comprising 39A11/57G8 or 3F2/50A10, respectively, also bound to B7-H4 expressed on MDA-MB-468 and MX-1 with similar affinity as their respective parental monospecific antibody (FIG. 25 and Table 15).

TABLE 15
EC50 values for binding of indicated antibodies and fusion
proteins to B7-H4 expressed on MDA-MB-468 and MX-1 cells.

| | 39A11/57G8 | 3F2/50A10 | 57G8/IL-15 | 50A10/IL-15 |
|---|---|---|---|---|
| Antibody | ✓ | ✓ | | |
| Fusion protein | | | ✓ | ✓ |
| MDA-MB-468 | 13.07 | 0.416 | 29.63 | 0.398 |
| EC50 [nM] | | | | |
| MX-1 cells | 8.87 | 0.120 | 24.79 | 0.162 |
| EC50 [nM] | | | | |

### Example 15: Fusion Proteins Comprising Anti-B7-H4 Antibodies Kill Tumor Cells in a Tumor Cell/hPBMC Co-Culture Model

The ability of fusion proteins comprising anti-B7-H-4 antibodies to kill tumor cells was assessed using a IncuCyte® immune cell killing assay. For this, an SKBR3/hPBMC co-culture model or an MDA-MB-468/hPBMC co-culture model was set-up for analyzing ADCC. Tumor cell killing was tested in lower effector cell and target cell ratio (5:1); cell growth was monitored by Incucyte up to 96 hours.

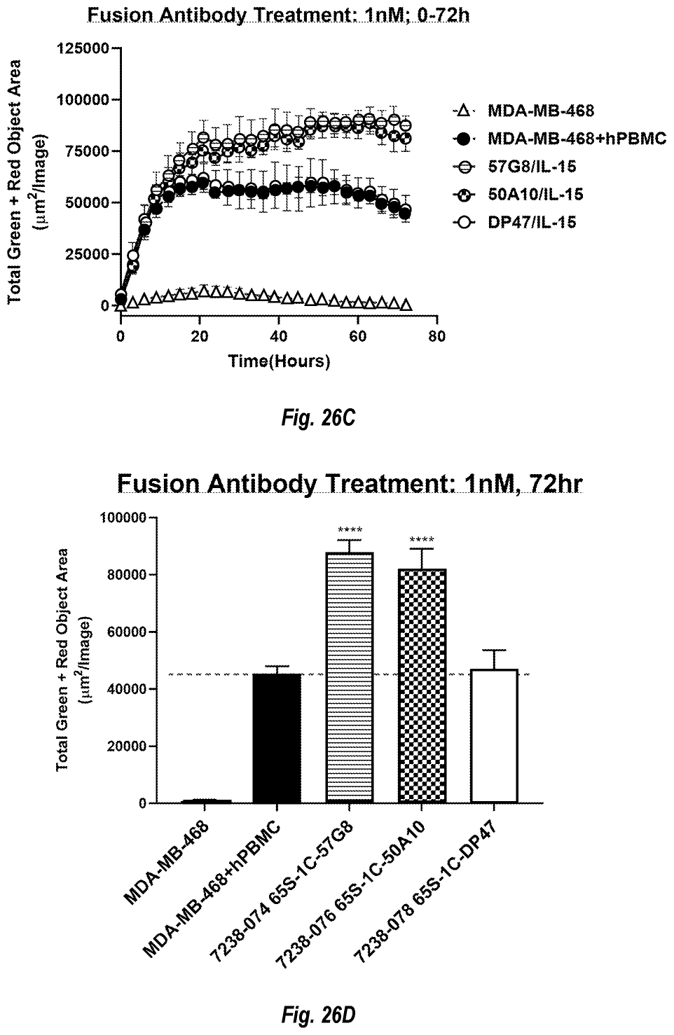

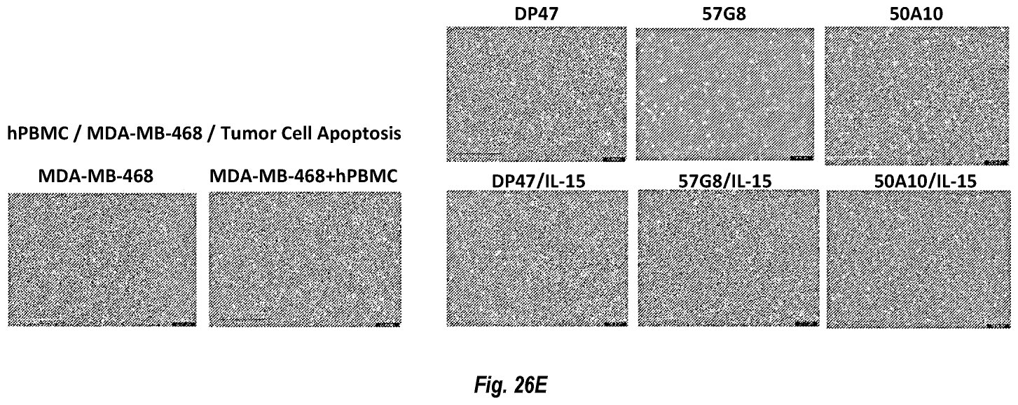

Fusion proteins 57G8/IL15 and 50A10/IL15 fusion showed stronger killing than their parental antibody 39A11/57G8 and 3F2/50A10, respectively. Fusion proteins 50A10/IL15 and 57G8/IL15 fusion showed stronger killing than the non-targeted fusion DP47/IL15 (FIG. 26).

After a 7-day treatment with fusion proteins 57G8/IL15 or 50A10/IL15, respectively, most of MDA-MB-468 cells were killed (FIG. 26E).

### Example 16: Effect of Fusion Proteins Comprising Anti-B7-H4 Antibodies on hPBMC Proliferation

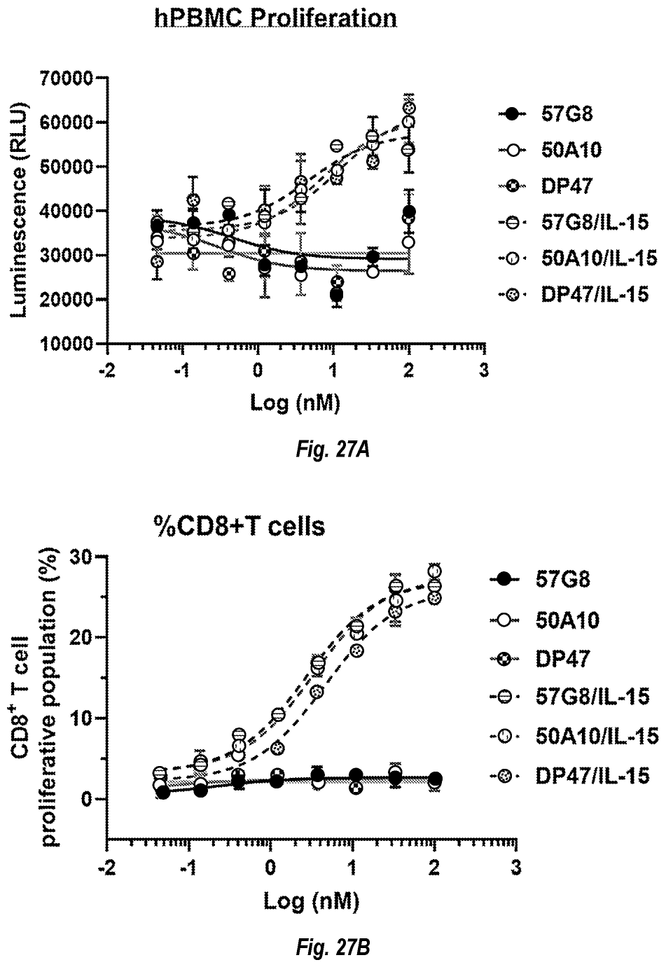

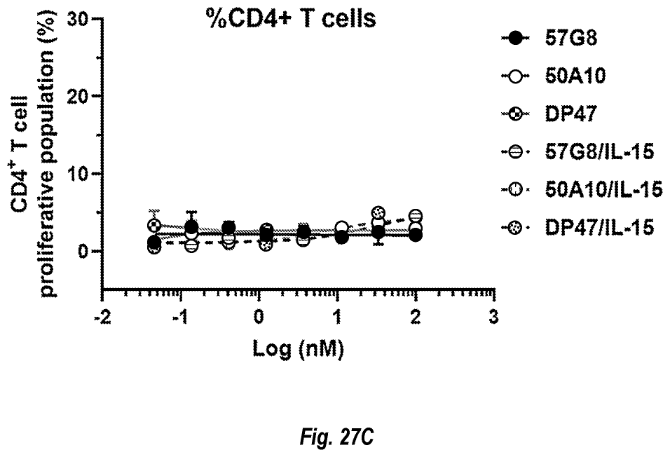

The effect of antibodies 39A11/57G8 and 3F2/50A10 as well as fusion proteins 57G8/IL15 and 50A10/IL15 on hPBMC proliferation was determined. Antibody DP47 and an IL-15 fusion protein comprising DP47 was used as a control. All fusions showed a similar ability to induce proliferation of lymphocytes in human PBMCs, which do not express B7-H4 (FIG. 27A). More CD8⁺ T cells were expanded than CD4⁺ T cells (FIGS. 27B and 27C).

### Example 17: Fusion Proteins Comprising Anti-B7-H4 Antibodies Activate STAT5 Signaling Pathway

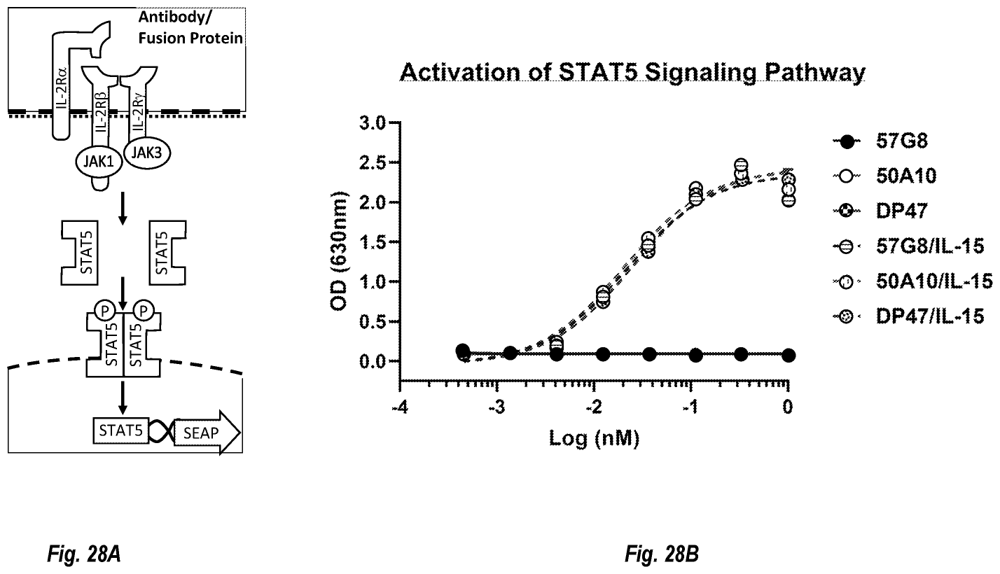

The ability of fusion proteins 57G8/IL15 and 50A10/IL115 to activate the p-STAT5 signal pathway through IL-2 receptors was examined. An IL-15 fusion protein comprising DP47 was used as a control. All fusions exhibited a similar potency to activate the STAT5 pathway downstream of IL-2Rβγ (FIG. 28).

### Example 18: Fusion Proteins Comprising Anti-B7-H4 Antibodies Kill Tumor Cells in a Promega Bioassay

The ability of fusion proteins 57G8/IL15 and 50A10/IL115 to kill tumor cells was assessed using an antibody-dependent cell-mediated cytotoxicity (ADCC) bioassay (Promega).

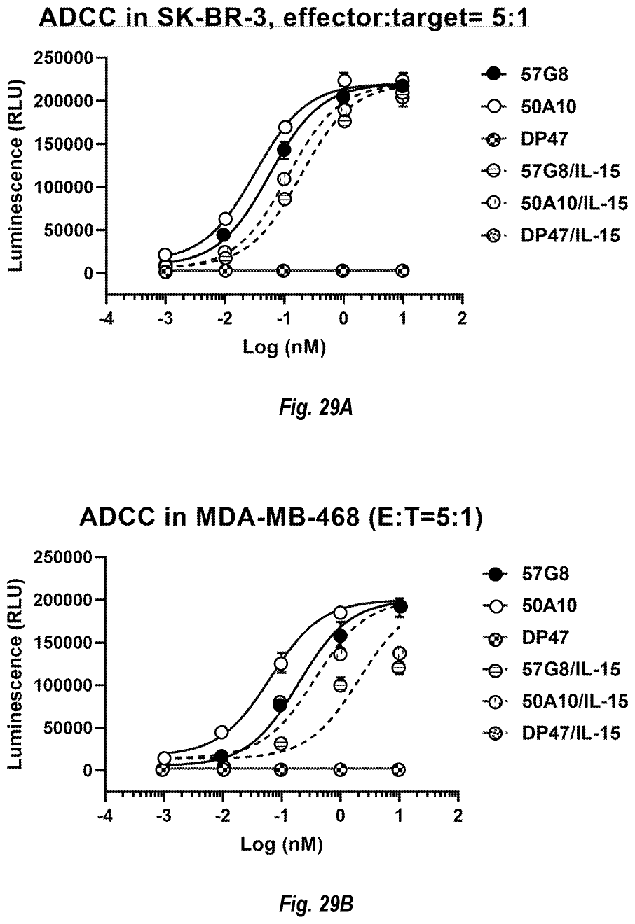

Antibodies 39A11/57G8 and 3F2/50A10 as well as fusion proteins 57G8/IL15 and 50A10/IL15 triggered the B7-H4 mediated ADCC of SK-Br-3 and MDA-MB-468 cells (FIG. 29). In MDA-MB-468 cells, 3F2/50A10 and 50A10/IL-15 showed stronger ADCC function than 39A11/57G8 and 57G8/IL-15 due to increased binding of the antibody to B7-H4 expressed on MDA-MB-468 cells.

### Example 19: Binding of Fusion Proteins (Comprising Anti-B7-H4 Antibodies and an LS Mutation) to Soluble B7-H4

The “LS” mutation (M428L/N434S) in the constant region of an antibody increases affinity to FcRn and lower the K_{off} rate at pH 6, thus leading to an extend serum half-life.

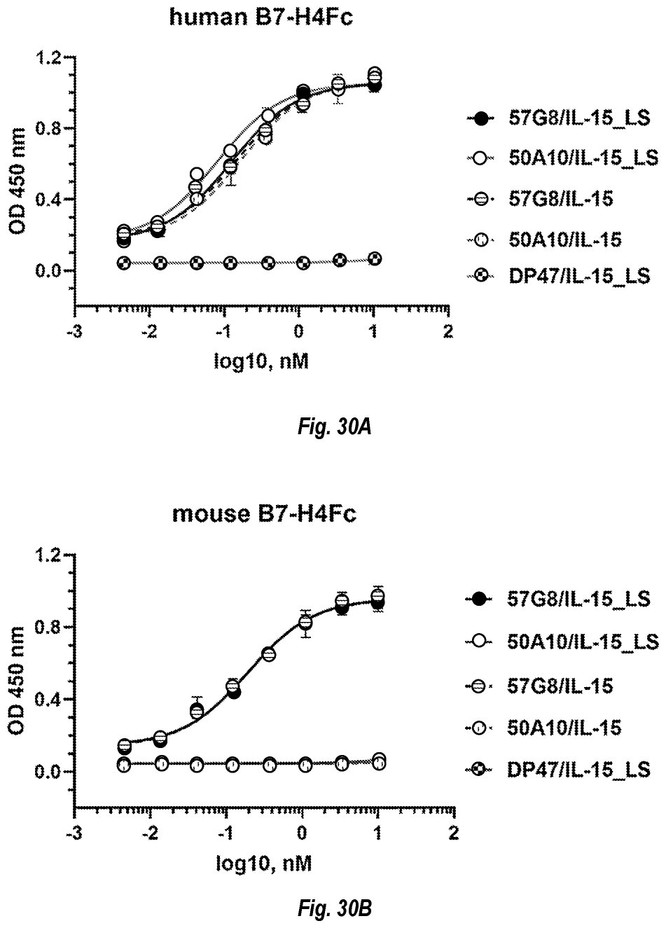

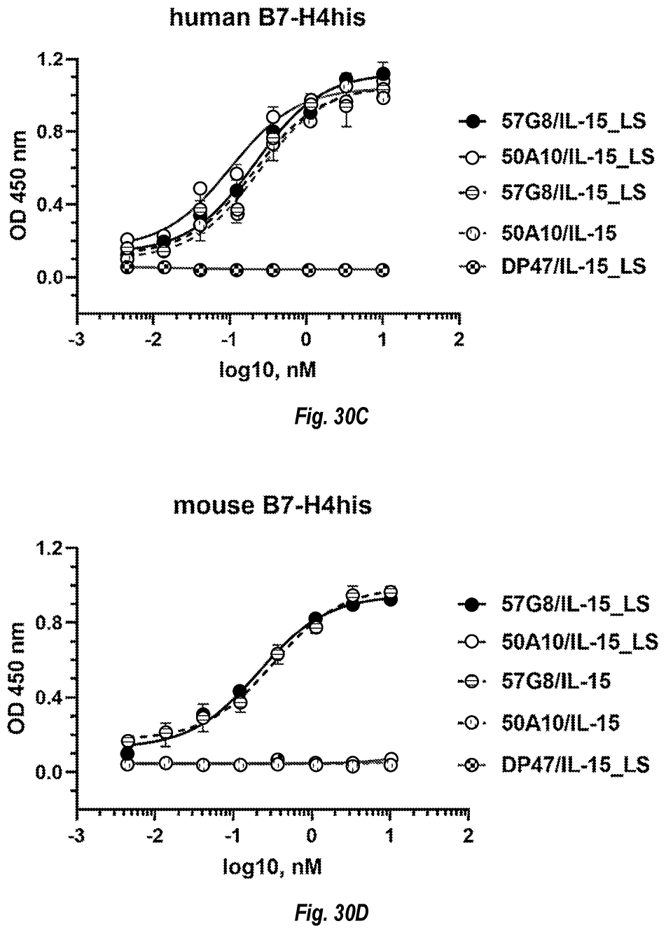

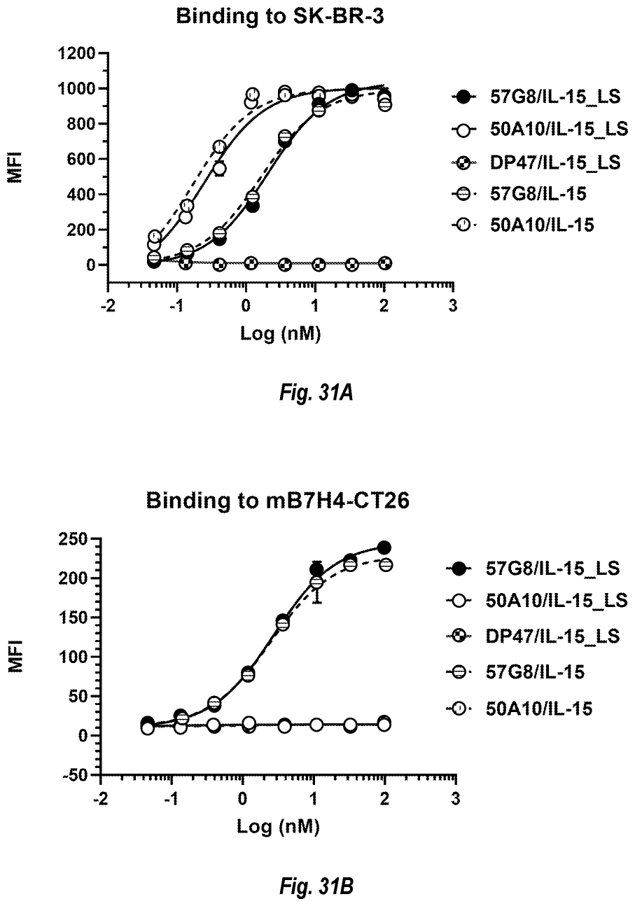

Fusion proteins 57G8/IL-15, 57G8/IL-15_LS, 50A10/IL-15 and 50A10/IL-15_LS bound to soluble human B7-H4Fc and human B7-H4his. No difference in binding was observed for these four fusion proteins (FIGS. 30A and 30C). Only fusion proteins 57G8/IL-15 and 57G8/IL-15_LS bound to soluble mouse B7-H4Fc and mouse B7-H4his. No difference in binding was observed for 57G8/IL-15_LS and 57G8/IL-15 (FIGS. 30B and 30D).

### Example 20: Binding of Fusion Proteins (Comprising Anti-B7-H4 Antibodies and an LS Mutation) to Cell-Expressed B7-H4

Binding of the fusion proteins to cell-expressed B7-H4 was assessed using FACS.

Fusion proteins 57G8/IL-15, 57G8/IL-15_LS, 50A10/IL-15 and 50A10/IL-15_LS all bound to human tumor cell expressed B7-H4. Fusion proteins 50A10/IL-15 and 50A10/IL-15_LS show stronger binding to SK-BR-3 cells than fusion proteins 57G8/IL-15 or 57G8/IL-15_LS (FIG. 31A). Fusion proteins 57G8/IL-15 and 57G8/IL-15_LS bound to mouse B7-H4 transfected in murine tumor cell line CT-26 (FIG. 31B). No difference in binding was observed for 57G8/IL-15_LS as compared to 57G8/IL-15 or for 50A10/IL-15_LS as compared to 50A10/IL-15 (FIG. 31).

### Example 21: Ability of Fusion Proteins (Comprising Anti-B7-114 Antibodies and an LS Mutation) to Induce Proliferation of IL-2-Dependent M07e Cells

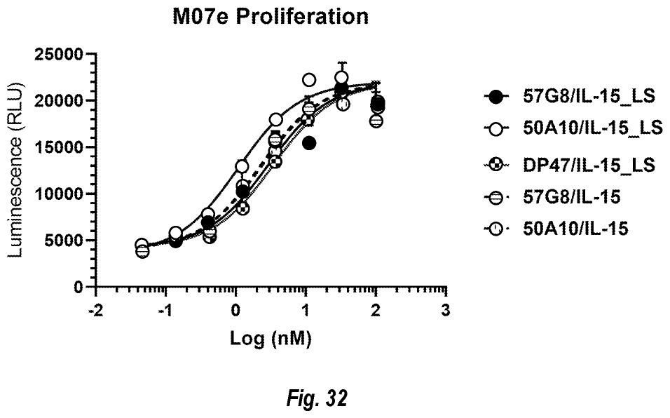

The ability of fusion proteins 57G8/IL-15, 57G8/IL-15_LS, 50A10/IL-15 and 50A10/IL-15_LS to induce proliferation of IL-2 dependent M07e cells was determined. All fusions were able to induce proliferation of M07e cells (FIG. 32).

Sequences

TABLE 16
Antibodies binding to IgV domain of
human B7H4 (3F2 and derivatives)

| | | | | | | 3F2 CDR |
|---|---|---|---|---|---|---|
| | 3F2 | | 3F2 | 49A2 | 50A10 | motif |
| | SEQ ID | | SEQ ID | SEQ ID | SEQ ID | SEQ ID |
| Type | NO: | Type | NO: | NO: | NO: | NO: |
| VH | 15 | VL | 19 | 65 | 69 | |
| CDR1H | 16 | CDR1L | 20 | 66 | 70 | 73 |
| CDR2H | 17 | CDR2L | 21 | 67 | 71 | 74 |
| CDR3H | 18 | CDR3L | 22 | 68 | 72 | 75 |

TABLE 17
Antibodies binding to IgV domain of
human B7H4 (1D3 and derivatives)

| | | | | | | 1D3 CDR |
|---|---|---|---|---|---|---|
| | 1D3 | | 1D3 | 45A2 | 47B2 | motif |
| | SEQ ID | | SEQ ID | SEQ ID | SEQ ID | SEQ ID |
| Type | NO: | Type | NO: | NO: | NO: | NO: |
| VH | 3 | VL | 7 | 76 | 80 | |
| CDR1H | 4 | CDR1L | 8 | 77 | 81 | 84 |
| CDR2H | 5 | CDR2L | 9 | 78 | 82 | 85 |
| CDR3H | 6 | CDR3L | 10 | 79 | 83 | 86 |

TABLE 18
Antibodies binding to IgC domain of human
and murine hB7-H4 (9D11 and derivatives)

| | | | | | | | | | | 9D11 |
|---|---|---|---|---|---|---|---|---|---|---|
| | | | | | | | | | | CDR |
| | 9D11 | | 9D11 | 67E12 | 67C6 | 67C3 | 67G3 | 67H9 | 68F5 | motif |
| | SEQ | | SEQ | SEQ | SEQ | SEQ | SEQ | SEQ | SEQ | SEQ |
| | ID | | ID | ID | ID | ID | ID | ID | ID | ID |
| Type | NO: | Type | NO: | NO: | NO: | NO: | NO: | NO: | NO: | NO: |
| VH | 45 | VL | 49 | 87 | 91 | 95 | 99 | 103 | 107 | |
| CDR1H | 46 | CDR1L | 50 | 88 | 92 | 96 | 100 | 104 | 108 | 111 |
| CDR2H | 47 | CDR2L | 51 | 89 | 93 | 97 | 101 | 105 | 109 | 112 |
| CDR3H | 48 | CDR3L | 52 | 90 | 94 | 98 | 102 | 106 | 110 | 113 |

TABLE 19
Antibodies binding to IgV domain of human
and murine hB7-H4 (39A11 and derivatives)

| | | | | | | | | | 39A11 |
|---|---|---|---|---|---|---|---|---|---|
| | | | | | | | | | CDR |
| | 39A11 | | 39A11 | 57H3 | 57G8 | 56A9 | 56H7 | 62F9 | motif |
| | SEQ | | SEQ | SEQ | SEQ | SEQ | SEQ | SEQ | SEQ |
| | ID | | ID | ID | ID | ID | ID | ID | ID |
| Type | NO: | Type | NO: | NO: | NO: | NO: | NO: | NO: | NO: |
| VH | 57 | VL | 61 | 114 | 118 | 122 | 126 | 130 | |
| CDR1H | 58 | CDR1L | 62 | 115 | 119 | 123 | 127 | 131 | 134 |
| CDR2H | 59 | CDR2L | 63 | 116 | 120 | 124 | 128 | 132 | 135 |
| CDR3H | 60 | CDR3L | 64 | 117 | 121 | 125 | 129 | 133 | 136 |

TABLE 20
Antibody sequences.

| | | | |
|---|---|---|---|
| | | SEQ | |
| | | ID | |
| Antibody | Description | NO: | Sequence |

Antibodies binding to IgV domain of human B7H4

| | | | |
|---|---|---|---|
| 1A12 | VH | 1 | EVQLLESGGGLVKPGGSLRLSCAASGFTFSSYAMHWVRQAPGKGLEWVAVISYDGSNKYYA |
| | | | DSVKGRFTISRDDSKNTLYLQMNSLKTEDTAVYYCARHGWDAFDIWGQGTLVTVSS |
| 1A12 | VL | 2 | DIQMTQSPSSLSASVGDRVTITCRASQDISNRLNWYQQKPGKAPKLLIYLASSLQGGVPSR |
| | | | FSGSGSGTDFTLTISSLQPEDFATYYCQQAISFPLTFGGGTKVEIK |
| 1D3 | VH | 3 | EVQLLESGGGLVQPGGSLRLSCAASGFTFNTHAMHWVRQAPGKGLEWVSAISGSGGSTYYA |
| | | | DSVKGRFTISRDISKNTLYLQMNSLRAEDTAVYYCARLLGRFGEYGMDVWGQGTTVTVSS |
| 1D3 | CDR1H | 4 | GFTFNTHAMH |
| 1D3 | CDR2H | 5 | AISGSGGSTYYADSVKG |
| 1D3 | CDR3H | 6 | LLGRFGEYGMDV |
| 1D3 | VL | 7 | DIQMTQSPSSLSASVGDRVTITCRASRSIYTWLAWYQQKPGKAPKLLIYDASTLQSGVPSR |
| | | | FSGSGSGTDFTLTISSLQPEDFATYYCQQSYSTPYTFGGGTKVEIK |
| 1D3 | CDR1L | 8 | RASRSIYTWLA |
| 1D3 | CDR2L | 9 | STLQS |
| 1D3 | CDR3L | 10 | QQSYSTPYT |
| 1F11 | VH | 11 | QVQLVQSGAEVKKPGSSVKVSCKASGDTFTTYQIHWVRQAPGQGLEWMGGIMPIFGTTKYA |
| | | | QNFQGRVTITADESTSTAYMELSSLRSEDTAVYYCARRNYGMDVWGQGTTVTVSS |
| 1F11 | VL | 12 | DIVMTQSPDSLAVSLGERATINCKSSQSLLYRPVNKNFLAWYQQKPGQPPKLLIYWASTRG |
| | | | SGVPDRFSGSGSGTDFTLTISSLQAEDVATYFCQQYYSTPITFGQGTKVEIK |
| 3D1 | VH | 13 | QVQLVQSGAEVKKPGSSVKVSCKASGYSFISYAITWVRQAPGQGLEWIGGINPIFGTAKYA |
| | | | QKFQGRVTITADESTSTAYMELSSLRSEDTAVYYCAKGQALIQHWGQGTLVTVSS |
| 3D1 | VL | 14 | DIVMTQSPDSLAVSLGERATINCKSSQSVLSSSNNKNYLAWYQQKPGQPPKLLIYWASTRE |
| | | | SGVPDRFSGSGSGTDFTLTISSLQAEDVAVYYCQQYYSTPLAFGQGTKVEIK |
| 3F2 | VH | 15 | QVQLVQSGAEVKKPGASVKVSCKASGYTFTAQYMYWVRQAPGQGLEWMGRINPTSGNTVYA |
| | | | QKLQGRVTMTRDTSTSTVYMELSSLRSEDTAVYYCARGINWFDPWGQGTLVTVSS |
| 3F2 | CDR1H (EU | 16 | GYTFTAQYMY |
| | index | | |
| | numbering) | | |
| 3F2 | CDR2H | 17 | RINPTSGNTVYAQKLQG |
| | (EU index | | |
| | numbering) | | |
| 3F2 | CDR3H (EU | 18 | GINWFDP |
| | index | | |
| | numbering) | | |
| 3F2 | CDR1H | 172 | AQYMY |
| | (Kabat EU | | |
| | index | | |
| | numbering) | | |
| 3F2 | CDR2H | 173 | RINPTSGNTVYAQKLQG |
| | (Kabat EU | | |
| | index | | |
| | numbering) | | |
| 3F2 | CDR3H | 174 | GINWFDP |
| | (Kabat EU | | |
| | index | | |
| | numbering) | | |
| 3F2 | CDR1H | 178 | GYTFTAQY |
| | (IGMT) | | |
| 3F2 | CDR2H | 179 | INPTSGNT |
| | (IGMT) | | |
| 3F2 | CDR3H | 180 | ARGINWFDP |
| | (IGMT) | | |
| 3F2 | CDR1H | 184 | GYTFTAQ |
| | (Chothia) | | |
| 3F2 | CDR2H | 185 | PTSG |
| | (Chothia) | | |
| 3F2 | CDR3H | 186 | INWFD |
| | (Chothia) | | |
| 3F2 | VL | 19 | DIQMTQSPSSLSASVGDRVTITCRASQSISSYLNWYQQKPGKAPKLLIYAASSLQSGVPSR |
| | | | FSGSGSGTDFTLTISSLQPEDFATYYCQQSYSTPLTFGGGTKVEIK |
| 3F2 | CDR1L | 20 | RASQSISSYLN |
| 3F2 | CDR2L | 21 | SSLQS |
| 3F2 | CDR3L | 22 | QQSYSTPLT |
| 4H6 | VH | 23 | EVQLLESGGGLVQPGGSLRLSCAASGFTFSSYGMHWVRQAPGKGLEWVSGISGSGGSTYYA |
| | | | DSVKGRFTISRDNSKNTLYLQMNSLRAEDTAVYYCARTQWRASLDPWGQGTLVNVSS |
| 4H6 | VL | 24 | DIQMTQSPSSLSASVGDRVTITCRASQDISYWLAWYQQKPGKAPKLLIYGASTLQSGVPSR |
| | | | FSGSGSGTDFTLTISSLQPEDFATYYCQQTTMSPYTFGQGTKVEIK |
| 6C3 | VH | 25 | EVQLLESGGGLVQPGGSLRLSCAASGFTFSSYAMHWVRQAPGKGLEWVASISGTGFTTRYA |
| | | | DSVKGRFTISRDNSKNTLYLQMNSLRAEDTAVYYCARGGWFRDTPVDYWGQGTLVTVSS |
| 6C3 | VL | 26 | DIVMTQSPLSLPVTPGEPASISCRSSQSLLHSNGYNYLDWYLQKPGQSPQLLIYGASSLRS |
| | | | GVPDRFSGSGSGTDFTLKISRVEAZDVGVYYCMQASHWPPTFGQGTRLEIK |
| 9H2 | VH | 27 | QVQLVQSGAEVKKPGSSVKVSCKASGGTFSTYAINWVRQAPGQGLEWMGGINPMFGTARYA |
| | | | QKFQGRVTITADESTSTAYMELSSLRSEDTAVYYCARGRLHRHWGQGTLVTVSS |
| 9H2 | VL | 28 | DIVMTQSPDSLAVSLGERATINCKSSQSVLYSSNNKNYLAWYQQKPGQPPKLLIYWASTRE |
| | | | SGVPDRFSGSGSGTDFTLTISSLQAEDVAVYYCQQYYTTPITFGQGTRLEIK |

Antibody binding to both IgV and IgC domains of human B7H4

| | | | |
|---|---|---|---|
| 4B9 | VH | 29 | QVQLVQSGAEVKKPGASVKVSCKASGYTFTSYYMHWVRQAPGQGLEWMGWMNPNSGNTGYA |
| | | | QKFQGRVTMTRDTSTSTVYMELSSLRSEDTAVYYCARGQGRYLNYYYMDVWGKGTTVTVSS |
| 4B9 | VL | 30 | DIQMTQSPSSLSASVGDRVTITCRVSQGISNYLAWYQQKPGKAPKLLIYDASSLQSGVPSR |
| | | | FSGSGSGTDFTLTISSLQPEDFATYYCQQSYSTPFTFGPGTKVDIK |

Antibodies binding to IgC domain of human and murine hB7-H4

| | | | |
|---|---|---|---|
| 15B11 | VH | 31 | QVQLVQSGAEVKKPGASVKVSCKASGYTFTGYYIHWVRQAPGQGLEWMGWMNPHSGHTGYA |
| | | | QNLQGRVTMTRDTSTSTVYMELSSLRSEDTAVYYCARVKVTTGFDYWGQGTLVTVSS |
| 15B11 | VL | 32 | DIVMTQSPLSLPVTPGEPASISCRSSQSLLHSNGYNYLDWYLQKPGQSPQLLIYLGSNRAS |
| | | | GVPDRFSGSGSGTDFTLKISRVEAEDVGVYYCMQGTHWPPTFGQGTKLEIK |
| 24B6 | VH | 33 | QVQLVQSGAEVKKPGASVKVSCKASGYTFTGYYMHWVRQAPGQGLEWIGWMNPNSGDTGYA |
| | | | QKFQGRVTMTRDTSTSTVYMELSSLRSEDTAVYYCAKVGAYGGMDVWGQGTTVTVSS |
| 24B6 | VL | 34 | DIVMTQSPLSLPVTPGEPASISCRSSQSLLHSNGYNYLDWYLQKPGQSPQLLIYGGSNRAS |
| | | | GVPDRFSGSGSGTDFTLKISRVEAEDVGVYYCMQGTHWPPTFGPGTKVDIK |
| 24F4 | VH | 35 | QVQLVQSGAEVKKPGSSVKVSCKASGYRFTGDYIHWVRQAPGQGLEWMGWMNPNSGNTGLA |
| | | | QKFQGRVTITADESTSTAYMELSSLRSEDTAVYYCARVLGFSGGMDVWGQGTTVTVSS |
| 24F4 | VL | 36 | DIVMTQSPLSLPVTPGEPASISCRSSQSLLHSNGYNYLDWYLQKPGQSPQLLIYAASSLRS |
| | | | GVPDRFSGSGSGTDFTLKISRVEAEDVGVYYCMQGTHWPPTFGQGTKLEIK |
| 30G4 | VH | 37 | QVQLVQSGAEVKKPGASVKVSCKASGYTFSSYYMHWVRQAPGQGLEWMGWMNPNSGDTGYA |
| | | | QKFQGRVTMTRDTSTSTVYMELSSLRSEDTAVYYCARVPETGGMDVWGQGTTVTVSS |
| 30G4 | VL | 38 | DIVMTQSPLSLPVTPGEPASISCRSSQSLLHSNGYNYLDWYLQKPGQSPQLLIYGASSLHS |
| | | | GVPDRFSGSGSGTDFTLKISRVEAEDVGVYYCMQGTHWPPTFGQGTKVEIK |
| 5E4 | VH | 39 | QVQLVQSGAEVKKPGASVKVSCKASGYTFTSYDINWVRQAPGQGLEWMGWMNPNSGNTGYA |
| | | | QKFQGRVTMTRDTSTSTVYMELSSLRSEDTAVYYCARGELWFGEFDYYYYYGMDVWGQGTT |
| | | | VTVSS |
| 5E4 | VL | 40 | DIQMTQSPSSLSASVGDRVTITCRASQSISSYLNWYQQKPGKAPKLLIYGASSLQSGVPSR |
| | | | FSGSGSGTDFTLTISSLQPEDFATYYCQQADRIPFTFGRGTKVEIK |
| 5F4 | VH | 41 | EVQLLESGGGLVKPGGSLRLSCAASGFTFSSYAMHWVRQAPGKGLEWVSAIGGSGRTTYYA |
| | | | DSVKGRFTISRDDSKNTLYLQMNSLKTEDTAVYYCARERGYSYGQIDYWGQGTLVTVSS |
| 5F4 | VL | 42 | DIVMTQSPLSLPVTPGEPASISCRSSQSLLHSNGYNYLDWYLQKPGQSPQLLIYAASSLQS |
| | | | GVPDRFSGSGSGTDFTLKISRVEAEDVGVYYCMQGAHWPLTFGQGTKVEIK |
| 5G6 | VH | 43 | QVQLVQSGAEVKKPGASVKVSCKASGYTFTSYYMHWVRQAPGQGLEWLGWVNPNTGTTRFA |
| | | | QKFQGRVTMTRDTSTSTVYMELSSLRSEDTAVYYCARDANYYYGMDVWGQGTMVTVSS |
| 5G6 | VL | 44 | DIVMTQSPDSLAVSLGERATINCKSSQSVLYSSNNKNYLAWYQQKPGQPPKLLIYWASTRE |
| | | | SGVPDRFSGSGSGTDFTLTISSLQAEDVATYYCQKYNSALPITFGQGTRLEIK |
| 9D11 | VH | 45 | QVQLVQSGAEVKKPGASVKVSCKASGYTFTSYGISWVRQAPGQGLEWVGVINPNGGTTTYA |
| | | | QTFQGRVTMTRDTSTSTVYMELSSLRSEDTAVYYCARTGYSSGWAFDYWGRGTLVTVSS |
| 9D11 | CDR1H | 46 | GYTFTSYGIS |
| 9D11 | CDR2H | 47 | VINPNGGTTTYAQTFQG |
| 9D11 | CDR3H | 48 | TGYSSGWAFDY |
| 9D11 | VL | 49 | DIVMTQSPDSLAVSLGERATINCKSSQSVLYSSNNKNYLAWYQQKPGQPPKLLIYWASTRE |
| | | | SGVPDRFSGSGSGTDFTLTISSLQAEDVAVYYCQQYFSTPSFGQGTKVEIK |
| 9D11 | CDR1L | 50 | KSSQSVLYSSNNKNYLA |
| 9D11 | CDR2L | 51 | STRES |
| 9D11 | CDR3L | 52 | QQYFSTPS |
| 9E1 | VH | 53 | QVQLVQSGAEVKKPGASVKVSCKASGYTFTGYYLHWVRQAPGQGLEWMGWINPNSGGTNSA |
| | | | QRFQGRVTMTRDTSTSTVYMELSSLRSEDTAVYYCARVRGYEGGMDVWGQGTTVTVSS |
| 9E1 | VL | 54 | DIVMTQSPLSLPVTPGEPASISCRSSQSLLHSNGYNYLDWYLQKPGQSPQLLIYLGSNRAS |
| | | | GVPDRFSGSGSGTDFTLKISRVEAEDVGVYYCMQGSHWPPTFGQGTRLEIK |

Antibodies binding to IgV domain of human and murine hB7-H4

| | | | |
|---|---|---|---|
| 31D7 | VH | 55 | EVQLLESGGGLVQPGGSXRLSCAASGFTFSSYGMHWVRQAPGKGLEWVSSISSSSSYIYYA |
| | | | DSVKGRFTISRDNSKNTLYLQMNSLRAEDTAVYYCARICSCCSLDYWGQGTLVTVSS |
| 31D7 | VL | 56 | DIQMTQSPSSLSASVGDRVTITCRASQSISNWLAWYQQKPGKAPKLLIYAASSLQSGVPSR |
| | | | FSGSGSGTDFTLTISSLQPEDFATYYCQQSYTIPITFGQGTRLEIK |
| 39A11 | VH | 57 | EVQLLESGGGLVQPGGSLRLSCAASGFTFSSYGMHWVRQAPGKGLEWVASISSSSGYIYYA |
| | | | DSVKGRFTISRDNSKNTLYLQMNSLRAEDTAVYYCARASSGMDVWGQGTTVTVSS |
| 39A11 | CDR1H | 58 | GFTFSSYGMH |
| 39A11 | CDR2H | 59 | SISSSSGYIYYADSVKG |
| 39A11 | CDR3H | 60 | ASSGMDV |
| 39A11 | VL | 61 | DIQMTQSPSSLSASVGDRVTITCRASQTIRSYLNWYQQKPGKAPKLLIYAASHLQSGVPSR |
| | | | FSGSGSGTDFTLTISSLQPEDFATYYCQQSYTTPYTFGQGTKLEIK |
| 39A11 | CDR1L | 62 | RASQTIRSYLN |
| 39A11 | CDR2L | 63 | SHLQS |
| 39A11 | CDR3L | 64 | QQSYTTPYT |

Antibodies binding to hB7-H4: 3F2 derivatives

| | | | |
|---|---|---|---|
| 49A2 | VL | 65 | DIQMTQSPSSLSASVGDRVTITCRASQNIDTYVNWYQQKPGKAPKLLIYAASRLHTVPSRF |
| | | | SGSGSGTDFTLTISSLQPEDFATYYCQQSYTSPFTFGGGTKVEIK |
| 49A2 | CDR1L | 66 | RASQNIDTYVN |
| 49A2 | CDR2L | 67 | SRLHT |
| 49A2 | CDR3L | 68 | QQSYTSPFT |
| 50A10 | VL | 69 | DIQMTQSPSSLSASVGDRVTITCQASQDISNYINWYQQKPGKAPKLLIYAASRLQSGVPSR |
| | | | FSGSGSGTDFTLTISSLQPEDFATYYCQQSYRSPFTFGQGTKLEIK |
| 50A10 | CDR1L (EU | 70 | QASQDISNYIN |
| | index | | |
| | numbering) | | |
| 50A10 | CDR2L | 71 | SRLQS |
| | (EU index | | |
| | numbering) | | |
| 50A10 | CDR3L (EU | 72 | QQSYRSPFT |
| | index | | |
| | numbering) | | |
| 50A10 | CDRIL | 175 | SQDISNY |
| | (Kabat EU | | |
| | index | | |
| | numbering) | | |
| 50A10 | CDR2L | 176 | AAS |
| | (Kabat EU | | |
| | index | | |
| | numbering) | | |
| 50A10 | CDR3L | 177 | SYRSPF |
| | (Kabat EU | | |
| | index | | |
| | numbering) | | |
| 50A10 | CDR1L | 181 | SQDISNY |
| | (IGMT) | | |
| 50A10 | CDR2L | 182 | AAS |
| | (IGMT) | | |
| 50A10 | CDR3L | 183 | SYRSPF |
| | (IGMT) | | |
| 50A10 | CDR1L | 187 | QASQDISNYIN |
| | (Chothia) | | |
| 50A10 | CDR2L | 188 | AASRLQS |
| | (Chothia) | | |
| 50A10 | CDR3L | 189 | QQSYRSPFT |
| | (Chothia) | | |
| 3F2 CDR motif | CDR1L | 73 | X₁ASQX₂IX₃X₄YX₅N |
| | | | Wherein: |
| | | | X₁ is R or Q |
| | | | X₂ is D, N, or S |
| | | | X₃ is D or S |
| | | | X₄ is N, S, or T |
| | | | X₅ is I, L, or V |
| 3F2 CDR motif | CDR2L | 74 | SX₆LX₇X₈ |
| | | | Wherein: |
| | | | X₆ is R or S |
| | | | X₇ is H or Q |
| | | | X₈ is S or T |
| 3F2 CDR motif | CDR3L | 75 | QQSYX₉X₁₀PX₁₁T |
| | | | Wherein: |
| | | | X₉ is R, S, or T |
| | | | X₁₀ is S or T |
| | | | X₁₁ is F or L |

Antibodies binding to hB7-H4: 1D3 derivatives

| | | | |
|---|---|---|---|
| 45A2 | VL | 76 | DIQMTQSPSSLSASVGDRVTITCRASQNIYTWLAWYQQKPGKAPKLLIYDATNLPTGVPSR |
| | | | FSGSGSGTDFTLTISSLQPEDFATYYCQQSYSTRWTFGGGTKVEIK |
| 45A2 | CDR1L | 77 | RASQNIYTWLA |
| 45A2 | CDR2L | 78 | TNLPT |
| 45A2 | CDR3L | 79 | QQSYSTRWT |
| 47B2 | VL | 80 | DIQMTQSPSSLSASVGDRVTITCRASQTVYTWLAWYQQKPGKAPKLLIYDATNLATGVPSR |
| | | | FSGSGSGTDFTLTISSLQPEDFATYYCQQSYSTSWTFGGGTKVEIK |
| 47B2 | CDR1L | 81 | RASQTVYTWLA |
| 47B2 | CDR2L | 82 | TNLAT |
| 47B2 | CDR3L | 83 | QQSYSTSWT |
| 1D3 CDR motif | CDR1L | 84 | RASX₁₂X₁₃X₁₄YTWLA |
| | | | Wherein: |
| | | | X₁₂ is Q or R |
| | | | X₁₃ is N, S, or T |
| | | | X₁₄ is I or V |
| 1D3 CDR motif | CDR2L | 85 | X₁₅X₁₆LX₁₇X₁₈ |
| | | | Wherein: |
| | | | X₁₅ is S or T |
| | | | X₁₆ is N or T |
| | | | X₁₇ is A, P, or Q |
| | | | X₁₈ is S or T |
| 1D3 CDR motif | CDR3L | 86 | QQSYSTX₁₉X₂₀T |
| | | | Wherein: |
| | | | X₁₉ is P, R, or S |
| | | | X₂₀ is W or Y |

Antibodies binding to IgC domain of human and murine hB7-H4: 9D11 derivatives

| | | | |
|---|---|---|---|
| 67E12 | VL | 87 | DIVMTQSPDSLAVSLGERATINCKSSQSVLYSSNNKNYLAWYQQKPGQPPKLLIYWASKRV |
| | | | SGVPDRFSGSGSGTDFTLTISSLQAEDVAVYYCQQYFDSPTFGQGTKVEIK |
| 67E12 | CDR1L | 88 | KSSQSVLYSSNNKNYLA |
| 67E12 | CDR2L | 89 | SKRVS |
| 67E12 | CDR3L | 90 | QQYFDSPT |
| 67C6 | VL | 91 | DIVMTQSPDSLAVSLGERATINCKSSQSVLSSSNNKNYLAWYQQKPGQPPKLLIYWASTRQ |
| | | | SGVPDRFSGSGSGTDFTLTISSLQAEDVAVYYCQQYYSDPTFGQGTKVEIK |
| 67C6 | CDR1L | 92 | KSSQSVLSSSNNKNYLA |
| 67C6 | CDR2L | 93 | STRQS |
| 67C6 | CDR3L | 94 | QQYYSDPT |
| 67C3 | VL | 95 | DIVMTQSPDSLAVSLGERATINCKSSQSVLYSSNNKNYLAWYQQKPGQPPKLLIYWASTRA |
| | | | SGVPDRFSGSGSGTDFTLTISSLQAEDVAVYYCQQYYDTPTFGQGTKVEIK |
| 67C3 | CDR1L | 96 | KSSQSVLYSSNNKNYLA |
| 67C3 | CDR2L | 97 | STRAS |
| 67C3 | CDR3L | 98 | QQYYDTPT |
| 67G3 | VL | 99 | DIVMTQSPDSLAVSLGERATINCKSSQSVLSSSNNKNYLAWYQQKPGQPPKLLIYWASTRQ |
| | | | SGVPDRFSGSGSGTDFTLTISSLQAEDVAVYYCQQYYTSPTFGQGTKVEIK |
| 67G3 | CDR1L | 100 | KSSQSVLSSSNNKNYLA |
| 67G3 | CDR2L | 101 | STRQS |
| 67G3 | CDR3L | 102 | QQYYTSPT |
| 67H9 | VL | 103 | DIVMTQSPDSLAVSLGERATINCRSSQSVLYSSNNKNYLAWYQQKPGQPPKLLIYWASNRK |
| | | | SGVPDRFSGSGSGTDFTLTISSLQAEDVAVYYCQQYYSAPTFGQGTKVEIK |
| 67H9 | CDR1L | 104 | RSSQSVLYSSNNKNYLA |
| 67H9 | CDR2L | 105 | SNRKS |
| 67H9 | CDR3L | 106 | QQYYSAPT |
| 68F5 | VL | 107 | DIVMTQSPDSLAVSLGERATINCKSSRSVLSRSNNKNYLAWYQQKPGQPPKLLIYWASTRQ |
| | | | FGVPDRFSGSGSGTDFTLTISSLQAEDVAVYYCQQYYDTPTFGQGTKVEIK |
| 68F5 | CDR1L | 108 | KSSRSVLSRSNNKNYLA |
| 68F5 | CDR2L | 109 | STRQF |
| 68F5 | CDR3L | 110 | QQYYDTPT |
| 9D11 CDR motif | CDR1L | 111 | X₂₁SSX₂₂SVLX₂₃X₂₄SNNKNYLA |
| | | | Wherein: |
| | | | X₂₁ is K or R |
| | | | X₂₂ is Q or R |
| | | | X₂₃ is S or Y |
| | | | X₂₄ is R or S |
| 9D11 CDR motif | CDR2L | 112 | SX₂₅RX₂₆X₂₇ Wherein: |
| | | | X₂₅ is K, N, T |
| | | | X₂₆ is A, E, K, Q, or V |
| | | | X₂₇ is F or S |
| 9D11 CDR motif | CDR3L | 113 | QQYX₂₈X₂₉X₃₀PX₃₁ |
| | | | Wherein: |
| | | | X₂₈ is F or Y |
| | | | X₂₉ is D, S, or T |
| | | | X₃₀ is A, D, S, or T |
| | | | X₃₁ is S or T |

Antibodies binding to IgV domain of human and murine hB7-H4: 39A11 derivatives

| | | | |
|---|---|---|---|
| 57H3 | VL | 114 | DIQMTQSPSSLSASVGDRVTITCQASQDIRKYLNWYQQKPGKAPKLLIYAASTRESGVPSR |
| | | | FSGSGSGTDFTLTISSLQPEDFATYYCQQYYSLPLTFGQGTKLEIK |
| 57H3 | CDR1L | 115 | QASQDIRKYLN |
| 57H3 | CDR2L | 116 | STRES |
| 57H3 | CDR3L | 117 | QQYYSLPLT |
| 57G8 | VL | 118 | DIQMTQSPSSLSASVGDRVTITCRASQSIRSYLNWYQQKPGKAPKLLIYYASSLQSGVPSR |
| | | | FSGSGSGTDFTLTISSLQPEDFATYYCQQYYSTPLTFGQGTKVEIK |
| 57G8 | CDR1L | 119 | RASQSIRSYLN |
| 57G8 | CDR2L | 120 | SSLQS |
| 57G8 | CDR3L | 121 | QQYYSTPLT |
| 56A9 | VL | 122 | DIQMTQSPSSLSASVGDRVTITCRASQSISSYLNWYQQKPGKAPKLLIYAASIRESGVPSR |
| | | | FSGSGSGTDFTLTISSLQPEDFATYYCQQYYTTPLTFGQGTKLEIK |
| 56A9 | CDR1L | 123 | RASQSISSYLN |
| 56A9 | CDR2L | 124 | SIRES |
| 56A9 | CDR3L | 125 | QQYYTTPLT |
| 56H7 | VL | 126 | DIQMTQSPSSLSASVGDRVTITCRASQSISSYLNWYQQKPGKAPKLLIYAASNLQSGVPSR |
| | | | FSGSGSGTDFTLTISSLQPEDFATYYCQQYYTTPLTFGQGTKLEIK |
| 56H7 | CDR1L | 127 | RASQSISSYLN |
| 56H7 | CDR2L | 128 | SNLQS |
| 56H7 | CDR3L | 129 | QQYYTTPLT |
| 62F9 | VL | 130 | DIQMTQSPSSLSASVGDRVTITCRASQSVSSAVAWYQQKPGKAPKLLIYAASIRESGVPSR |
| | | | FSGSGSGTDFTLTISSLQPEDFATYYCQQYYSTPLTFGQGTKLEIK |
| 62F9 | CDR1L | 131 | RASQSVSSAVA |
| 62F9 | CDR2L | 132 | SIRES |
| 62F9 | CDR3L | 133 | QQYYSTPLT |
| 39A11 CDR | CDR1L | 134 | X₃₂ASQX₃₃X₃₄X₃₅X₃₆X₃₇X₃₈X₃₉ |
| motif | | | Wherein: |
| | | | X₃₂ is Q or R |
| | | | X₃₃ is D, S, or T |
| | | | X₃₄ is I or V |
| | | | X₃₅ is R or S |
| | | | X₃₆ is K or S |
| | | | X₃₇ is A or Y |
| | | | X₃₈ is L or V |
| | | | X₃₉ is A or N |
| 39A11 CDR | CDR2L | 135 | SX₄₀X₄₁X₄₂S |
| motif | | | Wherein: |
| | | | X₄₀ is H, I, N, S, or T |
| | | | X₄₁ is L or R |
| | | | X₄₂ is E or Q |
| 39A11 CDR | CDR3L | 136 | QQX₄₃YX₄₄X₄₅PX₄₆T |
| motif | | | Wherein: |
| | | | X₄₃ is S or Y |
| | | | X₄₄ is S or T |
| | | | X₄₅ is L or T |
| | | | X₄₆ is L or Y |

Control antibodies

| | | | |
|---|---|---|---|
| Control antibody | VH | 137 | QLQLQESGPGLVKPSETLSLTCTVSGGSIKSGSYYWGWIRQPPGKGLEWIGNIYYSGSTYY |
| 1 | | | NPSLRSRVTISVDTSKNQFSLKLSSVTAADTAVYYCAREGSYPNQFDPWGQGTLVTVSS |
| (antibody 20502 | | | |
| described in | | | |
| US20190085080 | | | |
| A1) | | | |
| Control antibody | VL | 138 | EIVMTQSPATLSVSPGERATLSCRASQSVSSNLAWYQQKPGQAPRLLIYGASTRATGIPAR |
| 1 | | | FSGSGSGTEFTLTISSLQSEDFAVYYCQQYHSFPFTFGGGTKVEIK |
| (antibody 20502 | | | |
| described in | | | |
| US20190085080 | | | |
| A1) | | | |
| Control antibody | VH | 139 | EVQLQQSGPVLVKPGTSVKMSCKASGYTFTDYYMNWVKQSHGKSLEWIGVINPYNGDTTYN |
| 2 (antibody 6H3 | | | QKFKGKATLTVDKSSSTAYMEVNSLTFEDSAVYYCARYPESTYWGQGTLVTVSA |
| described in | | | |
| U.S. Pat. No. | | | |
| 9,574,000) | | | |
| Control antibody | VL | 140 | DVVMTQTPLSLPVSLGDQASISCRSSQSLVHINGNTYLHWYLQKPGQSPKVLIYKVSNRFS |
| 2 (antibody 6H3 | | | GVPDRFSGSGSGTDFTLKISRVEAEDLGVYFCSQSTHVPLTFGAGTKLELK |
| described in | | | |
| U.S. Pat. No. | | | |
| 9,574,000) | | | |
| DP47 | VH | 141 | EVQLLESGGGLVQPGGSLRLSCAASGFTFSSYAMSWVRQAPGKGLEWVSAISGSGGSTYYA |
| | | | DSVKGRFTISRDNSKNTLYLQMNSLRAEDTAVYYCAKGSGFDYWGQGTLVTVSS |
| DP47 | VL | 142 | EIVLTQSPGTLSLSPGERATLSCRASQSVSSSYLAWYQQKPGQAPRLLIYGASSRATGIPD |
| | | | RFSGSGSGTDFTLTISRLEPEDFAVYYCQQYGSSPLTFGQGTKVEIK |

TABLE 21
Fusion protein sequences.

| | VH | | VL | | HC1 | | HC2 | | LC | |
|---|---|---|---|---|---|---|---|---|---|---|
| | | SEQ | | SEQ | | SEQ | | SEQ | | SEQ |
| | | ID | | ID | | ID | | ID | | ID |
| Fusion protein | Name | NO | Name | NO | Name | NO | Name | NO | Name | NO |
| (+) Ab1/IL15 | 20502 VH | 137 | 20502 VL | 138 | 20502 HC1 | 147 | 20502 HC-2 | 154 | 20502 LC | 143 |
| 39A11/57G8-IL15 | 39A11 VH | 57 | 57G8 VL | 118 | 39A11 HC1 | 148 | 39A11-HC2 | 155 | 57G8 LC | 144 |
| (also called 57G8/IL15) | | | | | | | | | | |
| 39A11/57G8-IL15-LS | 39A11 VH | 57 | 57G8 VL | 118 | 39A11 HC1-LS | 149 | 39A11-HC2-LS | 156 | 57G8 LC | 144 |
| (also called 57G8/IL15- | | | | | | | | | | |
| LS) | | | | | | | | | | |
| 3F2/50A10-IL15 | 3F2 VH | 15 | 50A10 VL | 69 | 3F2 HC1 | 150 | 3F2 HC1 | 157 | 50A10 LC | 145 |
| (also called 50A10/IL15) | | | | | | | | | | |
| 3F2/50A10-IL15-LS | 3F2 VH | 15 | 50A10 VL | 69 | 3F2 HC1-LS | 151 | 3F2 HC2-LS | 158 | 50A10 LC | 145 |
| (also called 50A10/IL15- | | | | | | | | | | |
| LS) | | | | | | | | | | |
| DP47/IL15 | DP47 VH | 141 | DP47 VL | 142 | DP47 HC1 | 152 | DP47 HC2 | 159 | DP47 LC | 146 |
| DP47/IL15-LS | DP47 VH | 141 | DP47 VL | 142 | DP47 HC1-LS | 153 | DP47 HC2-LS | 160 | DP47 LC | 146 |

TABLE 22
Fusion protein sequences.

| | Used in | | | SEQ | |
|---|---|---|---|---|---|
| | which fusion | | | ID | |
| Description | protein | Type | Annotation | NO: | Sequence |
| 20502 LC | (+) Ab1/IL15 | LC | 20502 VL | 143 | EIVMTQSPATLSVSPGERATLSCRASQSVSSNLAWYQQKPGQAPRLLIYGA |
| (control | | | | | STRATGIPARFSGSGSGTEFTLTISSLQSEDFAVYYCQQYHSFPFTFGGGT |
| antibody 1) | | | | | KVEIKRTVAAPSVFIFPPSDEQLKSGTASVVCLLNNFYPREAKVQWKVDNA |
| | | | | | LQSGNSQESVTEQDSKDSTYSLSSTLTLSKADYEKHKVYACEVTHQGLSSP |
| | | | | | VTKSFNRGEC |
| 57G8 LC | 39A11/57G8- | LC | 57G8 VL | 144 | DIQMTQSPSSLSASVGDRVTITCRASQSIRSYLNWYQQKPGKAPKLLIYYA |
| | IL15 | | | | SSLQSGVPSRFSGSGSGTDFTLTISSLQPEDFATYYCQQYYSTPLTFGQGT |
| | and | | | | KVEIKRTVAAPSVFIFPPSDEQLKSGTASVVCLLNNFYPREAKVQWKVDNA |
| | 39A11/57G8- | | | | LQSGNSQESVTEQDSKDSTYSLSSTLTLSKADYEKHKVYACEVTHQGLSSP |
| | IL15-LS | | | | VTKSFNRGEC |
| 50A10 LC | 3F2/50A10- | LC | 50A10 VL | 145 | DIQMTQSPSSLSASVGDRVTITCQASQDISNYINWYQQKPGKAPKLLIYAA |
| | IL15 and | | | | SRLQSGVPSRFSGSGSGTDFTLTISSLQPEDFATYYCQQSYRSPFTFGQGT |
| | 3F2/50A10- | | | | KLEIKRTVAAPSVFIFPPSDEQLKSGTASVVCLLNNFYPREAKVQWKVDNA |
| | IL15-LS | | | | LQSGNSQESVTEQDSKDSTYSLSSTLTLSKADYEKHKVYACEVTHQGLSSP |
| | | | | | VTKSFNRGEC |
| DP47 LC | DP47/IL15 and | LC | DP47 VL | 146 | EIVLTQSPGTLSLSPGERATLSCRASQSVSSSYLAWYQQKPGQAPRLLIYG |
| | DP47/IL15-LS | | | | ASSRATGIPDRFSGSGSGTDFTLTISRLEPEDFAVYYCQQYGSSPLTFGQG |
| | | | | | TKVEIKRTVAAPSVFIFPPSDEQLKSGTASVVCLLNNFYPREAKVQWKVDN |
| | | | | | ALQSGNSQESVTEQDSKDSTYSLSSTLTLSKADYEKHKVYACEVTHQGLSS |
| | | | | | PVTKSFNRGEC |
| 20502 HC1 | (+) Ab1/IL15 | HC1 | 20502 VH | 147 | QLQLQESGPGLVKPSETLSLTCTVSGGSIKSGSYYWGWIRQPPGKGLEWIG |
| | | | | | NIYYSGSTYYNPSLRSRVTISVDTSKNQFSLKLSSVTAADTAVYYCAREGS |
| | | | | | YPNQFDPWGQGTLVTVSSASTKGPSVFPLAPSSKSTSGGTAALGCLVKDYF |
| | | | | | PEPVTVSWNSGALTSGVHTFPAVLQSSGLYSLSSVVTVPSSSLGTQTYICN |
| | | | | | VNHKPSNTKVDKKVEPKSCDKTHTCPPCPAPELLGGPSVFLFPPKPKDTLM |
| | | | | | ISRTPEVTCVVVDVSHEDPEVKFNWYVDGVEVHNAKTKPREEQYNSTYRVV |
| | | | | | SVLTVLHQDWLNGKEYKCKVSNKALPAPIEKTISKAKGQPREPQVYTLPPC |
| | | | | | RDELTKNQVSLWCLVKGFYPSDIAVEWESNGQPENNYKTTPPVLDSDGSFF |
| | | | | | LYSKLTVDKSRWQQGNVFSCSVMHEALHNHYTQKSLSLSPGK |
| 39A11 HC1 | 39A11/57G8- | HC1 | 39A11 VH | 148 | EVQLLESGGGLVQPGGSLRLSCAASGFTFSSYGMHWVRQAPGKGLEWVASI |
| | IL15 | | | | SSSSGYIYYADSVKGRFTISRDNSKNTLYLQMNSLRAEDTAVYYCARASSG |
| | | | | | MDVWGQGTTVTVSSASTKGPSVFPLAPSSKSTSGGTAALGCLVKDYFPEPV |
| | | | | | TVSWNSGALTSGVHTFPAVLQSSGLYSLSSVVTVPSSSLGTQTYICNVNHK |
| | | | | | PSNTKVDKKVEPKSCDKTHTCPPCPAPELLGGPSVFLFPPKPKDTLMISRT |
| | | | | | PEVTCVVVDVSHEDPEVKFNWYVDGVEVHNAKTKPREEQYNSTYRVVSVLT |
| | | | | | VLHQDWLNGKEYKCKVSNKALPAPIEKTISKAKGQPREPQVYTLPPCRDEL |
| | | | | | TKNQVSLWCLVKGFYPSDIAVEWESNGQPENNYKTTPPVLDSDGSFFLYSK |
| | | | | | LTVDKSRWQQGNVFSCSVMHEALHNHYTQKSLSLSPGK |
| 39A11 HC1- | 39A11/57G8- | HC1 | 39A11 VH | 149 | EVQLLESGGGLVQPGGSLRLSCAASGFTFSSYGMHWVRQAPGKGLEWVASI |
| LS | IL15-LS | | LS mutation | | SSSSGYIYYADSVKGRFTISRDNSKNTLYLQMNSLRAEDTAVYYCARASSG |
| | | | | | MDVWGQGTTVTVSSASTKGPSVFPLAPSSKSTSGGTAALGCLVKDYFPEPV |
| | | | | | TVSWNSGALTSGVHTFPAVLQSSGLYSLSSVVTVPSSSLGTQTYICNVNHK |
| | | | | | PSNTKVDKKVEPKSCDKTHTCPPCPAPELLGGPSVFLFPPKPKDTLMISRT |
| | | | | | PEVTCVVVDVSHEDPEVKFNWYVDGVEVHNAKTKPREEQYNSTYRVVSVLT |
| | | | | | VLHQDWLNGKEYKCKVSNKALPAPIEKTISKAKGQPREPQVYTLPPCRDEL |
| | | | | | TKNQVSLWCLVKGFYPSDIAVEWESNGQPENNYKTTPPVLDSDGSFFLYSK |
| | | | | | LTVDKSRWQQGNVFSCSVLHEALHSHYTQKSLSLSPGK |
| 3F2 HC1 | 3F2/50A10- | HC1 | 3F2 VH | 150 | QVQLVQSGAEVKKPGASVKVSCKASGYTFTAQYMYWVRQAPGQGLEWMGRI |
| | IL15 | | | | NPTSGNTVYAQKLQGRVTMTRDTSTSTVYMELSSLRSEDTAVYYCARGINW |
| | | | | | FDPWGQGTLVTVSSASTKGPSVFPLAPSSKSTSGGTAALGCLVKDYFPEPV |
| | | | | | TVSWNSGALTSGVHTFPAVLQSSGLYSLSSVVTVPSSSLGTQTYICNVNHK |
| | | | | | PSNTKVDKKVEPKSCDKTHTCPPCPAPELLGGPSVFLFPPKPKDTLMISRT |
| | | | | | PEVTCVVVDVSHEDPEVKFNWYVDGVEVHNAKTKPREEQYNSTYRVVSVLT |
| | | | | | VLHQDWLNGKEYKCKVSNKALPAPIEKTISKAKGQPREPQVYTLPPCRDEL |
| | | | | | TKNQVSLWCLVKGFYPSDIAVEWESNGQPENNYKTTPPVLDSDGSFFLYSK |
| | | | | | LTVDKSRWQQGNVFSCSVMHEALHNHYTQKSLSLSPGK |
| 3F2 HC1-LS | 3F2/50A10- | HC1 | 3F2 VH | 151 | QVQLVQSGAEVKKPGASVKVSCKASGYTFTAQYMYWVRQAPGQGLEWMGRI |
| | IL15-LS | | LS mutation | | NPTSGNTVYAQKLQGRVTMTRDTSTSTVYMELSSLRSEDTAVYYCARGINW |
| | | | | | FDPWGQGTLVTVSSASTKGPSVFPLAPSSKSTSGGTAALGCLVKDYFPEPV |
| | | | | | TVSWNSGALTSGVHTFPAVLQSSGLYSLSSVVTVPSSSLGTQTYICNVNHK |
| | | | | | PSNTKVDKKVEPKSCDKTHTCPPCPAPELLGGPSVFLFPPKPKDTLMISRT |
| | | | | | PEVTCVVVDVSHEDPEVKFNWYVDGVEVHNAKTKPREEQYNSTYRVVSVLT |
| | | | | | VLHQDWLNGKEYKCKVSNKALPAPIEKTISKAKGQPREPQVYTLPPCRDEL |
| | | | | | TKNQVSLWCLVKGFYPSDIAVEWESNGQPENNYKTTPPVLDSDGSFFLYSK |
| | | | | | LTVDKSRWQQGNVFSCSVLHEALHSHYTQKSLSLSPGK |
| DP47 HC1 | DP47/IL15 | HC1 | | 152 | EVQLLESGGGLVQPGGSLRLSCAASGFTFSSYAMSWVRQAPGKGLEWVSAI |
| | | | | | SGSGGSTYYADSVKGRFTISRDNSKNTLYLQMNSLRAEDTAVYYCAKGSGF |
| | | | | | DYWGQGTLVTVSSASTKGPSVFPLAPSSKSTSGGTAALGCLVKDYFPEPVT |
| | | | | | VSWNSGALTSGVHTFPAVLQSSGLYSLSSVVTVPSSSLGTQTYICNVNHKP |
| | | | | | SNTKVDKKVEPKSCDKTHTCPPCPAPELLGGPSVFLFPPKPKDTLMISRTP |
| | | | | | EVTCVVVDVSHEDPEVKFNWYVDGVEVHNAKTKPREEQYNSTYRVVSVLTV |
| | | | | | LHQDWLNGKEYKCKVSNKALPAPIEKTISKAKGQPREPQVYTLPPCRDELT |
| | | | | | KNQVSLWCLVKGFYPSDIAVEWESNGQPENNYKTTPPVLDSDGSFFLYSKL |
| | | | | | TVDKSRWQQGNVFSCSVMHEALHNHYTQKSLSLSPGK |
| DP47 HC1- | DP47/IL15-LS | HC1 | LS mutation | 153 | EVQLLESGGGLVQPGGSLRLSCAASGFTFSSYAMSWVRQAPGKGLEWVSAI |
| LS | | | | | SGSGGSTYYADSVKGRFTISRDNSKNTLYLQMNSLRAEDTAVYYCAKGSGF |
| | | | | | DYWGQGTLVTVSSASTKGPSVFPLAPSSKSTSGGTAALGCLVKDYFPEPVT |
| | | | | | VSWNSGALTSGVHTFPAVLQSSGLYSLSSVVTVPSSSLGTQTYICNVNHKP |
| | | | | | SNTKVDKKVEPKSCDKTHTCPPCPAPELLGGPSVFLFPPKPKDTLMISRTP |
| | | | | | EVTCVVVDVSHEDPEVKFNWYVDGVEVHNAKTKPREEQYNSTYRVVSVLTV |
| | | | | | LHQDWLNGKEYKCKVSNKALPAPIEKTISKAKGQPREPQVYTLPPCRDELT |
| | | | | | KNQVSLWCLVKGFYPSDIAVEWESNGQPENNYKTTPPVLDSDGSFFLYSKL |
| | | | | | TVDKSRWQQGNVFSCSVLHEALHSHYTQKSLSLSPGK |
| 20502 HC-2 | (+) Ab1/IL15 | HC | 20502 VH | 154 | [symbol] [symbol] [symbol] [symbol] |
| | | | IL-15Rα | | [symbol] [symbol] [symbol] [symbol] |
| | | | sushi | | [symbol] [symbol] ASTKGPSVFPLAPSSKSTSGGTAALGCLVKDYF |
| | | | domain | | PEPVTVSWNSGALTSGVHTFPAVLQSSGLYSLSSVVTVPSSSLGTQTYICN |
| | | | Linker | | VNHKPSNTKVDKKVEPKSCDKTHTCPPCPAPELLGGPSVFLFPPKPKDTLM |
| | | | IL-15 | | ISRTPEVTCVVVDVSHEDPEVKFNWYVDGVEVHNAKTKPREEQYNSTYRVV |
| | | | | | SVLTVLHQDWLNGKEYKCKVSNKALPAPIEKTISKAKGQPREPQVCTLPPS |
| | | | | | RDELTKNQVSLSCAVKGFYPSDIAVEWESNGQPENNYKTTPPVLDSDGSFF |
| | | | | | LVSKLTVDKSRWQQGNVFSCSVMHEALHNHYTQKSLSLSPGSCPPPMSVEH |
| | | | | | ADIWVKSYSLYSRERYICNSGFKRKAGTSSLTECVLNKATNVAHWTTPSLK |
| | | | | | CIRDPALVHQRPAPPSGGSGGGGSGGGSGGGGSLQNWVNVISDLKKIEDLI |
| | | | | | QSMHIDATLYTESDVHPSCKVTAMKCFLLELQVISLESGDASIHDTVESLI |
| | | | | | ILANNSLSSNGNVTESGCKECEELEEKNIKEFLQSFVHIVQMFINTS |
| 39A11-HC2 | 39A11/57G8- | HC2 | 39A11 VH | 155 | [symbol] [symbol] [symbol] [symbol] |
| | IL15 fusion | | IL-15Rα | | [symbol] [symbol] [symbol] |
| | | | sushi | | [symbol] ASTKGPSVFPLAPSSKSTSGGTAALGCLVKDYFPEPV |
| | | | domain | | TVSWNSGALTSGVHTFPAVLQSSGLYSLSSVVTVPSSSLGTQTYICNVNHK |
| | | | Linker | | PSNTKVDKKVEPKSCDKTHTCPPCPAPELLGGPSVFLFPPKPKDTLMISRT |
| | | | IL-15 | | PEVTCVVVDVSHEDPEVKFNWYVDGVEVHNAKTKPREEQYNSTYRVVSVLT |
| | | | | | VLHQDWLNGKEYKCKVSNKALPAPIEKTISKAKGQPREPQVCTLPPSRDEL |
| | | | | | TKNQVSLSCAVKGFYPSDIAVEWESNGQPENNYKTTPPVLDSDGSFFLVSK |
| | | | | | LTVDKSRWQQGNVFSCSVMHEALHNHYTQKSLSLSPGSCPPPMSVEHADIW |
| | | | | | VKSYSLYSRERYICNSGFKRKAGTSSLTECVLNKATNVAHWTTPSLKCIRD |
| | | | | | PALVHQRPAPPSGGSGGGGSGGGSGGGGSLQNWVNVISDLKKIEDLIQSMH |
| | | | | | IDATLYTESDVHPSCKVTAMKCFLLELQVISLESGDASIHDTVESLIILAN |
| | | | | | NSLSSNGNVTESGCKECEELEEKNIKEFLQSFVHIVQMFINTS |
| 39A11-HC2- | 39A11/57G8- | HC2 | 39A11 VH | 156 | [symbol] [symbol] [symbol] |
| LS | IL15-LS | | LS | | [symbol] [symbol] [symbol] |
| | fusion | | mutation | | [symbol] ASTKGPSVFPLAPSSKSTSGGTAALGCLVKDYFPEPV |
| | | | IL-15Rα | | TVSWNSGALTSGVHTFPAVLQSSGLYSLSSVVTVPSSSLGTQTYICNVNHK |
| | | | sushi | | PSNTKVDKKVEPKSCDKTHTCPPCPAPELLGGPSVFLFPPKPKDTLMISRT |
| | | | domain | | PEVTCVVVDVSHEDPEVKFNWYVDGVEVHNAKTKPREEQYNSTYRVVSVLT |
| | | | Linker | | VLHQDWLNGKEYKCKVSNKALPAPIEKTISKAKGQPREPQVCTLPPSRDEL |
| | | | IL-15 | | TKNQVSLSCAVKGFYPSDIAVEWESNGQPENNYKTTPPVLDSDGSFFLVSK |
| | | | | | LTVDKSRWQQGNVFSCSVLHEALHSHYTQKSLSLSPGSCPPPMSVEHADIW |
| | | | | | VKSYSLYSRERYICNSGFKRKAGTSSLTECVLNKATNVAHWTTPSLKCIRD |
| | | | | | PALVHQRPAPPSGGSGGGGSGGGSGGGGSLQNWVNVISDLKKIEDLIQSMH |
| | | | | | IDATLYTESDVHPSCKVTAMKCFLLELQVISLESGDASIHDTVESLIILAN |
| | | | | | NSLSSNGNVTESGCKECEELEEKNIKEFLQSFVHIVQMFINTS |
| 3F2 HC1 | 3F2/50A10- | HC2 | 3F2 VH | 157 | [symbol] [symbol] [symbol] [symbol] |
| | IL15 fusion | | IL-15Rα | | [symbol] [symbol] [symbol] [symbol] |
| | | | sushi | | [symbol] ASTKGPSVFPLAPSSKSTSGGTAALGCLVKDYFPEPV |
| | | | domain | | TVSWNSGALTSGVHTFPAVLQSSGLYSLSSVVTVPSSSLGTQTYICNVNHK |
| | | | Linker | | PSNTKVDKKVEPKSCDKTHTCPPCPAPELLGGPSVFLFPPKPKDTLMISRT |
| | | | IL-15 | | PEVTCVVVDVSHEDPEVKFNWYVDGVEVHNAKTKPREEQYNSTYRVVSVLT |
| | | | | | VLHQDWLNGKEYKCKVSNKALPAPIEKTISKAKGQPREPQVCTLPPSRDEL |
| | | | | | TKNQVSLSCAVKGFYPSDIAVEWESNGQPENNYKTTPPVLDSDGSFFLVSK |
| | | | | | LTVDKSRWQQGNVFSCSVMHEALHNHYTQKSLSLSPGSCPPPMSVEHADIW |
| | | | | | VKSYSLYSRERYICNSGFKRKAGTSSLTECVLNKATNVAHWTTPSLKCIRD |
| | | | | | PALVHQRPAPPSGGSGGGGSGGGSGGGGSLQNWVNVISDLKKIEDLIQSMH |
| | | | | | IDATLYTESDVHPSCKVTAMKCFLLELQVISLESGDASIHDTVESLIILAN |
| | | | | | NSLSSNGNVTESGCKECEELEEKNIKEFLQSFVHIVQMFINTS |
| 3F2 HC2-LS | 39A11/57G8- | HC2 | 3F2 VH | 158 | [symbol] [symbol] [symbol] [symbol] |
| | IL15-LS | | LS | | [symbol] [symbol] [symbol] [symbol] |
| | | | mutation | | [symbol] ASTKGPSVFPLAPSSKSTSGGTAALGCLVKDYFPEPV |
| | | | IL-15Rα | | TVSWNSGALTSGVHTFPAVLQSSGLYSLSSVVTVPSSSLGTQTYICNVNHK |
| | | | sushi | | PSNTKVDKKVEPKSCDKTHTCPPCPAPELLGGPSVFLFPPKPKDTLMISRT |
| | | | domain | | PEVTCVVVDVSHEDPEVKFNWYVDGVEVHNAKTKPREEQYNSTYRVVSVLT |
| | | | Linker | | VLHQDWLNGKEYKCKVSNKALPAPIEKTISKAKGQPREPQVCTLPPSRDEL |
| | | | IL-15 | | TKNQVSLSCAVKGFYPSDIAVEWESNGQPENNYKTTPPVLDSDGSFFLVSK |
| | | | | | LTVDKSRWQQGNVFSCSVLHEALHSHYTQKSLSLSPGSCPPPMSVEHADIW |
| | | | | | VKSYSLYSRERYICNSGFKRKAGTSSLTECVLNKATNVAHWTTPSLKCIRD |
| | | | | | PALVHQRPAPPSGGSGGGGSGGGSGGGGSLQNWVNVISDLKKIEDLIQSMH |
| | | | | | IDATLYTESDVHPSCKVTAMKCFLLELQVISLESGDASIHDTVESLIILAN |
| | | | | | NSLSSNGNVTESGCKECEELEEKNIKEFLQSFVHIVQMFINTS |
| DP47 HC2 | DP47/IL15 | HC2 | DP47 VH | 159 | [symbol] [symbol] [symbol] [symbol] |
| | | | IL-15Rα | | [symbol] [symbol] [symbol] [symbol] |
| | | | sushi | | [symbol] ASTKGPSVFPLAPSSKSTSGGTAALGCLVKDYFPEPVT |
| | | | domain | | VSWNSGALTSGVHTFPAVLQSSGLYSLSSVVTVPSSSLGTQTYICNVNHKP |
| | | | Linker | | SNTKVDKKVEPKSCDKTHTCPPCPAPELLGGPSVFLFPPKPKDTLMISRTP |
| | | | IL-15 | | EVTCVVVDVSHEDPEVKFNWYVDGVEVHNAKTKPREEQYNSTYRVVSVLTV |
| | | | | | LHQDWLNGKEYKCKVSNKALPAPIEKTISKAKGQPREPQVCTLPPSRDELT |
| | | | | | KNQVSLSCAVKGFYPSDIAVEWESNGQPENNYKTTPPVLDSDGSFFLVSKL |
| | | | | | TVDKSRWQQGNVFSCSVMHEALHNHYTQKSLSLSPGSCPPPMSVEHADIWV |
| | | | | | KSYSLYSRERYICNSGFKRKAGTSSLTECVLNKATNVAHWTTPSLKCIRDP |
| | | | | | ALVHQRPAPPSGGSGGGGSGGGSGGGGSLQNWVNVISDLKKIEDLIQSMHI |
| | | | | | DATLYTESDVHPSCKVTAMKCFLLELQVISLESGDASIHDTVESLIILANN |
| | | | | | SLSSNGNVTESGCKECEELEEKNIKEFLQSFVHIVQMFINTS |
| DP47 HC2- | DP47/IL15-LS | HC2 | LS | 160 | EVQLLESGGGLVQPGGSLRLSCAASGFTFSSYAMSWVRQAPGKGLEWVSAI |
| LS | | | mutation | | SGSGGSTYYADSVKGRFTISRDNSKNTLYLQMNSLRAEDTAVYYCAKGSGF |
| | | | IL-15Rα | | DYWGQGTLVTVSSASTKGPSVFPLAPSSKSTSGGTAALGCLVKDYFPEPVT |
| | | | sushi | | VSWNSGALTSGVHTFPAVLQSSGLYSLSSVVTVPSSSLGTQTYICNVNHKP |
| | | | domain | | SNTKVDKKVEPKSCDKTHTCPPCPAPELLGGPSVFLFPPKPKDTLMISRTP |
| | | | Linker | | EVTCVVVDVSHEDPEVKFNWYVDGVEVHNAKTKPREEQYNSTYRVVSVLTV |
| | | | IL-15 | | LHQDWLNGKEYKCKVSNKALPAPIEKTISKAKGQPREPQVCTLPPSRDELT |
| | | | | | KNQVSLSCAVKGFYPSDIAVEWESNGQPENNYKTTPPVLDSDGSFFLVSKL |
| | | | | | TVDKSRWQQGNVFSCSVLHEALHSHYTQKSLSLSPGSCPPPMSVEHADIWV |
| | | | | | KSYSLYSRERYICNSGFKRKAGTSSLTECVLNKATNVAHWTTPSLKCIRDP |
| | | | | | ALVHQRPAPPSGGSGGGGSGGGSGGGGSLQNWVNVISDLKKIEDLIQSMHI |
| | | | | | DATLYTESDVHPSCKVTAMKCFLLELQVISLESGDASIHDTVESLIILANN |
| | | | | | SLSSNGNVTESGCKECEELEEKNIKEFLQSFVHIVQMFINTS |

TABLE 23
Other protein sequences.

| | SEQ ID | |
|---|---|---|
| Protein | NO: | Sequence |
| human B7-H4 | 161 | MASLGQILFWSIISIIIILAGAIALIIGFGISGRHSITVTTVASAGNIGEDGILSCTFEPDIKLSDI |
| (NCBI accession | | VIQWLKEGVLGLVHEFKEGKDELSEQDEMFRGRTAVFADQVIVGNASLRLKNVQLTDAGTYKCYIIT |
| #: NP_078902) | | SKGKGNANLEYKTG [symbol] [symbol] [symbol] [symbol] |
| signal peptide | | [symbol] [symbol] [symbol] [symbol] [symbol] SLCVSSFFAI |
| IgV domain | | SWALLPLSPYLMLK |
| IgC domain | | |
| transmembrane | | |
| region | | |
| Murine B7-H4 | 162 | MASLGQIIFWSIIFWSIINIIIILAGAIALIIGFKGISTVTTFTSAGNIGEDGTLSCTFEPDIKLNGI |
| (NCBI accession#: | | VIQWLKEGIKGLVHEFKEGKDDLSQQHEMFRGRTAVFADQVVVGNASLRLKNVQLTDAGTYTCYIRT |
| Q7TSP5, Phe29- | | SKGKGNANLEYKTG [symbol] [symbol] [symbol] [symbol] |
| Pro258) | | [symbol] [symbol] [symbol] [symbol] |
| signal peptide | | AGWALLSLSCCLMLR |
| IgV domain | | |
| IgC domain | | |
| transmembrane region | | |
| IgG1 Fc | 163 | ASTKGPSVFPLAPSSKSTSGGTAALGCLVKDYFPEPVTVSWNSGALTSGVHTFPAVLQSSGLYSLSS |
| (NCBI accession | | VVTVPSSSLGTQTYICNVNHKPSNTKVDKKVEPKSCDKTHTCPPCPAPELLGGPSVFLFPPKPKDTL |
| #: P01857) | | MISRTPEVTCVVVDVSHEDPEVKFNWYVDGVEVHNAKTKPREEQYNSTYRVVSVLTVLHQDWLNGKE |
| Pro100-Lys330 | | YKCKVSNKALPAPIEKTISKAKGQPREPQVYTLPPSRDELTKNQVSLTCLVKGFYPSDIAVEWESNG |
| underlined | | QPENNYKTTPPVLDSDGSFFLYSKLTVDKSRWQQGNVFSCSVMHEALHNHYTQKSLSLSPGK |
| Full-length | 164 | MRISKPHLRSISIQCYLCLLLNSHFLTEAGIHVFILGCFSAGLPKTEANWVNVISDLKKIEDLIQSM |
| human Il-15 (NCBI | | HIDATLYTESDVHPSCKVTAMKCFLLELQVISLESGDASIHDTVENLIILANNSLSSNGNVTESGCK |
| accession #: | | ECEELEEKNIKEFLQSFVHIVQMFINTS |
| P40933) | | |
| mature protein | | |
| Human IL-15 | 165 | NWVNVISDLKKIEDLIQSMHIDATLYTESDVHPSCKVTAMKCFLLELQVISLESGDASIHDTVENLI |
| (mature protein, | | ILANNSLSSNGNVTESGCKECEELEEKNIKEFLQSFVHIVQMFINTS |
| residue N65 is | | |
| in bold) | | |
| Human IL-15 N65S | 166 | NWVNVISDLKKIEDLIQSMHIDATLYTESDVHPSCKVTAMKCFLLELQVISLESGDASIHDTVESLI |
| (mature protein, | | ILANNSLSSNGNVTESGCKECEELEEKNIKEFLQSFVHIVQMFINTS |
| residue N65S | | |
| is in bold) | | |
| IL-15Rα sushi domain | 167 | CPPPMSVEHADIWVKSYSLYSRERYICNSGFKRKAGTSSLTECVLNKATNVAHWTTPSLKC |
| (residues 33-93 of | | |
| NCBI accession #: | | |
| EAW86418.1) | | |
| Artificial sequence | 168 | SGGSGGGGSGGGSGGGGSLQ |
| IL-15Rα sushi | 169 | CPPPMSVEHADIWVKSYSLYSRERYICNSGFKRKAGTSSLTECVLNKATNVAHWTTPSLKCIRDPAL |
| domain + linker + | | VHQRPAPPSGGSGGGGSGGGSGGGGSLQNWVNVISDLKKIEDLIQSMHIDATLYTESDVHPSCKVTA |
| Il-15 N65S IL-15Rα | | MKCFLLELQVISLESGDASIHDTVESLIILANNSLSSNGNVTESGCKECEELEEKNIKEFLQSFVHI |
| sushi domain Linker | | VQMFINTS |
| IL-15 | | |
| Artificial sequence | 170 | IRDPALVHQRPAPPSGGSGGGGSGGGSGGGGSLQ |
| Artificial sequence | 171 | IRDPALVHQRPAPP |

TABLE 24
Examples of antibodies CDRs binding to IgV domain of human B7H4 (3F2 and 50A10)

| | | | Kabat EU index numbering | | |
|---|---|---|---|---|---|
| | | EU index numbering | alternative | IMGT | Chothia |
| 3F2 | CDR1H | GYTFTAQYMY | AQYMY | GYTFTAQY | GYTFTAQ |
| | | (SEQ ID NO: 16) | (SEQ ID NO: 172) | (SEQ ID NO: 178) | (SEQ ID NO: 184) |
| 3F2 | CDR2H | RINPTSGNTVYAQKLQG | RINPTSGNTVYAQKLQG | INPTSGNT | PTSG |
| | | (SEQ ID NO: 17) | (SEQ ID NO: 173) | (SEQ ID NO: 179) | (SEQ ID NO: 185) |
| 3F2 | CDR3H | GINWFDP | GINWFDP | ARGINWFDP | INWFD |
| | | (SEQ ID NO: 18) | (SEQ ID NO: 174) | (SEQ ID NO: 180) | (SEQ ID NO: 186) |
| 50A10 | CDR1L | QASQDISNYIN | SQDISNY | SQDISNY | QASQDISNYIN |
| | | (SEQ ID NO: 70) | (SEQ ID NO: 175) | (SEQ ID NO: 181) | (SEQ ID NO: 187) |
| 50A10 | CDR2L | SRLQS | AAS | AAS | AASRLQS |
| | | (SEQ ID NO: 71) | (SEQ ID NO: 176) | (SEQ ID NO: 182) | (SEQ ID NO: 188) |
| 50A10 | CDR3L | QQSYRSPFT | SYRSPF | SYRSPF | QQSYRSPFT |
| | | (SEQ ID NO: 72) | (SEQ ID NO: 177) | (SEQ ID NO: 183) | (SEQ ID NO: 189) |

1. An anti-B7-H4 antibody, or antigen-binding fragment thereof, wherein the antibody, or antigen-binding fragment thereof, comprises a heavy chain variable region and a light chain variable region, wherein each of the heavy chain and the light chain variable regions comprise a CDR1, CDR2, and CDR3, and wherein:
a. CDR1H comprises SEQ ID NO:16, CDR2H comprises SEQ ID NO:17, CDR3H comprises SEQ ID NO:18, and wherein:
i. CDR1L comprises SEQ ID NO:20, CDR2L comprises SEQ ID NO:21, and CDR3L comprises SEQ ID NO:22; or
ii. CDR1L comprises SEQ ID NO:66, CDR2L comprises SEQ ID NO:67, and CDR3L comprises SEQ ID NO:68; or
iii. CDR1L comprises SEQ ID NO:70, CDR2L comprises SEQ ID NO:71, and CDR3L comprises SEQ ID NO:72; or
b. CDR1H comprises SEQ ID NO:4, CDR2H comprises SEQ ID NO:5, CDR3H comprises SEQ ID NO:6, and wherein:
i. CDR1L comprises SEQ ID NO: 8, CDR2L comprises SEQ ID NO: 9, and CDR3L comprises SEQ ID NO: 10;
ii. CDR1L comprises SEQ ID NO: 77, CDR2L comprises SEQ ID NO: 78, and CDR3L comprises SEQ ID NO: 79; or
iii. CDR1L comprises SEQ ID NO: 81, CDR2L comprises SEQ ID NO:82, and CDR3L comprises SEQ ID NO:83
c. CDR1H comprises SEQ ID NO:46, CDR2H comprises SEQ ID NO:47, CDR3H comprises SEQ ID NO:48 and wherein:
i. CDR1L comprises SEQ ID NO:50, CDR2L comprises SEQ ID NO:51, and CDR3L comprises SEQ ID NO:52;
ii. CDR1L comprises SEQ ID NO:88, CDR2L comprises SEQ ID NO:89, and CDR3L comprises SEQ ID NO:90;
iii. CDR1L comprises SEQ ID NO:92, CDR2L comprises SEQ ID NO:93, and CDR3L comprises SEQ ID NO:94;
iv. CDR1L comprises SEQ ID NO:96, CDR2L comprises SEQ ID NO:97, and CDR3L comprises SEQ ID NO:98;
v. CDR1L comprises SEQ ID NO: 100, CDR2L comprises SEQ ID NO:101, and CDR3L comprises SEQ ID NO:102;
vi. CDR1L comprises SEQ ID NO:104, CDR2L comprises SEQ ID NO:105, and CDR3L comprises SEQ ID NO:106: or
vii. CDR1L comprises SEQ ID NO:10⁸, CDR2L comprises SEQ ID NO:109, and CDR3L comprises SEQ ID NO:110
d. CDR1H comprises SEQ ID NO:58, CDR2H comprises SEQ ID NO:59, CDR3H comprises SEQ ID NO:60, and wherein:
i. CDR1L comprises SEQ ID NO:62, CDR2L comprises SEQ ID NO:63, and CDR3L comprises SEQ ID NO:64;
ii. CDR1L comprises SEQ ID NO:115, CDR2L comprises SEQ ID NO:116, and CDR3L comprises SEQ ID NO:117;
iii. CDR1L comprises SEQ ID NO:119, CDR2L comprises SEQ ID NO:120, and CDR3L comprises SEQ ID NO:121:
iv. CDR1L comprises SEQ ID NO:123, CDR2L comprises SEQ ID NO:124, and CDR3L comprises SEQ ID NO:125;
v. CDR1L comprises SEQ ID NO:127, CDR2L comprises SEQ ID NO:128, and CDR3L comprises SEQ ID NO:129: or
vi. CDR1L comprises SEQ ID NO:131, CDR2L comprises SEQ ID NO:132, and CDR3L comprises SEQ ID NO:133.

2. The anti-B7-H4 antibody, or antigen-binding fragment thereof, of claim 1, wherein the CDR1H comprises SEQ ID NO:16, CDR2H comprises SEQ ID NO:17, CDR3H comprises SEQ ID NO:18, CDR1L comprises SEQ ID NO:70, CDR2L comprises SEQ ID NO:71, and CDR3L comprises SEQ ID NO:72.

3. The anti-B7-H4 antibody, or antigen-binding fragment thereof, of claim 1, wherein:
a. the heavy chain variable region comprises SEQ ID NO: 15, or a sequence that is at least 90% identical to SEQ ID NO: 15; and wherein
i. the light chain variable region comprises SEQ ID NO: 19, or a sequence that is at least 90% identical to SEQ ID NO: 19;
ii. the light chain variable region comprises SEQ ID NO: 65, or a sequence that is at least 90% identical to SEQ ID NO: 65; or
iii. the light chain variable region comprises SEQ ID NO: 69, or a sequence that is at least 90% identical to SEQ ID NO: 69; or
b. the heavy chain variable region comprises SEQ ID NO: 3, or a sequence that is at least 90% identical to SEQ ID NO: 3; and wherein:
i. the light chain variable region comprises SEQ ID NO: 7, or a sequence that is at least 90% identical to SEQ ID NO: 7;
ii. the light chain variable region comprises SEQ ID NO: 76, or a sequence that is at least 90% identical to SEQ ID NO: 76; or
iii. the light chain variable region comprises SEQ ID NO: 80, or a sequence that is at least 90% identical to SEQ ID NO: 80
c. the heavy chain variable region comprises SEQ ID NO:45, or a sequence that is at least 90% identical to SEQ ID NO:45 and wherein:
i. the light chain variable region comprises SEQ ID NO:49, or a sequence that is at least 90% identical to SEQ ID NO:49;
ii. the light chain variable region comprises SEQ ID NO:87, or a sequence that is at least 90% identical to SEQ ID NO:87;
iii. the light chain variable region comprises SEQ ID NO:91, or a sequence that is at least 90% identical to SEQ ID NO:91;
iv. the light chain variable region comprises SEQ ID NO:95, or a sequence that is at least 90% identical to SEQ ID NO:95;
v. the light chain variable region comprises SEQ ID NO:99, or a sequence that is at least 90% identical to SEQ ID NO:99;
vi. the light chain variable region comprises SEQ ID NO:103, or a sequence that is at least 90% identical to SEQ ID NO:103; or
vii. the light chain variable region comprises SEQ ID NO:107, or a sequence that is at least 90% identical to SEQ ID NO:107
d. the heavy chain variable region comprises SEQ ID NO:57, or a sequence that is at least 90% identical to SEQ ID NO:57 and wherein:
e. the light chain variable region comprises SEQ ID NO:61, or a sequence that is at least 90% identical to SEQ ID NO:61;
f. the light chain variable region comprises SEQ ID NO:114, or a sequence that is at least 90% identical to SEQ ID NO:114;
g. the light chain variable region comprises SEQ ID NO:118, or a sequence that is at least 90% identical to SEQ ID NO:118;
h. the light chain variable region comprises SEQ ID NO:122, or a sequence that is at least 90% identical to SEQ ID NO:122;
i. the light chain variable region comprises SEQ ID NO:126, or a sequence that is at least 90% identical to SEQ ID NO:126; or
j. the light chain variable region comprises SEQ ID NO:130, or a sequence that is at least 90% identical to SEQ ID NO:130.

4. (canceled)

5. (canceled)

6. The anti-B7-H4 antibody, or antigen-binding fragment thereof, of claim 1, wherein the heavy chain variable region comprises SEQ ID NO: 15 and the light chain variable region comprises SEQ ID NO: 69.

7. The anti-B7-H4 antibody, or antigen-binding fragment thereof, of claim 1, wherein the antibody, or antigen-binding fragment thereof, binds to (i) the IgV domain of human hB7-H4 or (ii) the IgV domain of human and murine hB7-H4.

8.-19. (canceled)

20. The anti-B7-H4 antibody, or antigen-binding fragment thereof, of claim 1, wherein the heavy chain variable region comprises SEQ ID NO:57 and the light chain variable region comprises SEQ ID NO:118.

21. (canceled)

22. (canceled)

23. The anti-B7-H4 antibody, or antigen-binding fragment thereof, of claim 1, wherein the antibody or antigen-binding fragment is:
a. a chimeric antibody, a CDR-grafted antibody, or a humanized antibody or antigen-binding fragment thereof;
b. a multispecific or a bispecific antibody or antigen-binding fragment thereof;
c. an scFv, Fv, Fab′, Fab, F(ab′)₂, or diabody;
d. an IgG class immunoglobulin.

24.-28. (canceled)

29. The anti-B7-H4 antibody, or antigen-binding fragment thereof, of claim 1, wherein the antibody or antigen-binding fragment comprises a first and a second heavy chain constant region, and wherein the antibody, or antigen-binding fragment thereof, comprises at least one modification in the CH3 domains of the first and the second heavy chain constant region causing heterodimerization.

30. (canceled)

31. (canceled)

32. The anti-B7-H4 antibody, or antigen-binding fragment thereof, of claim 29, wherein:
a. the first heavy chain constant region comprises one or more amino acid substitutions selected from the group consisting of S354C and T366W (Kabat EU index numbering) and the second heavy chain constant region comprises one or more amino acid substitutions selected from the group consisting of Y349C, T366S, L368A, and Y407V (Kabat EU index numbering); or
b. the second heavy chain constant region comprises one or more amino acid substitutions selected from the group consisting of S354C and T366W (Kabat EU index numbering) and the first heavy chain constant region comprises one or more amino acid substitutions selected from the group consisting of Y349C, T366S, L368A, and Y407V (Kabat EU index numbering).

33. (canceled)

34. (canceled)

35. A fusion protein comprising:
a. an anti-B7-H4 antibody, or antigen-binding fragment thereof,
b. an IL-15Rα sushi domain polypeptide comprising SEQ ID NO:167, or an amino acid sequence that at least 90% identical to SEQ ID NO:167; and
c. an IL-15 polypeptide comprising SEQ ID NO:166, or an amino acid sequence that is at least 90% identical to SEQ ID NO:166.

36. The fusion protein of claim 35 wherein the B7H4 antibody comprises a heavy chain CDR1 (CDR1H), CDR2 (CDR2H) and CDR3 (CDR3H), and a light chain CDR1 (CDR1L), CDR2 (CDR2L) and CDR3 (CDR3L), wherein:
a. CDR1H comprises SEQ ID NO:16, CDR2H comprises SEQ ID NO:17, CDR3H comprises SEQ ID NO:18, CDR1L comprises SEQ ID NO:70, CDR2L comprises SEQ ID NO:71, and CDR3L comprises SEQ ID NO:72,
b. CDR1H comprises SEQ ID NO:172, CDR2H comprises SEQ ID NO:173, CDR3H comprises SEQ ID NO:174, CDR1L comprises SEQ ID NO:175, CDR2L comprises SEQ ID NO:176, and CDR3L comprises SEQ ID NO:177;
c. CDR1H comprises SEQ ID NO:178, CDR2H comprises SEQ ID NO:179, CDR3H comprises SEQ ID NO:180, CDR1L comprises SEQ ID NO:181, CDR2L comprises SEQ ID NO:182, and CDR3L comprises SEQ ID NO:183;
d. CDR1H comprises SEQ ID NO:184, CDR2H comprises SEQ ID NO:185, CDR3H comprises SEQ ID NO:186, CDR1L comprises SEQ ID NO:187, CDR2L comprises SEQ ID NO:188, and CDR3L comprises SEQ ID NO:189.

37.-65. (canceled)

65. A nucleic acid sequence encoding the anti-B7-H4 antibody, or antigen-binding fragment thereof, of claim 1.

67. A cell comprising the nucleic acid of claim 66.

68.-72. (canceled)

73. A pharmaceutical composition comprising (i) anti-B7-H4 antibody, or antigen-binding fragment thereof, of claim 1 and (ii) a pharmaceutically acceptable carrier.

74. A method of producing an anti-B7-H4 antibody, or antigen-binding fragment thereof, the method comprising culturing the cell of claim 68 under conditions so that the anti-B7-H4 antibody, or antigen-binding fragment thereof is produced.

75.-78. (canceled)

79. A method of stimulating the immune system in a subject in need thereof, the method comprising administering to the subject an effective amount of the anti-B7-H4 antibody, or antigen-binding fragment thereof, of claim 1.

80. A method of treating cancer in a subject in need thereof, the method comprising administering to the subject an effective amount of the anti-B7-H4 antibody, or antigen-binding fragment thereof, of claim 1.

81.-94. (canceled)

95. A method of detecting B7-H4 in a sample, the method comprising contacting the sample with the anti-B7-H4 antibody, or antigen-binding fragment thereof, of any one of claim 1.

96. A method of treating cancer in a subject in need thereof, the method comprising administering to the subject an effective amount of the fusion protein of claim 35.
